(19) United States
(12) Patent Application Publication
(10) Pub. No.: US 2023/0279076 A1
(43) Pub. Date: Sep. 7, 2023

(54) T-Cell Modulatory Polypeptides with Conjugation Sites and Methods of Use Thereof

(71) Applicant: Cue Biopharma, Inc., Boston, MA (US)

(72) Inventors: Ronald D. SEIDEL, III, Natick, MA (US); Rodolfo J. CHAPARRO, Cambridge, MA (US); John F. ROSS, Arlington, MA (US); Chee Meng LOW, Boston, MA (US); Anish SURI, Boston, MA (US); Raymond J. MONIZ, Boston, MA (US); Gurpanna SAGGU, Westwood, MA (US)

(21) Appl. No.: 18/005,813

(22) PCT Filed: Jul. 14, 2021
(86) PCT No.: PCT/US21/41675

§ 371 Date: Jan. 17, 2023

Related U.S. Application Data
(60) Provisional application No. 63/051,472, filed on Jul. 14, 2020.
(60) Provisional application No. 63/110,929, filed on Nov. 6, 2020.
(60) Provisional application No. 63/120,677, filed on Dec. 2, 2020.

(51) Int. Cl. C07K 14/74; C07K 14/55

(52) U.S. Cl. CPC C07K 14/70539; C07K 14/55

(57) ABSTRACT

The present disclosure provides T cell modulatory polypeptides (T-Cell-MPs) comprising a chemical conjugation site and at least one immunomodulatory polypeptide sequence that may be selected to exhibit reduced binding affinity to its cognate co-immunomodulatory polypeptide. The unconjugated T-Cell-MPs may be conjugated to a molecule displaying an epitope to form a T-Cell-MP-epitope conjugate. The T-Cell-epitope conjugates are useful for modulating the activity (e.g., increasing proliferation or cytotoxic activity) of T cells specific to the conjugate epitope, and accordingly for use as therapeutics.

### INCORPORATION OF SEQUENCE LISTING

This application contains a sequence listing submitted electronically via EFS-web, which serves as both the paper copy and the computer readable form (CRF) and consists of a file entitled “123640-8021US02_seqlist.txt”, which was created on Jul. 13, 2021, which is 641,516 bytes in size, and which is herein incorporated by reference in its entirety.

### INTRODUCTION

The ability to induce an adaptive immune response involves the engagement of the T cell receptor (TCR) present on the surface of a T cell with a small peptide or non-peptide molecule (e.g., an epitope of a molecule such as a polypeptide) presented by a major histocompatibility complex (MHC; also referred to in humans as a human leukocyte antigen (HLA) complex) that is located on the surface of an antigen presenting cell (APC). This engagement represents the immune system's targeting mechanism and is a requisite molecular interaction for T cell modulation (activation or inhibition) and effector function. Following epitope-specific cell targeting, the response of targeted T cells is dictated by the presence of immunomodulatory molecules (some of which are found on the surface of the APC) that act through engagement of counterpart receptors on the T cells. Both signals—epitope/TCR binding and engagement of immunomodulatory molecules with their counterpart receptors on T cells—are required to drive activation or inhibition of target T cell functions. The TCR is specific for a given epitope; however, the counterpart receptors for immunomodulatory molecules are not epitope-specific, and instead, are generally expressed on all T cells or on large T cell subsets.

### SUMMARY

The present disclosure provides T cell modulatory polypeptides (a “T-Cell-MP” or multiple “T-Cell-MPs”) that find use in, among other things, methods of in vivo, ex vivo, and in vitro treatment of various diseases (e.g., cancers, viral infections and autoimmune disorders) and other disorders of mammals (e.g., humans) and the preparation of medicaments for such treatments. In one aspect, the T-Cell-MPs described herein comprise a portion of a class I MHC-H polypeptide, a β2M polypeptide, a chemical conjugation site for covalently attaching an epitope presenting molecule, and at least one immunomodulatory polypeptide (also referred to herein as a “MOD polypeptide” or, simply, a “MOD”). Any one or more of the MODs present in the T-Cell-MP may be wild-type (“wt.”) or a variant that exhibits an altered binding affinity to its cellular binding partner/receptor (e.g., T cell surface), referred to as a Co-MOD.

T-Cell-MPs may be unconjugated, in which case they comprise at least one chemical conjugation site at which a molecule comprising a target antigenic determinate (e.g., a peptide, glycopeptide, or non-peptide such as a carbohydrate presenting an epitope) may be covalently bound to form a T-Cell-MP-epitope conjugate for presentation to a cell bearing a T cell receptor. Unconjugated T-Cell-MPs comprising a chemical conjugation site for linking an epitope are useful for rapidly preparing T-Cell-MP-epitope conjugates that can modulate the activity of T cells specific to the epitope presented and, accordingly, for modulating an immune response involving those T cells in an individual.

The T-Cell-MPs described herein are suitable for production in cell-based expression systems where most, or substantially all (e.g., greater than 75%, 85% or 90%) or all, of the expressed unconjugated T-Cell-MP polypeptide/protein is in a soluble non-aggregated state that is suitably stable at 37° C. for production in tissue culture and use at least up to that temperature. The T-Cell-MPs can advantageously be produced as a single polypeptide encoded by a nucleic acid sequence contained in a single vector. The T-Cell-MPs may form higher order structures, such as duplexes (see, e.g., FIG. 1), which may be homodimeric as in FIG. 9, or heterodimeric when formed from two T-Cell-MPs, e.g., as illustrated in FIGS. 10 and 11. Unconjugated T-Cell-MPs can be expressed in high yield, e.g., greater than 25, 40, 60, or 80 mg/liter (e.g. about 25 to about 40, about 40 to about 60, or about 60 to about 80 mg/l in CHO cells). Yields can be high especially when a disulfide bond is present between the carboxyl end of the MHC-H chain α₁ helix and the MHC-H chain α₂₋₁ helix (e.g. a Y84C to A139C disulfide bond), and the linker between the MHC-H polypeptide sequence and the β2M polypeptide is of sufficient length (e.g., from about 10 to about 50 aa long). With the disulfide bond present between the α₁ and α₂ helices, unconjugated T-Cell-MP expression levels may exceed 80 mg/l (e.g., from about 80 to about 100, about 100 to about 120, about 120 to about 140, about 140 to about 160, about 160 to about 180, or about 180 to about 200 mg/1).

Once purified, most, substantially all (e.g., greater than 85% or 90% of the T-Cell-MP), or all of the expressed unconjugated T-Cell-MP protein remains in a soluble non-aggregated state even after conjugation to an epitope (e.g., peptide epitopes) and is similarly stable compared to the unconjugated T-Cell-MP. The unconjugated T-Cell-MPs and their epitope conjugates may additionally comprise a targeting sequence that can direct a T-Cell-MP epitope conjugate to a particular cell or tissue (e.g., a tumor). Payloads (e.g., bioactive substances or labels), such as a therapeutic (e.g., chemotherapeutic agents) for co-delivery with a specific target epitope, may also be covalently attached to a T-Cell-MP, such as by a crosslinking agent. Accordingly, T-Cell-MP-epitope conjugates may be considered a means by which to deliver MODs (e.g., IL-2, 4-1BBL, FasL, TGF-β, CD70, CD80, CD86, or variants thereof) and/or payloads (e.g., chemotherapeutics) to T cells in an epitope-specific manner optionally with the assistance of a targeting sequence.

The T-Cell-MPs may comprise modifications that assist in the stabilization of the unconjugated T-Cell-MP during intracellular trafficking and/or following secretion by cells expressing the multimeric polypeptide even in the absence of an associated epitope (e.g., a peptide epitope). One such modification is a bond (e.g., disulfide bond) formed between amino acid position 84 at the carboxyl end of the MHC class I α₁ helix (or its flanking amino acid sequences aac1 and aac2) and amino acid position 139 at the amino end of the MHC-class I α₂₋₁ helix (or its flanking amino acid sequences aac3 and aac4). For example, the insertion of cysteine residues at amino acids 84 (Y84C substitution) and 139 (A139C substitution) of MHC-H, or the equivalent positions (see, e.g., FIG. 3I), may form a disulfide linkage that helps stabilize the T-Cell-MP. See, e.g., Z. Hein et al. (2014), Journal of Cell Science 127:2885-2897.

One aspect of the T-Cell-MP molecules described herein is broadly directed to an unconjugated T-Cell-MP, the polypeptide comprising (e.g., from N-terminus to C-terminus):
(i) optionally one or more MOD polypeptide sequences (e.g., two or more MOD polypeptide sequences, such as in tandem, wherein when there are two or more MOD polypeptide sequences they are optionally joined to each other by independently selected L1 linkers);
(ii) an optional L2 linker polypeptide sequence joining the one or more optional MOD polypeptide sequences to a β2M polypeptide sequence;
(iii) the β2M polypeptide sequence;
(iv) an optional L3 linker polypeptide sequence (e.g., from 10-50 aa in length);
(v) a class I MHC-H polypeptide sequence;
(vi) an optional L4 linker polypeptide sequence;
(vii) a scaffold polypeptide sequence (e.g., an immunoglobulin Fc sequence);
(viii) an optional L5 linker polypeptide sequence; and
(ix) optionally one or more MOD polypeptide sequences (e.g., two or more MOD polypeptide sequences, such as in tandem, wherein when there are two or more MOD polypeptide sequences they are optionally joined to each other by independently selected L6 linkers);
wherein the unconjugated T cell modulatory polypeptide comprises at least one MOD polypeptide sequence (e.g., the MOD(s) of element (i) and/or (ix)); and
wherein at least one of the β2M polypeptide sequence, the L3 linker polypeptide sequence, and/or the MHC-H polypeptide sequence comprises a chemical conjugation site (e.g., provided by protein engineering, such as a cysteine substitution) for epitope conjugation.

It is understood that such unconjugated T-Cell-MPs do not comprise a covalently attached epitope (e.g., peptide epitope); however, the disclosure includes and provides for T-Cell-MP epitope conjugates that further comprise a covalently attached epitope. The covalently attached epitope can be positioned within the binding cleft of the MHC-H/β2M polypeptide sequences and presented to a TCR, thereby permitting use of the molecules as agents for clinical testing and diagnostics, and as therapeutics. The T-Cell-MPs and their epitope conjugates described herein represent scalable antigen presenting cell-independent (APC-independent) immunotherapeutics that enable clinically effective levels of antigen specific T cell modulation (e.g., inhibition or activation) depending on the MOD(s) present. Moreover, the scaffold portions of T-cell MPs, which may be immunoglobulin Fc domains, permit multivalent presentation of MHC-epitope conjugate and MOD moieties to cognate T cells sufficient for their activation.

### BRIEF DESCRIPTION OF THE DRAWINGS

FIG. 1 depicts preferential activation of T cells by an embodiment of a duplex T-Cell-MP-epitope conjugate with an indirect (via a linker) covalent attachment of the epitope to the β2M polypeptides and bearing MODs, which can be wt. and/or variant MODs (e.g., having reduced affinity for their receptors (Co-MODs)). The first, epitope-specific T cell is activated due to productive engagement of both the TCRs and Co-MODs. In contrast, the second, epitope-non-specific T cell is not activated as the epitope cannot engage the TCR, and thus the MODs by themselves do not lead to productive engagement. Linkers and the location of optional linkers are represented by black lines joining T-Cell-MP elements.

FIGS. 2A-2H provide amino acid sequences of immunoglobulin heavy chain polypeptides (including SEQ ID NOs. 1-13).

FIG. 2I provides the sequence of a human immunoglobulin J-chain (SEQ ID NO:14).

FIG. 2J provides the sequence of an Ig CH1 domain sequence (SEQ ID NO:15).

FIG. 2K provides sequences of Ig κ and Ig λ chains (SEQ ID NOs:16-17).

FIGS. 3A, 3B and 3C provide amino acid sequences of major histocompatibility complex class I heavy chain (MHC-H; also known as human leukocyte antigen (HLA) Class I heavy chain) polypeptides. Signal sequences, aas 1-24, are bolded and underlined. FIG. 3A entry: 3A.1 is the HLA-A heavy chain (HLA-A*01:01:01:01 or A*0101) (NCBI accession NP_001229687.1), SEQ ID NO:18; entry 3A.2 is HLA-A*1101, SEQ ID NO:19; entry 3A.3 is HLA-A*2402, SEQ ID NO:20, and entry 3A.4 is HLA-A*3303, SEQ ID NO:21. FIG. 3B provides the sequence for HLA-B*07:02:01 (HLA-B*0702) (NCBI GenBank Accession NP_005505.2), SEQ ID NO:22. FIG. 3C provides the sequence for HLA-C*0701 (GenBank Accession NP_001229971.1) (HLA-C*07:01:01:01 or HLA-Cw*070101), (HLA-Cw*07) (see GenBank Accession CA078194.1), SEQ ID NO:23.

FIG. 3D provides an alignment of all, or substantially all, of the α1, α2, and α3 domains of eleven mature MHC-H polypeptide sequences without all, or substantially all, of their leader, transmembrane and intracellular domain regions. The aligned sequences include human HLA-A*0101, SEQ ID NO:24 (see also SEQ ID NO:18); HLA-B*0702, SEQ ID NO:25; HLA-C, SEQ ID NO:26; HLA-A*0201, SEQ ID NO:27; a mouse H2K protein sequence, SEQ ID NO:28; three variants of HLA-A (var. 2, var. 2C [having Y84C and A139C substitutions], and var. 2CP), SEQ ID NOs:29-31; 3 human HLA-A molecules (HLA-A*1101 (HLA-A11), SEQ ID NO:32; HLA-A*2402 (HLA-A24), SEQ ID NO:33; and HLA-A*3303 (HLA-A33), SEQ ID NO:34). HLA-A*0201 is a variant of HLA-A. The Y84A and A236C variant of HLA-A is marked as HLA-A (var. 2). The seventh HLA-A sequence, marked as HLA-A (var. 2C), shows HLA-A substituted with C residues at positions 84, 139 and 236, and the eighth sequence adds one additional proline to the C-terminus of the preceding sequence. The ninth through the eleventh sequences are from HLA-A11 (HLA-A*1101); HLA-A24 (HLA-A*2402); and HLA-A33 (HLA-A*3303), respectively, which are prevalent in certain Asian populations. Indicated in the alignment are the locations (84 and 139 of the mature proteins) where cysteine residues may be inserted in place of the aa at that position for the formation of a disulfide bond to stabilize the MHC-H −β2M complex in the absence of a bound peptide epitope. Also shown in the alignment is position 236 (of the mature polypeptide), which may be replaced by a cysteine residue that can form an interchain disulfide bond with β2M (e.g., at aa 12 of the mature polypeptide forming, for example, an HLA-A*0201 A236C to β2M R12C disulfide bond). An arrow appears above each of those locations and the residues are bolded. The boxes flanking residues 84, 139 and 236 show the groups of five aas on either side of those six sets of five residues, denoted aa clusters 1, 2, 3, 4, 5, and 6 (shown in the figure as aac 1 through aac 6, respectively), that may be replaced by 1 to 5 aas selected independently from (i) any naturally occurring aa or (ii) any naturally occurring aa except proline or glycine.

FIGS. 3E-3G provide alignments of the aa sequences of all, or substantially all, of the α1, α2, and α3 domains of several mature HLA-A, -B, and -C class I heavy chains, respectively. The sequences are provided for a portion of the mature proteins (without all or substantially all of their leader sequences, transmembrane domains or intracellular domains). As described in FIG. 3D, the positions of aa residues 84, 139, and 236 and their flanking residues (aac 1 to aac 6) that may be replaced by 1 to 5 aas selected independently from (i) any naturally occurring aa or (ii) any naturally occurring aa except proline or glycine are also shown. A consensus sequence is also provided for each group of HLA alleles provided in the figures showing the variable aa positions as “X” residues sequentially numbered and the locations of aas 84, 139 and 236 double underlined.

FIG. 3H provides a consensus sequence for all, or substantially all, of the α1, α2, and α3 domains of each of HLA-E, -F, and -G polypeptides with the variable aa positions indicated as “X” residues sequentially numbered and the locations of aas 84, 139 and 236 double underlined.

FIG. 3I provides an alignment of the consensus aa sequences for HLA-A, -B, -C, -E, -F, and -G, which are given in FIGS. 3E to 3H (SEQ ID NOs: 39, 47, and 57-60). The alignment shows the correspondence of aas between the different sequences. Variable residues in each sequence are listed as “X” with the sequential numbering removed. The permissible aas at each variable residue can be determined by reference to FIGS. 3E-3H. As indicated in FIG. 3D, the locations of aas 84, 139 and 236 with their flanking five-aa clusters that may be replaced by 1 to 5 aas selected independently from (i) any naturally occurring aa or (ii) any naturally occurring aa except proline or glycine are also shown.

FIG. 4 provides a multiple aa sequence alignment of β2M precursors (i.e., including the leader sequence) from Homo sapiens (NP_004039.1; SEQ ID NO:61), Pan troglodytes (NP_001009066.1; SEQ ID NO:62), Macaca mulatta (NP_001040602.1; SEQ ID NO:63), Bos Taurus (NP_776318.1; SEQ ID NO:64) and Mus musculus (NP_033865.2; SEQ ID NO:65). Underlined aas 1-20 are the signal peptide (sometime referred to as a leader sequence). The mature β2M sequences starts at aa 21.

FIG. 5 provides six unconjugated T-Cell-MP embodiments (structures) marked as A through F. In each case the T-Cell-MPs comprise: at least one MOD polypeptide sequence; a core structure that comprises the elements, in the N-terminus to C-terminus direction: a β2M polypeptide sequence, a Class I MHC-H polypeptide sequence comprising MHC-H α1, α2, and α3 domain sequences; and a scaffold polypeptide sequence (e.g., an Ig Fc polypeptide sequence). In the embodiments shown the α1 and α2 polypeptide sequences are linked by an intra-peptide bond between cysteines substituted, for example, with Tyr 84 and Ala 139 (Y84C and A139C substitutions). One or more MODs are located at the amino and/or carboxyl side of the core structure. Optional linker polypeptides that are selected independently, denoted as L1 to L6, are indicated by the line segments. The optional linker polypeptides may appear at either the ends of the T-Cell-MP polypeptide or joining the indicated polypeptide sequences. While the chemical conjugation site for coupling the epitope can be located at any location on the T-Cell-MP, potential locations in the β2M polypeptide sequence and the MHC-H polypeptide sequence for the chemical conjugation sites are indicated by asterisks. Although not shown, chemical conjugation sites may also be located in the L3 linker joining the β2M polypeptide sequence and MHC-H polypeptide sequence.

FIG. 6 provides six embodiments of unconjugated T-Cell-MPs, marked as A through F, that parallel the embodiments in FIG. 5. In the embodiments shown, the chemical conjugation site is indicated as being present in the β2M polypeptide sequence (e.g., comprising an E44C substitution) and the scaffold is an immunoglobulin Fc region, which may be interspecific, thereby permitting two different unconjugated T-Cell-MPs to specifically combine to form a heteroduplex.

FIG. 7 provides examples of unconjugated T-Cell-MPs having different MOD substitutions (e.g., tandem IL-2 MODs in structure A). The chemical conjugation sites are indicated as being present in the β2M polypeptide sequence (e.g., an E44C substitution); however, they could be in the MHC-H polypeptide (the α1, α2, and α3 sequence), or in the linker joining the β2M and MHC polypeptides. The Fc scaffold may be replaced by any other scaffold sequence such as an interspecific Fc polypeptide sequence that can form a heterodimer with its counterpart sequence, and the specific linkers listed are only exemplary and may be replaced by other linker polypeptide sequences.

FIG. 8 shows some schematics of epitopes having a maleimide group appended for conjugation to a free nucleophile (e.g., cysteine) present in a T-Cell-MP to form an epitope conjugate. In “a” the maleimide group is attached by an optional linker (e.g., a peptide linker sequence) to the epitope. In “b”-“e,” the linker is a glycine serine polypeptide GGGGS (SEQ ID NO:139) repeated n times, where n is 1-5 when present, and n is 0 when the linker is absent. In “c”-“e” the attachment of a maleimide group is through a lysine (K) on the end of the (GGGGS)n linker, such as through the epsilon amino group of the lysine. In “d” and “e” the maleimide group is linked to the peptide through an alkyl amide formed with the epsilon amino group of a lysine (K) residue, where m is 1-7.

FIG. 9 depicts the formation of a conjugated T-Cell-MP homoduplex from an unconjugated T-Cell-MP having a scaffold (in this case an Ig Fc scaffold) shown at (A). The conjugated T-Cell-MP polypeptide from (A) forms a homoduplex as shown in (B) via interactions between the scaffold sequences. The unconjugated homoduplex may be isolated from cells stably or transiently expressing the T-Cell-MP protein. The unconjugated homoduplex, generally in a purified form, is subjected to chemical conjugation by coupling an epitope to the conjugation sites, which is exemplified by the reaction between a cysteine in the β2M polypeptide sequence (e.g., comprising an E44C substitution) and a maleimide labeled peptide to yield the T-Cell-MP-epitope conjugate shown in (C). Excess reactive peptide can be removed or substoichiometric amounts of the reactive epitope (relative to the amount of conjugation sites) can be utilized to produce the conjugated T-Cell-MP homoduplex. The constructs are not limited to the linker sequences shown, which are exemplary of the linkers that may be employed.

FIG. 10 depicts the formation of a conjugated T-Cell-MP heteroduplex from unconjugated T-Cell-MPs having scaffolds that selectively form heteroduplexes (in this case interspecific knob-in-hole Ig Fc scaffolds) shown at (A). The conjugated T-Cell-MP polypeptides form a heteroduplex as shown in (B) via interactions between the interspecific scaffold sequences. The unconjugated heteroduplex may be isolated from cells stably or transiently expressing the protein. The unconjugated heteroduplex, generally in a purified form, is subjected to chemical conjugation by coupling an epitope to the conjugation sites, which is exemplified by the reaction between a cysteine in the β2M polypeptide sequence (e.g., an E44C substitution) and a maleimide labeled peptide to yield the T-Cell-MP-epitope conjugate shown in (C). Excess reactive peptide can be removed or substoichiometric amounts of the reactive epitope (relative to the amount of conjugation sites) can be utilized to produce the conjugated T-Cell-MP heteroduplex, which as shown may comprise different MODs on each of the T-Cell-MP polypeptides. The constructs are not limited to the linker sequences shown, which are exemplary of the linkers that may be employed.

FIG. 11 shows three heterodimeric T-Cell-MP-epitope conjugate duplexes. Each has a scaffold comprising an interspecific Ig Fc polypeptide pair; however, the scaffold polypeptides may be replaced by any other interspecific polypeptide pair. The constructs are not limited to the linker sequences shown, which are exemplary of the linkers that may be employed.

FIG. 12 shows comparative results for the expression of a series of molecules including T-Cell-MPs in cultured CHO cells, described in Example 1, with the molecules (constructs) having varied substitutions in the L3 linker and at other locations. The overall structure of the molecules is provided at A, B, and C. The titer (amount of protein) of the molecules and fraction of the molecules that are unaggregated (e.g., existing as soluble duplexes) are provided in histograms D and E respectively.

FIG. 13 shows the production and stability in culture of an unconjugated T-Cell-MP (construct 3861, which has an L3 linker consisting of a Gly₄Ser repeated three times) at 2, 4, and 6 million cells per ml at both 32 and 28° over several days (A and B). The chromatograms show protein A purified material from a culture before (C) and after (D) further purification by size exclusion chromatography. The coomassie blue gel (E) shows that materials run against molecular weight standards (Mw) at 103128 Daltons for reduced (R) and 206213 Daltons for non-reduced samples. See Example 2 for details.

FIG. 14 at A demonstrates the specificity of the T-Cell-MP-epitope conjugates for T cells specific to the conjugated epitope. At B, FIG. 14 shows an electrophoresis gel of non-reduced and reduced samples of epitope conjugates. See Example 3 for details.

FIG. 15 and FIG. 16 show the response of CD8+ T cells present in Leukopak samples from CMV and MART-1 response donors to T-Cell-MP epitope conjugates and control treatments as described in Example 4.

FIG. 17 shows the effect of L3 linker length on the CHO cell expression of two series of unconjugated T-Cell-MPs, providing the titer in culture media by Octet analysis at A, and the fraction of unaggregated (duplex) molecules present in the samples at B following purification on protein A magnetic beads.

FIG. 18 depicts a method of directing T cells (e.g., CD 8+ cytotoxic T-cells) to a target cell (e.g., a cancer cell as shown) and directing the T cell's response to the target cell.

FIG. 19 provides the amino acid sequences of certain constructs discussed in this disclosure. Linker sequences (e.g., AAAGG and GGGGS) may be bolded, italicized and underlined to permit their identification. The indicated single amino acid substitutions in the MHC class I heavy chain are shown in bold with underlining Human IL2 sequences are indicated by hIL2, beta-2-microglobin sequences are indicated by β2M, and HLA-A02 sequences are indicated by HLA-A*0201, with each bearing the indicated aa substitutions.

### DEFINITIONS

The term T-Cell-MP is generic to, and includes, both unconjugated T-Cell-MPs and T-Cell-MP-epitope conjugates. The term “unconjugated T-Cell-MP (or “MPs” when plural) refers to T-Cell-MPs that have not been conjugated (covalently linked) to an epitope and/or payload (e.g., a non-epitope molecule such as a label), and therefore comprise at least one chemical conjugation site. Unconjugated T-Cell-MP polypeptides also do not comprise a fused peptide epitope that can be positioned within the MHC-H binding cleft and in conjunction with the β2M polypeptide sequence and presented to a TCR. The terms “T-Cell-MP-epitope conjugate” (or “conjugates” when plural) refers to T-Cell-MPs that have been conjugated (covalently linked) to an epitope at a chemical conjugation site that permits the covalently linked epitope to be present in the MHC binding cleft and presented to a TCR with specificity for the epitope expressed on a T Cell (an epitope specific T cell). “T-Cell-MP-payload conjugate” and “T-Cell-MP-payload conjugates” refer to T-Cell-MPs that have been conjugated (covalently linked) to one or more independently selected payloads. The term “T-Cell-MP” also includes unconjugated T-Cell-MPs and T-Cell MP-epitope conjugates that either comprise one or more independently selected MODs or are MOD-less. In those instances where this disclosure specifically refers to a T-Cell-MP that does not contain a MOD, terms such as “MOD-less T-Cell-MP” or a “T-Cell-MP without a MOD” and the like are employed. The term “T-Cell-MP” also includes unconjugated T-Cell-MPs and T-Cell MP-epitope conjugates that comprise either one or more independently selected targeting sequences (discussed below).

The terms “polynucleotide” and “nucleic acid,” used interchangeably herein, refer to a polymeric form of nucleotides of any length, either ribonucleotides or deoxyribonucleotides. Thus, these terms include, but are not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

The terms “polypeptide” and “protein,” used interchangeably herein, refer to a polymeric form of amino acids, which unless stated otherwise are the naturally occurring proteinogenic L-amino acids that are incorporated biosynthetically into proteins during translation in a mammalian cell.

A nucleic acid or polypeptide has a certain percent “sequence identity” to another nucleic acid or polypeptide, meaning that, when aligned, that percentage of bases or amino acids are the same, and in the same relative position, when comparing the two sequences. Sequence identity can be determined in a number of different ways. To determine sequence identity, sequences can be aligned using various convenient methods and computer programs (e.g., BLAST, T-COFFEE, MUSCLE, MAFFT, etc.), available over the world wide web at sites including blast.ncbi.nlm.nih.gov/Blast.cgi for BLAST+2.10.0, ebi.ac.uk/Tools/msa/tcoffee/, ebi.ac.uk/Tools/msa/muscle/, and mafft.cbrc.jp/alignment/software/. See, e.g., Altschul et al. (1990), J. Mol. Biol. 215:403-10.

As used herein amino acid (“aa” singular or “aas” plural) means the naturally occurring proteinogenic amino acids incorporated into polypeptides and proteins in mammalian cell translation. Unless stated otherwise, these are: L (Leu, leucine), A (Ala, alanine), G (Gly, glycine), S (Ser, serine), V (Val, valine), F (Phe, phenylalanine), Y (Tyr, tyrosine), H (His, histidine), R (Arg, arginine), N (Asn, asparagine), E (Glu, glutamic acid), D (Asp, aspartic acid), C (Cys, cysteine), Q (Gln, glutamine), I (Ile, isoleucine), M (Met, methionine), P (Pro, proline), T (Thr, threonine), K (Lys, lysine), and W (Trp, tryptophan). Amino acid also includes the amino acids, hydroxyproline and selenocysteine, which appear in some proteins found in mammalian cells; however, unless their presence is expressly indicated they are not understood to be included.

The term “conservative amino acid substitution” refers to the interchangeability in proteins of aa residues having similar side chains. For example, a group of aas having aliphatic side chains consists of glycine, alanine, valine, leucine, and isoleucine; a group of aas having aliphatic-hydroxyl side chains consists of serine and threonine; a group of aas having amide containing side chains consists of asparagine and glutamine; a group of aas having aromatic side chains consists of phenylalanine, tyrosine, and tryptophan; a group of aas having basic side chains consists of lysine, arginine, and histidine; a group of aas having acidic side chains consists of glutamate and aspartate; and a group of aas having sulfur containing side chains consists of cysteine and methionine. Exemplary conservative aa substitution groups are: valine-leucine-isoleucine, phenylalanine-tyrosine, lysine-arginine, alanine-valine-glycine, and asparagine-glutamine.

The term “binding” (or “bound”) refers generically to a direct association between molecules and/or atoms, due to, for example, covalent, electrostatic, hydrophobic, ionic and/or hydrogen-bond interactions, including interactions such as salt bridges and water bridges.

The term “binding” (or “bound”) as used with reference to a T-Cell-MP binding to a polypeptide (e.g., a T cell receptor on a T cell) refers to a non-covalent interaction between two molecules. A non-covalent interaction refers to a direct association between two molecules, due to, for example, electrostatic, hydrophobic, ionic, and/or hydrogen-bond interactions, including interactions such as salt bridges and water bridges. Non-covalent binding interactions are generally characterized by a dissociation constant (K_{D}) of less than 10⁻⁶ M, less than 10⁻⁷ M, less than 10⁻⁸ M, less than 10⁻⁹ M, less than 10⁻¹⁰ M, less than 10⁻¹¹ M, less than 10⁻¹² M, less than 10⁻¹³ M, less than 10⁻¹⁴ M, or less than 10⁻¹⁵ M. “Covalent bonding” or “covalent binding” as used herein refers to the formation of one or more covalent chemical bonds between two different molecules.

“Affinity” as used herein generally refers to the strength of non-covalent binding, increased binding affinity being correlated with a lower K_{D}. As used herein, the term “affinity” may be described by the dissociation constant (K_{D}) for the reversible binding of two agents (e.g., an antibody and an antigen). Affinity can be at least 1-fold greater to at least 1,000-fold greater (e.g., at least 2-fold to at least 5-fold greater, at least 3-fold to at least 6-fold greater, at least 4-fold to at least 8-fold greater, at least 5-fold to at least 10-fold greater, at least 6-fold to at least 15-fold greater, at least 7-fold to at least 20-fold greater, at least 8-fold to at least 30-fold greater, at least 9-fold to at least 35-fold greater, at least 10-fold to at least 40-fold greater, at least 20-fold to at least 60-fold greater, at least 40-fold to at least 80-fold greater, at least 60-fold to at least 180-fold greater, at least 80-fold to at least 240-fold greater, at least 100-fold to at least 1,000-fold greater, or at least 1,000-fold greater) than the affinity of an antibody or receptor for an unrelated aa sequence (e.g., ligand). Affinity of an antibody to a target protein can be, for example, from about 100 nanomolar (nM) to about 0.1 nM, from about 100 nM to about 1 picomolar (pM), or from about 100 nM to about 1 femtomolar (fM) or more. As used herein, the term “avidity” refers to the resistance of a complex of two or more agents to dissociation after dilution.

The term “immunological synapse” or “immune synapse” as used herein generally refers to the natural interface between two interacting immune cells of an adaptive immune response including, e.g., the interface between an antigen-presenting cell (APC) or target cell and an effector cell, e.g., a lymphocyte, an effector T cell, a natural killer cell, and the like. An immunological synapse between an APC and a T cell is generally initiated by the interaction of a T cell antigen receptor and MHC molecules, e.g., as described in Bromley et al., Ann. Rev. Immunol. 2001; 19:375-96; the disclosure of which is incorporated herein by reference in its entirety.

“T cell” includes all types of immune cells expressing CD3, including T-helper cells (CD4⁺ cells), cytotoxic T cells (CD8⁺ cells), regulatory T cells (T reg), and NK-T cells.

The term “immunomodulatory polypeptide” (also referred to as a “costimulatory polypeptide” or, as noted above, a “MOD”) as used herein includes a polypeptide or portion thereof (e.g., an ectodomain) on an APC (e.g., a dendritic cell, a B cell, and the like), or otherwise available to interact with the T cell, that specifically binds a cognate co-immunomodulatory polypeptide (“Co-MOD”) present on a T cell, thereby providing a signal. The signal provided by the MOD engaging its Co-MOD, in addition to the primary signal provided by, for instance, binding of a TCR/CD3 complex with a MHC polypeptide loaded with a peptide epitope, mediates (e.g., directs) a T cell response. The responses include, but are not limited to, proliferation, activation, differentiation, and the like. A MOD can include, but is not limited to, CD7, B7-1 (CD80), B7-2 (CD86), PD-L1, PD-L2, 4-1BBL, OX40L, Fas ligand (FasL), inducible costimulatory ligand (ICOS-L), intercellular adhesion molecule (ICAM), CD30L, CD40, CD70, CD83, HLA-G, lymphotoxin beta receptor, 3/TR6, ILT3, ILT4, HVEM, an agonist or antibody that binds Toll-Like Receptor (TLR), and a ligand that specifically binds with B7-H3. A MOD also encompasses, inter alia, an antibody or antibody fragment that specifically binds with and activates a Co-MOD molecule present on a T cell such as, but not limited, to antibodies against the receptors for any of IL-2, CD27, CD28, 4-1BB, OX40, CD30, CD40, PD-1, ICOS, lymphocyte function-associated antigen-1 (LFA-1), CD2, LIGHT (also known as tumor necrosis factor superfamily member 14 (TNFSF14)), NKG2C, B7-DC, B7-H2, B7-H3, and CD83.

“Recombinant” as used herein means that a particular nucleic acid (DNA or RNA) is the product of various combinations of cloning, restriction, polymerase chain reaction (PCR) and/or ligation steps resulting in a construct having a structural coding or non-coding sequence distinguishable from endogenous nucleic acids found in natural systems. DNA sequences encoding polypeptides can be assembled from cDNA fragments or from a series of synthetic oligonucleotides, to provide a synthetic nucleic acid which is capable of being expressed from a recombinant transcriptional unit contained in a cell or in a cell-free transcription and translation system.

The terms “recombinant expression vector” or “DNA construct,” used interchangeably herein, refer to a DNA molecule comprising a vector and at least one insert. Recombinant expression vectors are usually generated for the purpose of expressing and/or propagating the insert(s), or for the construction of other recombinant nucleotide sequences. The insert(s) may or may not be operably linked to a promoter sequence and may or may not be operably linked to DNA regulatory sequences.

The terms “treatment,” “treating” and the like are used herein to generally mean obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment” as used herein covers any treatment of a disease or symptom in a mammal, and includes: (a) preventing the disease or symptom from occurring in a subject which may be predisposed to acquiring the disease or symptom but has not yet been diagnosed as having it; (b) inhibiting the disease or symptom, i.e., arresting its development; and/or (c) relieving the disease, i.e., causing regression of the disease. The therapeutic agent may be administered before, during or after the onset of disease or injury. The treatment of ongoing disease, where the treatment stabilizes or reduces the undesirable clinical symptoms of the patient, is of particular interest. Such treatment is desirably performed prior to complete loss of function in the affected tissues. The subject therapy will desirably be administered during the symptomatic stage of the disease and, in some cases, after the symptomatic stage of the disease.

The terms “individual,” “subject,” “host,” and “patient” are used interchangeably herein and refer to any mammalian subject for whom diagnosis, treatment, or therapy is desired. Mammals include humans and non-human primates, and in addition include rodents (e.g., rats; mice), lagomorphs (e.g., rabbits), ungulates (e.g., cows, sheep, pigs, horses, goats, and the like), felines, canines, etc.

Before the present invention is further described, it is to be understood that this invention is not limited to the particular embodiments described, as such may, of course, vary. It is also to be understood that the terminology used herein is for the purpose of describing particular embodiments only, and is not intended to limit the scope of the invention.

Where a range of values is provided, it is understood that each intervening value between the upper and lower limit of that range to a tenth of the lower limit of the range is encompassed within the disclosure along with any other stated or intervening value in the range. Upper and lower limits may independently be included in smaller ranges that are also encompassed within the disclosure subject to any specifically excluded limit in the stated range. Where the stated range has a value (e.g., an upper or lower limit), ranges excluding those values are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that, as used herein and in the appended claims, the singular forms “a,” “an,” and “the” include plural referents unless the context clearly dictates otherwise. Thus, for example, reference to “a T reg” includes a plurality of such T regs and reference to “the MHC Class I heavy chain” includes reference to one or more MHC Class I heavy chains and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

The publications discussed herein are provided solely for their disclosure prior to the filing date of the present application. Nothing herein is to be construed as an admission that the present invention is not entitled to antedate such publications by virtue of prior invention. Further, the dates of publication provided may be different from the actual publication dates which may need to be independently confirmed.

### DETAILED DESCRIPTION

### I. T-Cell Modulatory Polypeptides (T-Cell-MPs) with Chemical Conjugation Sites for Epitope Binding

The present disclosure includes and provides for T-Cell-MPs (both unconjugated T-Cell-MPs having a chemical conjugation site suitable for attaching an epitope and T-Cell-MP-epitope conjugates to which an epitope has been conjugated). Such T-Cell-MPs are useful for modulating the activity of T cells to, for example, modulate an immune response in vitro, ex vivo, or in vivo, and accordingly to effect therapeutic treatments. The present disclosure specifically provides methods of T-Cell MP-epitope conjugate preparation and use in modulating an immune response in vitro, ex vivo, or in vivo in an individual that may be a human or non-human test subject or patient. The human or non-human test subject or patient may be suffering from one or more tumors, one or more cancers, and/or one or more infections (e.g., bacterial and viral infections). In addition to the other elements present the T-Cell-MPs may comprise one or more independently selected wt. and/or variant MOD polypeptides that exhibit reduced binding affinity to their Co-MODs and one or more payloads.

Included in this disclosure are T-Cell-MPs that are homodimeric, comprising identical first and second T-Cell-MP polypeptides. Also included in this disclosure are T-Cell-MPs that are heterodimeric, comprising a first and a second T-Cell-MP polypeptide, wherein at least one of those polypeptides comprises a chemical conjugation site for the attachment of an epitope. Optionally at least one of the heterodimers may comprise a payload such as a chemotherapeutic agent and/or a targeting sequence. Included in this disclosure are T-Cell-MPs which have been chemically conjugated to an epitope to form a T-Cell-MP-epitope conjugate and which optionally comprise a targeting sequence and/or a payload.

Depending on the type of MOD(s) present in a T-Cell-MP-epitope conjugate, a T cell bearing a TCR specific to the epitope is present on a T-Cell-MP can respond by undergoing activation including, for example, clonal expansion (e.g., when activating MODs such as wt. and/or variants of IL-2, 4-1BBL and/or CD80 that are incorporated into the T-Cell-MP). Alternatively, the T cell may undergo inhibition that down regulates T cell activity when MODs such as wt. and/or variants of FASL and/or PD-L1 are incorporated into the T-Cell-MPs. The incorporation of combinations of MODs such as wt. and/or variants of IL-2 and CD80 or IL2 and PD-L1 into T-Cell-MPs (e.g., T-Cell-MP-epitope conjugates) may lead to synergistic effects where the T cell response more than exceeds the sum of the responses of T cells to otherwise identical T-Cell-MPs lacking one of the MODs. Because MODs are not specific to any epitope, activation or inhibition of T cells can be biased toward epitope-specific interactions by incorporating variant MODs having reduced affinity for their Co-MOD into the T-Cell-MPs such that the binding of a T-Cell-MP to a T cell is strongly affected by, or even dominated by, the MHC-epitope-TCR interaction.

A T-Cell-MP-epitope conjugate bearing MODs may be considered to function as a surrogate APC and, by interacting with a T-Cell, mimic the presentation of epitope in an adaptive immune response. The T-Cell-MP-epitope conjugate does so by engaging and presenting to a TCR present on the surface of a T cell with a covalently bound epitope (e.g., a peptide presenting an epitope). This engagement provides the T-Cell-MP-epitope conjugate with the ability to achieve epitope-specific cell targeting. In embodiments described herein, T-Cell-MP-epitope conjugates also possess at least one MOD that engages a counterpart costimulatory protein (Co-MOD) on the T cell. Both signals—epitope/MHC binding to a TCR and MOD binding to a Co-MOD—then drive both the desired T cell specificity and either inhibition/apoptosis or activation/proliferation.

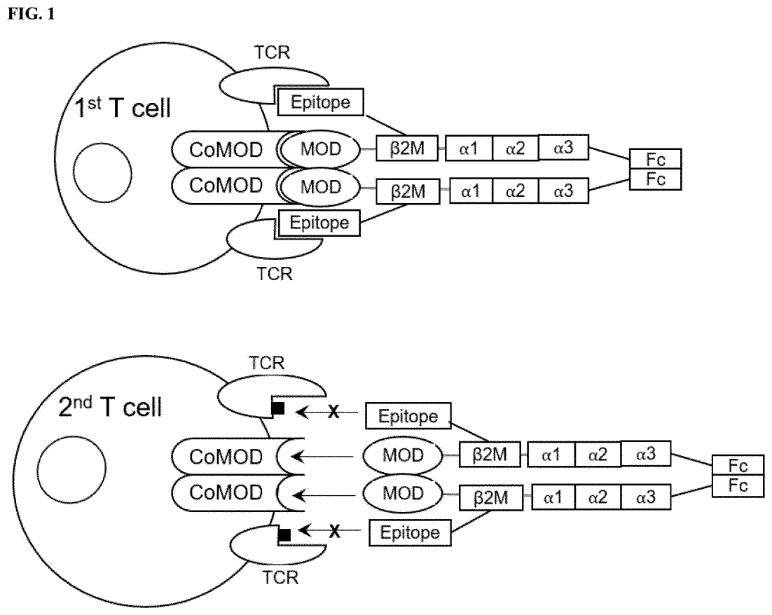

Unconjugated T-Cell-MPs, which have chemical conjugation sites, find use as a platform into which different epitopes may be introduced, either alone or in combination with one or more additional payloads added to the T-Cell-MP, in order to prepare materials for therapeutic, diagnostic and research applications. Because T-Cell-MPs, including duplexes comprised of homodimers, and higher order homomeric complexes require only a single polypeptide sequence, they can advantageously be introduced and expressed by cells using a single vector with a single expression cassette. Similarly, heterodimeric duplex T-Cell-MPs can be introduced into cells using a single vector with two separate expression cassettes or a bicistronic expression cassette (e.g. with the proteins separated by a 2A protein sequence or internal ribosome entry sequence (IRES)), or by using two vectors each bearing a cassette coding one heterodimeric subunit. Where duplex or higher order T-Cell-MPs contain interspecific scaffold sequences, the different T-Cell-MPs may bear different MODs permitting the duplex or higher order structure to contain different MODs, or MODs at different locations on each polypeptide of the heterodimer. The modular nature of T-Cell-MPs enables the rapid preparation and testing of diagnostic and therapeutic candidates by coupling an epitope containing molecule (e.g., a peptide) into prepared T-Cell-MP polypeptides that can then be tested for activation or inhibition of T cells bearing TCRs specific to the epitope. The ability to construct unconjugated T-Cell-MPs, and in particular heterodimer T-Cell-MP duplexes with different MODs, permits rapid assembly and assessment of different combinations of MODs with one or more epitope relevant to a disease state or condition. Further to the foregoing, the ability to rapidly attach and access the effectiveness of various payloads, such as antiviral agents, chemotherapeutics, and/or targeting sequences, to the T-Cell-MP facilitates preparation of T-Cell-MPs both for screening and as therapeutics.

Where one or more activating wt. MOD or variant MOD polypeptide sequences are incorporated into a T-Cell-MP epitope conjugate, contacting the T cells with a TCR specific to the epitope with at least one concentration of the T-Cell-MP-epitope conjugate can result in T cell activation. T cell activation may result in one or more of the following: an increase in the activity of ZAP-70 protein kinase activity, induction in the proliferation of the T-cell(s), granule-dependent effector actions (e.g., the release of granzymes, perforin, and/or granulysin from cytotoxic T-cells), and/or release of T cell cytokines (e.g., interferon γ from CD8+ cells). Where the MOD polypeptide sequence(s) induces T cell proliferation, the T-Cell-MP epitope conjugate may induce at least a twofold (e.g., at least a 2, 3, 4, 5, 10, 20, 30, 50, 75, or 100 fold) difference in the activation of T cells having a TCR specific to the epitope as compared to T cells contacted with the same concentration of the T-Cell-MP-epitope conjugate that do not have a TCR specific to the epitope (see FIG. 1). Activation of T-cells may be measured by, for example, ZAP-70 activity or T cell proliferation (see, e.g., Wang, et al., Cold Spring Harbor perspectives in biology 2.5 (2010): a002279), or cytokine release. Where one or more wt. or variant MOD polypeptide sequences that inhibit T cell activation are incorporated into a T-Cell-MP-epitope conjugate, contacting the T cells having a TCR specific to the epitope with at least one concentration of the T-Cell-MP-epitope conjugate may result in one or more of the following: prevention or inhibition of the T cell's activation, reduction in the response of activated T cells, and/or down regulation of the epitope-specific T-Cell. In some cases, inhibitory MODs present in a T-Cell-MP-epitope conjugate may result in apoptosis of T cell(s) with a TCR specific to the epitope. The effects of inhibitory MOD sequences may be measured by, for example, one or more of their: effect on T cell proliferation, ZAP-70 activity, reduction in granule-dependent effector actions, and/or cell death.

The specificity of T-Cell-MP-epitope conjugates depends on the relative contributions of the epitope and its MODs to the binding. Where the affinity of the MOD(s) for the Co-MOD(s) is relatively high such that the MOD(s) dominate the T-Cell-MPs in the binding interactions, the specificity of the T-Cell-MP-epitope conjugates will be reduced relative to T-Cell-MP complexes where the epitope dominates the binding interactions by contributing more to the overall binding energy than the MODs. The greater the contribution of binding energy between an epitope and a TCR specific to the epitope, the greater the specificity of the T-Cell-MP will be for the T cell bearing that type of TCR. Where an epitope MHC complex has strong affinity for its TCR, the use of wt. MODs that have relatively low affinity and/or variant MODs with reduced affinity for their Co-MODs will favor epitope selective interactions of the T-Cell-MP-epitope conjugates with specific T cells, and also facilitate selective delivery of any payload that may be conjugated to the T-Cell-MP-epitope conjugate to the T cell and/or locations where the T cell is located.

The present disclosure provides T-Cell-MP-epitope conjugates presenting cancer and/or infectious agent (e.g., viral, bacterial) epitopes that are useful for modulating the activity of T cells in an epitope-specific manner and, accordingly, for modulating an immune response to those disease states in an individual. The T-Cell-MPs comprise one or more MODs that are wt. and/or exhibit reduced binding affinity to a Co-MOD.

A. Unconjugated T-Cell-MPs and T-Cell-MP-Epitope Conjugates

1 The Structure and Composition of Unconjugated T-Cell-MPs and T-Cell-MP-Epitope Conjugate Components

The unconjugated T-Cell-MPs described herein comprise a chemical conjugation site for coupling an epitope directly, or indirectly through a linker. The chemical conjugation site can be situated at any location on the T-Cell-MP. One aspect of the disclosure is directed to T-Cell-MPs that comprise a chemical conjugation site for the attachment of a peptide epitope within the scaffold (e.g., Ig Fc), β2M, or MHC-H polypeptide sequences, or within the linker (L3) joining the β2M and MHC-H polypeptide sequences, and higher order complexes of those T-Cell-MPs. Another aspect of the disclosure is directed to T-Cell-MPs that comprise a chemical conjugation site for the attachment of a peptide epitope within the β2M, or MHC-H polypeptide sequences, or within the linker (L3) joining the β2M and MHC-H polypeptide sequences, and higher order complexes of those T-Cell-MPs. A chemical conjugation site for coupling an epitope directly, or indirectly through a linker, can be situated in the β2M polypeptide sequence. A chemical conjugation site for coupling an epitope directly, or indirectly through a linker, can be situated in the MHC-H polypeptide sequence. A chemical conjugation site for coupling an epitope directly, or indirectly through a linker, can be situated in the linker (L3) joining the β2M polypeptide sequence and MHC-H polypeptide sequence. A chemical conjugation site for coupling an epitope directly, or indirectly through a linker, can be situated within the scaffold (e.g., Ig Fc). Where a chemical conjugation site for coupling an epitope to an unconjugated T-Cell-MP appears in a scaffold (e.g., an Ig Fc), β2M, or MHC-H polypeptide sequence, the chemical conjugation site may be limited to an amino acid or sequence of amino acids not naturally appearing in any of those sequences, and may involve one or more amino acids introduced into one of those sequences (e.g., one or more aas introduced into an aa sequence position at which the one or more aas do not appear in the naturally occurring sequence). In addition, while it is possible to utilize the N-terminal amino group or C-terminal carboxyl group of a T-Cell-MP polypeptide as a chemical conjugation site for epitope attachment, those sites may be excluded as conjugation sites from any of the T-Cell-MPs or their higher order complexes described herein. Indeed, the chemical conjugation site of a T-Cell-MP may be excluded from the N-terminal 10 or 20 aas and/or the C-terminal 10 or 20 aas.

T-Cell-MPs may form higher order complexes (e.g., duplexes, triplexes, etc.). The higher order complexes may be homomeric (e.g., homodimers or homoduplexes) or heteromeric (e.g., heterodimers or heteroduplexes). Pairs of interspecific sequences may be employed as scaffold sequences where the complexes are intended to be heterodimeric as they permit two different T-Cell-MPs to form a specific heteroduplex, as opposed to a mixture of homoduplexes and heteroduplexes that can form if two T-Cell-MPs not having a pair of interspecific binding sequences are mixed.

A first group of T-Cell-MP molecules described herein is broadly directed to T-Cell-MPs that may form a duplex that associates through interactions in their scaffold sequences. Such T-Cell-MPs may have at least a first T-Cell-MP polypeptide sequence (e.g., duplexed as a homodimer), or non-identical first and second T-Cell-MP polypeptide sequences (e.g., duplexed as a heterodimer), with one or both of the T-Cell-MPs comprising (e.g., from N-terminus to C-terminus):
(i) optionally one or more MOD polypeptide sequences (e.g., two or more MOD polypeptide sequences, such as in tandem, wherein when there are two or more MOD polypeptide sequences they are optionally joined to each other by independently selected L1 linkers);
(ii) an optional L2 linker polypeptide sequence joining the one or more MOD polypeptide sequences to a β2M polypeptide sequence;
(iii) the β2M polypeptide sequence;
(iv) an optional L3 linker polypeptide sequence (e.g., from 10-50 aa in length);
(v) a class I MHC-H polypeptide sequence;
(vi) an optional L4 linker polypeptide sequence;
(vii) a scaffold polypeptide sequence (e.g., an immunoglobulin Fc sequence);
(viii) an optional L5 linker polypeptide sequence; and
(ix) optionally one or more MOD polypeptide sequence (e.g., two or more MOD polypeptide sequences, such as in tandem, wherein when there are two or more MOD polypeptide sequences they are optionally joined to each other by independently selected L6 linkers);
wherein the unconjugated T-Cell-MP comprises at least one MOD polypeptide sequence (e.g., the MOD(s) of element (i) and/or (ix)); and
wherein at least one of the β2M polypeptide sequence, the L3 linker polypeptide sequence, and/or the MHC-H polypeptide sequence comprises at least one chemical conjugation site.

A second group of unconjugated T-Cell-MPs described herein may form a duplex between a first T-Cell-MP and a second T-Cell-MP that associate through interactions in their scaffold sequences. Such unconjugated duplex T-Cell-MPs may have an identical first and second T-Cell-MP polypeptide sequence duplexed as a homodimer, or non-identical first and second T-Cell-MP polypeptide sequences duplexed as a heterodimer, with one or both of the T-Cell-MPs comprising from N-terminus to C-terminus:
(i) optionally one or more MOD polypeptide sequences (e.g., two or more MOD polypeptide sequences, such as in tandem, wherein when there are two or more MOD polypeptide sequences they are optionally joined to each other by independently selected L1 linkers);
(ii) an optional L2 linker polypeptide sequence joining the one or more optional MOD polypeptide sequences to a β2M polypeptide sequence;
(iii) the β2M polypeptide sequence;
(iv) an optional L3 linker polypeptide sequence (e.g., from 10-50 aa in length);
(v) a class I MHC-H polypeptide sequence;
(vi) an optional L4 linker polypeptide sequence;
(vii) a scaffold polypeptide sequence (e.g., an immunoglobulin Fc sequence);
(viii) an optional L5 linker polypeptide sequence; and
(ix) optionally one or more MOD polypeptide sequence (e.g., two or more MOD polypeptide sequences, such as in tandem, wherein when there are two or more MOD polypeptide sequences they are optionally joined to each other by independently selected L6 linkers);
wherein the unconjugated T cell modulatory polypeptide comprises at least one MOD polypeptide sequence (e.g., the MOD(s) of element (i) and/or (ix)); and
wherein at least one of the β2M polypeptide sequence, the L3 linker polypeptide sequence, and/or the MHC-H polypeptide sequence comprises at least one chemical conjugation site, e.g., for epitope conjugation and/or payload conjugation.

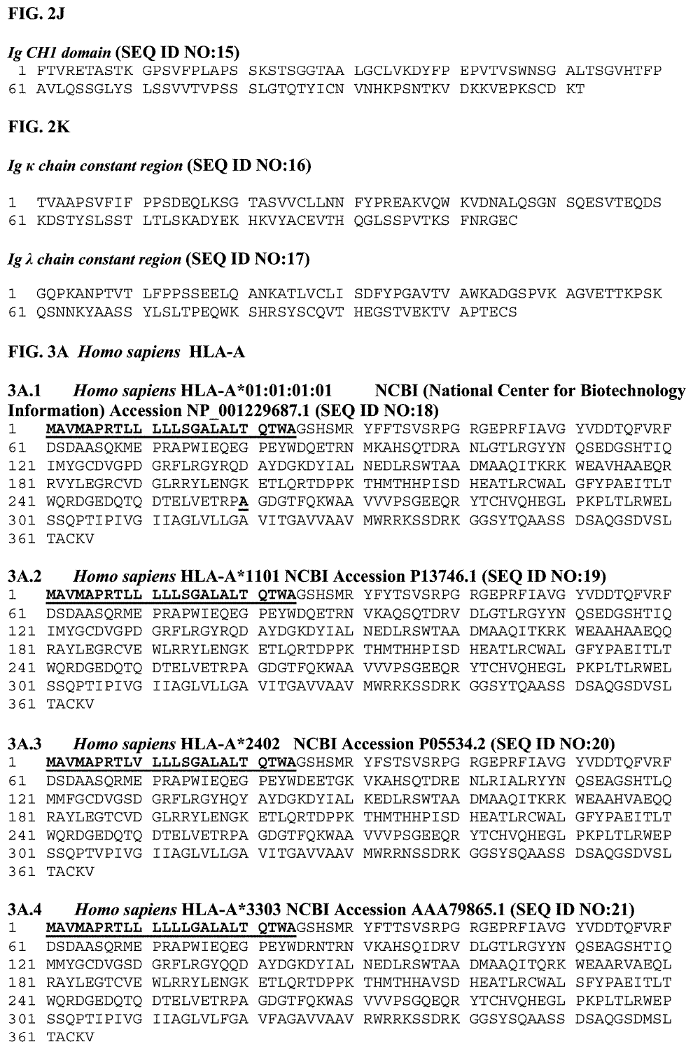

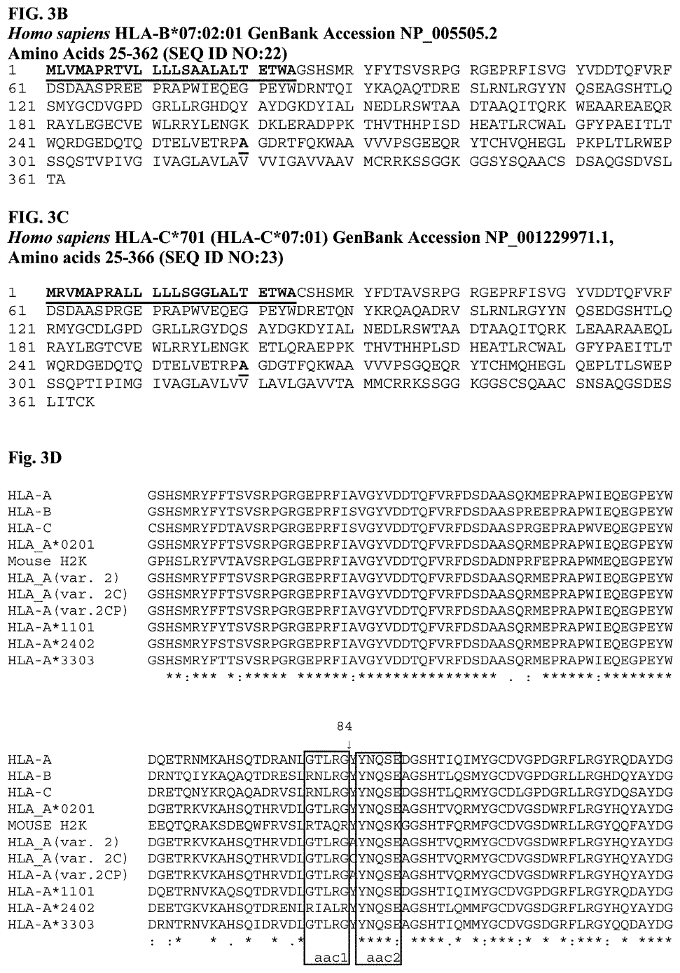

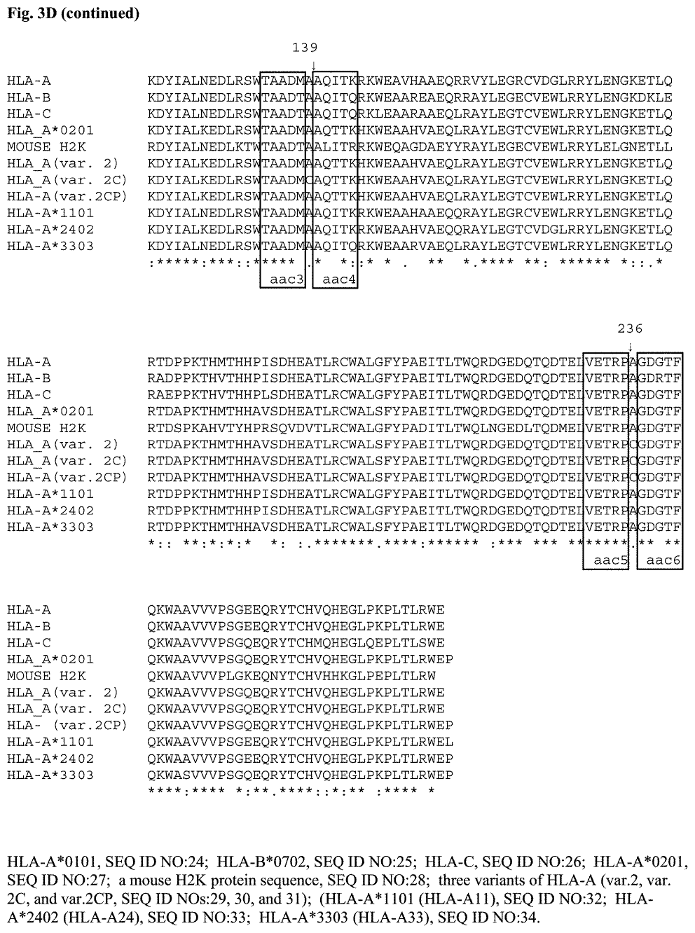

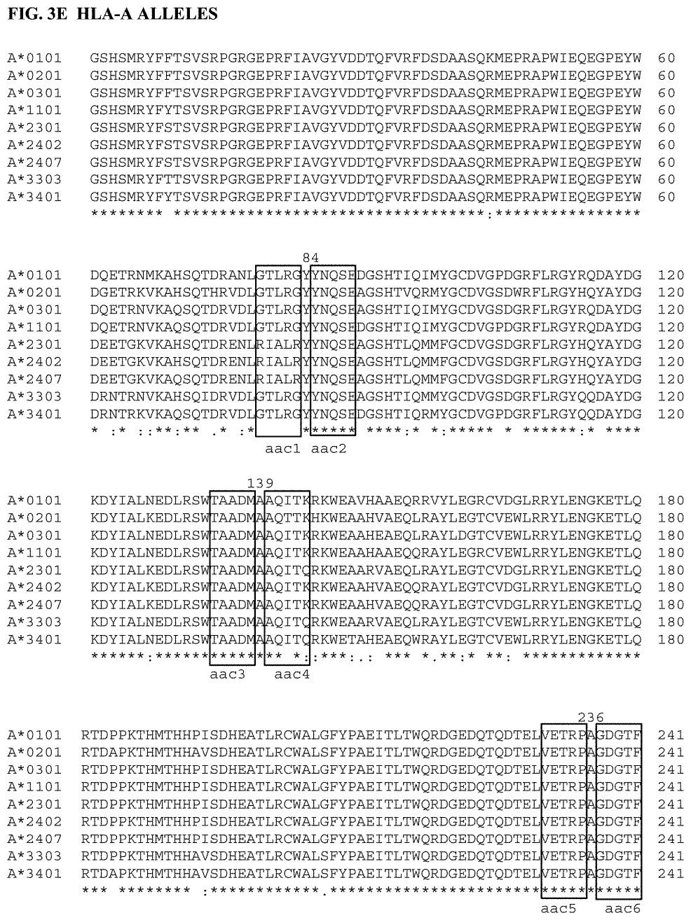

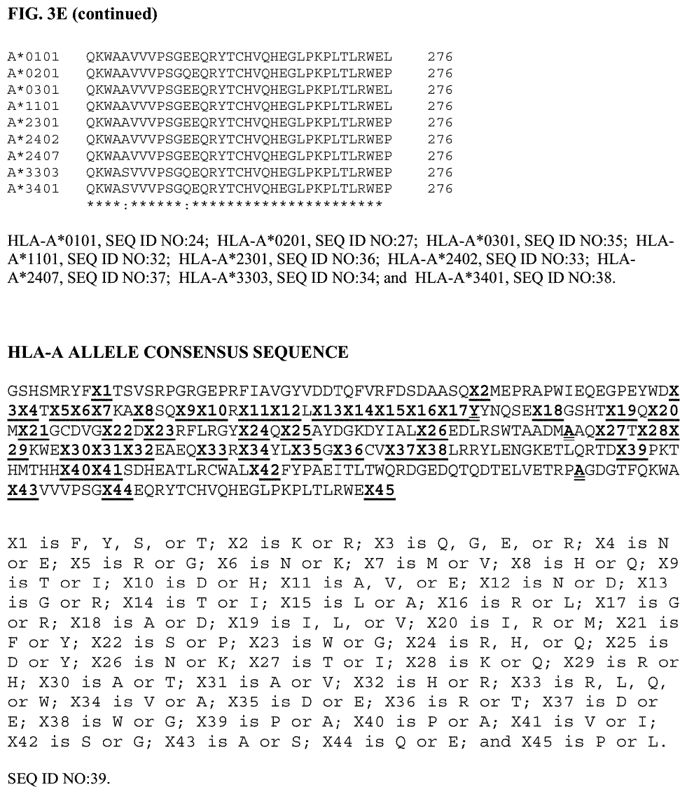

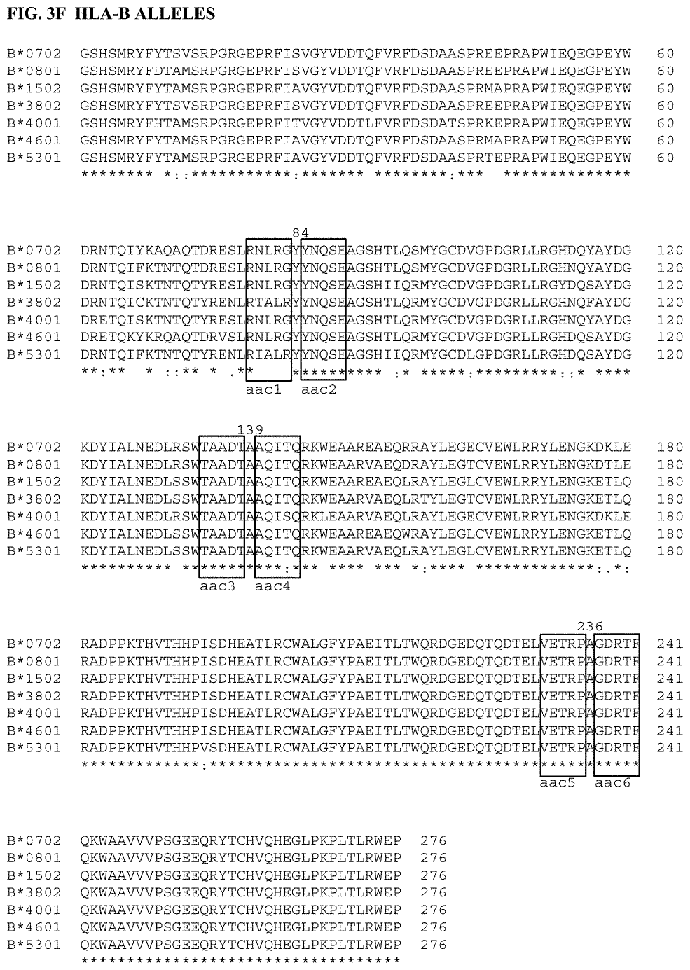

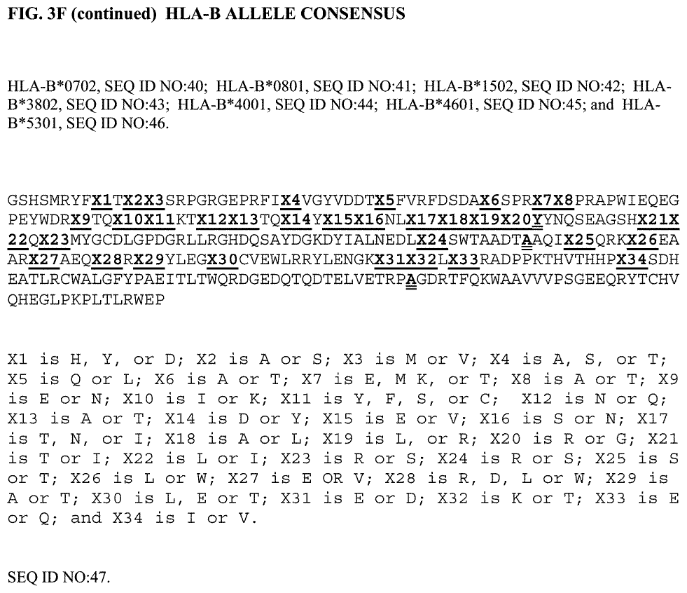

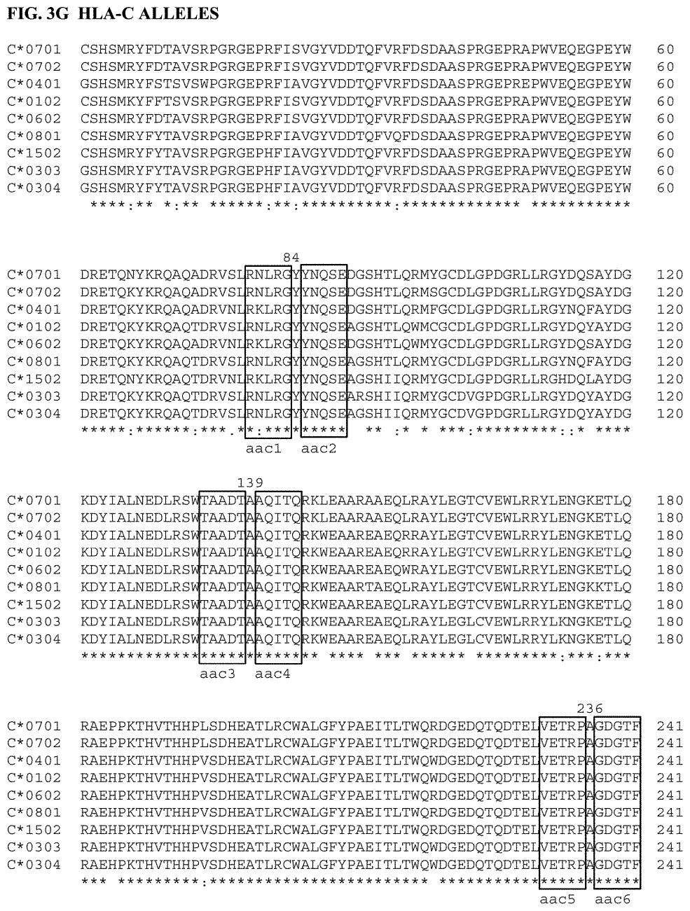

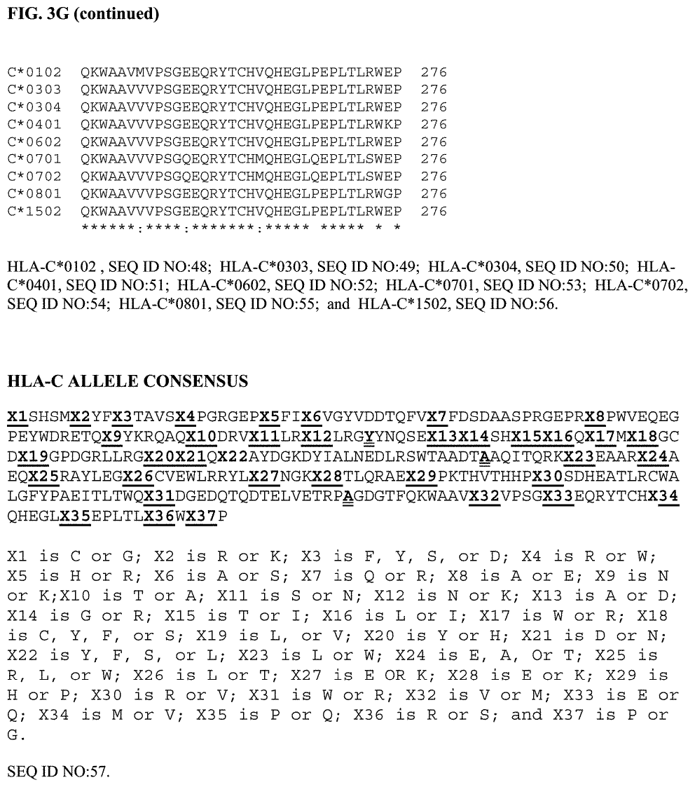

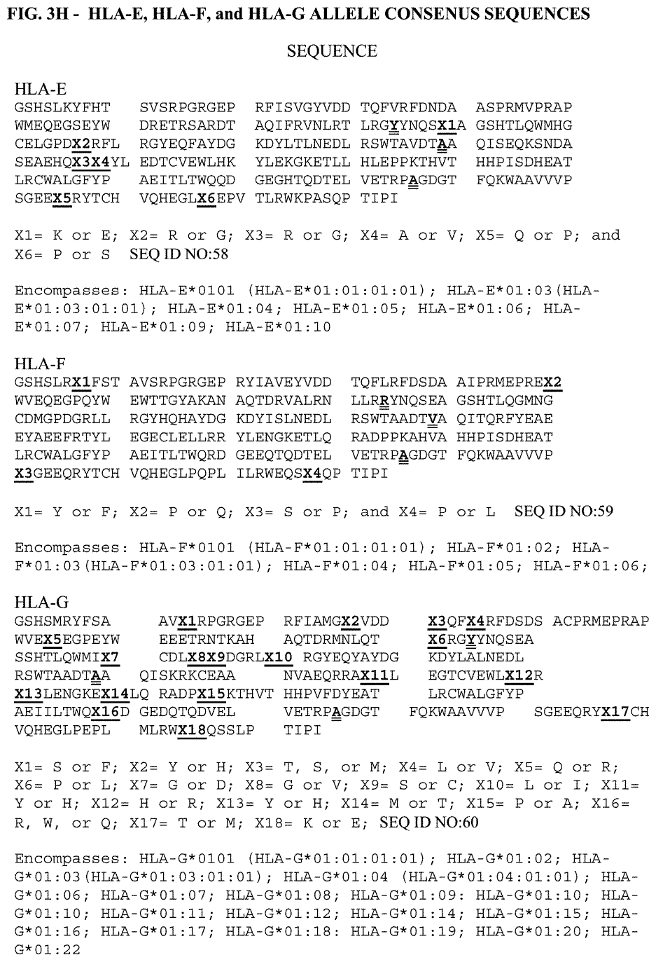

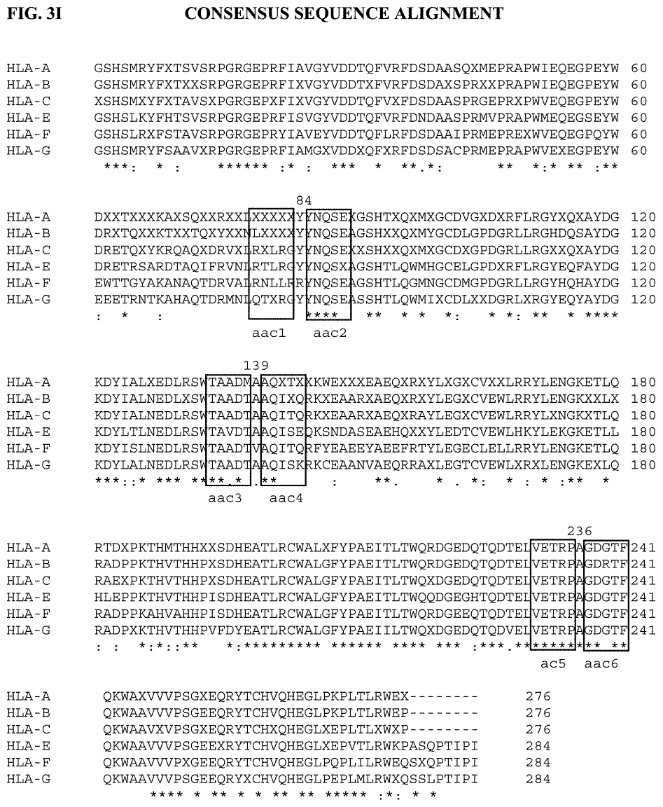

A third group of unconjugated T-Cell-MPs described herein appears as a duplex between a first T-Cell-MP and a second T-Cell-MP that associate through interactions in their scaffold sequences. Such unconjugated duplex T-Cell-MPs may have an identical first and second T-Cell-MP polypeptide sequence duplexed as a homodimer, or non-identical first and second T-Cell-MP polypeptide sequences duplexed as a heterodimer, with one or both of the T-Cell-MPs comprising from N-terminus to C-terminus:
(i) optionally one or more MOD polypeptide sequences (e.g., two or more MOD polypeptide sequences, such as in tandem, wherein when there are two or more MOD polypeptide sequences they are optionally joined to each other by independently selected L1 linkers);
(ii) an optional L2 polypeptide sequence joining the one or more optional MOD polypeptide sequences to a β2M polypeptide sequence;
(iii) the β2M polypeptide sequence;
(iv) an L3 linker polypeptide sequence comprising from 10 to 50 amino acids;
(v) a class I MHC-H polypeptide sequence comprising cysteines substituted at positions 84 and 139 (see FIGS. 3E-3H, e.g., Y84C and A139C substitutions) and forming a disulfide bond;
(vi) an L4 linker polypeptide sequence;
(vii) an interspecific or non-interspecific immunoglobulin Fc scaffold sequence;
(viii) an L5 linker polypeptide sequence; and
(ix) optionally one or more MOD polypeptide sequence (e.g., two or more MOD polypeptide sequences, such as in tandem, wherein when there are two or more MOD polypeptide sequences they are optionally joined to each other by independently selected L6 linkers);
wherein at least one of the β2M polypeptide sequence, the L3 linker polypeptide sequence, and/or the MHC-H polypeptide sequence comprises at least one chemical conjugation site, e.g., for epitope conjugation and/or payload conjugation; wherein at least one of the β2M polypeptide sequence, the L3 linker polypeptide sequence, or the MHC-H polypeptide sequence comprises a chemical conjugation site that does not appear in a wt. sequence; and
wherein the first and second T-Cell-MPs are optionally covalently linked through at least one disulfide bond between their Ig Fc scaffold sequence. The chemical conjugation site should be suitable for epitope conjugation in that it does not interfere with the interactions of the T-Cell-MP with a TCR and is preferably solvent accessible permitting its conjugation to the epitope.

The chemical conjugation sites for epitope conjugation to T-Cell-MPs, including those of the above-mentioned first, second, and third groups of unconjugated T-Cell-MPs, permit the covalent attachment of an epitope presenting molecule (e.g., a peptide epitope) to the T-Cell-MP such that it can be bound (located in the binding cleft) by the MHC-H polypeptide and presented to a TCR. The chemical conjugation sites of an unconjugated T-Cell-MP may be one that does not appear in a wt. sequence (e.g., they are created using the techniques of protein engineering based in biochemistry and/or molecular biology). The chemical conjugation site should also be suitable for epitope conjugation in that it does not interfere with the interactions of the T-Cell-MP with a TCR, and is preferably solvent accessible, permitting its conjugation to the epitope.

It is understood that the unconjugated T-Cell-MPs do not comprise a peptide epitope (either covalently attached to, or as a fusion with, the T-Cell-MP polypeptide) that can be located in the binding cleft of the MHC-H/β2M polypeptide sequences and presented to a TCR. The disclosure does, however, include and provide for T-Cell-MP-epitope conjugates further comprising a molecule presenting an epitope that is directly or indirectly (e.g., through a peptide or non-peptide linker) covalently attached to the T-Cell-MP at a chemical conjugation site; where the epitope can also be associated with (located in or positioned in) the binding cleft of the T-Cell-MP MHC-H polypeptide sequence and functionally presented to a T cell bearing a TCR specific for the epitope, leading to TCR mediated activation or inhibition of the T cell.

The disclosure also provides T-Cell-MPs in which the epitope present in a T-Cell-MP-epitope conjugate of the present disclosure may bind to a TCR (e.g., on a T cell) with an affinity of at least 100 micro molar (μM) (e.g., at least 10 μM, at least 1 μM, at least 100 nM, at least 10 nM, or at least 1 nM).

A T-Cell-MP-epitope conjugate may bind to a first T cell with an affinity that is higher than the affinity with which the T-Cell-MP-epitope conjugate binds to a second T cell; where the first T cell expresses on its surface a Co-MOD and a TCR that binds the epitope, and where the second T cell expresses on its surface the same Co-MOD present on the first T cell, but does not express on its surface a TCR that binds the epitope (e.g., as tightly as the TCR of the first cell if it binds at all). See FIG. 1. The increased affinity may be measured in binding assays or inferred from the concentration of the T-Cell-MP epitope conjugate required to stimulate the first as compared to the second T cell. The increased affinity for epitope-specific T cells permits the use of the epitope conjugates as agents for clinical testing, diagnostics, and as therapeutics capable of directing epitope-specific T cell actions.

MODs present in T cell-MPs are independently selected wt. MODs and/or variant MODs. Where the T cell-MP forms a heteromeric complex, such as through the use of interspecific scaffold polypeptide sequences, the MODs presented in at least one of the T-Cell-MPs of the heteromer may be selected independently from the other T-Cell-MPs of the heteromeric complex. Accordingly, a heterodimeric duplex T-Cell-MP may have independently selected MODs that are different in the first and second T-Cell-MPs of the duplex. MODs in one aspect are selected to be one or more activating wt. MODs and/or variant MODs capable of stimulating epitope-specific T cell activation/proliferation (e.g., wt. and/or variant IL-2, 4-1BBL and/or CD80). In another embodiment, the MODs are one or more inhibitory wt. MODs and/or variant MODs capable of inhibiting T cell activation/proliferation (e.g., FAS-L and/or PD-L1). When used in conjunction with a T-Cell-MP bearing a suitable epitope, such activating or inhibitory MODs are capable of epitope-specific T cell action, particularly where the MODs are variant MODs and the MHC-epitope-TCR interaction is sufficiently strong to dominate the interaction of the T-Cell-MP with the T cells.

### 2 Chemical Conjugation Sites of Unconjugated T-Cell-MPs

The term “chemical conjugation site” means any suitable site of a T-Cell-MP that permits the selective formation of a direct or indirect (through an intervening linker or spacer) covalent linkage between the T-Cell-MP and an epitope- or payload-containing molecule. Chemical conjugation sites of unconjugated T-Cell-MPs may be (i) active, i.e., capable of forming a direct or indirect (through an intervening linker or spacer) covalent linkage between the T-Cell-MP and an epitope or payload without an additional chemical reaction or transformation of the chemical conjugation site (e.g., a solvent-accessible cysteine sulfhydryl), or (ii) nascent, i.e., requiring a further chemical reaction or enzymatic transformation of the chemical conjugation site to become an active chemical conjugation site (e.g., a sulfatase sequence not yet activated by an fGly enzyme).

The term “selective formation” means that when an epitope- or payload-containing molecule bearing a moiety that is reactive with an active chemical conjugation site of a T-Cell-MP, the epitope- or payload-containing molecule will be covalently bound to the chemical conjugation site in an amount higher than to any other site in the T-Cell-MP.

Chemical conjugation sites may be introduced into a T-Cell-MP using protein engineering techniques (e.g., by use of an appropriate nucleic acid sequence) to achieve a T-Cell-MP having a desired aa sequence. Chemical conjugation sites can be individual aas (e.g., a cysteine or lysine) or aa sequences (e.g., sulfatase, sortase or transglutaminase sequences) in a protein or polypeptide sequence of the T-Cell-MP.

Where the protein or polypeptide sequence of the T-Cell-MP is derived from a naturally occurring protein (e.g., the B2M, MHC-H or an IgG scaffold), the chemical conjugation site may be a site not appearing in the naturally occurring sequence, such as a site resulting from amino acid substitutions (e.g., cysteine substitutions), insertions, and or deletions. The chemical conjugation site may also be a sequence, or part of a sequence, that is not derived from a naturally occurring protein, such as a linker sequence (e.g., the L3 linker of a T-Cell-MP connecting the β2M and MHC-H polypeptide sequences of a T-Cell-MP).

In some embodiments, there is only one chemical conjugation site (e.g., one chemical conjugation site added by protein engineering) in each unconjugated T-Cell-MP polypeptide that permits an epitope to be covalently attached such that it can be located in the MHC polypeptide binding cleft and presented to a TCR. Each individual unconjugated T-Cell-MP may comprise more than one chemical conjugation sites that are selected to be either the same or different types of chemical conjugation sites, thereby permitting the same or different molecules (e.g., an epitope and one or more payloads) to be selectively conjugated to each of the chemical conjugation sites. Accordingly, each individual or duplexed unconjugated T-Cell-MP may comprise one or more chemical conjugations sites that are selected to be either the same or different types of chemical conjugation sites, thereby permitting the same or different molecules to be selectively conjugated to each of the chemical conjugation sites. The chemical conjugations sites (e.g., for the conjugation of epitope) generally will be the same (e.g., of the same type) so that epitope presenting molecules can be covalently attached to all of the desired sites in, for example, a duplex unconjugated T-Cell-MP, using a single reaction. T-Cell-MPs may contain chemical conjugation sites in addition to those for the conjugation to an epitope, including conjugation sites for the incorporation of, for example, targeting sequences and/or payloads such as labels.

Chemical conjugation sites used to incorporate molecules other than epitope presenting molecules will, in most instances, be of a different type (e.g., utilize different chemical reactions) and in different locations than the sites used to incorporate epitopes, thereby permitting different molecules to be selectively conjugated to each of the polypeptides. Where a T-Cell-MP is to comprise a targeting sequence and/or one or more payload molecules, the unconjugated T-Cell-MP may comprise more than one copy of a chemical conjugation site (e.g., chemical conjugation sites added by protein engineering) to permit attachment to multiple molecules of targeting sequence and/or payload.

Chemical conjugation sites that may be incorporated into unconjugated T cell-MP polypeptides, include, but are not limited to:
a) peptide sequences that act as enzyme modification sequences (e.g., sulfatase, sortase, and/or transglutaminase sequences);
b) non-natural aas and/or selenocysteines;
c) chemical conjugation sites comprising individual amino acids;
d) carbohydrate or oligosaccharide moieties; and
e) IgG nucleotide binding sites.

a. Sulfatase Motifs

In those embodiments where enzymatic modification is chosen as the means of chemical conjugation, the chemical conjugation site(s) may comprise a sulfatase motif. Sulfatase motifs are usually 5 or 6 aas in length, and are described, for example, in U.S. Pat. No. 9,540,438 and U.S. Pat. Pub. No. 2017/0166639 A1, which are incorporated by reference. Insertion of the motif results in the formation of a protein or polypeptide that is sometimes referred to as aldehyde tagged or having an aldehyde tag. The motif may be acted on by formylglycine generating enzyme(s) (“FGE” or “FGEs”) to convert a cysteine or serine in the motif to a formylglycine residue (“fGly” although sometimes denoted “FGly”), which is an aldehyde containing aa, sometimes referred to as oxoalanine, that may be utilized for selective (e.g., site specific) chemical conjugation reactions. Accordingly, as used herein, “aldehyde tag” or “aldehyde tagged” polypeptides refer to an aa sequence comprising an unconverted sulfatase motif, as well as to an aa sequence comprising a sulfatase motif in which the cysteine or the serine residue of the motif has been converted to fGly by action of an FGE. Where the term sulfatase motif is utilized in the context of an aa sequence, both the nascent chemical conjugation sequence (e.g., a polypeptide containing the unconverted motif) as well as its fGly containing the active chemical conjugation site counterpart are disclosed. Once present in a polypeptide (e.g., of a T-Cell-MP), a fGly residue may be reacted with molecules (e.g., peptide epitopes with or without an intervening linker) comprising a variety of reactive groups including, but not limited to, thiosemicarbazide, aminooxy, hydrazide, and hydrazino groups to form a conjugate (e.g., a T-Cell-MP-epitope conjugate) having a covalent bond between the peptide and the molecule via the fGly residue. Sulfatase motifs may be used to incorporate not only epitopes (e.g., epitope presenting peptides), but also targeting sequences and/or payloads (e.g., in the formation of conjugates with drugs and diagnostic molecules).

In embodiments, the sulfatase motif is at least 5 or 6 aa residues, but can be, for example, from 5 to 16 (e.g., 6-16, 5-14, 6-14, 5-12, 6-12, 5-10, 6-10, 5-8, or 6-8) aas in length. The sulfatase motif may be limited to a length less than 16, 14, 12, 10, or 8 aa residues.

In an embodiment, the sulfatase motif comprises the sequence of Formula (I): X1Z1X2Z2X3Z3 (SEQ ID NO:66), where
Z1 is cysteine or serine;
Z2 is either a proline or alanine residue (which can also be represented by “P/A”);
Z3 is a basic aa (arginine, lysine, or histidine, usually lysine), or an aliphatic aa (alanine, glycine, leucine, valine, isoleucine, or proline, usually A, G, L, V, or I);
X1 is present or absent and, when present, can be any aa, though usually an aliphatic aa, a sulfur-containing aa, or a polar uncharged aa (e.g., other than an aromatic aa or a charged aa), usually L, M, V, S or T, more usually L, M, S or V, with the proviso that, when the sulfatase motif is at the N-terminus of the target polypeptide, X1 is present; and
X2 and X3 independently can be any aa, though usually an aliphatic aa, a polar, uncharged aa, or a sulfur containing aa (e.g., other than an aromatic aa or a charged aa), usually S, T, A, V, G or C, more usually S, T, A, V or G.

As indicated above, a sulfatase motif of an aldehyde tag is at least 5 or 6 aa residues, but can be, for example, from 5 to 16 aas in length. The motif can contain additional residues at one or both of the N- and C-termini, such that the aldehyde tag includes both a sulfatase motif and an “auxiliary motif.” In an embodiment, the sulfatase motif includes a C-terminal auxiliary motif (i.e., following the Z3 position of the motif).

A variety of FGEs may be employed for the conversion (oxidation) of cysteine or serine in a sulfatase motif to fGly. As used herein, the term formylglycine generating enzyme, or FGE, refers to fGly-generating enzymes that catalyze the conversion of a cysteine or serine of a sulfatase motif to fGly. As discussed in U.S. Pat. No. 9,540,438, the literature often uses the term formylglycine-generating enzymes for those enzymes that convert a cysteine of the motif to fGly, whereas enzymes that convert a serine in a sulfatase motif to fGly are referred to as Ats-B-like.

Sulfatase motifs of Formula (I) amenable to conversion by a prokaryotic FGE often contain a cysteine or serine at Z1 and a proline at Z2 that may be modified either by the “SUMP I-type” FGE or the “Ats-B-like” FGE, respectively. Prokaryotic FGE enzymes that may be employed include the enzymes from Clostridium perfringens (a cysteine type enzyme), Klebsiella pneumoniae (a Serine-type enzyme) or the FGE of Mycobacterium tuberculosis. Where peptides containing a sulfatase motif are being prepared for conversion into fGly-containing peptides by a eukaryotic FGE, for example by expression and conversion of the peptide in a eukaryotic cell or cell-free system using a eukaryotic FGE, sulfatase motifs amenable to conversion by a eukaryotic FGE may advantageously be employed.

Host cells for production of polypeptides with unconverted sulfatase motifs, or where the cell expresses a suitable FGE for converting fGly-containing polypeptide sequences, include those of a prokaryotic and eukaryotic organism. Non-limiting examples include Escherichia coli strains, Bacillus spp. (e.g., B. subtilis, and the like), yeast or fungi (e.g., S. cerevisiae, Pichia spp., and the like). Examples of other host cells, including those derived from a higher organism such as insects and vertebrates, particularly mammals, include, but are not limited to, HeLa cells (e.g., American Type Culture Collection (ATCC) No. CCL-2), CHO cells (e.g., ATCC Nos. CRL9618 and CRL9096), CHO DG44 cells, CHO-K1 cells (ATCC CCL-61), 293 cells (e.g., ATCC No. CRL-1573), Vero cells, NIH 3T3 cells (e.g., ATCC No. CRL-1658), Hnh-7 cells, BHK cells (e.g., ATCC No. CCL1O), PC12 cells (ATCC No. CRL1721), COS cells, COS-7 cells (ATCC No. CRL1651), RAT1 cells, mouse L cells (ATCC No. CCLI.3), human embryonic kidney (HEK) cells (ATCC No. CRL1573), HLHepG2 cells, and the like.

Sulfatase motifs may be incorporated into any desired location of a T-Cell-MP. In an embodiment they may be excluded from the amino or carboxyl terminal 10 or 20 amino acids. In an embodiment, a sulfatase motif may be added in (e.g., at or near the terminus) of any T-Cell-MP element, including the MHC-H or β2M polypeptide sequences or any linker sequence joining them (the L3 linker). Sulfatase motifs may also be added to the scaffold polypeptide (e.g., the Ig Fc) or any of the linkers present in the T-Cell-MP (e.g., L1 to L6).

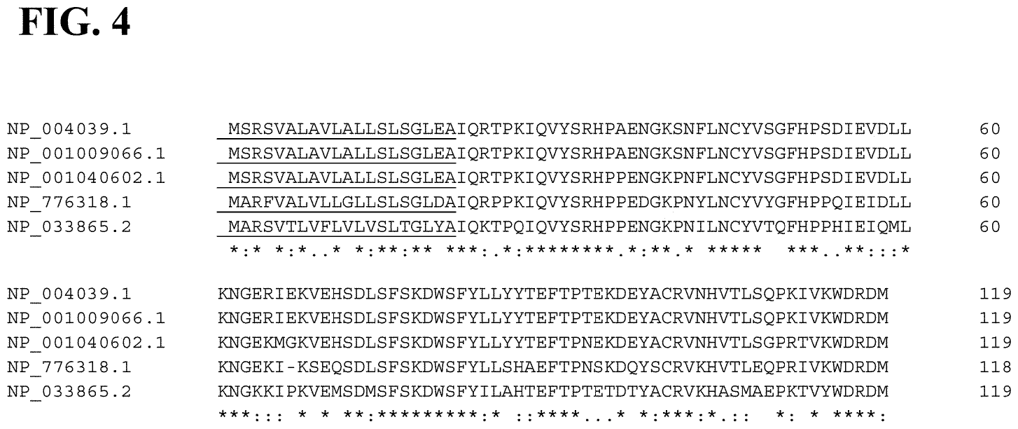

A sulfatase motif may be incorporated into, or attached to (e.g., via a peptide linker), a β2M polypeptide in a T-Cell-MP with a sequence having at least 85% (e.g., at least 90%, 95%, 98% or 99%, or even 100%) aa sequence identity to at least 50 (e.g., at least 60, 70, 80, 90, 96, 97, or 98 or all) contiguous aas of a mature β2M polypeptide sequence shown in FIG. 4 (e.g., the sequences shown in FIG. 4 starting at aa 21 and ending at their C-terminus). The mature human β2M polypeptide sequence in FIG. 4 may be selected for incorporation of the sulfatase motif. Sequence identity to the β2M polypeptides is determined relative to the corresponding portion of a β2M polypeptide in FIG. 4 without consideration of the added sulfatase motif or any linker or other sequences present.

In an embodiment, a sulfatase motif may be incorporated into a β2M polypeptide sequence having 1 to 15 (e.g., 1, 2, 3, 4, 5, 6, 7, 8, 9, 10, 11, 12, 13, 14, or 15) aa deletions, insertions and/or changes compared with a sequence shown in FIG. 4 (either an entire sequence shown in FIG. 4, or the sequence of a mature polypeptide starting at aa 21 and ending at its C-terminus). Changes are assessed without consideration of the aas of the sulfatase motif and any linker sequences present. In one such embodiment a sulfatase motif may be placed and/or be inserted within aas 1-15, 15-35, 35-55, 40-50, or 50-70 of a mature β2M sequence, such as those shown in FIG. 4. In one embodiment, sulfatase motifs may be located between aas 35-55 (e.g., between aas 40 to 50) of the human mature β2M polypeptide sequence of FIG. 4 and may have 0 to 15 aa substitutions compared with a sequence shown in FIG. 4 (either an entire sequence shown in FIG. 4, or the sequence of a mature polypeptides starting at aa 21 and ending at its C-terminus).

A sulfatase motif may be incorporated into, or attached to (e.g., via a peptide linker), a MHC Class I heavy chain polypeptide sequence having at least 85% (e.g., at least 90%, 95%, 98% or 99%, or even 100%) aa sequence identity to at least 150, 175, 200, or 225 contiguous aas of a MHC-H sequence shown in FIGS. 3A to 3I before the addition of the sulfatase motif.

In an embodiment, the added sulfatase motif is attached to the N- or C-terminus of a T-Cell-MP or, if present, attached to or within a linker located at the N- or C-terminus of the T-Cell-MP.

U.S. Pat. No. 9,540,438 discusses the incorporation of sulfatase motifs into the various immunoglobulin sequences, including Fc region polypeptides, and is herein incorporated by reference for its teachings on sulfatase motifs and modification of Fc polypeptides and other polypeptides. That patent is also incorporated by reference for its guidance on FGE enzymes, and their use in forming fGly residues, as well as the chemistry related to the coupling of molecules such as epitopes and payloads to fGly residues.

The incorporation of a sulfatase motif may be accomplished by incorporating a nucleic acid sequence encoding the motif at the desired location in a nucleic acid encoding a T-Cell-MP. As discussed below, the nucleic acid sequence may be placed under the control of a transcriptional regulatory sequence(s) (a promoter) and provided with regulatory elements that direct its expression. The expressed protein may be treated with one or more FGEs after expression and partial or complete purification. Alternatively, expression of the nucleic acid in cells that express a FGE that recognizes the sulfatase motif results in the conversion of the cysteine or serine of the motif to fGly.

In view of the foregoing, this disclosure provides for T-Cell-MPs comprising one or more fGly residues incorporated into a T-Cell-MP polypeptide chain as discussed above. The fGly residues may, for example, be in the context of the sequence X1(fGly)X2Z2X3Z3, where: fGly is the formylglycine residue; and Z2, Z3, X1, X2 and X3 are as defined in Formula (I) above. Epitopes and/or payloads may be conjugated either directly or indirectly to the reactive formyl glycine of the sulfatase motif directly or through a peptide or chemical linker. After chemical conjugation the T-Cell-MPs comprise one or more fGly′ residues incorporated in the context of the sequence X1(fGly′)X2Z2X3Z3, where the fGly′ residue is formylglycine that has undergone a chemical reaction and now has a covalently attached epitope or payload.

A number of chemistries and commercially available reagents can be utilized to conjugate a molecule (e.g., an epitope or payload) to a fGly residue, including, but not limited to, the use of thiosemicarbazide, aminooxy, hydrazide, or hydrazino derivatives of the molecules to be coupled at a fGly-containing chemical conjugation site. For example, epitopes (e.g., peptide epitopes) and/or payloads bearing thiosemicarbazide, aminooxy, hydrazide, hydrazino or hydrazinyl functional groups (e.g., attached directly to an aa of a peptide or via a linker such as a PEG) can be reacted with fGly-containing T-Cell-MP polypeptides to form a covalently linked epitope. Similarly, targeting sequences and/or payloads such as drugs and therapeutics can be incorporated using, for example, biotin hydrazide as a linking agent.

The disclosure provides for methods of preparing conjugated T-Cell-MPs including T-Cell-MP-epitope conjugates and/or T-Cell-MP-payload conjugates comprising:
a) incorporating a nucleotide sequence encoding a sulfatase motif including a serine or cysteine (e.g., a sulfatase motif of Formula (I) or (II) such as X1CX2PX3Z3 (SEQ ID NO:67); CX1PX2Z3 (SEQ ID NO:68) discussed above) into a nucleic acid encoding an unconjugated T-Cell-MP;
b) expressing the sulfatase motif-containing unconjugated T-Cell-MP polypeptide in a cell that
   i) expresses a FGE and converts the serine or cysteine of the sulfatase motif to a fGly and partially or completely purifying the fGly-containing unconjugated T-Cell-MP, or
   ii) does not express a FGE that converts a serine or cysteine of the sulfatase motif to a fGly, and purifying or partially purifying the T-Cell-MP containing the sulfatase motif and contacting the purified or partially purified T-Cell-MP with a FGE that converts the serine or cysteine of the sulfatase motif into a fGly residue; and
c) contacting the fGly-containing polypeptides with an epitope and/or payload that has been functionalized with a group that forms a covalent bond between the aldehyde of the fGly and the epitope and/or payload;
thereby forming a T-Cell-MP-epitope conjugate and/or T-Cell-MP payload conjugate.
   In such methods the epitope (epitope containing molecule) and/or payload may be functionalized by any suitable function group that reacts selectively with an aldehyde group. Such groups may, for example, be selected from the group consisting of thiosemicarbazide, aminooxy, hydrazide, and hydrazino. In an embodiment a sulfatase motif is incorporated into a second T-Cell-MP polypeptide comprising a β2M aa sequence with at least 85% (e.g., at least 90%, 95%, 98% or 99%, or even 100%) sequence identity to at least 60, 70, 80 or 90 contiguous aas of a β2M sequence shown in FIG. 4 (e.g., a mature β2M polypeptide with identity calculated without including or before the addition of the sulfatase motif sequence).

In an embodiment of the method of preparing a T-Cell-MP-epitope conjugate and/or T-Cell-MP payload conjugate, a sulfatase motif is incorporated into a polypeptide comprising a sequence having at least 85% (e.g., at least 90%, 95%, 98% or 99%, or even 100%) aa sequence identity to at least 150, 175, 200, or 225 contiguous aas of a sequence shown in FIGS. 3A-3I, with sequence identity calculated without including the addition of the sulfatase motif sequence).

b. Sortase A Enzyme Sites

Epitopes (e.g., peptides comprising the sequence of an epitope) and payloads may be attached at the N- and/or C-termini T-Cell-MP by incorporating sites for Sortase A conjugation at those locations.

Sortase A recognizes a C-terminal pentapeptide sequence LP(X5)TG/A (SEQ ID NO:69, with X5 being any single amino acid, and G/A being a glycine or alanine), and creates an amide bond between the threonine within the sequence and glycine or alanine in the N-terminus of the conjugation partner.

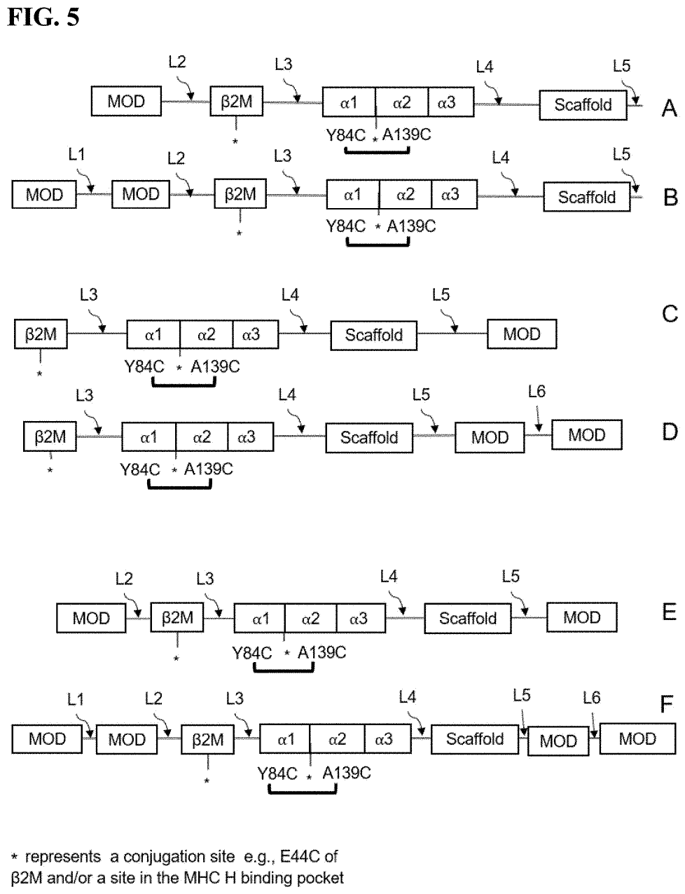

For attachment of epitopes or payloads to the C-terminal portion of a T-Cell-MP polypeptide a LP(X5)TG/A is provided in the carboxy terminal portion of the desired polypeptide(s), such as in an exposed L5 linker (see FIG. 5 structure A). An exposed stretch of glycines or alanines (e.g., (G)₃₋₅ (SEQ ID NOs:70 and 71 when using Sortase A from Staphylococcus aureus or alanines (A)₃₋₅, SEQ ID NOs:72 and 73 when using Sortase A from Streptococcus pyogenes) is provided at the N-terminus of a peptide that comprises an epitope (e.g., in a linker attached to the epitope), a peptide payload (or a linker attached thereto), or a peptide covalently attached to a non-peptide epitope or payload.

For attachment of epitopes or payloads to the amino terminus of a T-Cell-MP polypeptide, an aa sequence comprising an exposed stretch of glycines (e.g., (G)_{2, 3, 4, or 5}) or alanines (e.g., (A)_{2, 3, 4, or 5}) is provided at the N-terminus, and a LP(X5)TG/A is provided in the carboxy terminal portion of a peptide that comprises an epitope (or a linker attached thereto), a peptide payload (or a linker attached thereto), or a peptide covalently attached to a non-peptide epitope or payload.

Combining Sortase A with the amino and carboxy modified peptides described above results in a cleavage between the Thr and Gly/Ala residues in the LP(X5)TG/A sequence and formation of a covalently coupled complex of the form: carboxy-modified polypeptide-LP(X5)T*G/A-amino-modified polypeptide, where the “*” represents the bond formed between the threonine of the LP(X5)TG/A motif and the glycine or alanine of the N-terminal modified peptide.

In place of LP(X5)TG/A, a LPETGG (SEQ ID NO:74) peptide may be used for S. aureus Sortase A coupling, or a LPETAA (SEQ ID NO:75) peptide may be used for S. pyogenes Sortase A coupling. The conjugation reaction still occurs between the threonine and the amino terminal oligoglycine or oligoalanine peptide to yield a carboxy-modified polypeptide-LP(X5)T*G/A-amino-modified polypeptide, where the “*” represents the bond formed between the threonine and the glycine or alanine of the N-terminal modified peptide.

c. Transglutaminase Enzyme Sites

Transglutaminases (mTGs) catalyze the formation of a covalent bond between the amide group on the side chain of a glutamine residue and a primary amine donor (e.g., a primary alkyl amine, such as is found on the side chain of a lysine residue in a polypeptide). Transglutaminases may be employed to conjugate epitopes and payloads to T-Cell-MPs, either directly through a free amine, or indirectly via a linker comprising a free amine. As such, glutamine residues added to a T-Cell-MP in the context of a transglutaminase site may be considered as chemical conjugation sites when they can be accessed by enzymes such as Streptoverticillium mobaraense transglutaminase. That enzyme (EC 2.3.2.13) is a stable, calcium-independent enzyme catalyzing the γ-acyl transfer of glutamine to the ε-amino group of lysine. Glutamine residues appearing in a sequence are, however, not always accessible for enzymatic modification. The limited accessibility can be advantageous as it limits the number of locations where modification may occur. For example, bacterial mTGs are generally unable to modify glutamine residues in native IgG1s; however, Schibli and co-workers (Jeger, S., et al. Angew Chem (Int Engl). 2010; 49:99957 and Dennler P, et al. Bioconjug Chem. 2014; 25(3):569-78) found that deglycosylating IgG1 s at N297 rendered glutamine residue Q295 accessible and permitted enzymatic ligation to create an antibody drug conjugate. Further, by producing a N297 to Q297 IgG1 mutant, they introduced two sites for enzymatic labeling by transglutaminase. Modification at N297 also offers the potential to reduce the interaction of the IgG Fc reaction with complement C1q protein.

Where a T-Cell-MP does not contain a glutamine that may be employed as a chemical conjugation site (e.g., it is not accessible to a transglutaminase or not placed in the desired location), a glutamine residue may be added to a sequence to form a transglutaminase site, or a sequence comprising a transglutaminase accessible glutamine (sometimes referred to as a “glutamine tag” or a “Q-tag”), may be incorporated through protein engineering into the polypeptide. The added glutamine or Q-tag may act as a chemical conjugation site for epitopes or payloads. US Pat. Pub. No. 2017/0043033 A1 describes the incorporation of glutamine residues and Q-tags and the use of transglutaminase for modifying polypeptides and is incorporated herein for those teachings.

Incorporation of glutamine residues and Q-tags may be accomplished chemically where the peptide is synthesized, or by modifying a nucleic acid that encodes the polypeptide and expressing the modified nucleic acid in a cell or cell-free system. In embodiments, the glutamine-containing Q-tag comprises an aa sequence selected from the group consisting of LQG, LLQGG (SEQ ID NO:76), LLQG (SEQ ID NO:77), LSLSQG (SEQ ID NO:78), and LLQLQG (SEQ ID NO:79) (numerous others are available).

Glutamine residues and Q-tags may be incorporated into any desired location of a T-Cell-MP. In an embodiment, a glutamine residue or Q-tag may be added in (e.g., at or near the terminus of) any T-Cell-MP element, including the MHC-H or β2M polypeptide sequences or any linker sequence joining them (the L3 linker). Glutamine residues and Q-tags may also be added to the scaffold polypeptide (e.g., the Ig Fc) or any of the linkers present in the T-Cell-MP (e.g., L1 to L6).

A glutamine residue or Q-tag may be incorporated into, or attached to (e.g., via a peptide linker), a β2M polypeptide in a T-Cell-MP with a sequence having at least 85% (e.g., at least 90%, 95%, 98% or 99%, or even 100%) aa sequence identity to at least 50 (e.g., at least 60, 70, 80, 90, 96, 97, or 98 or all) contiguous aas of a mature β2M polypeptide sequence shown in FIG. 4 (e.g., the sequences shown in FIG. 4 starting at aa 21 and ending at their C-terminus). The mature human β2M polypeptide sequence in FIG. 4 may be selected for incorporation of the glutamine residue or Q-tag. Sequence identity to the β2M polypeptides is determined relative to the corresponding portion of a β2M polypeptide in FIG. 4 without consideration of the added glutamine residue, Q-tag, or any linker or other sequences present.

In an embodiment, a glutamine residue or Q-tag may be incorporated into a β2M polypeptide sequence having 1 to 15 (e.g., 1, 2, 3, 4, 5, 6, 7, 8, 9, 10, 11, 12, 13, 14, or 15) aa deletions, insertions and/or changes compared with a sequence shown in FIG. 4 (either an entire sequence shown in FIG. 4, or the sequence of a mature polypeptide starting at aa 21 and ending at its C-terminus). Changes are assessed without consideration of the aas of the glutamine residue, Q-tag and any linker sequences present. In one such embodiment a glutamine residue or Q-tag may be placed and/or be inserted within aas 1-15, 15-35, 35-55, 40-50, or 50-70 of a mature β2M sequence, such as those shown in FIG. 4. In one embodiment, a glutamine residue or Q-tag may be located between aas 35-55 (e.g., 40 to 50) of the human mature β2M polypeptide sequence of FIG. 4 and may have 0 to 15 aa substitutions.

A glutamine residue or Q-tag may be incorporated into, or attached to (e.g., via a peptide linker), a MHC Class I heavy chain polypeptide sequence having at least 85% (e.g., at least 90%, 95%, 98% or 99%, or even 100%) aa sequence identity to at least 150, 175, 200, or 225 contiguous aas of a MHC-H sequence shown in FIGS. 3A to 3I before the addition of the glutamine residue or Q-tag.

In an embodiment, the added glutamine residue or Q-tag is attached to the N- or C-terminus of a T-Cell-MP or, if present, attached to or within a linker located at the N- or C-terminus of the T-Cell-MP.

Payloads and epitopes that contain, or have been modified to contain, a primary amine group may be used as the amine donor in a transglutaminase-catalyzed reaction forming a covalent bond between a glutamine residue (e.g., a glutamine residue in a Q-tag) and the epitope or payload.

Where an epitope or payload does not comprise a suitable primary amine to permit it to act as the amine donor, the epitope or payload may be chemically modified to incorporate an amine group (e.g., modified to incorporate a primary amine by linkage to a lysine, aminocaproic acid, cadaverine etc.). Where an epitope or payload comprises a peptide and requires a primary amine to act as the amine donor, a lysine or another primary amine that a transglutaminase can act on may be incorporated into the peptide. Other amine containing compounds that may provide a primary amine group and that may be incorporated into, or at the end of, an alpha amino acid chain include, but are not limited to, homolysine, 2,7-diaminoheptanoic acid, and aminoheptanoic acid. Alternatively, the epitope or payload may be attached to a peptide or non-peptide linker that comprises a suitable amine group. Examples of suitable non-peptide linkers include an alkyl linker and a PEG (polyethylene glycol) linker.

Transglutaminase can be obtained from a variety of sources, including enzymes from: mammalian liver (e.g., guinea pig liver); fungi (e.g., Oomycetes, Actinomycetes, Saccharomyces, Candida, Cryptococcus, Monascus, or Rhizopus transglutaminases); myxomycetes (e.g., Physarum polycephalum transglutaminase); and/or bacteria including a variety of Streptoverticillium, Streptomyces, Actinomadura sp., Bacillus, and the like.

Q-tags may be created by inserting a glutamine or by modifying the aa sequence around a glutamine residue appearing in a Ig Fc, β2M, and/or MHC-H chain sequence appearing in a T-Cell-MP and used as a chemical conjugation site for addition of an epitope or payload. Similarly, Q-tags may be incorporated into the Ig Fc region as chemical conjugation sites that may be used for the conjugation of, for example, epitopes and/or payloads either directly or indirectly through a peptide or chemical linker bearing a primary amine d. Selenocysteine and Non-Natural Amino Acids as Chemical Conjugation Sites

One strategy for providing site-specific chemical conjugation sites into a T-Cell-MP polypeptide employs the insertion of aas with reactivity distinct from the naturally occurring proteinogenic L-amino acids present in the polypeptide. Such aas include, but are not limited to, selenocysteine (Sec), and the non-natural aas: acetylphenylalanine (p-acetyl-L-phenylalanine, pAcPhe); parazido phenylalanine; and propynyl-tyrosine. Thanos et al. in US Pat. Publication No. 20140051836 A1 discuss some other non-natural aas including O-methyl-L-tyrosine, O-4-allyl-L-tyrosine, tri-O-acetyl-GlcNAcβ-serine, isopropyl-L-phenylalanine, p-benzoyl-L-phenylalanine, L-phosphoserine, and p-propargyloxy-phenylalanine. Other non-natural aas include reactive groups such as, for example, amino, carboxy, acetyl, hydrazino, hydrazido, semicarbazido, sulfanyl, azido and alkynyl. See, e.g., US Pat. Publication No. 20140046030 A1.

In addition to directly synthesizing polypeptides in the laboratory, two methods utilizing stop codons have been developed to incorporate non-natural aas into proteins and polypeptides utilizing transcription-translation systems. The first incorporates selenocysteine (Sec) by pairing the opal stop codon, UGA, with a Sec insertion sequence. The second incorporates non-natural aas into a polypeptide generally through the use of amber, ochre, or opal stop codons. The use of other types of codons such as a unique codon, a rare codon, an unnatural codon, a five-base codon, and a four-base codon, and the use of nonsense and frameshift suppression have also been reported. See, e.g., US Pat. Publication No. 20140046030 A1 and Rodriguez et al., PNAS 103(23)8650-8655(2006). By way of example, the non-natural amino acid acetylphenylalanine may be incorporated at an amber codon using a tRNA/aminoacyl tRNA synthetase pair in an in vivo or cell-free transcription-translation system.

Incorporation of both selenocysteine and non-natural aas requires engineering the necessary stop codon(s) into the nucleic acid coding sequence of the T-Cell MP polypeptide at the desired location(s), after which the coding sequence is used to express the T-Cell-MP in an in vivo or cell-free transcription-translation system.

In vivo systems generally rely on engineered cell-lines to incorporate non-natural aas that act as bio-orthogonal chemical conjugation sites into polypeptides and proteins. See, e.g., International Published Application No. 2002/085923 entitled “In vivo incorporation of unnatural amino acids.” In vivo non-natural aa incorporation relies on a tRNA and an aminoacyl tRNA synthetase pair that is orthogonal to all the endogenous tRNAs and synthetases in the host cell. The non-natural aa of choice is supplemented to the media during cell culture or fermentation, making cell-permeability and stability important considerations.

Various cell-free synthesis systems provided with the charged tRNA may also be utilized to incorporate non-natural aas. Such systems include those described in US Pat. Publication No. 20160115487A1; Gubens et al., RNA. 2010 August; 16(8): 1660-1672; Kim, D. M. and Swartz, J. R. Biotechnol. Bioeng. 66:180-8 (1999); Kim, D. M. and Swartz, J. R. Biotechnol. Prog. 16:385-90 (2000); Kim, D. M. and Swartz, J. R. Biotechnol. Bioeng. 74:309-16 (2001); Swartz et al, Methods Mol. Biol. 267:169-82 (2004); Kim, D. M. and Swartz, J. R. Biotechnol. Bioeng. 85:122-29 (2004); Jewett, M. C. and Swartz, J. R., Biotechnol. Bioeng. 86:19-26 (2004); Yin, G. and Swartz, J. R., Biotechnol. Bioeng. 86:188-95 (2004); Jewett, M. C. and Swartz, J. R., Biotechnol. Bioeng. 87:465-72 (2004); Voloshin, A. M. and Swartz, J. R., Biotechnol. Bioeng. 91:516-21 (2005).

Once incorporated into the T-Cell-MP, epitopes and/or payload bearing groups reactive with the incorporated selenocysteine or non-natural aa are brought into contact with the T-Cell-MP under suitable conditions to form a covalent bond. By way of example, the keto group of the pAcPhe is reactive towards alkoxyamines, and via oxime coupling can be conjugated directly to alkoxyamine containing epitopes and/or payloads or indirectly to epitopes and payloads via an alkoxyamine containing linker. Selenocysteine reacts with, for example, primary alkyl iodides (e.g., iodoacetamide which can be used as a linker), maleimides, and methylsulfone phenyloxadiazole groups. Accordingly, epitopes and/or payloads bearing those groups or bound to linkers bearing those groups can be covalently bound to polypeptide chains bearing selenocysteines.

As discussed above for other chemical conjugation sites, selenocysteines and/or non-natural aas may be incorporated into any desired location in the T-Cell-MP. In an embodiment, selenocysteines and/or non-natural aas may be added in (e.g., at or near the terminus of) any T-Cell-MP element, including the MHC-H or β2M polypeptide sequences or any linker sequence joining them (the L3 linker). Selenocysteines and/or non-natural aas may also be added to the scaffold polypeptide (e.g., the Ig Fc) or any of the linkers present in the T-Cell-MP (e.g., L1 to L6).

Selenocysteines and non-natural aas may be incorporated into, or attached to (e.g., via a peptide linker), a β2M polypeptide in a T-Cell-MP with a sequence having at least 85% (e.g., at least 90%, 95%, 98% or 99%, or even 100%) aa sequence identity to at least 50 (e.g., at least 60, 70, 80, 90, 96, 97, or 98 or all) contiguous aas of a mature β2M polypeptide sequence shown in FIG. 4 (e.g., the sequences shown in FIG. 4 starting at aa 21 and ending at their C-terminus). The mature human β2M polypeptide sequence in FIG. 4, may be selected for incorporation of the selenocysteines and non-natural aas. Sequence identity to the β2M polypeptides is determined relative to the corresponding portion of a β2M polypeptide in FIG. 4 without consideration of the added selenocysteines, non-natural aas, or any linker or other sequences present.

In an embodiment, a selenocysteine(s) or non-natural aa(s) may be incorporated into a β2M polypeptide sequence having 1 to 15 (e.g., 1, 2, 3, 4, 5, 6, 7, 8, 9, 10, 11, 12, 13, 14, or 15) aa deletions, insertions and/or changes compared with a sequence shown in FIG. 4 (either an entire sequence shown in FIG. 4, or the sequence of a mature polypeptide starting at aa 21 and ending at its C-terminus). Changes are assessed without consideration of the selenocysteine(s), non-natural aa(s), and any linker sequences present. In one such embodiment, a selenocysteines or non-natural aa may be placed and/or be inserted within aas 1-15, 15-35, 35-55, 40-50, or 50-70 of a mature β2M sequence, such as those shown in FIG. 4. In one embodiment, selenocysteines or non-natural aas may be located between aas 35-55 (e.g., 40 to 50) of the human mature β2M polypeptide sequence of FIG. 4 and may have 0 to 15 aa substitutions.

A selenocysteine or non-natural aa may be incorporated into, or attached to (e.g., via a peptide linker), a MHC Class I heavy chain polypeptide sequence having at least 85% (e.g., at least 90%, 95%, 98% or 99%, or even 100%) aa sequence identity to at least 150, 175, 200, or 225 contiguous aas of a MHC-H sequence shown in FIGS. 3A to 3I before the addition of the selenocysteines or non-natural aas.

In an embodiment, the added selenocysteine(s) or non-natural aa(s) is attached to the N- or C-terminus of a T-Cell-MP or, if present, attached to or within a linker located at the N- or C-terminus of the T-Cell-MP. In one such embodiment they may be utilized as sites for the conjugation of, for example, epitopes, targeting sequences, and/or payloads conjugated to the T-Cell-MP either directly or indirectly through a peptide or chemical linker.

e. Amino Acid Chemical Conjugation Sites

Any of the variety of functionalities (e.g., —SH, —NH₃, —OH, —COOH and the like) present in the side chains of naturally occurring amino acids, or at the termini of polypeptides, can be used as chemical conjugation sites. This includes the side chains of lysine and cysteine, which are readily modifiable by reagents including N-hydroxysuccinimide and maleimide functionalities, respectively. The main disadvantages of utilizing such amino acid residues is the potential variability and heterogeneity of the products. For example, an IgG has over 80 lysines, with over 20 at solvent-accessible sites. See, e.g., McComb and Owen, AAPS J. 117(2): 339-351. Cysteines tend to be less widely distributed; they tend to be engaged in disulfide bonds, and may be inaccessible (e.g., not accessible by solvent or to molecules used to modify the cysteines), and not located where it is desirable to place a chemical conjugation site. It is, however, possible to selectively modify T-Cell-MP polypeptides to provide naturally occurring and, as discussed above, non-naturally occurring amino acids at the desired locations for placement of a chemical conjugation site. Modification may take the form of direct chemical synthesis of the polypeptides (e.g., by coupling appropriately blocked amino acids) and/or by modifying the sequence of a nucleic acid encoding the polypeptide following expression in a cell or cell-free system. Accordingly, this disclosure includes and provides for the preparation of the T-Cell-MP polypeptides by transcription/translation systems capable of incorporating a non-natural aa or natural aa (including selenocysteine) to be used as a chemical conjugation site for epitope or payload conjugation.

This disclosure includes and provides for the preparation of a portion of a T-Cell-MP by transcription/translation systems and joining to its C- or N-terminus a polypeptide bearing a non-natural aa or natural aa (including selenocysteine) prepared by, for example, chemical synthesis. The polypeptide, which may include a linker, may be joined by any suitable method including the use of a sortase as described above for peptide epitopes. In an embodiment, the polypeptide may comprise a sequence of 2, 3, 4, or 5 alanines or glycines that may serve for sortase conjugation and/or as part of a linker sequence.

A naturally occurring aa (e.g., a cysteine) to be used as a chemical conjugation site may be provided at any desired location of a T-Cell-MP. In an embodiment, the naturally occurring aa may be provided in (e.g., at or near the terminus of) any T-Cell-MP element, including the MHC-H or β2M polypeptide sequences or any linker sequence joining them (the L3 linker). Naturally occurring aa(s) may also be provided in the scaffold polypeptide (e.g., the Ig Fc) or any of the linkers present in the T-Cell-MP (e.g., L1 to L6).

A naturally occurring aa (e.g., a cysteine) may also be provided in (e.g., via protein engineering), or attached to (e.g., via a peptide linker), a β2M polypeptide in a T-Cell-MP with a sequence having at least 85% (e.g., at least 90%, 95%, 98% or 99%, or even 100%) aa sequence identity to at least 50 (e.g., at least 60, 70, 80, 90, 96, 97, or 98 or all) contiguous aas of a mature β2M polypeptide sequence shown in FIG. 4 (e.g., the sequences shown in FIG. 4 starting at aa 21 and ending at their C-terminus). The mature human β2M polypeptide sequence in FIG. 4 may be selected for incorporation of the naturally occurring aa. Sequence identity to the β2M polypeptides is determined relative to the corresponding portion of a β2M polypeptide in FIG. 4 without consideration of the added naturally occurring aa, any linker, or any other sequences present.

In an embodiment, a naturally occurring aa (e.g., a cysteine) may be provided, e.g., via protein engineering in a β2M polypeptide sequence having 1 to 15 (e.g., 1, 2, 3, 4, 5, 6, 7, 8, 9, 10, 11, 12, 13, 14, or 15) aa deletions, insertions and/or changes compared with a sequence shown in FIG. 4 (either an entire sequence shown in FIG. 4, or the sequence of a mature polypeptide starting at aa 21 and ending at its C-terminus). Changes are assessed without consideration of the aas of the naturally occurring aa, any linker, or other sequences present. In one such embodiment a naturally occurring aa (e.g., a cysteine) may be engineered (e.g., using the techniques of molecular biology) within aas 1-15, 15-35, 35-55, 40-50, or 50-70 of a mature β2M sequence, such as those shown in FIG. 4. In one embodiment, a naturally occurring aa (e.g., a cysteine) may be provided between aas 35-55 (e.g., between 40 and 50, between 42 and 48, between 43 and 45, or at aa 44) of the human mature β2M polypeptide sequence of FIG. 4 and may have 0 to 15 aa substitutions.

A naturally occurring aa (e.g., a cysteine) may be provided in, or attached to (e.g., via a peptide linker), a MHC Class I heavy chain polypeptide sequence having at least 85% (e.g., at least 90%, 95%, 98% or 99%, or even 100%) aa sequence identity to at least 150, 175, 200, or 225 contiguous aas of a MHC-H sequence shown in FIGS. 3A to 3I before the addition of the naturally occurring aa.

In an embodiment, the naturally occurring aa (e.g., a cysteine) may be attached to the N- or C-terminus of a T-Cell-MP, or attached to or within a linker, if present, located at the N- or C-terminus of the T-Cell-MP.

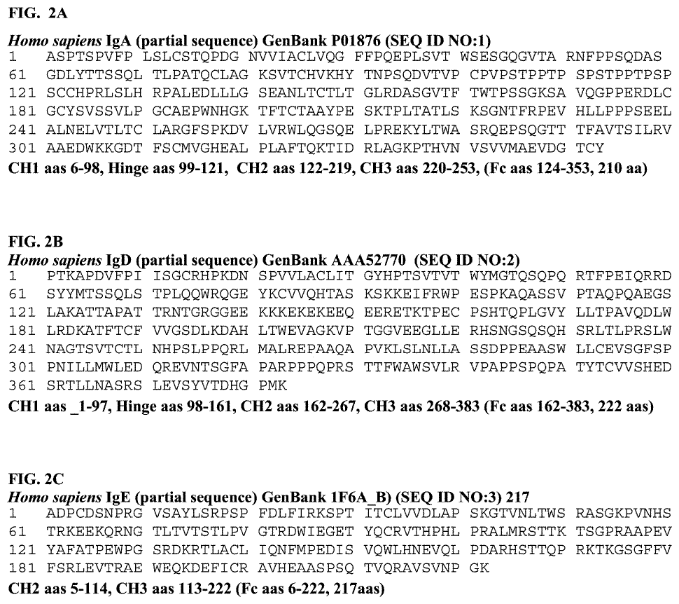

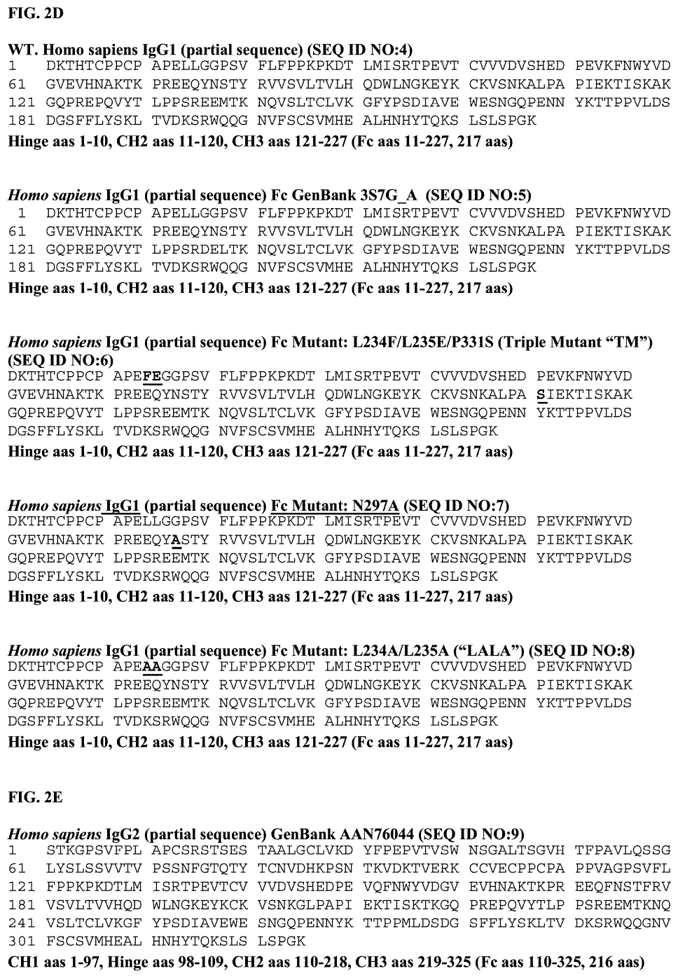

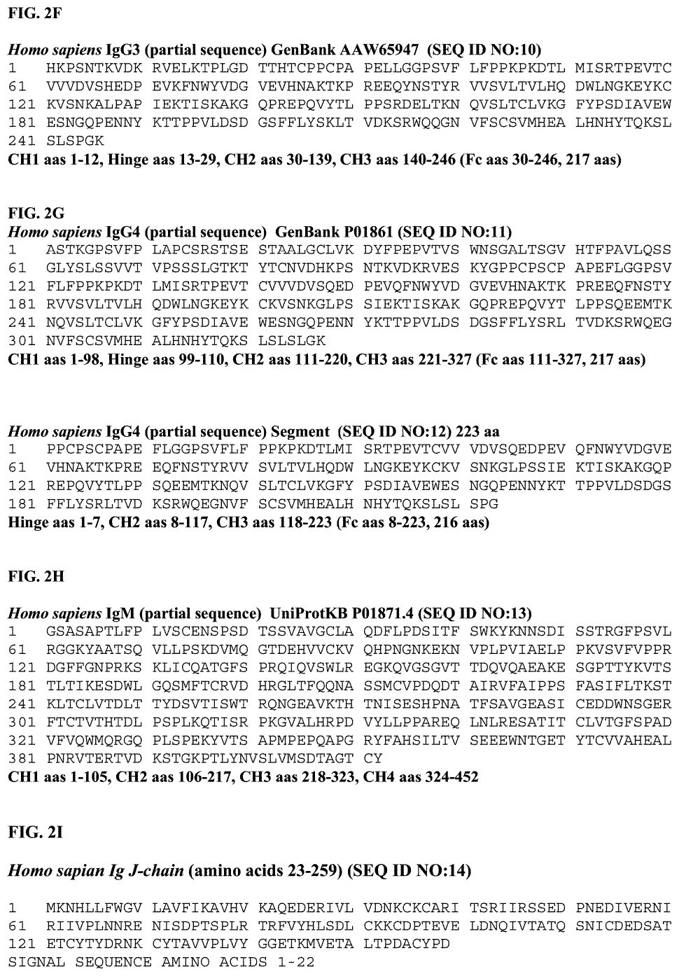

In one embodiment, a T-Cell-MP contains at least one naturally occurring aa (e.g., a cysteine) to be used as a chemical conjugation site provided, e.g., via protein engineering, in a β2M sequence as shown in FIG. 4, an Ig Fc sequence as shown in any of FIGS. 2A-G, or a MHC Class I heavy chain polypeptide as shown in FIGS. 3A-3I. In an embodiment, at least one naturally occurring aa to be used as a chemical conjugation site is provided in a polypeptide having at least 85% (e.g., at least 90%, 95%, 98% or 99%, or even 100%) aa sequence identity to at least 50 (e.g., at least 60, 70, 80, 90, 96, 97, or 98 or all) contiguous aas of a mature β2M sequence as shown in FIG. 4, an Ig Fc sequence as shown in FIG. 2, or at least 85% (e.g., at least 90%, 95%, 98% or 99%, or even 100%) aa sequence identity to at least 150, 175, 200, or 225 contiguous aas of a MHC Class I heavy chain polypeptide as shown in any of FIGS. 3A-3I. At least one naturally occurring aa (e.g., a cysteine) may be provided as a chemical conjugation site in a T-Cell-MP β2M aa sequence having at least 90% (e.g., at least 93%, 95%, 98% or 99%, or even 100%) aa sequence identity with at least the amino terminal 10, 20, 30, 40, 50, 60 or 70 aas of a mature β2M sequence as shown in FIG. 4. At least one naturally occurring aa (e.g., a cysteine) may be provided as a chemical conjugation site in a T-Cell-MP Ig Fc sequence (e.g., as shown in any of FIGS. 2A-2G). At least one naturally occurring aa (e.g., a cysteine) may be provided as a chemical conjugation site in a T-Cell-MP MHC Class I heavy chain polypeptide sequence having at least 85% (e.g., at least 90%, 95%, 98% or 99%, or even 100%) aa sequence identity to at least 150, 175, 200, or 225 contiguous aas of a MHC H polypeptide sequence provided in any of FIGS. 3A to 3I. In another embodiment, at least one naturally occurring aa to be used as a chemical conjugation site is provided in a T-Cell-MP polypeptide comprising at least 30, 40, 50, 60, 70, 80, 90, or 100 contiguous aas having 100% aa sequence identity to a MHC Class I heavy chain sequence as shown in any of FIGS. 3A to 3I or a mature β2M sequence as shown in FIG. 4.

In any of the embodiments mentioned above where a naturally occurring aa is provided, e.g., via protein engineering, in a polypeptide, the aa may be selected from the group consisting of arginine, lysine, cysteine, serine, threonine, glutamic acid, glutamine, aspartic acid, and asparagine. Alternatively, the aa provided as a conjugation site is selected from the group consisting of lysine, cysteine, serine, threonine, and glutamine. The aa provided as a conjugation site may also be selected from the group consisting of lysine, glutamine, and cysteine. In one instance, the provided aa is cysteine. In another instance, the provided aa is lysine. In still another instance, the provided aa is glutamine.

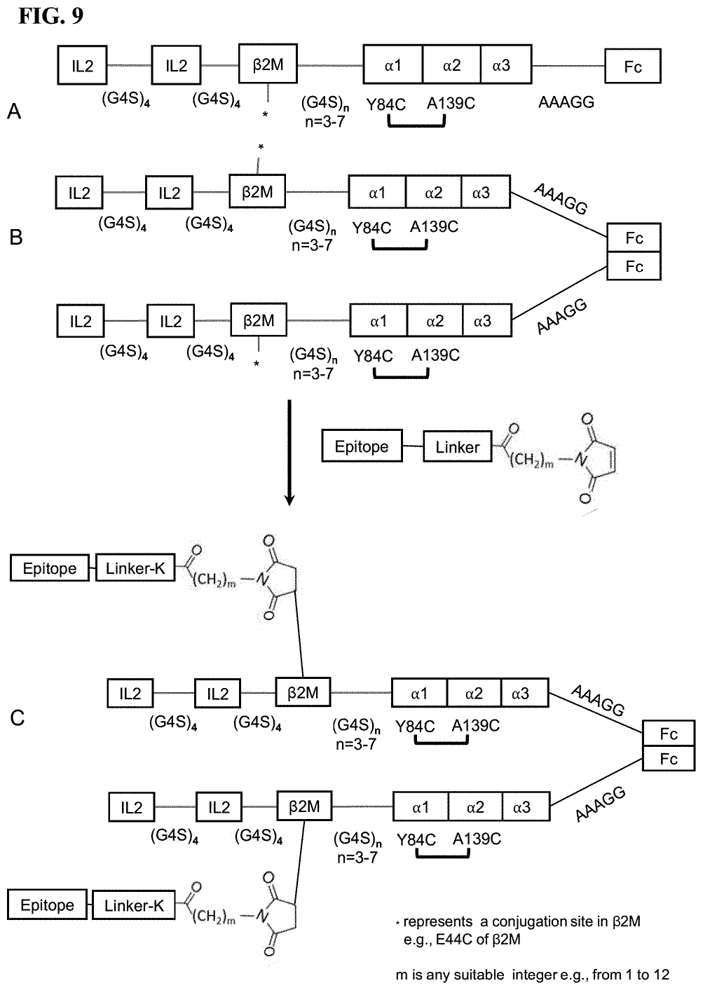

Any method known in the art may be used to couple payloads or epitopes to amino acids provided in an unconjugated T-Cell-MP. By way of example, maleimides may be utilized to couple to sulfhydryls, N-hydroxysuccinimide may be utilized to couple to amine groups, acid anhydrides or chlorides may be used to couple to alcohols or amines, and dehydrating agents may be used to couple alcohols or amines to carboxylic acid groups. Accordingly, using such chemistry an epitope or payload may be coupled directly, or indirectly through a linker (e.g., a homo- or hetero-bifunctional crosslinker), to a location on an unconjugated T-Cell-MP polypeptide. A number of bifunctional crosslinkers may be utilized, including, but not limited to, those described for linking a payload to a T-Cell-MP described herein below. For example, a peptide epitope (or a peptide-containing payload) including a maleimide group attached by way of a homo- or hetero-bifunctional linker (see, e.g., FIG. 9) or a maleimide amino acid can be conjugated to a sulfhydryl of a chemical conjugation site (e.g., a cysteine residue) that is naturally occurring or provided in a T-Cell-MP.

Maleimido amino acids can be incorporated directly into peptides (e.g., peptide epitopes) using a Diels-Alder/retro-Diels-Alder protecting scheme as part of a solid phase peptide synthesis. See, e.g., Koehler, Kenneth Christopher (2012), “Development and Implementation of Clickable Amino Acids,” Chemical & Biological Engineering Graduate Theses & Dissertations, 31, https://scholar.colorado.edu/chbe_gradetds/31.

A maleimide group may also be appended to an epitope (e.g., a peptide epitope) using a homo- or hetero-bifunctional linker (sometimes referred to as a crosslinker) that attaches a maleimide directly (or indirectly, e.g., through an intervening linker that may comprise additional aas bound to the epitope) to the epitope (e.g., peptide epitope). For example, a heterobifunctional N-hydroxysuccinimide-maleimide crosslinker can attach maleimide to an amine group of a peptide lysine. Some specific crosslinkers include molecules with a maleimide functionality and either a N-hydroxysuccinimide ester (NHS) or N-succinimidyl group that can attach a maleimide to an amine (e.g., an epsilon amino group of lysine). Examples of such crosslinkers include, but are not limited to, NHS-PEG4-maleimide, γ-maleimide butyric acid N-succinimidyl ester (GMBS); ε-maleimidocaproic acid N-hydroxysuccinimide ester (EMCS); m-maleimide benzoyl-N-hydroxysuccinimide ester (MBS); and N-(α-maleimidoacetoxy)-succinimide ester (AMAS), which offer different lengths and properties for peptide immobilization. Other amine reactive crosslinkers that incorporate a maleimide group include N-succinimidyl 4-(2-pyridyldithio) butanoate (SPDB). Additional crosslinkers (bifunctional agents) are recited below. In an embodiment the epitopes coupled to the T-Cell-MP have a maleimido alkyl carboxylic acid coupled to the peptide by an optional linker (see, e.g., FIG. 9), coupled, for example, by an amide formed with the epsilon amino group of a lysine. The maleimido carboxylic acid can be, for example, a maleimido ethanoic, propanoic, butanoic, pentanoic, hexanoic, heptanoic, or octanoic acid.

A peptide epitope may be coupled to a naturally occurring cysteine present or provided in (e.g., engineered into), for example, the binding pocket of a T-Cell-MP through a bifunctional linker comprising a maleimide or a maleimide amino acid incorporated into the peptide, thereby forming a T-Cell-MP epitope conjugate. A peptide epitope may be conjugated (e.g., by one or two maleimide amino acids or at least one maleimide containing bifunctional linker) to a MHC heavy chain having cysteine residues at any one or more locations within or adjacent to the MHC-H binding pocket. By way of example, a peptide epitope comprising maleimido amino acids or bearing a maleimide group as part of a crosslinker attached to the peptide may be covalently attached at 1 or 2 aas (e.g., cysteines) at MHC-H positions 2, 5, 7, 59, 84, 116, 139, 167, 168, 170, and/or 171 (e.g., Y7C, Y59C, Y116C, A139C, W167C, L168C, R170C, and Y171C substitutions) with the numbering as in FIGS. 3D-3I. A peptide epitope may also be conjugated (e.g., by one or two maleimide amino acids or at least one maleimide containing bifunctional linker) to a MHC heavy chain having cysteine residues at any one or more (e.g., 1 or 2) aa positions selected from positions 7 and/or 116, (e.g., Y7C and Y116C substitutions) with the numbering as in FIGS. 3D-3H. Cysteine substitution at positions 116 (e.g., Y116C) and/or 167 (e.g., W167C), with the numbering as in FIGS. 3D-3H, may be used separately or in combination to anchor epitopes (e.g., peptide epitopes) with one or two bonds formed through maleimide groups (e.g., at one or both of the ends of the epitope containing peptide).

Peptide epitopes may also be coupled to a naturally occurring cysteine present or provided in (e.g., engineered into) a β2M polypeptide sequence having at least 85% (e.g., at least 90%, 95% 97% or 100%) sequence identity to at least 60 contiguous amino acids (e.g., at least 70, 80, 90 or all contiguous aas) of a mature β2M polypeptide sequence set forth in FIG. 4. Some solvent accessible positions of mature β2M polypeptides that may be substituted by a cysteine to create a chemical conjugation site include: 2, 14, 16, 34, 36, 44, 45, 47, 48, 50, 58, 74, 77, 85, 88, 89, 91, 94, and 98 (Gln 2, Pro 14, Glu 16, Asp 34, Glu 36, Glu 44, Arg 45, Glu 47, Arg 48, Glu 50, Lys 58, Glu 74, Glu 77, Val 85, Ser 88, Gln 89, Lys 91, Lys 94, and Asp 98) of the mature peptide from NP_004039.1, or their corresponding amino acids in other β2M sequences (see the sequence alignment in FIG. 4). For example, epitopes may be conjugated to cysteines at positions 2, 44, 50, 77, 85, 88, 91, or 98 of the mature β2M polypeptides (aas 22, 64, 70, 97, 105, 108, 111, or 118 of the mature β2M sequences as shown in FIG. 4). Accordingly, the β2M sequences of a T-Cell-MP may contain cysteine chemical conjugation sites provided (e.g., by protein engineering) in the mature β2M sequence selected from Q2C, E44C, E50C, E77C, V85V, S88C, K91C, and D98C. The cysteine chemical conjugation sites in β2M sequences may also be combined with MHC-H Y84C and A139C substitutions made to stabilize the MHC H by forming an intrachain disulfide bond between MHC-H sequences. In one instance, the cysteine chemical conjugation site provided in the mature β2M is located at E44 (an E44C substitution). In another instance, the cysteine chemical conjugation site provided in the mature β2M is located at E44 (an E44C substitution) and the β2M sequence also comprises MHC-H Y84C and A139C substitutions that form an intrachain disulfide bond.

Where conjugation of an epitope, targeting sequences and/or payload is to be conducted through a cysteine chemical conjugation site present in an unconjugated T-cell-MP (e.g., using a maleimide modified epitope or payload) a variety of process conditions may affect the conjugation efficiency and the quality (e.g., the amount/fraction of unaggregated duplex T-Cell-MP epitope conjugate resulting from the reaction) of conjugated T-Cell-MP resulting from the conjugation reaction. Conjugation process conditions that may be individually optimized include but are not limited to (i) prior to conjugation unblocking of cysteine sulfhydryls (e.g., potential blocking groups may be present and removed), (ii) the ratio of the T-Cell-MP to the epitope or payload, (iii) the reaction pH, (iv) the buffer employed, (v) additives present in the reaction, (vi) the reaction temperature, and (vii) the reaction time.

Prior to conjugation T-Cell-MPs may be treated with a disulfide reducing agent such as dithiothreitol (DTT), mercaptoethanol, or tris(2-carboxyethyl)phosphine (TCEP) to reduce and free cysteine sulfhydryls that may be blocked. Treatment may be conducted using relatively low amounts of reducing agent, for example from about 0.5 to 2.0 reducing equivalents per cysteine conjugation site for relatively short periods, and the cysteine chemical conjugation site of the unconjugated T-Cell MP may be available as a reactive nucleophile for conjugation from about 10 minutes to about 1 hour, or from about 1 hour to 5 hours.

The ratio of the unconjugated T-Cell-MP to the epitope or payload being conjugated may be varied from about 1:2 to about 1:100, such as from about 1:2 to about 1:3, from about 1:3 to about 1:10, from about 1:10 to about 1:20, from about 1:20 to about 1:40, or from about 1:40 to about 1:100. The use of sequential additions of the reactive epitope or payload may be made to drive the coupling reaction to completion (e.g., multiple does of maleimide or N-hydroxy succinimide modified epitopes may be added to react with the T-Cell-MP).

As previously indicated, the conjugation reaction may be affected by the buffer, its pH, and additives that may be present. For maleimide coupling to reactive cysteines present in a T-Cell-MP the reactions are typically carried out from about pH 6.5 to about pH 8.5 (e.g., from about pH 6.5 to about pH 7.0, from about pH 7.0 to about pH 7.5, from about pH 7.5 to about pH 8.0, or from about pH 8.0 to about pH 8.5). Any suitable buffer not containing active nucleophiles (e.g., reactive thiols) and preferably degassed to avoid reoxidation of the sulfhydryl may be employed for the reaction. Some suitable traditional buffers include phosphate buffered saline (PBS), Tris-HCl, and (4-(2-hydroxyethyl)-1-piperazineethanesulfonic acid) HEPES. As an alternative to traditional buffers, maleimide conjugation reactions may be conducted in buffers/reaction mixtures comprising amino acids such as arginine, glycine, lysine, or histidine. The use of high concentrations of amino acids, e.g., from about 0.1 M (molar) to about 1.5 M (e.g., from about 0.1 to about 0.25, from about 0.25 to about 0.5 from about 0.3 to about 0.6, from about 0.4 to about 0.7, from about 0.5 to about 0.75, from about 0.75 to about 1.0, from about 1.0 to about 1.25 M, or from about 1.25 to about 1.5 M) may stabilize the conjugated and/or unconjugated T-Cell-MP.

Additives useful for maleimide and other conjugation reactions include, but are not limited to: protease inhibitors; metal chelators (e.g., EDTA) that can block unwanted side reactions and inhibit metal dependent proteases if they are present; detergents (e.g., polysorbate 80 sold as TWEEN 80®, or nonylphenoxypolyethoxyethanol sold under the names NP40 and Tergitol™ NP); and polyols such a sucrose or glycerol that can add to protein stability.

Conjugation of T-Cell-MPs with epitopes, targeting sequences and/or payloads, and particularly conjugation at cysteines using maleimide chemistry, can be conducted over a range of temperatures, such as 0° to 40° C. For example, conjugation reactions, including cysteine-maleimide reactions, can be conducted from about 0° to about 10° C., from about 10° to about 20° C., from about 20° to about 30° C., from about 25° to about 37° C., or from about 30° to about 40° C. (e.g., at about 20° C., at about 30° C. or at about 37° C.).

Where a pair of sulfhydryl groups are present, they may be employed simultaneously for chemical conjugation to a T-Cell-MP. In such an embodiment, an unconjugated T-Cell-MP that has a disulfide bond, or that has two cysteines (or selenocysteines) provided at locations proximate to each other, may be utilized as a chemical conjugation site by incorporation of bis-thiol linkers. Bis-thiol linkers, described by Godwin and co-workers, avoid the instability associated with reducing a disulfide bond by forming a bridging group in its place and at the same time permit the incorporation of another molecule, which can be an epitope or payload. See, e.g., Badescu G, et al., (2014), Bioconjug Chem., 25(6):1124-36, entitled Bridging disulfides for stable and defined antibody drug conjugates, describing the use of bis-sulfone reagents, which incorporate a hydrophilic linker (e.g., PEG (polyethylene glycol) linker).

Generally, stoichiometric or near stoichiometric amounts of dithiol reducing agents (e.g., dithiothreitol) are employed to reduce the disulfide bond and allow the bis-thiol linker to react with both cysteine and/or selenocysteine residues. Where multiple disulfide bonds are present, the use of stoichiometric or near stoichiometric amounts of reducing agents may allow for selective modification at one site. See, e.g., Brocchini, et al., Adv. Drug. Delivery Rev. (2008) 60:3-12. Where a T-Cell-MP or duplexed T-Cell-MP does not comprise a pair of cysteines and/or selenocysteines (e.g., a selenocysteine and a cysteine), they may be provided in the polypeptide (by introducing one or both of the cysteines or selenocysteines) to provide a pair of residues that can interact with a bis-thiol linker. The cysteines and/or selenocysteines should be located such that a bis-thiol linker can bridge them (e.g., at a location where two cysteines could form a disulfide bond). Any combination of cysteines and selenocysteines may be employed (i.e. two cysteines, two selenocysteines, or a selenocysteine and a cysteine). The cysteines and/or selenocysteines may both be present on a T-Cell-MP. Alternatively, in a duplex T-Cell-MP the first cysteine and/or selenocysteine is present in the first T-Cell-MP of the duplex and a second cysteine and/or selenocysteine is present in the second T-Cell-MP of the duplex, with the bis-thiol linker acting as a covalent bridge between the duplexed T-Cell-MPs.

In an embodiment, a pair of cysteine and/or selenocysteine residues is incorporated into a β2M sequence of a T-Cell-MP having at least 85% (e.g., at least 90%, 95%, 98% or 99%, or even 100%) aa sequence identity to at least 50 (e.g., at least 60, 70, 80, 90, 96, 97, or 98 or all) contiguous aas of a mature β2M polypeptide sequence shown in FIG. 4 before the addition of the pair of cysteines and/or selenocysteines, and/or into an L2 or L3 peptide linker attached to one of those sequences. In one such embodiment the pair of cysteines and/or selenocysteines may be utilized as a bis-thiol linker coupling site for the conjugation of an epitope and/or payload through a peptide or chemical linker attached to the bis-thiol group.

In another embodiment, a pair of cysteines and/or selenocysteines is incorporated into a MHC-H polypeptide sequence of a T-Cell-MP as a chemical conjugation site. In an embodiment, a pair of cysteines and/or selenocysteines is incorporated into a polypeptide comprising a sequence having at least 85% (e.g., at least 90%, 95%, 98% or 99%, or even 100%) aa sequence identity to a sequence having at least 150, 175, 200, or 225 contiguous aas of a MHC-H sequence shown in any of FIGS. 3A-3I before the addition of a pair of cysteines or selenocysteines, or into a peptide linker attached to one of those sequences. In one such embodiment the pair of cysteines and/or selenocysteines may be utilized as a bis-thiol linker coupling site for the conjugation of an epitope and/or payload through a peptide or chemical linker attached to the bis-thiol linker. Where the MHC-H sequence includes a Y84C and A139C substitutions the bis-thiol linker may be used to form a covalent bridge between those sites for the covalent coupling of an epitope (e.g., a peptide epitope).

In another embodiment, a pair of cysteines and/or selenocysteines is incorporated into an Ig Fc sequence of a T-Cell-MP to provide a chemical conjugation site. In an embodiment a pair of cysteines and/or selenocysteines is incorporated into a polypeptide comprising an Ig Fc sequence having at least 85% (e.g., at least 90%, 95%, 98% or 99%, or even 100%) aa sequence identity to a sequence shown in any of the Fc sequences of FIGS. 2A-2G before the addition of the pair of cysteines or selenocysteines. In one such embodiment the pair of cysteines and/or selenocysteines is utilized as a bis-thiol linker coupling site for the conjugation of an epitope and/or payload through a peptide or chemical linker attached to the bis-thiol group. The bis-thiol linker may be used to form a covalent bridge between scaffold polypeptides of a duplex T-Cell-MP. In such a case the cysteines of the lower hinge region that form interchain disulfide bonds, if present in the Ig Fc scaffold polypeptide sequence, may be used to insert the bis-thiol linker.

f. Other Chemical Conjugation Sites

(i) Carbohydrate Chemical Conjugation Sites

Many proteins prepared by cellular expression contain added carbohydrates (e.g., oligosaccharides of the type added to antibodies expressed in mammalian cells). Accordingly, where a T-Cell-MP is prepared by cellular expression, carbohydrates may be present and available as selective chemical conjugation sites in, for example, glycol-conjugation reactions, particularly where the T-Cell-MP comprises an Ig Fc scaffold. McCombs and Owen, AAPS Journal, (2015) 17(2): 339-351, and references cited therein, describe the use of carbohydrate residues for glycol-conjugation of molecules to antibodies.

The addition and modification of carbohydrate residues may also be conducted ex vivo, through the use of chemicals that alter the carbohydrates (e.g., periodate, which introduces aldehyde groups), or by the action of enzymes (e.g., fucosyltransferases) that can incorporate chemically reactive carbohydrates or carbohydrate analogs for use as chemical conjugation sites. In an embodiment, the incorporation of an Ig Fc scaffold with known glycosylation sites may be used to introduce site specific chemical conjugation sites.

This disclosure includes and provides for T-Cell-MPs having carbohydrates as chemical conjugation (e.g., glycol-conjugation) sites.

The disclosure also includes and provides for the use of such molecules in forming conjugates with epitopes and with other molecules such as targeting sequences, drugs, and diagnostic agent payloads.

(ii) Nucleotide Binding Sites

Nucleotide binding sites offer site-specific functionalization through the use of a UV-reactive moiety that can covalently link to the binding site. Bilgicer et al., Bioconjug Chem. (2014) 25(7):1198-202, reported the use of an indole-3-butyric acid (IBA) moiety that can be covalently linked to an IgG at a nucleotide binding site. By incorporation of the sequences required to form a nucleotide binding site, chemical conjugates of T-Cell-MP with suitably modified epitopes and/or other molecules (e.g., payload drugs or diagnostic agents) bearing a reactive nucleotide may be employed to prepare T-Cell-MP-epitope conjugates. The epitope or payload may be coupled to the nucleotide binding site through the reactive entity (e.g., an IBA moiety) either directly or indirectly through an interposed linker.

This disclosure includes and provides for T-Cell-MPs having nucleotide binding sites as chemical conjugation sites. The disclosure also includes and provides for the use of such molecules in forming conjugates with epitopes and with other molecules such as drugs and diagnostic agents, and the use of those molecules in methods of treatment and diagnosis.

### 3 MHC Polypeptides of T-Cell-MPs

As noted above, T-Cell-MPs include MHC polypeptides. For the purposes of the instant disclosure, the term “major histocompatibility complex (MHC) polypeptides” is meant to include MHC Class I polypeptides of various species, including human MHC (also referred to as human leukocyte antigen (HLA)) polypeptides, rodent (e.g., mouse, rat, etc.) MHC polypeptides, and MHC polypeptides of other mammalian species (e.g., lagomorphs, non-human primates, canines, felines, ungulates (e.g., equines, bovines, ovines, caprines, etc.), and the like. The term “MHC polypeptide” is meant to include Class I MHC polypeptides (e.g., β-2 microglobulin and MHC Class I heavy chain and/or portions thereof). Both the β2M and MHC-H chain sequences in a T-Cell-MP (may be of human origin. Unless expressly stated otherwise, the T-Cell-MPs and the T-Cell-MP-epitope conjugates described herein are not intended to include membrane anchoring domains (transmembrane regions) of a MHC-H chain, or a part of that molecule sufficient to anchor a T-Cell-MP, or a peptide thereof, to a cell (e.g., eukaryotic cell such as a mammalian cell) in which it is expressed. In addition, the MHC-H chain present in T-Cell-MPs does not include a signal peptide, a transmembrane domain, or an intracellular domain (cytoplasmic tail) associated with a native MHC Class I heavy chain. Thus, e.g., in some cases, the MHC-H chain present in a T-Cell-MP includes only the α1, α2, and α3 domains of a MHC Class I heavy chain. The MHC Class I heavy chain present in a T-Cell-MP may have a length of from about 270 amino acids (aa) to about 290 aa. The MHC Class I heavy chain present in a T-Cell-MP may have a length of 270 aa, 271 aa, 272 aa, 273 aa, 274 aa, 275 aa, 276 aa, 277 aa, 278 aa, 279 aa, 280 aa, 281 aa, 282 aa, 283 aa, 284 aa, 285 aa, 286 aa, 287 aa, 288 aa, 289 aa, or 290 aa.

In some cases, the MHC-H and/or β2M polypeptide of a T-Cell-MP is a humanized or human MHC polypeptide, where human MHC polypeptides are also referred to as “human leukocyte antigen” (“HLA”) polypeptides, more specifically, a Class I HLA polypeptide, e.g., a β2M polypeptide, or a Class I HLA heavy chain polypeptide. Class I HLA heavy chain polypeptides that can be included in T-Cell-MPs include HLA-A, -B, -C, -E, -F, and/or -G heavy chain polypeptides. The Class I HLA heavy chain polypeptides of T-Cell-MPs may comprise polypeptide sequences having at least 75%, at least 80%, at least 85%, at least 90%, at least 95%, at least 98%, at least 99%, or 100% aa sequence identity to all or part (e.g., 50, 75, 100, 150, 200, 225, 250, or 260 contiguous aas) of the aa sequence of any of the human HLA heavy chain polypeptides depicted in FIGS. 3A to 3I (e.g., the sequences encompassing the α1, α2, and α3 domains). For example, they may comprise 1-30, 1-5, 5-10, 10-15, 15-20, 20-25 or 25-30 aa insertions, deletions, and/or substitutions (in addition to those locations indicated as being variable in the heavy chain consensus sequences of FIGS. 3E to 3I).

As an example, a MHC Class I heavy chain polypeptide of a multimeric polypeptide can comprise an aa sequence having at least 75%, at least 80%, at least 85%, at least 90%, at least 95%, at least 98%, at least 99%, or 100% aa sequence identity to aas 25-300 (lacking all, or substantially all, of the leader, transmembrane and cytoplasmic sequences) or 25-365 (lacking the leader) of the human HLA-A heavy chain polypeptides depicted in FIGS. 3A, 3B and/or 3C.

a. MHC Class I Heavy Chains

Class I human MHC polypeptides may be drawn from the classical HLA alleles (HLA-A, B, and C), or the non-classical HLA alleles (e.g., HLA-E, F and G). The following are non-limiting examples of MHC-H alleles and variants of those alleles that may be incorporated into T-Cell-MPs and their epitope conjugates.

(i) HLA-A Heavy Chains

The HLA-A heavy chain peptide sequences, or portions thereof, that may be incorporated into a T-Cell-MP include, but are not limited to, the alleles: A*0101, A*0201, A*0301, A*1101, A*2301, A*2402, A*2407, A*3303, and A*3401, which are aligned without all, or substantially all, of the leader, transmembrane and cytoplasmic sequences in FIG. 3E. Any of those alleles may further comprise a substitution at one or more of positions 84 and/or 139 (as shown in FIG. 3E) selected from: a tyrosine to alanine at position 84 (Y84A); a tyrosine to cysteine at position 84 (Y84C); and an alanine to cysteine at position 139 (A139C). In addition, a HLA-A sequence having at least 75% (e.g., at least 80%, at least 85%, at least 90%, at least 95%, at least 98%, at least 99% or 100%) aa sequence identity to all or part (e.g., 50, 75, 100, 150, 200, 225, 250, or 260 contiguous aas) of the sequence of those HLA-A alleles may also be incorporated into a T-Cell-MP (e.g., it may comprise 1-30, 1-5, 5-10, 10-15, 15-20, 20-25, or 25-30 aa insertions, deletions, and/or substitutions). The HLA-A heavy chain polypeptide sequence of a T-Cell-MP may comprise the Y84C and A139C substitutions.

(a) HLA-A*0101 (HLA-A*01:01:01:01)

A MHC Class I heavy chain polypeptide of a T-Cell-MP or a T-Cell-MP-epitope conjugate may comprise aa sequence of HLA-A*01:01:01:01 (HLA-A*0101, or HLA-A*01:01 listed as HLA-A in FIG. 3D (SEQ ID NO:24) and in FIG. 3E), or a sequence having at least 75% (at least 80%, at least 85%, at least 90%, at least 95%, at least 98%, at least 99% or 100%) aa sequence identity to all or part (e.g., 50, 75, 100, 150, 200, 225, 250, or 260 contiguous aas) of that sequence (e.g., it may comprise 1-30, 1-5, 5-10, 10-15, 15-20, 20-25, or 25-30 aa insertions, deletions, and/or substitutions). In an embodiment, where the HLA-A heavy chain polypeptide of a T-Cell-MP has less than 100% identity to the sequence labeled HLA-A in FIG. 3D, it may comprise a substitution at one or more of positions 84 and/or 139 selected from: a tyrosine to alanine at position 84 (Y84A); a tyrosine to cysteine at position 84 (Y84C); and an alanine to cysteine at position 139 (A139C). The HLA-A*0101 heavy chain polypeptide sequence of a T-Cell-MP may comprise the Y84C and A139C substitutions.

(b) HLA-A*0201 (HLA-A*02:01)

A MHC Class I heavy chain polypeptide of a T-Cell-MP or a T-Cell-MP-epitope conjugate may comprise an aa sequence of HLA-A*0201 (SEQ ID NO:27) provided in FIG. 3D or FIG. 3E, or a sequence having at least 75% (e.g., at least 80%, at least 85%, at least 90%, at least 95%, at least 98%, at least 99% or 100%) aa sequence identity to all or part (e.g., 50, 75, 100, 150, 200, 225, 250, or 260 contiguous aas) of that sequence (e.g., it may comprise 1-30, 1-5, 5-10, 10-15, 15-20, 20-25, or 25-30 aa insertions, deletions, and/or substitutions). In an embodiment, where the HLA-A*0201 heavy chain polypeptide of a T-Cell-MP has less than 100% identity to the sequence labeled HLA-A*0201 in FIG. 3D or 3E, it may comprise a substitution at one or more of positions 84 and/or 139 selected from: a tyrosine to alanine at position 84 (Y84A); a tyrosine to cysteine at position 84 (Y84C); and an alanine to cysteine at position 139 (A139C). The HLA-A*0201 heavy chain polypeptide sequence of a T-Cell-MP may comprise the Y84C and A139C substitutions.

(c) HLA-A*1101 (HLA-A*11:01)

A MHC Class I heavy chain polypeptide of a T-Cell-MP or a T-Cell-MP-epitope conjugate may comprise an aa sequence of HLA-A*1101 (SEQ ID NO:32) provided in FIG. 3D or 3E, or a sequence having at least 75% (e.g., at least 80%, at least 85%, at least 90%, at least 95%, at least 98%, at least 99% or 100%) aa sequence identity to all or part (e.g., 50, 75, 100, 150, 200, 225, 250, or 260 contiguous aas) of that sequence (e.g., it may comprise 1-30, 1-5, 5-10, 10-15, 15-20, 20-25, or 25-30 aa insertions, deletions, and/or substitutions). The HLA-A*1101 heavy chain allele may be prominent in Asian populations, including populations of individuals of Asian descent.

In an embodiment, where the HLA-A*1101 heavy chain polypeptide of a T-Cell-MP has less than 100% identity to the sequence labeled HLA-A*1101 in FIG. 3D or 3E, it may comprise a substitution at one or more of positions 84 and/or 139 selected from: a tyrosine to alanine at position 84 (Y84A); a tyrosine to cysteine at position 84 (Y84C); and an alanine to cysteine at position 139 (A139C). The HLA-A*1101 heavy chain polypeptide sequence of a T-Cell-MP may comprise the Y84C and A139C substitutions.

(d) HLA-A*2402 (HLA-A*24:02)

A MHC Class I heavy chain polypeptide of a T-Cell-MP or a T-Cell-MP-epitope conjugate may comprise an aa sequence of HLA-A*2402 (SEQ ID NO:33) provided in FIG. 3D or 3E, or a sequence having at least 75% (e.g., at least 80%, at least 85%, at least 90%, at least 95%, at least 98%, at least 99% or 100%) aa sequence identity to all or part (e.g., 50, 75, 100, 150, 200, 225, 250, or 260 contiguous aas) of that sequence (e.g., it may comprise 1-30, 1-5, 5-10, 10-15, 15-20, 20-25, or 25-30 aa insertions, deletions, and/or substitutions). The HLA-A*2402 heavy chain allele may be prominent in Asian populations, including populations of individuals of Asian descent.

In an embodiment, where the HLA-A*2402 heavy chain polypeptide of a T-Cell-MP has less than 100% identity to the sequence labeled HLA-A*2402 in FIG. 3D or 3E, it may comprise a substitution at one or more of positions 84 and/or selected from: a tyrosine to alanine at position 84 (Y84A); a tyrosine to cysteine at position 84 (Y84C); and an alanine to cysteine at position 139 (A139C). The HLA-A*2402 heavy chain polypeptide sequence of a T-Cell-MP may comprise the Y84C and A139C substitutions.

(e) HLA-A*3303 (HLA-A*33:03) or HLA-A*3401 (HLA-A*34:01)

A MHC Class I heavy chain polypeptide of a T-Cell-MP or a T-Cell-MP-epitope conjugate may comprise an aa sequence of HLA-A*3303 (SEQ ID NO:34) or HLA-A*3401 (SEQ ID NO:38) provided in FIG. 3D or 3E, or a sequence having at least 75% (e.g., at least 80%, at least 85%, at least 90%, at least 95%, at least 98%, or at least 99%) or 100% aa sequence identity to all or part (e.g., 50, 75, 100, 150, 200, 225, 250, or 260 contiguous aas) of that sequence (e.g., it may comprise 1-25, 1-5, 5-10, 10-15, 15-20, 20-25, or 25-30 aa insertions, deletions, and/or substitutions). The HLA-A*3303 heavy chain allele may be prominent in Asian populations, including populations of individuals of Asian descent.

In an embodiment, where the HLA-A*3303 or HLA-A*3401 heavy chain polypeptide of a T-Cell-MP has less than 100% identity to the sequence labeled HLA-A*3303 in FIG. 3D, it may comprise a substitution at one or more of positions 84 and/or 139 selected from: a tyrosine to alanine at position 84 (Y84A); a tyrosine to cysteine at position 84 (Y84C); and an alanine to cysteine at position 139 (A139C). The HLA-A*3303 or HLA-A*3401 heavy chain polypeptide sequence of a T-Cell-MP may comprise the Y84C and A139C substitutions.

(ii) HLA-B heavy chains.

The HLA-B heavy chain peptide sequences, or portions thereof, that may be incorporated into a T-Cell-MP include, but are not limited to, the alleles: B*0702, B*0801, B*1502, B*3802, B*4001, B*4601, and B*5301, which are aligned without all, or substantially all, of the leader, transmembrane and cytoplasmic sequences in FIG. 3F. Any of those alleles may comprise a substitution at one or more of positions 84 and/or 139 (as shown in FIG. 3F) selected from: a tyrosine to alanine at position 84 (Y84A); a tyrosine to cysteine at position 84 (Y84C); and an alanine to cysteine at position 139 (A139C). In addition, a HLA-B sequence having at least 75% (e.g., at least 80%, at least 85%, at least 90%, at least 95%, at least 98%, at least 99%) or 100% aa sequence identity to all or part (e.g., 50, 75, 100, 150, 200, 225, 250, or 260 contiguous aas) of the sequence of those HLA-B alleles may also be incorporated into a T-Cell-MP (e.g., it may comprise 1-25, 1-5, 5-10, 10-15, 15-20, 20-25, or 25-30 aa insertions, deletions, and/or substitutions). The HLA-B heavy chain polypeptide sequence of a T-Cell-MP may comprise the Y84C and A139C substitutions.

(a) HLA-B*0702 (HLA-B*07:02)

A MHC Class I heavy chain polypeptide of a T-Cell-MP or a T-Cell-MP-epitope conjugate may comprise an aa sequence of HLA-B*0702 (SEQ ID NO:25) in FIG. 3D (labeled HLA-B in FIG. 3D), HLA-B*03501, HLA-B*4402, HLA-B*4403, HLA-B*5801 or a sequence having at least 75% (e.g., at least 80%, at least 85%, at least 90%, at least 95%, at least 98%, at least 99%) or 100% aa sequence identity to all or part (e.g., 50, 75, 100, 150, 200, 225, 250, or 260 contiguous aas) of any of those sequences (e.g., it may comprise 1-25, 1-5, 5-10, 10-15, 15-20, 20-25, or 25-30 aa insertions, deletions, and/or substitutions). In an embodiment, where the HLA-B heavy chain polypeptide of a T-Cell-MP has less than 100% identity to the sequence labeled HLA-B in FIG. 3D, it may comprise a substitution at one or more of positions 84 and/or 139 selected from: a tyrosine to alanine at position 84 (Y84A); a tyrosine to cysteine at position 84 (Y84C); and an alanine to cysteine at position 139 (A139C). The HLA-B*0702 heavy chain polypeptide sequence of a T-Cell-MP may comprise the Y84C and A139C substitutions.

(b) HLA-B*3501 (HLA-B*35:01)

A MHC Class I heavy chain polypeptide of a T-Cell-MP or a T-Cell-MP-epitope conjugate may comprise an aa sequence of HLA-B*3501: GSHSMRYFYTAMSRPGRGEPRFIAVGYVDDTQ-FVRFDSDAASPRTEPRAPWIEQEGPEYWDRNTQIFKTNTQTYRESLRNLRGYYNQSEAGSHIIQR MYGCDLGPDGRLLRGHDQSAYDGKDYIALNEDLSSWTAADTAAQITQRKWEAARVAEQLRAY LEGLCVEWLRRYLENGKETLQRADPPKTHVTHHPVSDHEATLRCWALGFYPAEITLTWQRDGE DQTQDTELVETRPAGDRTFQKWAAVVVPSGEEQRYTCHVQHEGLPKPLTLRWEP (shown lacking its signal sequence and transmembrane/intracellular regions SEQ ID NO:80), or a sequence having at least 75% (e.g., at least 80%, at least 85%, at least 90%, at least 95%, at least 98%, at least 99%) or 100% aa sequence identity to all or part (e.g., 50, 75, 100, 150, 200, 225, 250, or 260 contiguous aas) of that sequence (e.g., it may comprise 1-25, 1-5, 5-10, 10-15, 15-20, 20-25, or 25-30 aa insertions, deletions, and/or substitutions). In an embodiment, the sequence may comprise a substitution at one or more of positions 84 and/or 139 selected from: a tyrosine to alanine at position 84 (Y84A); a tyrosine to cysteine at position 84 (Y84C); and an alanine to cysteine at position 139 (A139C). The HLA-B*3501 heavy chain polypeptide sequence of a T-Cell-MP may comprise the Y84C and A139C substitutions.

(c) HLA-B*4402 (HLA-B*44:02)

A MHC Class I heavy chain polypeptide of a T-Cell-MP or a T-Cell-MP-epitope conjugate may comprise an aa sequence of HLA-B*4402: GSHSMRYFYTAMSRPGRGEPRFITVGYVDDTL-FVRFDSDATSPRKEPRAPWIEQEGPEYWDRETQISKTNTQTYRENLRTALRYYNQSEAGSHIIQR MYGCDVGPDGRLLRGYDQDAYDGKDYIALNEDLSSWTAADTAAQITQRKWEAARVAEQDRA YLEGLCVESLRRYLENGKETLQRADPPKTHVTHHPISDHEVTLRCWALGFYPAEITLTWQRDGE DQTQDTELVETRPAGDRTFQKWAAVVVPSGEEQRYTCHVQHEGLPKPLTLRWEP (shown lacking its signal sequence and transmembrane/intracellular regions SEQ ID NO:81), or a sequence having at least 75% (e.g., at least 80%, at least 85%, at least 90%, at least 95%, at least 98%, at least 99%) or 100% aa sequence identity to all or part (e.g., 50, 75, 100, 150, 200, 225, 250, or 260 contiguous aas) of that sequence (e.g., it may comprise 1-25, 1-5, 5-10, 10-15, 15-20, 20-25, or 25-30 aa insertions, deletions, and/or substitutions). In an embodiment, the sequence may comprise a substitution at one or more of positions 84 and/or 139 selected from: a tyrosine to alanine at position 84 (Y84A); a tyrosine to cysteine at position 84 (Y84C); and an alanine to cysteine at position 139 (A139C). The HLA-B*4402 heavy chain polypeptide sequence of a T-Cell-MP may comprise the Y84C and A139C substitutions.

(d) HLA-B*4403 (HLA-B*44:03)

A MHC Class I heavy chain polypeptide of a T-Cell-MP or a T-Cell-MP-epitope conjugate may comprise an aa sequence of HLA-B*4403: GSHSMRYFYTAMSRPGRGEPRFITVGYVDDT-LFVRFDSDATSPRKEPRAPWIEQEGPEYWDRETQISKTNTQTYRENLRTALRYYNQSEAGSHIIQR MYGCDVGPDGRLLRGYDQDAYDGKDYIALNEDLSSWTAADTAAQITQRKWEAARVAEQLRA YLEGLCVESLRRYLENGKETLQRADPPKTHVTHHPISDHEVTLRCWALGFYPAEITLTWQRDGE DQTQDTELVETRPAGDRTFQKWAAVVVPSGEEQRYTCHVQHEGLPKPLTLRWEP (shown lacking its signal sequence and transmembrane/intracellular regions SEQ ID NO:82), or a sequence having at least 75% (e.g., at least 80%, at least 85%, at least 90%, at least 95%, at least 98%, at least 99%) or 100% aa sequence identity to all or part (e.g., 50, 75, 100, 150, 200, 225, 250, or 260 contiguous aas) of that sequence (e.g., it may comprise 1-25, 1-5, 5-10, 10-15, 15-20, 20-25, or 25-30 aa insertions, deletions, and/or substitutions). In an embodiment, the sequence may comprise a substitution at one or more of positions 84 and/or 139 selected from: a tyrosine to alanine at position 84 (Y84A); a tyrosine to cysteine at position 84 (Y84C); and an alanine to cysteine at position 139 (A139C). The HLA-B*4403 heavy chain polypeptide sequence of a T-Cell-MP may comprise the Y84C and A139C substitutions.

(e) HLA-B*5801 (HLA-B*58:01)

A MHC Class I heavy chain polypeptide of a T-Cell-MP or a T-Cell-MP-epitope conjugate may comprise an aa sequence of HLA-B*58:01: GSHSMRYFYTAMSRPGRGEPRFIAVGYVDDTQFVRFDSDAASPRTEPRAPWIEQEGPEYWDGET RNMKASAQTYRENLRIALRYYNQSEAGSHIIQRMYGCDLGPDGRLLRGHDQSAYDGKDYIALN EDLSSWTAADTAAQITQRKWEAARVAEQLRAYLEGLCVEWLRRYLENGKETLQRADPPKTHVT HHPVSDHEATLRCWALGFYPAEITLTWQRDGEDQTQDTELVETRPAGDRTFQKWAAVVVPSGE EQRYTCHVQHEGLPKPLTLRWEP (shown lacking its signal sequence and transmembrane/intracellular regions SEQ ID NO:83), or a sequence having at least 75% (e.g., at least 80%, at least 85%, at least 90%, at least 95%, at least 98%, at least 99%) or 100% aa sequence identity to all or part (e.g., 50, 75, 100, 150, 200, 225, 250, or 260 contiguous aas) of that sequence (e.g., it may comprise 1-25, 1-5, 5-10, 10-15, 15-20, 20-25, or 25-30 aa insertions, deletions, and/or substitutions). In an embodiment, the sequence may comprise a substitution at one or more of positions 84 and/or 139 selected from: a tyrosine to alanine at position 84 (Y84A); a tyrosine to cysteine at position 84 (Y84C); and an alanine to cysteine at position 139 (A139C). The HLA-B*5901 heavy chain polypeptide sequence of a T-Cell-MP may comprise the Y84C and A139C substitutions.

(iii) HLA-C Heavy Chains

The HLA-C heavy chain peptide sequences, or portions thereof, that may be incorporated into a T-Cell-MP include, but are not limited to, the alleles: C*0102, C*0303, C*0304, C*0401, C*0602, C*0701, C*0702, C*0801, and C*1502, which are aligned without all, or substantially all, of the leader, transmembrane and cytoplasmic sequences in FIG. 3G. Any of those alleles may comprise a substitution at one or more of positions 84, 139 and/or 236 (as shown in FIG. 3G) selected from: a tyrosine to alanine at position 84 (Y84A); a tyrosine to cysteine at position 84 (Y84C); and an alanine to cysteine at position 139 (A139C). In addition, an HLA-C sequence having at least 75% (e.g., at least 80%, at least 85%, at least 90%, at least 95%, at least 98%, at least 99%) or 100% aa sequence identity to all or part (e.g., 50, 75, 100, 150, 200, 225, 250, or 260 contiguous aas) of the sequence of those HLA-C alleles may also be incorporated into a T-Cell-MP (e.g., it may comprise 1-25, 1-5, 5-10, 10-15, 15-20, 20-25, or 25-30 aa insertions, deletions, and/or substitutions). The HLA-C heavy chain polypeptide sequence of a T-Cell-MP may comprise the Y84C and A139C substitutions.

(a) HLA-C*701 (HLA-C*07:01) and HLA-C*702 (HLA-C*07:02)

A MHC Class I heavy chain polypeptide of a T-Cell-MP or a T-Cell-MP-epitope conjugate may comprise an aa sequence of HLA-C*701 (SEQ ID NO:23) or HLA-C*702 (SEQ ID NO:54) in FIG. 3G (labeled HLA-C in FIG. 3D), or a sequence having at least 75% (e.g., at least 80%, at least 85%, at least 90%, at least 95%, at least 98%, at least 99%) or 100% aa sequence identity to all or part (e.g., 50, 75, 100, 150, 200, 225, 250, or 260 contiguous aas) of those sequences (e.g., it may comprise 1-25, 1-5, 5-10, 10-15, 15-20, 20-25, or 25-30 aa insertions, deletions, and/or substitutions relative to those sequences). In an embodiment, where the HLA-C heavy chain polypeptide of a T-Cell-MP has less than 100% identity to the sequence labeled HLA-C in FIG. 3D, it may comprise a substitution at one or more of positions 84 and/or 139 selected from: a tyrosine to alanine at position 84 (Y84A); a tyrosine to cysteine at position 84 (Y84C); and an alanine to cysteine at position 139 (A139C). The HLA-C*701 or HLA-C*0702 heavy chain polypeptide sequence of a T-Cell-MP may comprise the Y84C and A139C substitutions.

(iv) Non-Classical HLA-E, F and G Heavy Chains

The non-classical HLA heavy chain peptide sequences, or portions thereof, that may be incorporated into a T-Cell-MP include, but are not limited to, those of the HLA-E, F, and/or G alleles. Sequences for those alleles, (and the HLA-A, B and C alleles) may be found on the World Wide Web at, for example, hla.alleles.org/nomenclature/index.html, the European Bioinformatics Institute (www.ebi.ac.uk), which is part of the European Molecular Biology Laboratory (EMBL), and the National Center for Biotechnology Information (www.ncbi.nlm.nih.gov).

Some suitable HLA-E alleles include, but are not limited to, HLA-E*0101 (HLA-E*01:01:01:01), HLA-E*01:03(HLA-E*01:03:01:01), HLA-E*01:04, HLA-E*01:05, HLA-E*01:06, HLA-E*01:07, HLA-E*01:09, and HLA-E*01:10. Some suitable HLA-F alleles include, but are not limited to, HLA-F*0101 (HLA-F*01:01:01:01), HLA-F*01:02, HLA-F*01:03(HLA-F*01:03:01:01), HLA-F*01:04, HLA-F*01:05, and HLA-F*01:06. Some suitable HLA-G alleles include, but are not limited to, HLA-G*0101 (HLA-G*01:01:01:01), HLA-G*01:02, HLA-G*01:03(HLA-G*01:03:01:01), HLA-G*01:04 (HLA-G*01:04:01:01), HLA-G*01:06, HLA-G*01:07, HLA-G*01:08, HLA-G*01:09: HLA-G*01:10, HLA-G*01:11, HLA-G*01:12, HLA-G*01:14, HLA-G*01:15, HLA-G*01:16, HLA-G*01:17, HLA-G*01:18: HLA-G*01:19, HLA-G*01:20, and HLA-G*01:22. Consensus sequences for those HLA-E, -F, and -G alleles without all, or substantially all, of the leader, transmembrane and cytoplasmic sequences are provided in FIG. 3H, and aligned with consensus sequences of the above-mentioned HLA-A, -B, and -C alleles provided in FIGS. 3E-3G and in FIG. 3I.

Any of the above-mentioned HLA-E, F and/or G alleles may comprise a substitution at one or more of positions 84 and/or 139 as shown in FIG. 3I for the consensus sequences. In an embodiment, the substitutions may be selected from: a position 84 tyrosine to alanine (Y84A) or cysteine (Y84C), or in the case of HLA-F a R84A or R84C substitution; and/or a position 139 alanine to cysteine (A139C), or in the case of HLA-F a V139C substitution. In addition, HLA-E, -F, and/or -G sequences having at least 75% (e.g., at least 80%, at least 85%, at least 90%, at least 95%, at least 98%, at least 99%) or 100% aa sequence identity to all or part (e.g., 50, 75, 100, 150, 200, 225, 250, or 260 contiguous aas) of any of the consensus sequences set forth in FIG. 3I may also be employed (e.g., the sequences may comprise 1-25, 1-5, 5-10, 10-15, 15-20, 20-25, or 25-30 aa insertions, deletions, and/or substitutions in addition to changes at variable residues listed therein). The HLA-E, F, or G heavy chain polypeptide sequence of a T-Cell-MP may comprise a cysteine at both position 84 and 139.

(v) Mouse H2K

A MHC Class I heavy chain polypeptide of a T-Cell-MP or a T-Cell-MP-epitope conjugate may comprise an aa sequence of MOUSE H2K (SEQ ID NO:28) (MOUSE H2K in FIG. 3D), or a sequence having at least 75%, at least 80%, at least 85%, at least 90%, at least 95%, at least 98%, at least 99%, or 100% aa sequence identity to all or part (e.g., 50, 75, 100, 150, 200, 225, 250, or 260 contiguous aas) of that sequence (e.g., it may comprise 1-30, 1-5, 5-10, 10-15, 15-20, 20-25, or 25-30 aa insertions, deletions, and/or substitutions). In an embodiment, where the MOUSE H2K heavy chain polypeptide of a T-Cell-MP has less than 100% identity to the sequence labeled MOUSE H2K in FIG. 3D, it may comprise a substitution at one or more of positions 84 and/or 139 selected from: a tyrosine to alanine at position 84 (Y84A); a tyrosine to cysteine at position 84 (Y84C); and an alanine to cysteine at position 139 (A139C). The MOUSE H2K heavy chain polypeptide sequence of a T-Cell-MP may comprise the Y84C and A139C substitutions.

(vi) The Effect of Amino Acid Substitutions in MHC Polypeptides on T-Cell-MPs

(a) Substitutions at Positions 84 and 139

Substitution of position 84 of the MHC H chain (see FIG. 3I), particularly when it is a tyrosine residue, with a small amino acid such as alanine (Y84A) tends to open one end of the MHC binding pocket, allowing a linker (e.g., attached to a peptide epitope) to “thread” through the end of the pocket, and accordingly, permits a greater variation in the size of the epitope (e.g., longer peptides bearing epitope sequences) that can fit into the MHC pocket and be presented by the T-Cell-MP. Alternatively, the MHC-H (e.g., HLA-heavy chain) of a T-Cell-MP may be substituted with cysteines to form an intrachain disulfide bond between a cysteine substituted into the carboxyl end portion of the α1 helix and a cysteine in the amino end portion of the α2-1 helix (e.g., amino acids 84 and 139). Such disulfide bonds stabilize the MHC-H polypeptide sequence of a T-Cell-MP, and permit its translation, cellular processing, and excretion from eukaryotic cells in the absence of a bound peptide epitope (or null peptide). Any combination of substitutions provided in the table provide below at residues 84 and 130 may be combined with any combination of substitutions in the epitope binding cleft, such as those described at positions 116 and 167.

(b) Substitutions at Positions 116 and 167

Any MHC Class I heavy chain sequences (including those disclosed above for: the HLA-A*0101; HLA-A*0201; HLA-A*1101; HLA-A*2402; HLA-A*3303; HLA-B; HLA-C; Mouse H2K, or any of the other HLA-A, B, C, E, F, and/or G sequence disclosed herein) may further comprise a cysteine substitution at position 116 (e.g., Y116C) or at position 167.

As with aa position 84 substitutions that open one end of the MHC-H binding pocket (e.g., Y84A or its equivalent), substitution of an alanine or glycine at position 167 (e.g., a W167A substitution or its equivalent) opens the other end of the MHC binding pocket, creating a groove that permits greater variation (e.g., longer length) of the peptide epitopes that may be presented by the T-Cell-MP-epitope conjugates. Substitutions at positions 84 and/or 167, or their equivalent (e.g., Y84A in combination with W167A or W167G) may be used in combination to modify the binding pocket of MHC-H chains. A cysteine substitution at positions 116 (e.g., Y116C) and/or 167 (e.g., W167C) may be used separately or in combination to anchor epitopes (e.g., peptide epitopes) in one or two locations (e.g., the ends of the epitope containing peptide). Substitutions at positions 116 and/or 167 may be combined with substitutions including those at positions 84 and/or 139 described above.

The Table below lists some MHC heavy chain sequence modifications that may be incorporated into a T-Cell-MPs.

Some Combinations of MHC Class 1 Heavy Chain Sequence Modifications that May be Incorporated into a T-Cell-MP or its Epitope Conjugate

| | HLA Heavy | | Substitutions at | Substitutions at |
|---|---|---|---|---|
| | Chain Sequence | | aa positions 84 | positions 116 |
| Entry | From FIGs. 3D-H | Sequence Identity Range □ | and/or 139 | and/or 167 |
| 1 | HLA-A | 75%-99.8%, 80%-99.8%, 85%-99.8%, | None; Y84C; | None; Y116C; |
| | Consensus | 90%-99.8%, 95%-99.8%, 98%-99.8%, | Y84A; A139C; | W167A; W167C; |
| | FIG. 3E | or 99%-99.8%; or 1-25, 1-5, 5-10, | or (Y84C & | or (Y116C & |
| | | 10-15, 15-20, or 20-25 aa insertions, | A139C) | W167C) |
| | | deletions, and/or substitutions (not | | |
| | | counting variable residues) | | |
| 2 | A*0101, A*0201, | 75%-99.8%, 80%-99.8%, 85%-99.8%, | None; Y84C; | None; Y116C; |
| | A*0301, A*1101, | 90%-99.8%, 95%-99.8%, 98%-99.8%, | Y84A; A139C; | W167A; W167C; |
| | A*2402, A*2301, | or 99%-99.8%; or 1-25, 1-5, 5-10, | or (Y84C & | or (Y116C & |
| | A*2402, A*2407, | 10-15, 15-20, or 20-25 aa insertions, | A139C) | W167C) |
| | A*3303, or | deletions, and/or substitutions | | |
| | A*3401 | | | |
| 3 | HLA-B | 75%-99.8%, 80%-99.8%, 85%-99.8%, | None; Y84C; | None; Y116C; |
| | Consensus | 90%-99.8%, 95%-99.8%, 98%-99.8%, | Y84A; A139C; | W167A; W167C; |
| | FIG. 3F | or 99%-99.8%; or 1-25, 1-5, 5-10, | or (Y84C & | or (Y116C & |
| | | 10-15, 15-20, or 20-25 aa insertions, | A139C) | W167C) |
| | | deletions, and/or substitutions (not | | |
| | | counting variable residues) | | |
| 4 | B*0702, B*0801, | 75%-99.8%, 80%-99.8%, 85%-99.8%, | None; Y84C; | None; Y116C; |
| | B*1502, B*3501, | 90%-99.8%, 95%-99.8%, 98%-99.8%, | Y84A; A139C; | W167A; W167C; |
| | B*3802, B*4001, | or 99%-99.8%; or 1-25, 1-5, 5-10, | or (Y84C & | or (Y116C & |
| | B*4402, B*4403, | 10-15, 15-20, or 20-25 aa insertions, | A139C) | W167C) |
| | B*4601, B*5301, | deletions, and/or substitutions | | |
| | or B*5801 | | | |
| 5 | HLA-C | 75%-99.8%, 80%-99.8%, 85%-99.8%, | None; Y84C; | None; Y116C; |
| | Consensus | 90%-99.8%, 95%-99.8%, 98%-99.8%, | Y84A; A139C; | W167A; W167C; |
| | FIG. 3G | or 99%-99.8%; or 1-25, 1-5, 5-10, | or (Y84C & | or (Y116C & |
| | | 10-15, 15-20, or 20-25 aa insertions, | A139C) | W167C) |
| | | deletions, and/or substitutions (not | | |
| | | counting variable residues) | | |
| 6 | C*0102, C*0303, | 75%-99.8%, 80%-99.8%, 85%-99.8%, | None; Y84C; | None; Y116C; |
| | C*0304, C*0401, | 90%-99.8%, 95%-99.8%, 98%-99.8%, | Y84A; A139C; | W167A; W167C; |
| | C*0602, C*0701, | or 99%-99.8%; or 1-25, 1-5, 5-10, | or (Y84C & | or (Y116C & |
| | C*702, C*0801, | 10-15, 15-20, or 20-25 aa insertions, | A139C) | W167C) |
| | or C*1502 | deletions, and/or substitutions | | |
| 7 | HLA-E, | 75%-99.8%, 80%-99.8%, 85%-99.8%, | None; Y84C; | None; Y116C; |
| | F, or G | 90%-99.8%, 95%-99.8%, 98%-99.8%, | Y84A; A139C; | W167A; W167C; |
| | Consensus | or 99%-99.8%; or 1-25, 1-5, 5-10, | or (Y84C & | or (Y116C & |
| | FIG. 3H | 10-15, 15-20, or 20-25 aa insertions, | A139C) | W167C) |
| | | deletions, and/or substitutions (not | | |
| | | counting variable residues) | | |
| 8 | MOUSE H2K | 75%-99.8%, 80%-99.8%, 85%-99.8%, | None; Y84C; | None; Y116C; |
| | | 90%-99.8%, 95%-99.8%, 98%-99.8%, | Y84A; A139C; | W167A; W167C; |
| | | or 99%-99.8%; or 1-25, 1-5, 5-10, | or (Y84C & | or (Y116C & |
| | | 10-15, 15-20, or 20-25 aa insertions, | A139C) | W167C) |
| | | deletions, and/or substitutions | | |

□ The Sequence Identity Range is the permissible range in sequence identity of a MHC-H polypeptide sequence incorporated into a T-Cell-MP relative to the corresponding portion of the sequences listed in FIG. 3D-3H not counting the variable residues when the consensus sequences are used for the comparison.

b. MHC Class I β2-Microglobins and Combinations with MHC-H Polypeptides

A β2M polypeptide of a T-Cell-MP can be a human β2M polypeptide, a non-human primate β2M polypeptide, a murine β2M polypeptide, and the like. In some instances, a β2M polypeptide comprises an aa sequence having at least 75%, at least 80%, at least 85%, at least 90%, at least 95%, at least 98%, at least 99%, or 100% aa sequence identity to a β2M aa sequence (e.g., a mature β2M sequence) depicted in FIG. 4. The β2M polypeptide of a T-Cell-MP may comprise an aa sequence having at least 75%, at least 80%, at least 85%, at least 90%, at least 95%, at least 98%, at least 99%, or 100% aa sequence identity to aas 21 to 119 of a β2M aa sequence depicted in FIG. 4, which may include a cysteine or other aa substitution or insertion as a chemical conjugation site for epitope attachment (e.g., and E44C substitution) when the identity is less than 100%. Chemical conjugation sites may be located at, for example, solvent accessible locations in the β2M polypeptide sequence.

The β2M polypeptide sequence of a T-Cell-MP may have at least 90% (e.g., at least 95% or 98%) or 100% sequence identity to at least 70 (e.g., at least 80, 90, 96, 97, 98 or all) contiguous aas of a mature human β2M polypeptide (e.g., aas 21-119 of NCBI accession number NP_004039.1 provided in FIG. 4). By way of example, a β2M polypeptide sequence of a T-Cell-MP may have up to six (e.g., 1, 2, 3, 4, 5, or 6) aa substitutions within an aa segment of at least 70 (e.g., at least 80, 90, 96, 97, or 98 or all) contiguous aas of a mature human β2M polypeptide (e.g., aas 21-119 of NCBI accession number NP_004039.1 provided in FIG. 4), and may comprise the chemical conjugation site for attachment of an epitope (e.g., an E44C substitution in the mature peptide). As noted above, in such β2M polypeptide sequences the chemical conjugation sites of epitopes may be located at a variety of locations including solvent accessible aa positions. For example, a cysteine or other amino acid substitution or insertion at a solvent accessible amino acid position can provide a chemical conjugation site for direct or indirect (e.g., through a peptide linker) attachment of an epitope.

Some solvent accessible positions of mature β2M polypeptides lacking their leader sequence include aa positions 2, 14, 16, 34, 36, 44, 45, 47, 48, 50, 58, 74, 77, 85, 88, 89, 91, 94, and 98 (Gln 2, Pro 14, Glu 16, Asp 34, Glu 36, Glu 44, Arg 45, Glu 47, Arg 48, Glu 50, Lys 58, Glu 74, Glu 77, Val 85, Ser 88, Gln 89, Lys 91, Lys 94, and Asp 98) of the mature peptide from NP_004039.1, or their corresponding amino acids in other β2M sequences (see the sequence alignment in FIG. 4). The solvent accessible locations for chemical conjugation sites (e.g., a cysteine or another reactive aa substitution) may be selected from positions 2, 44, 50, 77, 85, 88, 91, or 98 of a mature β2M polypeptide sequence such as NP_004039.1, or the corresponding aa positions in other β2M sequences such as those in FIG. 4. The solvent accessible locations for chemical conjugation sites (e.g., a cysteine or another reactive aa substitution) may also be selected from positions 2, 44, 50, or 98 of a mature β2M polypeptide sequence such as NP_004039.1, or the corresponding aa positions in other β2M sequences such as those in FIG. 4. The solvent accessible locations for chemical conjugation sites (e.g., a cysteine or another reactive aa substitution) may be selected from positions 2 or 44 (Glu 2 or Glu 44) of a mature β2M polypeptide sequence such as NP_004039.1, or the corresponding aa positions in other β2M sequences such as those in FIG. 4.

A β2M polypeptide sequence may comprise a single cysteine substituted into a wt. β2M polypeptide (e.g., a β2M sequence in FIG. 4). Such cysteine residues, when present in a T-Cell-MP polypeptide, can act as a chemical conjugation site for the covalent coupling of an epitope (either directly or indirectly through a linker). The covalent attachment may be in the form of a bond made to a reactive group in or attached to the epitope, such as a maleimide group incorporated into the epitope or a linker attached to the peptide epitope, or in the form of a disulfide bond. For example, in some cases, one of amino acids 43, 44, or 45 of the mature β2M lacking its signal sequence (residues 63, 64, and 65 of the unprocessed proteins as shown with their signal sequences in FIG. 4) may be substituted with a cysteine residue. The aa position substituted with a cysteine may be position 44 (e.g., an E44C substitution of the mature human protein NP_004039.1 or a corresponding aa substitution in a β2M sequence such as those in FIG. 4). Alternatively, the aa position substituted with a cysteine may be position 2 (e.g., a Q44C substitution of the mature human protein NP_004039.1 or a corresponding aa substitution in a β2M sequence such as those in FIG. 4).

c. Some Combinations of Substitutions in the MHC-H and the β2M Polypeptide Sequences

Separately, or in addition to, any cysteine residues inserted into the MHC-H or β2M polypeptide sequence of a T-Cell-MP that may function as a chemical conjugation site for an epitope or a payload (e.g., an E44C substitution in a β2M polypeptide sequence that provides a chemical conjugation site for an epitope), a T-Cell-MP may comprise an intrachain disulfide bond between a cysteine substituted into the carboxyl end portion of the α1 helix and a cysteine in the amino end portion of the α2-1 helix (e.g., amino acids at aa positions 84 and 139, such as Y84C and A139C). The carboxyl end portion of the α1 helix is from about aa position 79 to about aa position 89 and the amino end portion of the α2-1 helix is from about aa position 134 to about aa position 144 of the MHC-H chain (the aa positions are determined based on the sequence of the heavy chains without their leader sequence (see, e.g., FIGS. 3D-3H). Accordingly, a disulfide bond may be between a cysteine located at positions 83, 84, or 85 and a cysteine located at any of positions 138, 139 or 140 of the MHC-H polypeptide sequence. For example, in a T-Cell-MP a disulfide bond may be formed between a cysteine inserted at position 84 and a cysteine inserted at any of positions 138, 139 or 140 of the MHC-H polypeptide sequence. In one aspect, the MHC-H intrachain disulfide bond is between cysteines substituted at positions 84 and 139 of any of the heavy chain sequences set forth in FIGS. 3D-3H.

A T-Cell-MP may comprise a combination of: (i) a mature β2M polypeptide sequence having at least 90% (e.g., at least 95% or 98%) sequence identity to at least 70 (e.g., at least 80, 90, 96, 97, 98 or all) of aas 21-119 of NP_004039.1 with an E44C (or another cysteine substitution) as a chemical conjugation site for an epitope; and (ii) a HLA Class I heavy chain polypeptide sequence having at least 90% sequence identity (e.g., at least 95%, 98%, or 100% sequence identity) excluding variable aa clusters 1-4 to: GSHSMRYFFTSVSRPGRGEPRFIAVGYVDDTQFVRFDSDAASQRMEPRAPWIEQEGPEYWD GETRKVKAHSQTHRVDL(aa cluster 1){C}(aa cluster 2)AGSHTVQRMYGCDVGSDWRFLRGYHQ YAYDGKDYIALKEDLRSW(aa cluster 3){C}(aa cluster 4) (a 2×hIL2(F42A,H16A)-(G4S)-GMGGSG GGGS-(G4S)-β2M (E44C)-(G4S)3-HLA-A02(Y84C,A139C)-AAAGG-hIgG1(L234A,L235A)HKWEA AHVAEQLRAYLEGTCVEWLRRYLENGKETLQRTDAPKTHMTHHAVSDHEATLRCWALSFYPA EITLTWQRDGEDQTQDTELVETRPAGDGTFQKWAAVVVPSGQEQRYTCHVQHEGLPKPLTLRW EP (SEQ ID NO:84);

where the cysteine residues indicated as {C} form a disulfide bond between the α1 and α2-1 helices.

Each occurrence of aa cluster 1, aa cluster 2, aa cluster 3, aa cluster 4, aa cluster 5, and aa cluster 6 is independently selected to be 1-5 aa residues, wherein the aa residues are each selected independently from i) any naturally occurring (proteinogenic) aa or ii) any naturally occurring aa except proline or glycine. The MHC-H polypeptide sequence may be an HLA-A chain, wherein:
aa cluster 1 may be the amino acid sequence GTLRG (SEQ ID NO:85) or that sequence with one or two aas deleted or substituted with other naturally occurring aas (e.g., L replaced by I, V, A or F);
aa cluster 2 may be the amino acid sequence YNQSE (SEQ ID NO:86) or that sequence with one or two aas deleted or substituted with other naturally occurring aas (e.g., N replaced by Q, Q replaced by N, and/or E replaced by D);
aa cluster 3 may be the amino acid sequence TAADM (SEQ ID NO:87) or that sequence with one or two aas deleted or substituted with other naturally occurring aas (e.g., T replaced by S, A replaced by G, D replaced by E, and/or M replaced by L, V, or I); and/or aa cluster 4 may be the amino acid sequence AQTTK (SEQ ID NO:88) or that sequence with one or two aas deleted or substituted with other naturally occurring aas (e.g., A replaced by G, Q replaced by N, or T replaced by S, and or K replaced by R or Q).

As noted above, any of the MHC-H intrachain disulfide bonds, including a disulfide bond between cysteines at 84 and 139 (a Y84C and A139C disulfide), may be combined with substitutions that permit incorporation of a peptide epitope into a T-Cell-MP. Accordingly, the present disclosure includes and provides for T-Cell-MPs and their higher order complexes (e.g., duplexes) comprising one or more T-Cell-MP polypeptides having a MHC-H polypeptide sequence with an intrachain Y84C A139C disulfide bond and an E44C substitution in the β2M polypeptide sequence. T-Cell-MPs and their higher order complexes (e.g., duplexes) may comprise: (i) a mature β2M polypeptide sequence with an E44C substitution having at least 90% (e.g., at least 95% or 98%) sequence identity to at least 70 (e.g., at least 80, 90, 96, 97, 98 or all) of aas 21-119 of any one of NP_004039.1, NP_001009066.1, NP_001040602.1, NP_776318.1, or NP_033865.2 (SEQ ID NOs:61 to 65, see FIG. 4); and (ii) a MHC-H sequence with Y84C and A139C substitutions (that form a disulfide bond) may have at least 85% (e.g., at least 90%, at least 95% or 98%) or 100% sequence identity to at least 200 (e.g., at least 225, at least 250, at least 260, or at least 275) contiguous aas of the α1, α2, and α3 domains an HLA-A, -B, -C, -E, -F, or -G sequences in FIGS. 3D-3H. The MHC-H polypeptide sequence may be a HLA-A*0101, HLA-A*0201, HLA-A*1101, HLA-A*2402, HLA-A*3303, or HLA-A*3401 polypeptide sequence having Y84C and A139C substitutions (see FIG. 3E). The MHC-H polypeptide sequence may be a HLA-B*0702, HLA-B*0801, HLA-B*1502, B27 (subtypes HLA-B*2701-2759), HLA-B*3802, HLA-B*4001, HLA-B*4601, or HLA-B*5301 polypeptide sequence having Y84C and A139C substitutions (see, e.g., FIG. 3F). The MHC-H polypeptide sequence may be a HLA-C*0102, HLA-C*0303, HLA-C*0304, HLA-C*0401, HLA-C*0602, HLA-C*0701, HLA-C*0702, HLA-C*0801, or HLA-C*1502 polypeptide sequence having Y84C and A139C substitutions (see, e.g., FIG. 3G).

### 4 Scaffold Polypeptides

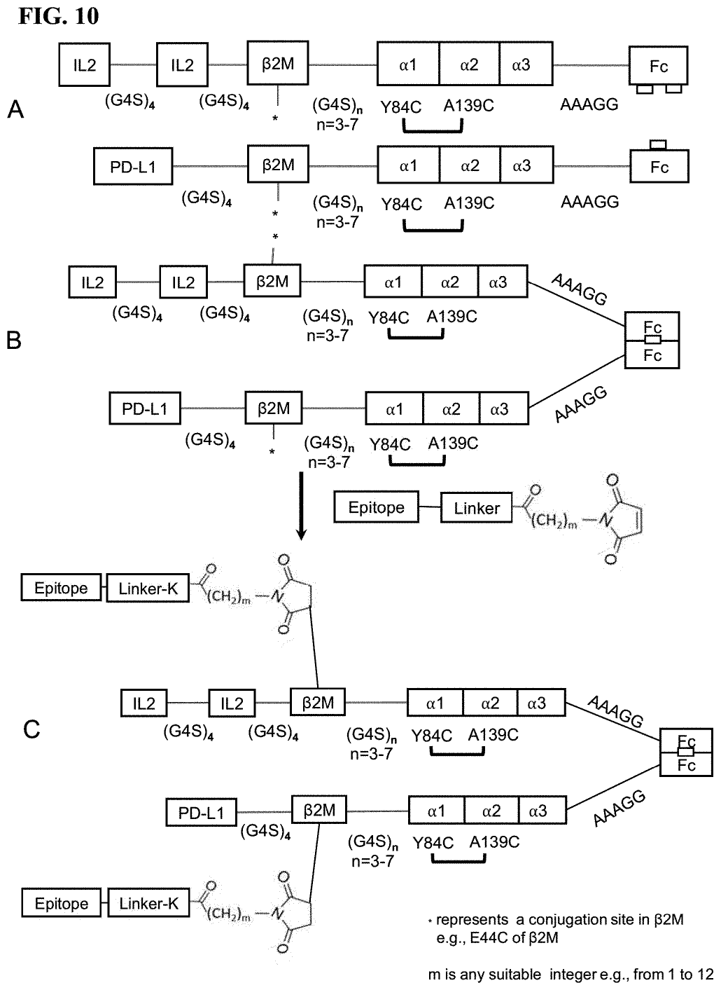

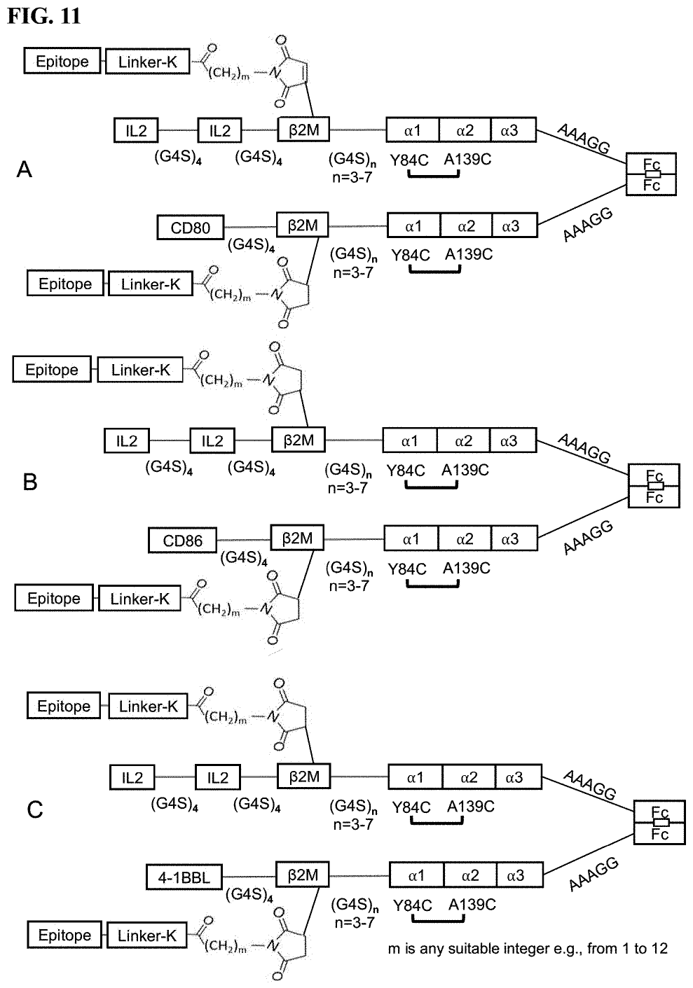

T-Cell-MPs and T-Cell-MP-epitope conjugates may comprise an immunoglobulin heavy chain constant region (“Ig Fc” or “Fc”) polypeptide, or may comprise another suitable scaffold polypeptide. Where scaffold polypeptide sequences are identical and pair or multimerize (e.g., some Ig Fc sequences or leucine zipper sequences), they can form symmetrical pairs or multimers (e.g., homodimers, see e.g., FIG. 9 with an Fc scaffold). In contrast, where an asymmetric pairing between two T-Cell-MP molecules is desired (e.g., to produce a duplex T-Cell-MP with each bearing one or more different MODs), the scaffold polypeptides present in the T-Cell-MP may comprise interspecific binding sequences. Interspecific binding sequences are non-identical polypeptide sequences that selectively interact with their specific complementary counterpart sequence to form asymmetric pairs (heterodimers, see e.g., FIG. 10 with an interspecific Fc scaffold). Interspecific binding sequences may in some instances form some amount of homodimers, but preferentially dimerize by binding more strongly) with their counterpart interspecific binding sequence. Accordingly, specific heterodimers tend to be formed when an interspecific dimerization sequence and its counterpart interspecific binding sequence are incorporated into a pair of polypeptides. By way of example, where an interspecific dimerization sequence and its counterpart are incorporated into a pair of polypeptides they may selectively form greater than 70%, 80%, 90%, 95%, 98% or 99% heterodimers when an equimolar mixture of the polypeptides are combined. The remainder of the polypeptides may be present as monomers or homodimers, which may be separated from the heterodimer. Moreover, because interspecific sequences are selective for their counterpart sequence, they can limit the interaction with other proteins expressed by cells (e.g., in culture or in a subject) particularly where the interspecific sequences are not naturally occurring or are variants of naturally occurring protein sequences.

Scaffold polypeptide sequences generally may be less than 300 aa (e.g., about 100 to about 300 aa). Scaffold polypeptide sequences may be less than 250 aa (e.g., about 75 to about 250 aa). Scaffold polypeptide sequences may be less than 200 aa (e.g., about 60 to about 200 aa). Scaffold polypeptide sequences may be less than 150 aa (e.g., about 50 to about 150 aa).

Scaffold polypeptide sequences include, but are not limited to, interspecific and non-interspecific Ig Fc polypeptide sequences, however, polypeptide sequences other than Ig Fc polypeptide sequences (non-Immunoglobulin sequences) may be used as scaffolds.

a. Non-Immunoglobulin Fc Scaffold Polypeptides

Non-immunoglobulin Fc scaffold polypeptides include, but are not limited to: albumin, XTEN (extended recombinant); transferrin; Fc receptor, elastin-like; albumin-binding; silk-like (see, e.g., Valluzzi et al. (2002) Philos Trans R Soc Loud B Biol Sci. 357:165); a silk-elastin-like (SELP; see, e.g., Megeed et al. (2002) Adv Drug Deliv Rev. 54:1075) polypeptides; and the like. Suitable XTEN polypeptides include, e.g., those disclosed in WO 2009/023270, WO 2010/091122, WO 2007/103515, US 2010/0189682, and US 2009/0092582; see, also, Schellenberger et al. (2009) Nat Biotechnol. 27:1186). Suitable albumin polypeptides include, e.g., human serum albumin. Suitable elastin-like polypeptides are described, for example, in Hassouneh et al. (2012) Methods Enzymol. 502:215.

Other non-immunoglobulin Fc scaffold polypeptide sequences include but are not limited to: polypeptides of the collectin family (e.g., ACRP30 or ACRP30-like proteins) that contain collagen domains consisting of collagen repeats Gly-Xaa-Yaa and/or Gly-Xaa-Pro (which may be repeated from 10-40 times); coiled-coil domains; leucine-zipper domains; Fos/Jun binding pairs; Ig CH1 and light chain constant region C_{L} sequences (Ig CH1/C_{L} pairs such as a Ig CH1 sequence paired with a Ig C_{L}, κ or C_{L} λ light chain constant region sequence).

Non-immunoglobulin Fc scaffold polypeptides can be interspecific or non-interspecific in nature. For example, both Fos/Jun binding pairs and Ig CH1 polypeptide sequences and light chain constant region C_{L} sequences form interspecific binding pairs. Coiled-coil sequences, including leucine zipper sequences, can be either interspecific leucine zipper or non-interspecific leucine zipper sequences. See e.g., Zeng et al., (1997) PNAS (USA) 94:3673-3678; and Li et al., (2012), Nature Comms. 3:662.

The scaffold polypeptides of a duplex T-Cell-MP may each comprise a leucine zipper polypeptide sequence. The leucine zipper polypeptides bind to one another to form a dimer. Non-limiting examples of leucine-zipper polypeptides include a peptide comprising any one of the following aa sequences: RMKQIEDKIEEILSKIYHIENEIARIKKLIGER (SEQ ID NO:89); LSSIEKKQEEQTS-WLIWISNELTLIRNELAQS (SEQ ID NO:90); LSSIEKKLEEITSQLIQISNELTLIRNELAQ (SEQ ID NO:91; LSSIEKKLEEITSQLIQIRNELTLIRNELAQ (SEQ ID NO:92); LSSIEKKLEEITSQLQQ-IRNELTLIRNELAQ (SEQ ID NO:93); LSSLEKKLEELTSQLIQLRNELTLLRNELAQ (SEQ ID NO:94); ISSLEKKIEELTSQIQQLRNEITLLRNEIAQ (SEQ ID NO:95). In some cases, a leucine zipper polypeptide comprises the following aa sequence: LEIEAAFLERENTALETRVAELRQRVQRLRNRV-SQYRTRYGPLGGGK (SEQ ID NO:96). Additional leucine-zipper polypeptides are known in the art, a number of which are suitable for use as scaffold polypeptide sequences.

The scaffold polypeptide of a T-Cell-MP may comprise a coiled-coil polypeptide sequence that forms a dimer. Non-limiting examples of coiled-coil polypeptides include, for example, a peptide of any one of the following aa sequences: LKSVENRLAVVENQLKTVIEELKTVKDLLSN (SEQ ID NO:97); LARIEEKLKTIKAQLSEIASTLNMIREQLAQ (SEQ ID NO:98); VSRLEEKVKTLKSQVTELAS-TVSLLREQVAQ (SEQ ID NO:99); IQSEKKIEDISSLIGQIQSEITLIRNEIAQ (SEQ ID NO:100); and LMSLEKKLEELTQTLMQLQNELSMLKNELAQ (SEQ ID NO:101).

The T-Cell-MPs of a T cell MP duplex may comprise a pair of scaffold polypeptide sequences that each comprise at least one cysteine residue that can form a disulfide bond permitting homodimerization or heterodimerization of those polypeptides stabilized by an interchain disulfide bond between the cysteine residues. Examples of such aa sequences include: VDLEGSTSNGRQCAGIRL (SEQ ID NO:102); EDDVTTTEELAPALVPPPKGTCAGWMA (SEQ ID NO:103); and GHDQE-TTTQGPGVLLPLPKGACTGQMA (SEQ ID NO:104).

Some scaffold polypeptide sequences permit formation of T-Cell-MP complexes of higher order than duplexes, such as triplexes, tetraplexes, pentaplexes or hexaplexes. Such aa sequences include, but are not limited to, IgM constant regions (discussed below). Collagen domains, which form trimers, can also be employed. Collagen domains may comprise the three aa sequence Gly-Xaa-Xaa and/or GlyXaaYaa, where Xaa and Yaa are independently any aa, with the sequence appear or are repeated multiple times (e.g., from 10 to 40 times). In Gly-Xaa-Yaa sequences, Xaa and Yaa are frequently proline and hydroxyproline respectively in greater than 25%, 50%, 75%, 80% 90% or 95% of the Gly-Xaa-Yaa occurrences, or in each of the Gly-Xaa-Yaa occurrences. In some cases, a collagen domain comprises the sequence Gly-Xaa-Pro repeated from 10 to 40 times. A collagen oligomerization peptide can comprise the following aa sequence: VTAFSNMDDMLQKAHLVIEGTFIYLRDSTEFFIRVRDGW-KKLQLGELIPIPADSPPPPALSSNP (SEQ ID NO:105).

b. Immunoglobulin Fc Scaffold Polypeptides

(i) Non-Interspecific Immunoglobulin Fc Scaffold Polypeptides

The scaffold polypeptide sequences of a T-Cell-MP or its corresponding T-Cell-MP-epitope conjugate may comprise a Fc polypeptide. The Fc polypeptide of a T-Cell-MP or T-Cell-MP-epitope conjugate can be, for example, from an IgA, IgD, IgE, IgG, or IgM, any of which may be a human polypeptide sequence, a humanized polypeptide sequence, a Fc region polypeptide of a synthetic heavy chain constant region, or a consensus heavy chain constant region. In embodiments, the Fc polypeptide can be from a human IgG1 Fc, a human IgG2 Fc, a human IgG3 Fc, a human IgG4 Fc, a human IgA Fc, a human IgD Fc, a human IgE Fc, a human IgM Fc, etc. In some cases, the Fc polypeptide comprises an aa sequence having at least about 70% (e.g., at least about 75%, 80%, 85%, 90%, 95%, 98%, or 99%), or 100% aa sequence identity to at least 125 contiguous aas (e.g., at least 150, at least 175, at least 200, or at least 210 contiguous aas), or all aas of an aa sequence of a Fc region depicted in FIGS. 2A-2H. Such immunoglobulin sequences can interact forming a duplex or higher order structure from T-Cell-MP molecules. In some instances, the Fc scaffold polypeptide sequences include naturally occurring cysteine residues (or non-naturally occurring cysteine residues provided by protein engineering) that are capable of forming interchain disulfide bonds covalently linking two T-Cell-MP polypeptides together. Unless stated otherwise, the Fc polypeptides used in the T-Cell-MPs and their epitope conjugates do not comprise a transmembrane anchoring domain or a portion thereof sufficient to anchor the T-Cell-MP to a cell membrane.

Most immunoglobulin Fc scaffold polypeptides, particularly those comprising only or largely wt. sequences, may spontaneously link together via disulfide bonds to form homodimers resulting in duplex T-Cell-MPs. In the case of IgM heavy chain constant regions, in the presences of a J-chains, higher order complexes may be formed. Scaffold polypeptides may comprise an aa sequence having 100% aa sequence identity to the wt. human IgG1 Fc polypeptide depicted in FIG. 2D. A scaffold polypeptide may comprise an aa sequence having at least about 70% (e.g., at least about 80%, 90%, 95%, 98%, or 99%) or 100% aa sequence identity to at least 125 contiguous aas (e.g., at least 150, at least 175, at least 200, or at least 210 contiguous aas), or all aas, of the wt. human IgG1 Fc polypeptide depicted in FIG. 2D. Such scaffold sequences may include a substitution of N297 (N77 as numbered in FIG. 2D, SEQ ID NO:4) with an aa other than asparagine. In one case, N297 is substituted by alanine, (N297A). Substitutions at N297 lead to the removal of carbohydrate modifications and result antibody sequences with reduced complement component 1q (“C1q”) binding compared to the wt. protein, and accordingly a reduction in complement-dependent cytotoxicity (CDC). K322 (e.g., K322A) substitutions shows a substantial reduction in reduction in FcγR binding affinity and ADCC, with the C1q binding and CDC functions substantially or completely eliminated. Hezareh et al., (2001) J. Virol. 75:12161-168.

Amino acid L234 and other aas in the lower hinge region (e.g., aas 234 to 239, such as L235, G236, G237, P238, 5239) which correspond to aas 14-19 of SEQ ID NO:8) of IgG are involved in binding to the Fc gamma receptor (FcγR), and accordingly, mutations at that location reduce binding to the receptor (relative to the wt. protein) and resulting in a reduction in antibody-dependent cellular cytotoxicity (ADCC). Hezareh et al., (2001) have demonstrated that the double mutant (L234A, L235A) does not effectively bind either FcγR or C1q, and both ADCC and CDC functions were substantially or completely abolished. A scaffold polypeptide with a substitution in the lower hinge region may comprise an aa sequence having at least about 70% (e.g., at least about 80%, 90%, 95%, 98%, or 99%) aa sequence identity to at least 125 contiguous aas (e.g., at least 150, at least 175, at least 200, or at least 210 contiguous aas), or all aas, of the wt. human IgG1 Fc polypeptide depicted in FIG. 2D, that includes a substitution of L234 (L14 of the aa sequence depicted in FIG. 2D) with an aa other than leucine.

A scaffold polypeptide with a substitution in the lower hinge region may comprise an aa sequence having at least about 70% (e.g., at least about 80%, 90%, 95%, 98%, or 99%) aa sequence identity to at least 125 contiguous aas (e.g., at least 150, at least 175, at least 200, or at least 210 contiguous aas), or all aas, of the wt. human IgG1 Fc polypeptide depicted in FIG. 2D, that includes a substitution of L235 (L15 of the aa sequence depicted in FIG. 22D) with an aa other than leucine. In some cases, the scaffold polypeptide present in a T-Cell-MP with substitutions in the lower hinge region includes L234A and L235A (“LALA”) substitutions (the positions corresponding to positions 14 and 15 of the wt. aa sequence depicted in FIG. 2D; see, e.g., SEQ ID NO:8).

A scaffold polypeptide with a substitution in the lower hinge region may comprise an aa sequence having at least about 70% (e.g., at least about 80%, 90%, 95%, 98%, or 99%) aa sequence identity to at least 125 contiguous aas (e.g., at least 150, at least 175, at least 200, or at least 210 contiguous aas), or all aas of the wt. human IgG1 Fc polypeptide depicted in FIG. 2D, that includes a substitution of P331 (P111 of the aa sequence depicted in FIG. 2D) with an aa other than proline. Substitutions at P331, like those at N297, lead to reduced binding to C1q relative to the wt. protein, and thus a reduction in complement dependent cytotoxicity. In one embodiment, the substitution is a P331S substitution. In another embodiment, the substitution is a P331A substitution.

A scaffold polypeptide may comprise an aa sequence having at least about 70% (e.g., at least about 80%, 90%, 95%, 98%, or 99%) aa sequence identity to at least 125 contiguous aas (e.g., at least 150, at least 175, at least 200, or at least 210 contiguous aas), or all aas, of the wt. human IgG1 Fc polypeptide depicted in FIG. 2D, and include substitutions of D270, K322, and/or P329 (corresponding to D50, K102, and P109 of SEQ ID NO:4 in FIG. 2D) that reduce binding to C1q protein relative to the wt. proteins.

A scaffold polypeptide may comprise an aa sequence having at least about 70% (e.g., at least about 80%, 90%, 95%, 98%, or 99%) aa sequence identity to at least 125 contiguous aas (e.g., at least 150, at least 175, at least 200, or at least 210 contiguous aas), or all aas, of the wt. human IgG1 Fc polypeptide depicted in FIG. 2D, including substitutions at L234 and/or L235 (L14 and/or L15 of the aa sequence depicted in FIG. 2D) with aas other than leucine (such as L234A and L235A substitutions), and a substitution of P331 (P111 of the aa sequence depicted in FIG. 2D) with an aa other than proline such as P331S. In one instance, a scaffold polypeptide present in a T-Cell-MP comprises the “Triple Mutant” aa sequence (SEQ ID NO:6) depicted in FIG. 2D (human IgG1 Fc) having L234F, L235E, and P331S substitutions (corresponding to aa positions 14, 15, and 111 of the aa sequence depicted in FIG. 2D).

The scaffold Fc polypeptide of a T-Cell-MP may comprise an aa sequence having at least about 70% (e.g., at least about 75%, 80%, 85%, 90%, 95%, 98%, or 99%), or 100% aa, sequence identity to at least 125 contiguous aas (e.g., at least 150, at least 175, at least 200, or at least 210 contiguous aas), or all aas, of a human IgG2 Fc polypeptide depicted in FIG. 2E. The scaffold Fc polypeptide of a T-Cell-MP may comprise an aa sequence having at least about 70% (e.g., at least about 75%, 80%, 85%, 90%, 95%, 98%, or 99%), or 100% aa, sequence identity to at least 125 contiguous aas (e.g., at least 150, at least 175, at least 200, or at least 210 contiguous aas), or all aas, of a human IgG3 Fc polypeptide depicted in FIG. 2F. The scaffold Fc polypeptide of a T-Cell-MP may comprise an aa sequence having at least about 70% (e.g., at least about 75%, 80%, 85%, 90%, 95%, 98%, or 99%), or 100% aa, sequence identity to at least 125 contiguous aas (e.g., at least 150, at least 175, at least 200, or at least 210 contiguous aas), or all aas, of a human IgG4 Fc polypeptide depicted in FIG. 2G. The scaffold Fc polypeptide of a T-Cell-MP may comprise an aa sequence having at least about 70% (e.g., at least about 75%, 80%, 85%, 90%, 95%, 98%, or 99%), or 100% aa sequence identity to at least 125 contiguous aas (e.g., at least 150, at least 175, at least 200, or at least 210 contiguous aas e.g., aas 99 to 327 or 111 to 327), or all of the GenBank P01861 human IgG4 Fc polypeptide depicted in FIG. 2G.

The scaffold Fc polypeptide of a T-Cell-MP may comprise IgM heavy chain constant regions (see e.g., FIG. 2H), which forms hexamer, or pentamers (particularly when combined with a mature j-chain peptide lacking a signal sequence such as that provided in FIG. 2I.

(ii) Interspecific Immunoglobulin Fc Scaffold Polypeptides

Where an asymmetric pairing between two T-Cell-MP molecules is desired (e.g., to produce a duplex T-Cell-MP with different MODs), a scaffold polypeptide present in a T-Cell-MP may comprise, consist essentially of, or consist of an interspecific Ig Fc polypeptides) sequence variants. Such interspecific polypeptide sequences include, but are not limited to, knob-in-hole without (KiH) or with (KiHs-s) a stabilizing disulfide bond, HA-TF, ZW-1, 7.8.60, DD-KK, EW-RVT, EW-RVTs-s, and A107 sequences. One interspecific binding pair comprises a T366Y and Y407T mutant pair in the CH3 domain interface of IgG1, or the corresponding residues of other immunoglobulins. See Ridgway et al., Protein Engineering 9:7, 617-621 (1996). A second interspecific binding pair involves the formation of a knob by a T366W substitution, and a hole by the triple substitutions T366S, L368A and Y407V on the complementary Ig Fc sequence. See Xu et al. mAbs 7:1, 231-242 (2015). Another interspecific binding pair has a first Ig Fc polypeptide with Y349C, T366S, L368A, and Y407V substitutions and a second Ig Fc polypeptide with S354C, and T366W substitutions (disulfide bonds can form between the Y349C and the S354C). See e.g., Brinkmann and Konthermann, mAbs 9:2, 182-212 (2015). Ig Fc polypeptide sequences, either with or without knob-in-hole modifications, can be stabilized by the formation of disulfide bonds between the Ig Fc polypeptides (e.g., the hinge region disulfide bonds). Several interspecific binding sequences based upon immunoglobulin sequences are summarized in the table that follows, with cross reference to the numbering of the aa positions as they appear in the wt. IgG1 sequence (SEQ ID NO:4) set forth in FIG. 2D shown in brackets “{ }”.

TABLE 1
Interspecific immunoglobulin sequences and their cognate counterpart interspecific sequences

| | Substitutions in the first | Substitutions in the second | |
|---|---|---|---|
| Interspecific | interspecific polypeptide | (counterpart) interspecific | |
| Pair Name | sequence | polypeptide sequence | Comments |
| KiH | T366W | T366S/L368A/Y407V | Hydrophobic/steric |
| | {T146W} | {T146S/L148A/Y187V} | complementarity |
| KiHs-s | T366W/S354C* | T366S/L368A/Y407V/Y349C | KiH + inter-CH3 |
| | {T146W/S134C*} | {T146S/L148A/Y187V/Y129C} | domain S—S bond |
| HA-TF | S364H/F405A | Y349T/T394F | Hydrophobic/steric |
| | {S144H/F185A} | {Y129T/T174F} | complementarity |
| ZW1 | T350V/L351Y/F405A/Y407V | T350V/T366L/K392L/T394W | Hydrophobic/steric |
| | {T130V/L131Y/F185A/Y187V} | {T130V/T146L/K172L/T174W} | complementarity |
| 7.8.60 | K360D/D399M/Y407A | E345R/Q347R/T366V/K409V | Hydrophobic/steric |
| | {K140D/D179M/Y187A} | {E125R/Q127R/T146V/K189V} | complementarity + |
| | | | electrostatic |
| | | | complementarity |
| DD-KK | K409D/K392D | D399K/E356K | Electrostatic |
| | {K189D/K172D} | {D179K/E136K} | complementarity |
| EW-RVT | K360E/K409W | Q347R/D399V/F405T | Hydrophobic/steric |
| | {K140E/K189W} | {Q127R/D179V/F185T} | complementarity & |
| | | | long-range electro- |
| | | | static interaction |
| EW-RVTs-s | K360E/K409W/Y349C* | Q347R/D399V/F405T/S354C | EW-RVT + inter- |
| | {K140E/K189W/Y129C*} | {Q127R/D179V/F185T/S134C} | CH3 domain SS |
| | | | bond |
| A107 | K370E/K409W | E357N/D399V/F405T | Hydrophobic/steric |
| | {K150E/K189W} | {E137N/D179V/F185T} | complementarity + |
| | | | hydrogen bonding |
| | | | complementarity |

Table 1 is modified from Ha et al., Frontiers in Immunol. 7: 1-16 (2016).
*aa forms a stabilizing disulfide bond.

In addition to the interspecific pairs of sequences in Table 1, scaffold polypeptides may include interspecific “SEED” sequences having 45 residues derived from IgA in an IgG1 CH3 domain of the interspecific sequence, and 57 residues derived from IgG1 in the IgA CH3 in its counterpart interspecific sequence. See Ha et al., Frontiers in Immunol. 7:1-16 (2016).

Interspecific immunoglobulin sequences my include substitutions described above for non-interspecific immunoglobulin sequences that inhibit binding either or both of the FcγR or C1q, and reduce or abolish ADCC and CDC function.

In an embodiment, a scaffold polypeptide found in a T-Cell-MP may comprise an interspecific binding sequence or its counterpart interspecific binding sequence selected from the group consisting of: knob-in-hole (KiH); knob-in-hole with a stabilizing disulfide (KiHs-s); HA-TF; ZW-1; 7.8.60; DD-KK; EW-RVT; EW-RVTs-s; A107; or SEED sequences.

In an embodiment, a T-Cell-MP comprises a scaffold polypeptide comprising an IgG1 sequence with a T146W KiH sequence substitution, and its counterpart interspecific binding partner polypeptide comprises an IgG1 sequence having T146W, L148A, and Y187V KiH sequence substitutions, where the scaffold polypeptides comprises a sequence having at least 80%, at least 90%, at least 95%, or at least 97% sequence identity to at least 100 (e.g., at least 125, 150, 170, 180, 190, 200, 210, 220, or all 227) contiguous aas of the wt. IgG1 of FIG. 2D. Scaffold polypeptides optionally comprise substitutions at one of more of: L234 and L235 (e.g., L234A/L235A “LALA” or L234F/L235E); N297 (e.g., N297A); P331 (e.g. P331S); L351 (e.g., L351K); T366 (e.g., T366S); P395 (e.g., P395V); F405 (e.g., F405R); Y407 (e.g., Y407A); and K409 (e.g., K409Y). Those substitutions appear at: L14 and L15 (e.g., L14A/L15A “LALA” or L14F/L15E); N77 (e.g., N77A); P111 (e.g. P111S) L131 (e.g., L131K); T146 (e.g., T146S); P175 (e.g., P175V); F185 (e.g., F185R); Y187 (e.g., Y187A); and K189 (e.g., K189Y) in the wt. IgG1 sequence of FIG. 2D.

In an embodiment, a T-Cell-MP or duplex T-Cell-MP comprises a scaffold polypeptide comprising an IgG1 sequence with a T146W KiH sequence substitution, and its counterpart interspecific binding partner polypeptide comprises an IgG1 sequence having T146S, L148A, and Y187V KiH sequence substitutions, where the scaffold polypeptides comprise a sequence having at least 80%, at least 90%, at least 95%, or at least 97% sequence identity to at least 100 (e.g., at least 125, 150, 170, 180, 190, 200, 210, 220, or all 227) contiguous aas of the wt. IgG1 of FIG. 2D; where one or both (in the case of duplex T-Cell-MP) scaffold polypeptide sequence(s) may comprise additional substitutions such as L14 and/or L15 substitutions (e.g., “LALA” substitutions L234A and L235A), and/or N77 (N297 e.g., N297A or N297G).

In an embodiment, a T-Cell-MP or duplex T-Cell-MP comprises a scaffold polypeptide comprising an IgG1 sequence with a T146W and S134C KiHs-s substitutions, and its counterpart interspecific binding partner polypeptide comprises an IgG1 sequence having T146S, L148A, Y187V and Y129C KiHs-s substitutions, where the scaffold polypeptides comprise a sequence having at least 80%, at least 90%, at least 95%, or at least 97% sequence identity to at least 100 (e.g., at least 125, 150, 170, 180, 190, 200, 210, 220, or all 227) contiguous aas of the wt. IgG1 of FIG. 2D; where one or both (in the case of duplex T-Cell-MP) scaffold polypeptide sequence(s) sequences may comprise additional substitutions such as L14 and/or L15 substitutions (e.g., “LALA” substitutions L234A and L235A), and/or N77 (N297 e.g., N297A or N297G).

In an embodiment, a T-Cell-MP comprises a scaffold polypeptide comprising an IgG1 sequence with a S144H and F185A HA-TF substitutions, and its counterpart interspecific binding partner polypeptide comprises an IgG1 sequence having Y129T and T174F HA-TF substitutions, where the scaffold polypeptides comprise a sequence having at least 80%, at least 90%, at least 95%, or at least 97% sequence identity to at least 100 (e.g., at least 125, 150, 170, 180, 190, 200, 210, 220, or all 227) contiguous aas of the wt. IgG1 of FIG. 2D; where one or both (in the case of duplex T-Cell-MP) scaffold polypeptide sequence(s) may comprise additional substitutions such as L14 and/or L15 substitutions (e.g., “LALA” substitutions L234A and L235A), and/or N77 (N297 e.g., N297A or N297G).

In an embodiment, a T-Cell-MP or duplex T-Cell-MP comprises a scaffold polypeptide comprising an IgG1 sequence with a T130V, L131Y, F185A, and Y187V ZW1 substitutions, and its counterpart interspecific binding partner polypeptide comprises an IgG1 sequence having T130V, T146L, K172L, and T174W ZW1 substitutions, where the scaffold polypeptides comprise a sequence having at least 80%, at least 90%, at least 95%, or at least 97% sequence identity to at least 100 (e.g., at least 125, 150, 170, 180, 190, 200, 210, 220, or all 227) contiguous aas of the wt. IgG1 of FIG. 2D; where one or both (in the case of duplex T-Cell-MP) scaffold polypeptide sequence(s) may comprise additional substitutions such as L14 and/or L15 substitutions (e.g., “LALA” substitutions L234A and L235A), and/or N77 (N297 e.g., N297A or N297G).

In an embodiment, a T-Cell-MP or duplex T-Cell-MP comprises a scaffold polypeptide comprising an IgG1 sequence with a K140D, D179M, and Y187A 7.8.60 substitutions, and its counterpart interspecific binding partner polypeptide comprises an IgG1 sequence having T130V E125R, Q127R, T146V, and K189V 7.8.60 substitutions, where the scaffold polypeptides comprise a sequence having at least 80%, at least 90%, at least 95%, or at least 97% sequence identity to at least 100 (e.g., at least 125, 150, 170, 180, 190, 200, 210, 220, or all 227) contiguous aas of the wt. IgG1 of FIG. 2D; where one or both (in the case of duplex T-Cell-MP) scaffold polypeptide sequence(s) may comprise additional substitutions such as L14 and/or L15 substitutions (e.g., “LALA” substitutions L234A and L235A), and/or N77 (N297 e.g., N297A or N297G).

In an embodiment, a T-Cell-MP or duplex T-Cell-MP comprises a scaffold polypeptide comprising an IgG1 sequence with a K189D, and K172D DD-KK substitutions, and its counterpart interspecific binding partner polypeptide comprises an IgG1 sequence having T130V D179K and E136K DD-KK substitutions, where the scaffold polypeptides comprise a sequence having at least 80%, at least 90%, at least 95%, or at least 97% sequence identity to at least 100 (e.g., at least 125, 150, 170, 180, 190, 200, 210, 220, or all 227) contiguous aas of the wt. IgG1 of FIG. 2D; where one or both (in the case of duplex T-Cell-MP) scaffold polypeptide sequence(s) may comprise additional substitutions such as L14 and/or L15 substitutions (e.g., “LALA” substitutions L234A and L235A), and/or N77 (N297 e.g., N297A or N297G).

In an embodiment, a T-Cell-MP or duplex T-Cell-MP comprises a scaffold polypeptide comprising an IgG1 sequence with a K140E and K189W EW-RVT substitutions, its counterpart interspecific binding partner polypeptide comprises an IgG1 sequence having T130V Q127R, D179V, and F185T EW-RVT substitutions, where the scaffold polypeptides comprise a sequence having at least 80%, at least 90%, at least 95%, or at least 97% sequence identity to at least 100 (e.g., at least 125, 150, 170, 180, 190, 200, 210, 220, or all 227) contiguous aas of the wt. IgG1 of FIG. 2D; where one or both (in the case of duplex T-Cell-MP) scaffold polypeptide sequence(s) may comprise additional substitutions such as L14 and/or L15 substitutions (e.g., “LALA” substitutions L234A and L235A), and/or N77 (N297 e.g., N297A or N297G).

In an embodiment, a T-Cell-MP or duplex T-Cell-MP comprises a scaffold polypeptide comprising an IgG1 sequence with a K140E, K189W, and Y129C EW-RVTs-s substitutions, its counterpart interspecific binding partner polypeptide comprises an IgG1 sequence having T130V Q127R, D179V, F185T, and S134C EW-RVTs-s substitutions, where the scaffold polypeptides comprise a sequence having at least 80%, at least 90%, at least 95%, or at least 97% sequence identity to at least 100 (e.g., at least 125, 150, 170, 180, 190, 200, 210, 220, or all 227) contiguous aas of the wt. IgG1 of FIG. 2D; where one or both (in the case of duplex T-Cell-MP) scaffold polypeptide sequence(s) may comprise additional substitutions such as L14 and/or L15 substitutions (e.g., “LALA” substitutions L234A and L235A), and/or N77 (N297 e.g., N297A or N297G).

In an embodiment, a T-Cell-MP or duplex T-Cell-MP comprises a scaffold polypeptide comprising an IgG1 sequence with a K150E and K189W A107 substitutions, its counterpart interspecific binding partner polypeptide comprises an IgG1 sequence having T130V E137N, D179V, and F185T A107 substitutions, where the scaffold polypeptides comprise a sequence having at least 80%, at least 90%, at least 95%, or at least 97% sequence identity to at least 100 (e.g., at least 125, 150, 170, 180, 190, 200, 210, 220, or all 227) contiguous aas of the wt. IgG1 of FIG. 2D; where one or both (in the case of duplex T-Cell-MP) scaffold polypeptide sequence(s) may comprise additional substitutions such as L14 and/or L15 substitutions (e.g., “LALA” substitutions L234A and L235A), and/or N77 (N297 e.g., N297A or N297G).

As an alternative to the use of immunoglobulin CH2 and CH3 heavy chain constant regions as scaffold sequences, immunoglobulin light chain constant regions (See FIG. 2K) can be paired with Ig CH1 sequences (See, e.g., FIG. 2J) as interspecific scaffold sequences.

In an embodiment, a T-Cell-MP scaffold polypeptide comprises an Ig CH1 domain (e.g., the polypeptide of FIG. 2J), and the sequence with which it will form a complex (its counterpart binding partner) comprises is an Ig κ chain constant region sequence, where the scaffold polypeptide comprise a sequence having at least 80%, 85%, 90%, 95%, 98%, 99%, or 100% sequence identity to at least 70, at least 80, at least 90, at least 100, or at least 110 contiguous aas of SEQ ID NOs: 16 and/or 17 resp. See FIG. 2K. The Ig CH1 and Ig κ sequences may be modified to increase their affinity for each other, and accordingly the stability of any heterodimer formed utilizing them. Among the substitutions that increase the stability of CH1-Ig κ heterodimers are those identified as the MD13 combination in Chen et al., MAbs, 8(4):761-774 (2016). In the MD13 combination two substitutions are introduced into to each of the IgCH1 and Ig κ sequences. The Ig CH1 sequence is modified to contain S64E and S66V substitutions (570E and S72V of the sequence shown in FIG. 2J). The Ig κ sequence is modified to contain S69L and T715 substitutions (S68L and T705 of the sequence shown in FIG. 2K).

In another embodiment, a scaffold polypeptide of a T-Cell-MP comprises an Ig CH1 domain (e.g., the polypeptide of FIG. 2J SEQ ID NO:15), and its counterpart sequence comprises an Ig chain constant region sequence such as is shown in FIG. 2K (SEQ ID NO:17), where the scaffold polypeptide comprises a sequence having at least 80%, 85%, 90%, 95%, 98%, 99%, or 100% sequence identity to at least 70 (e.g., at least 80, at least 90, or at least 100) contiguous aas of the sequences shown in FIG. 2K.

c. Effects on Stability and Half-Life

Suitable scaffold polypeptides (e.g., those with an Ig Fc scaffold sequence) will in some cases extend the half-life of T-Cell-MP polypeptides and their higher order complexes. In some cases, a suitable scaffold polypeptide increases the in vivo half-life (e.g., the serum half-life) of the T-Cell-MP or duplex T-Cell-MP, compared to a control T-Cell-MP or duplex T-Cell-MP lacking the scaffold polypeptide or comprising a control scaffold polypeptide. For example, in some cases, a scaffold polypeptide increases the in vivo half-life (e.g. serum half-life) of a conjugated or unconjugated T-Cell-MP or duplex T-Cell-MP, compared to an otherwise identical control lacking the scaffold polypeptide, or having a control scaffold polypeptide, by at least about 10%, at least about 20%, at least about 30%, at least about 50%, at least about 2-fold, at least about 5-fold, at least about 10-fold, at least about 25-fold, at least about 50-fold, at least about 100-fold, or more than 100-fold.

5 Immunomodulatory Polypeptides (“MODs”)

MODs that are suitable for inclusion in a T-Cell-MP of the present disclosure include, but are not limited to, wt. and variants of the following immunomodulatory polypeptides IL-1, IL-2, IL-4, IL-6, IL-7, IL-10, IL-12, IL-15, IL-17, IL-21, IL-23, CD7, CD30L, CD40, CD70, CD80, (B7-1), CD83, CD86 (B7-2), HVEM (CD270), ILT3 (immunoglobulin-like transcript 3), ILT4(immunoglobulin-like transcript 4), Fas ligand (FasL), ICAM (intercellular adhesion molecule), ICOS-L (inducible costimulatory ligand), JAG1 (CD339), lymphotoxin beta receptor, 3/TR6, OX40L (CD252), PD-L1, PD-L2, TGF-β1, TGF-β2, TGF-β3, 4-1BBL, and fragments of any thereof, such as ectodomain fragments, capable of engaging and signaling through their cognate receptor). Unless stated otherwise, it is understood that the MODs employed in the T-Cell-MPs of this disclosure may be either wt. and/or variants of wt. immunomodulatory polypeptides, e.g., a variant that selectively binds to a particular Co-MODs and/or has reduced affinity to a particular Co-MOD. Some MOD polypeptides suitable for inclusion in a T-Cell-MP of the present disclosure and their Co-MOD or Co-MODs (“co-immunomodulatory polypeptides” or cognate costimulatory receptors) include polypeptide sequences with T cell modulatory activity from the protein pairs recited in the following table:

Exemplary Pairs of MODs and Co-MODs

| | |
|---|---|
| a) | 4-1BBL (MOD) and 4-1BB (Co-MOD); |
| b) | PD-L1 (MOD) and PD1 (Co-MOD); |
| c) | IL-2 (MOD) and IL-2 receptor (Co-MOD); |
| d) | CD80 (MOD) and CD28 (Co-MOD); |
| e) | CD86 (MOD) and CD28 (Co-MOD); |
| f) | OX40L (CD252) (MOD) and OX40 (CD134) (Co-MOD); |
| g) | Fas ligand (MOD) and Fas (Co-MOD); |
| h) | ICOS-L (MOD) and ICOS (Co-MOD); |
| i) | ICAM (MOD) and LFA-1 (Co-MOD); |
| j) | CD30L (MOD) and CD30 (Co-MOD); |
| k) | CD40 (MOD) and CD40L (Co-MOD); |
| l) | CD83 (MOD) and CD83L (Co-MOD); |
| m) | HVEM (CD270) (MOD) and CD160 (Co-MOD); |
| n) | JAG1 (CD339) (MOD) and Notch (Co-MOD); |
| o) | JAG1 (CD339) (MOD) and CD46 (Co-MOD); |
| p) | CD70 (MOD) and CD27 (Co-MOD); |
| q) | CD80 (MOD) and CTLA4 (Co-MOD); |
| r) | CD86 (MOD) and CTLA4 (Co-MOD); |
| s) | PD-L1(MOD) and CD-80 (Co-MOD); and |
| t) | TGF-β1, TGF-β2, and/or TGF-β3 (MODs) and TGF-β Receptor |
| | (e.g., TGFBR1 and/or TGFBR2) (Co-MOD) |

In some cases, the MOD is selected from a wt. or variant of an IL-2 polypeptide, a 4-1BBL polypeptide, a B7-1 polypeptide; a B7-2 polypeptide, an ICOS-L polypeptide, an OX-40L polypeptide, a CD80 polypeptide, a CD86 polypeptide, a PD-L1 polypeptide, a FasL polypeptide, a TGFβ polypeptide, and a PD-L2 polypeptide. In some cases, the T-Cell-MP or duplex T-Cell-MP comprises two different MODs, such as an IL-2 MOD or IL-2 variant MOD polypeptide and either a wt. or variant of a CD80 or CD86 MOD polypeptide. In another instance, the T-Cell-MP or duplex T-Cell-MP comprises an IL-2 MOD or IL-2 variant MOD polypeptide and a wt. or variant of a PD-L1 MOD polypeptide. In some case MODs, which may be the same or different, are present in a T-Cell-MP or duplex T-Cell-MP in tandem. When MODs are presented in tandem, their sequences are immediately adjacent to each other on a single polypeptide, either without any intervening sequence or separated by only a linker polypeptide (e.g., no MHC sequences or epitope sequences intervene). The MOD polypeptide may comprise all or part of the extracellular portion of a full-length MOD. Thus, for example, the MOD can in some cases exclude one or more of a signal peptide, a transmembrane domain, and an intracellular domain normally found in a naturally-occurring MOD. Unless stated otherwise, a MOD present in a T-Cell-MP or duplex T-Cell-MP does not comprise the signal peptide, intracellular domain, or a sufficient portion of the transmembrane domain to anchor a substantial amount (e.g., more than 5% or 10%) of a T-Cell-MP or duplex T-Cell-MP into a mammalian cell membrane.

In some cases, a MOD suitable for inclusion in a T-Cell-MP comprises all or a portion of (e.g., an extracellular portion of) the aa sequence of a naturally occurring MOD. In other instances, a MOD suitable for inclusion in a T-Cell-MP is a variant MOD that comprises at least one aa substitution compared to the aa sequence of a naturally occurring MOD. In some instances, a variant MOD exhibits a binding affinity for a Co-MOD that is lower than the affinity of a corresponding naturally-occurring MOD (e.g., a MOD not comprising the aa substitution(s) present in the variant) for the Co-MOD. Suitable variations in MOD polypeptide sequence that alter affinity may be identified by scanning (making aa substitution e.g., alanine substitutions or “alanine scanning” or charged residue changes) along the length of a peptide and testing its affinity. Once key aa positions altering affinity are identified those positions can be subject to a vertical scan in which the effect of one or more aa substitutions other than alanine are tested. The affinity may be determined by BLI as described below

a. MODS and Variant MODs with Reduced Affinity

Suitable immunomodulatory domains that exhibit reduced affinity for a co-immunomodulatory domain can have from 1 aa to 20 aa differences from a wt. immunomodulatory domain. For example, in some cases, a variant MOD present in a T-Cell-MP differs in aa sequence by 1 aa to 10 aa, or by 11 aa to 20 aa from a corresponding wt. MOD. A variant MOD present in a T-Cell-MP may include a single aa substitution compared to a corresponding reference (e.g., wt.) MOD. A variant MOD present in a T-Cell-MP may include 2 aa substitutions compared to a corresponding reference (e.g., wt.) MOD. A variant MOD present in a T-Cell-MP may include 3 aa substitutions compared to a corresponding reference (e.g., wt.) MOD. A variant MOD present in a T-Cell-MP may include 4 aa substitutions compared to a corresponding reference (e.g., wt.) MOD. A variant MOD present in a T-Cell-MP may include 5 aa substitutions compared to a corresponding reference (e.g., wt.) MOD. A variant MOD present in a T-Cell-MP may include 6 aa or 7 aa substitutions compared to a corresponding reference (e.g., wt.) MOD. A variant MOD present in a T-Cell-MP may include 8 aa, 9 aa, or 10 aa substitutions compared to a corresponding reference (e.g., wt.) MOD. A variant MOD present in a T-Cell-MP may include 11, 12, 13, 14, or 15 aa substitutions compared to a corresponding reference (e.g., wt.) MOD. A variant MOD present in a T-Cell-MP may include 16, 17, 18, 19, or 20 aa substitutions compared to a corresponding reference (e.g., wt.) MOD.

As discussed above, a variant MOD suitable for inclusion in a T-Cell-MP of the present disclosure may exhibit reduced affinity for a cognate Co-MOD, compared to the affinity of a corresponding wt. MOD for the cognate Co-MOD. In some cases, a variant MOD present in a T-Cell-MP has a binding affinity for a cognate Co-MOD that is from 100 nM to 100 μM. For example, in some cases, a variant MOD present in a T-Cell-MP has a binding affinity for a cognate Co-MOD that is from about 100 nM to about 200 nM, from about 200 nM to about 300 nM, from about 300 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, from about 1 μM to about 5 μM, from about 5 μM to about 10 μM, from about 10 μM to about 20 μM, from about 20 μM to about 30 μM, from about 30 μM to about 50 μM, from about 50 μM to about 75 μM, or from about 75 μM to about 100 μM.

Alternatively, or additionally to reduced affinity binding, the MOD may be a variant that exhibits selective binding to a Co-MOD. In one aspect, where a MOD can bind to more than one Co-MOD, a variant may be chosen that selectively binds to at least one Co-MOD. For example, wt. PD-L1 binds to both PD-1 and CD80 (also known as B7-1). In such case, a variant PD-L1 MOD may be chosen that selectively (preferentially) binds either to PD-1 or CD80. Likewise, where a wt. MOD may bind to multiple polypeptides within a Co-MOD, a variant may be chosen to selectively bind to only the desired polypeptides with the Co-MOD. For example, IL-2 binds to the alpha, beta and gamma chains of IL-2R. A variant of IL-2 can be chosen that either binds with reduced affinity, or does not bind, to one of the polypeptides, e.g., the alpha chain of IL-2R, or even to two of the chains.

(i) Determining Binding Affinity

Binding affinity between a MOD and its cognate Co-MOD can be determined by bio-layer interferometry (BLI) using purified MOD and purified cognate Co-MOD. Binding affinity between a T-Cell-MP and its cognate Co-MOD can also be determined by BLI using purified T-Cell-MP and the cognate Co-MOD. BLI methods are well known to those skilled in the art. See, e.g., Lad et al. (2015) J. Biomol. Screen. 20(4):498-507; and Shah and Duncan (2014) J. Vis. Exp. 18:e51383. The specific and relative binding affinities described in this disclosure between a MOD and its cognate Co-MOD, or between a T-Cell-MP having a MOD and its cognate Co-MOD, can be determined using the following procedures.

To determine binding affinity between a T-Cell-MP and its cognate Co-MOD, a BLI assay can be carried out using an Octet RED 96 (Pal FortéBio) instrument, or a similar instrument, as follows. A T-Cell-MP (e.g., a control T-Cell-MP comprising a wt. MOD)) is immobilized onto an insoluble support (a “biosensor”). The immobilized T-Cell-MP is the “target” Immobilization can be effected by immobilizing a capture antibody onto the insoluble support, where the capture antibody immobilizes the T-Cell-MP. For example, immobilization can be effected by immobilizing anti-Fc (e.g., anti-human IgG Fc) antibodies onto the insoluble support, where the immobilized anti-Fc antibodies bind to and immobilize the T-Cell-MP (where the T-Cell-MP comprises an Ig Fc polypeptide). A Co-MOD is applied, at several different concentrations, to the immobilized T-Cell-MP, and the instrument's response recorded. Assays are conducted in a liquid medium comprising 25 mM HEPES pH 6.8, 5% poly(ethylene glycol) 6000, 50 mM KCl, 0.1% bovine serum albumin, and 0.02% Tween 20 nonionic detergent. Binding of the Co-MOD to the immobilized T-Cell-MP is conducted at 30° C. As a positive control for binding affinity, an anti-MHC Class I monoclonal antibody can be used depending on the class of the MHC polypeptides in the T-Cell-MP. For example, anti-HLA Class I monoclonal antibody (mAb) W6/32 (American Type Culture Collection No. HB-95; Parham et al. (1979) J. Immunol. 123:342), which has a KD of 7 nM, or an anti-HLD-DR3 monoclonal antibody such as the 16-23 antibody (Sigma; also referred to as “16.23”; see, e.g., Pious et al. (1985) J. Exp. Med. 162:1193; Mellins et al. (1991) J. Exp. Med. 174:1607; ECACC hybridoma collection 16-23, ECACC 99043001) can be used as a positive control for binding affinity. A standard curve can be generated using serial dilutions of the anti-MHC Class I monoclonal antibody. The Co-MOD, or the anti-MHC mAb, is the “analyte.” BLI analyzes the interference pattern of white light reflected from two surfaces: i) the immobilized polypeptide (“target”); and ii) an internal reference layer. A change in the number of molecules (“analyte”; e.g., Co-MOD; anti-HLA antibody) bound to the biosensor tip causes a shift in the interference pattern; this shift in interference pattern can be measured in real time. The two kinetic terms that describe the affinity of the target/analyte interaction are the association constant (kₐ) and dissociation constant (k_{d}). The ratio of these two terms (k_{d}/kₐ) gives rise to the affinity constant K_{D}.

As noted above, determining binding affinity between a MOD (e.g., IL-2 or an IL-2 variant) and its cognate Co-MOD (e.g., IL-2R) also can be determined by BLI. The assay is similar to that described above for the T-Cell-MP. A BLI assay can be carried out using an Octet RED 96 (Pal FortéBio) instrument, or a similar instrument, as follows. A component MOD of a T-Cell-MP (e.g., a variant IL-2 polypeptide of the present disclosure); and a control MOD (where a control MOD comprises a wt. MOD, e.g. wt. IL-2)) are immobilized onto insoluble supports (each a “biosensor”). The MOD is the “target.” Immobilization can be effected by immobilizing a capture antibody onto the insoluble support, where the capture antibody immobilizes the MOD. For example, if the target is fused to an immuno-affinity tag (e.g. FLAG, human IgG Fc), immobilization can be effected by immobilizing with the appropriate antibody to the immuno-affinity tag (e.g. anti-human IgG Fc) onto the insoluble support, where the immobilized antibodies bind to and immobilize the MOD (where the MOD comprises an Ig Fc polypeptide). A Co-MOD (or polypeptide) is applied, at several different concentrations, to the immobilized MOD, and the instrument's response recorded. Alternatively, a Co-MOD (or polypeptide) is immobilized to the biosensor (e.g., for the IL-2 receptor heterotrimer, as a monomeric subunit, heterodimeric subcomplex, or the complete heterotrimer) and the MOD is applied, at several different concentrations, to the immobilized Co-MOD(s), and the instrument's response is recorded. Assays are conducted in a liquid medium comprising 25 mM HEPES pH 6.8, 5% poly(ethylene glycol) 6000, 50 mM KCl, 0.1% bovine serum albumin, and 0.02% Tween 20 nonionic detergent. Binding of the Co-MOD to the immobilized MOD is conducted at 30° C. BLI analyzes the interference pattern of white light reflected from two surfaces: i) from the immobilized polypeptide (“target”); and ii) an internal reference layer. A change in the number of molecules (“analyte”; e.g., Co-MOD) bound to the biosensor tip causes a shift in the interference pattern; this shift in interference pattern can be measured in real time. The two kinetic terms that describe the affinity of the target/analyte interaction are the association constant (10 and dissociation constant (k_{d}). The ratio of these two terms (k_{d}/a) gives rise to the affinity constant K_{D}. Determining the binding affinity of both a wt. MOD (e.g., IL-2) for its receptor (e.g., IL-2R) and a variant MOD (e.g., an IL-2 variant as disclosed herein) for its cognate Co-MOD (e.g., its receptor; (e.g., IL-2R) thus allows one to determine the relative binding affinity of the variant Co-MOD, as compared to the wt. Co-MOD, for the cognate Co-MOD. That is, one can determine whether the binding affinity of a variant MOD for its receptor (its cognate Co-MOD) is reduced as compared to the binding affinity of the wt. MOD for the same cognate Co-MOD, and, if so, what is the percentage reduction from the binding affinity of the wt. Co-MOD.

The BLI assay may be carried out in a multi-well plate. To run the assay, the plate layout is defined, the assay steps are defined, and biosensors are assigned in the Octet Data Acquisition software. The biosensor assembly is hydrated. The hydrated biosensor assembly and the assay plate are equilibrated for 10 minutes on the Octet instrument. Once the data are acquired, the acquired data are loaded into the Octet Data Analysis software. The data are processed in the Processing window by specifying method for reference subtraction, y-axis alignment, inter-step correction, and Savitzky-Golay filtering. Data are analyzed in the Analysis window by specifying steps to analyze (Association and Dissociation), selecting curve fit model (1:1), fitting method (global), and window of interest (in seconds). The quality of fit is evaluated. K_{D} values for each data trace (analyte concentration) can be averaged if within a 3-fold range. K_{D} error values should be within one order of magnitude of the affinity constant values; R² values should be above 0.95. See, e.g., Abdiche et al. (2008) J. Anal. Biochem. 377:209.

Unless otherwise stated herein, the affinity of a T-Cell-MP-epitope conjugate of the present disclosure for a Co-MOD, or the affinity of a control T-Cell-MP-epitope conjugate (where a control T-Cell-MP-epitope conjugate comprises a wt. MOD) for a Co-MOD, is determined using BLI, as described above. Likewise, the affinity of a MOD and its Co-MOD polypeptide can be determined using BLI as described above.

A variant MOD present in a T-Cell-MP of the present disclosure may bind to its Co-MOD with an affinity that is at least 10% less, at least 15% less, at least 20% less, at least 25% less, at least 30% less, at least 35% less, at least 40% less, at least 45% less, at least 50% less, at least 55% less, at least 60% less, at least 65% less, at least 70% less, at least 75% less, at least 80% less, at least 85% less, at least 90% less, at least 95% less, or more than 95% less, than the affinity of a corresponding wt. MOD for the Co-MOD.

In some cases, a variant MOD present in a T-Cell-MP of the present disclosure has a binding affinity for a Co-MOD that is from 1 nM to 100 nM, or from 100 nM to 100 μM. For example, in some cases, a variant MOD present in a T-Cell-MP has a binding affinity for a Co-MOD that is from about 1 nM to about 5 nM, from about 5 nM to about 10 nM, from about 10 nM to about 50 nM, from about 50 nM to about 100 nM, from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, from about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM. In some cases, a variant MOD present in a T-Cell-MP has a binding affinity for a Co-MOD that is from about 1 nM to about 5 nM, from about 5 nM to about 10 nM, from about 10 nM to about 50 nM, or from about 50 nM to about 100 nM.

Binding affinity of a T-Cell-MP-epitope conjugate of the present disclosure to a target T cell can be measured in the following manner A) contacting a T-Cell-MP-epitope conjugate of the present disclosure with a target T cell expressing on its surface: i) a Co-MOD that binds to the parental wt. MOD; and ii) a TCR that binds to the epitope, where the T-Cell-MP-epitope conjugate comprises an epitope tag or fluorescent label (e.g., a fluorescent payload or fluorescent protein label, such as green fluorescent protein, as part of the T-Cell-MP), such that the T-Cell-MP-epitope conjugate binds to the target T cell; B) if the T-Cell-MP-epitope conjugate is unlabeled, contacting the target T cell-bound T-Cell-MP-epitope conjugate with a fluorescently labeled binding agent (e.g., a fluorescently labeled antibody) that binds to the epitope tag, generating a T-Cell-MP-epitope conjugate/target T cell/binding agent complex; and C) measuring the mean fluorescence intensity (MFI) of the T-Cell-MP-epitope conjugate/target T cell/binding agent complex using flow cytometry. The epitope tag can be, e.g., a FLAG tag, a hemagglutinin tag, a c-myc tag, a poly(histidine) tag, etc. The MFI measured over a range of concentrations of the T-Cell-MP-epitope conjugate (library member) provides a measure of the affinity. The MFI measured over a range of concentrations of the T-Cell-MP-epitope conjugate (library member) provides a half maximal effective concentration (EC₅₀) of the T-Cell-MP-epitope conjugate. In some cases, the EC₅₀ of a T-Cell-MP-epitope conjugate of the present disclosure for a target T cell is in the nM range; and the EC₅₀ of the T-Cell-MP-epitope conjugate for a control T cell (where a control T cell expresses on its surface: i) a Co-MOD that binds the parental wt. MOD; and ii) a T cell receptor that does not bind to the epitope present in the T-Cell-MP-epitope conjugate) is in the μM range. The ratio of the EC₅₀ of a T-Cell-MP-epitope conjugate of the present disclosure for a control T cell to the EC₅₀ of the T-Cell-MP-epitope conjugate for a target T cell may be at least 1.5:1, at least 2:1, at least 5:1, at least 10:1, at least 15:1, at least 20:1, at least 25:1, at least 50:1, at least 100:1, at least 500:1, at least 10^{b}:1, at least 5×10^{b}:1, at least 10³:1, at least 5×10³:1, at least 10⁴:1, at lease 10⁵:1, or at least 10⁶:1. The ratio of the EC₅₀ of a T-Cell-MP-epitope conjugate of the present disclosure for a control T cell to the EC₅₀ of the T-Cell-MP-epitope conjugate for a target T cell is an expression of the selectivity of the T-Cell-MP-epitope conjugate.

In some cases, when measured as described in the preceding paragraph, a T-Cell-MP-epitope conjugate of the present disclosure exhibits selective binding to a target T cell, compared to binding of the T-Cell-MP-epitope conjugate (library member) to a control T cell that comprises: i) the Co-MOD that binds the parental wt. MOD; and ii) a TCR that binds to an epitope other than the epitope present in the T-Cell-MP-epitope conjugate (library member).

The ratio of: i) the binding affinity of a control T-Cell-MP (where the control T-Cell-MP comprises a wt. MOD) to a cognate Co-MOD to ii) the binding affinity of a T-Cell-MP comprising a variant of the wt. MOD to the cognate Co-MOD, when measured by BLI (as described above), may be at least 1.5:1, at least 2:1, at least 5:1, at least 10:1, at least 15:1, at least 20:1, at least 25:1, at least 50:1, at least 100:1, at least 500:1, at least 10^{b}:1, at least 5×10^{b}:1, at least 10³:1, at least 5×10³:1, at least 10⁴:1, at least 10⁵:1, or at least 10⁶:1. The ratio of: i) the binding affinity of a control T-Cell-MP (where the control T-Cell-MP comprises a wt. MOD) to a cognate Co-MOD to ii) the binding affinity of a T-Cell-MP comprising a variant of the wt. MOD to the cognate Co-MOD, when measured by BLI, may be in a range of from 1.5:1 to 10⁶:1, e.g., from 1.5:1 to 10:1, from 10:1 to 50:1, from 50:1 to 10^{b}:1, from 10^{b}:1 to 10³:1, from 10³:1 to 10⁴:1, from 10⁴:1 to 10⁵:1, or from 10⁵:1 to 10⁶:1.

As an example, where a control T-Cell-MP-epitope conjugate comprises a wt. IL-2 polypeptide, and where a T-Cell-MP-epitope conjugate of the present disclosure comprises a variant IL-2 polypeptide (comprising from 1 to 10 aa substitutions relative to the aa sequence of the wt. IL-2 polypeptide) as the MOD, the ratio of: i) the binding affinity of the control T-Cell-MP-epitope conjugate to an IL-2 receptor (the Co-MOD) to ii) the binding affinity of the T-Cell-MP-epitope conjugate of the present disclosure to the IL-2 receptor (the Co-MOD), when measured by BLI, is at least 1.5:1, at least 2:1, at least 5:1, at least 10:1, at least 15:1, at least 20:1, at least 25:1, at least 50:1, at least 100:1, at least 500:1, at least 10^{b}:1, at least 5×10^{b}:1, at least 10³:1, at least 5×10³:1, at least 10⁴:1, at least 10⁵:1, or at least 10⁶:1. Where a control T-Cell-MP-epitope conjugate comprises a wt. IL-2 polypeptide, and where a T-Cell-MP-epitope conjugate of the present disclosure comprises a variant IL-2 polypeptide (comprising from 1 to 10 aa substitutions relative to the aa sequence of the wt. IL-2 polypeptide) as the MOD, the ratio of: i) the binding affinity of the control T-Cell-MP-epitope conjugate to the IL-2 receptor (the Co-MOD) to ii) the binding affinity of the T-Cell-MP-epitope conjugate of the present disclosure to the IL-2 receptor, when measured by BLI, may be in a range of from 1.5:1 to 10⁶:1, e.g., from 1.5:1 to 10:1, from 10:1 to 50:1, from 50:1 to 10^{b}:1, from 10^{b}:1 to 10³:1, from 10³:1 to 10⁴:1, from 10⁴:1 to 10⁵:1, or from 10⁵:1 to 10⁶:1. Other examples that may have the same ratios of binding affinities include T-Cell-MPs bearing a wt. MOD and T-Cell-MPs bearing a variant MOD where the wt. and variant MODs are selected from: wt. CD80 and variant CD80; wt. CD86 and a variant CD86; wt. PD-L1 and a variant PD-L1; wt. CTLA4 and a variant CTLA4; or wt. 4-1BBL and variant 4-1BBL.

A variant MOD present in a T-Cell-MP of the present disclosure may have a binding affinity for a cognate Co-MOD that is from 1 nM to 100 nM, or from 100 nM to 250 μM. For example, a variant MOD present in a T-Cell-MP may have a binding affinity for a cognate Co-MOD that is from about 1 nM to about 10 nM, from about 10 nM to about 100 nM, from about 100 nM to about 500 nM, from about 500 nM to about 750 nM, from about 750 nM to about 1 μM, from about 1 μM to about 5 μM, from about 5 μM to about 10 μM, from about 10 μM to about 25 μM, from about 25 μM to about 50 μM, from about 50 μM to about 100 μM, or from about 100 μM to about 250 μM. A variant MOD present in a T-Cell-MP may have a binding affinity for a cognate Co-MOD that is from about 1 nM to about 5 nM, from about 5 nM to about 10 nM, from about 10 nM to about 50 nM, or from about 50 nM to about 100 nM.

The combination of the reduced affinity of the MOD for its Co-MOD and the affinity of the epitope for a TCR provides for enhanced selectivity of a T-Cell-MP-epitope conjugate of the present disclosure, while still allowing for activity of the MOD. Thus, a T-Cell-MP-epitope conjugate of the present disclosure may bind selectively to a first T cell that displays both: i) a TCR specific for the epitope present in the T-Cell-MP-epitope conjugate; and ii) a Co-MOD that binds to the MOD present in the T-Cell-MP-epitope conjugate, compared to binding to a second T cell that displays: i) a TCR specific for an epitope other than the epitope present in the T-Cell-MP-epitope conjugate; and ii) a Co-MOD that binds to the MOD present in the T-Cell-MP-epitope conjugate. For example, a T-Cell-MP-epitope conjugate of the present disclosure may bind to the first T cell with an affinity that is at least 10%, at least 15%, at least 20%, at least 25%, at least 30%, at least 40%, at least 50%, at least 60%, at least 70%, at least 80%, at least 90%, at least 200% (2-fold), at least 250% (2.5-fold), at least 500% (5-fold), at least 1,000% (10-fold), at least 1,500% (15-fold), at least 2,000% (20-fold), at least 2,500% (25-fold), at least 5,000% (50-fold), at least 10,000% (100-fold), or more than 100-fold, higher than the affinity to which it binds the second T cell. See e.g., FIG. 1

b. IL-2 and its Variants

As one non-limiting example, a wt. MOD or variant MOD present in a T-Cell-MP is an IL-2 or variant IL-2 polypeptide. In some cases, a variant MOD present in a T-Cell-MP is a variant IL-2 polypeptide. Wild-type IL-2 binds to an IL-2 receptor (IL-2R). A wt. IL-2 aa sequence can be as follows: APTSSSTKKT QLQLEHLLLD LQMILNGINN YKNPKLTRML TFKFYMPKKA TELKHLQCLE EELKPLEEVL NLAQSKNFHL RPRDLISNIN VIVLELKGSE TTFMCEYADE TATIVEFLNR WITFCQSIIS TLT (aa 21-153 of UniProt P60568, SEQ ID NO:106).

Wild-type IL2 binds to an IL2 receptor (IL2R) on the surface of a cell. An IL2 receptor is in some cases a heterotrimeric polypeptide comprising an alpha chain (IL-2Rα; also referred to as CD25), a beta chain (IL-2Rβ; also referred to as CD122) and a gamma chain (IL-2Rγ; also referred to as CD132). Amino acid sequences of human IL-2Rα, IL2Rβ, and IL-2Rγ can be as follows.

Human IL-2Rα:
(SEQ ID NO: 107)
ELCDDDPPE IPHATFKAMA YKEGTMLNCE CKRGFRRIKS
GSLYMLCTGN SSHSSWDNQC QCTSSATRNT TKQVTPQPEE
QKERKTTEMQ SPMQPVDQAS LPGHCREPPP WENEATERIY
HFVVGQMVYY QCVQGYRALH RGPAESVCKM THGKTRWTQP
QLICTGEMET SQFPGEEKPQ ASPEGRPESE TSCLVTTTDF
QIQTEMAATM ETSIFTTEYQ VAVAGCVFLL ISVLLLSGLT
WQRRQRKSRR TI.
Human IL-2Rβ:
(SEQ ID: 108)
VNG TSQFTCFYNS RANISCVWSQ DGALQDTSCQ VHAWPDRRRW
NQTCELLPVS QASWACNLIL GAPDSQKLIT VDIVTLRVLC
REGVRWRVMA IQDFKPFENL RLMAPISLQV VHVETHRCNI
SWEISQASHY FERHLEFEAR TLSPGHTWEE APLLILKQKQ
EWICLETLTP DTQYEFQVRV KPLQGEFTTW SPWSQPLAFR
TKPAALGKDT IPWLGHLLVG LSGAFGFIIL VYLLINCRNT
GPWLKKVLKC NTPDPSKFFS QLSSEHGGDV QKWLSSPFPS
SSFSPGGLAP EISPLEVLER DKVTQLLLQQ DKVPEPASLS
SNHSLTSCFT NQGYFFFHLP DALEIEACQV YFTYDPYSEE
DPDEGVAGAP TGSSPQPLQP LSGEDDAYCT FPSRDDLLLF
SPSLLGGPSP PSTAPGGSGA GEERMPPSLQ ERVPRDWDPQ
PLGPPTPGVP DLVDFQPPPE LVLREAGEEV PDAGPREGVS
FPWSRPPGQG EFRALNARLP LNTDAYLSLQ ELQGQDPTHL V.
Human IL-2Rγ:
(SEQ ID NO: 109)
LNTTILTP NGNEDTTADF FLTTMPTDSL SVSTLPLPEV
QCFVFNVEYM NCTWNSSSEP QPTNLTLHYW YKNSDNDKVQ
KCSHYLFSEE ITSGCQLQKK EIHLYQTFVV QLQDPREPRR
QATQMLKLQN LVIPWAPENL TLHKLSESQL ELNWNNRFLN
HCLEHLVQYR TDWDHSWTEQ SVDYRHKFSL PSVDGQKRYT
FRVRSRFNPL CGSAQHWSEW SHPIHWGSNT SKENPFLFAL
EAVVISVGSM GLIISLLCVY FWLERTMPRI PTLKNLEDLV
TEYHGNFSAW SGVSKGLAES LQPDYSERLC LVSEIPPKGG
ALGEGPGASP CNQHSPYWAP PCYTLKPET.

In some cases, where a T-Cell-MP comprises a variant IL-2 polypeptide, a cognate Co-MOD is an IL-2R comprising polypeptides comprising the aa sequences of any one of SEQ ID NO:107, SEQ ID: 108, and SEQ ID NO:109.

In some cases, a variant IL-2 polypeptide exhibits reduced binding affinity to IL-2R, compared to the binding affinity of an IL-2 polypeptide comprising the aa sequence set forth in SEQ ID NO:106. For example, in some cases, a variant IL-2 polypeptide binds IL-2R with a binding affinity that is at least 10% less, at least 20% less, at least 30% less, at least 40% less, at least 50% less, at least 60% less, at least 70% less, at least 80% less, at least 90% less, at least 95% less, or more than 95% less, than the binding affinity of an IL-2 polypeptide comprising the aa sequence set forth in SEQ ID NO:106 for an IL-2R (e.g., an IL-2R comprising polypeptides comprising the aa sequence set forth in SEQ ID NOs:107-109), when assayed under the same conditions.

In some cases, a variant IL-2 polypeptide (e.g., a variant of SEQ ID NO:106) has a binding affinity to IL-2R (e.g., of SEQ ID NOs:107-109) that is from 100 nM to 100 μM. As another example, in some cases, a variant IL-2 polypeptide (e.g., a variant of SEQ ID NO:106) has a binding affinity for IL-2R (e.g., an IL-2R comprising polypeptides comprising the aa sequence set forth in SEQ ID NOs:107-109) that is from about 100 nM to about 200 nM, from about 200 nM to about 400 nM, from about 400 nM to about 600 nM, from about 600 nM to about 800 nM, from about 800 nM to about 1 μM, from about 1 μM to about 5 μM, from about 5 μM to about 10 μM, from about 10 μM to about 20 μM, from about 20 μM to about 40 μM, from about 40 μM to about 75 μM, or from about 75 μM to about 100 μM.

In some cases, a variant IL-2 polypeptide has a single aa substitution compared to the IL-2 aa sequence set forth in SEQ ID NO:106. In some cases, a variant IL-2 polypeptide has from 2 to 10 aa substitutions compared to the IL-2 aa sequence set forth in SEQ ID NO:106. In some cases, a variant IL-2 polypeptide has 2 aa substitutions compared to the IL-2 aa sequence set forth in SEQ ID NO:106. In some cases, a variant IL-2 polypeptide has 3 aa substitutions compared to the IL-2 aa sequence set forth in SEQ ID NO:106. In some cases, a variant IL-2 polypeptide has 4 aa substitutions compared to the IL-2 aa sequence set forth in SEQ ID NO:106. In some cases, a variant IL-2 polypeptide has 5 aa substitutions compared to the IL-2 aa sequence set forth in SEQ ID NO:106. In some cases, a variant IL-2 polypeptide has 6 or 7 aa substitutions compared to the IL-2 aa sequence set forth in SEQ ID NO:106. In some cases, a variant IL-2 polypeptide has 8, 9, or 10 aa substitutions compared to the IL-2 aa sequence set forth in SEQ ID NO:106.

Suitable variant IL-2 polypeptide sequences include polypeptide sequences comprising an aa sequence having at least 80% (e.g., at least 85%, at least 90%, at least 95%, at least 98%, at least 99%, or 100%) aa sequence identity to at least 80 (e.g., 90, 100, 110, 120, 130 or 133) contiguous aas of SEQ ID NO:106.

IL-2 variants include polypeptides having at least 90% (e.g., at least 95%, 98%, or 99%) aa sequence identity to at least 80 (e.g., at least 90, 100, 110, 120, or 130) contiguous aas of SEQ ID NO:106, wherein the aa at position 15 is an aa other than E. In one case, the position of H16 is substituted by Ala (H16A). In one case, the position of E15 is substituted by Ala (E15A).

IL-2 variants include polypeptides having at least 90% (e.g., at least 95%, 98%, or 99%) aa sequence identity to at least 80 (e.g., at least 90, 100, 110, 120, or 130) contiguous aas of SEQ ID NO:106, wherein the aa at position 16 is an aa other than H. In one case, the position of H16 is substituted by Asn, Cys, Gln, Met, Val, or Trp. In one case, the position of H16 is substituted by Ala. In another case, the position of H16 is substituted by Thr.

IL-2 variants include polypeptides having at least 90% (e.g., at least 95%, 98%, or 99%) aa sequence identity to at least 80 (e.g., at least 90, 100, 110, 120, or 130) contiguous aas of SEQ ID NO:106, wherein the aa at position 20 is an aa other than D. In one case, the position of D20 is substituted by Ala.

IL-2 variants include polypeptides having at least 90% (e.g., at least 95%, 98%, or 99%) aa sequence identity to at least 80 (e.g., at least 90, 100, 110, 120, or 130) contiguous aas of SEQ ID NO:106, wherein the aa at position 42 is an aa other than F. In one case, the position of F42 is substituted by Met, Pro, Ser, Thr, Trp, Tyr, Val, or His. In one case, the position of F42 is substituted by Ala. IL-2 variants include polypeptides having at least 90% (e.g., at least 95%, 98%, or 99%) aa sequence identity to at least 80 (e.g., at least 90, 100, 110, 120, or 130) contiguous aas of SEQ ID NO:106, wherein the aa at position 45 is an aa other than Y. In one case, the position of Y45 is substituted by Ala.

IL-2 variants include polypeptides having at least 90% (e.g., at least 95%, 98%, or 99%) aa sequence identity to at least 80 (e.g., at least 90, 100, 110, 120, or 130) contiguous aas of SEQ ID NO:106, wherein the aa at position 88 is an aa other than N. In one case, the position of N88 is substituted by Ala. In another case, the position of N88 is substituted by Arg.

IL-2 variants include polypeptides having at least 90% (e.g., at least 95%, 98%, or 99%) aa sequence identity to at least 80 (e.g., at least 90, 100, 110, 120, or 130) contiguous aas of SEQ ID NO:106, wherein the aa at position 126 is an aa other than Q. In one case, the position of Q126 is substituted by Ala (Q126A).

IL-2 variants include polypeptides having at least 90% (e.g., at least 95%, 98%, or 99%) aa sequence identity to at least 80 (e.g., at least 100, 110, 120, or 130) contiguous aas of SEQ ID NO:106, wherein the aa at position 16 is an aa other than H and the aa at position 42 is other than F. In one case, the position of H16 is substituted by Ala or Thr and the position of F42 is substituted by Ala or Thr. In one case, the position of H16 is substituted by Ala and the position of F42 is substituted by Ala (an H16A and F42A variant). In one case, the position of H16 is substituted by Thr and the position of F42 is substituted by Ala (an H16T and F42A variant).

An IL-2 variant may comprise an aa sequence having at least 80%, at least 85%, at least 90%, at least 95%, or at least 98% aa sequence identity to the sequence: APTSSSTKKT QLQLE [symbol] LLLD LQMILNGINN YKNPKLTRML T [symbol] KFYMPKKA TELKHLQCLE EELKPLEEVL NLAQSKNFHL RPRDLISNIN VIVLELKGSE TTFMCEYADE TATIVEFLNR WITFCQSIIS TLT (SEQ ID NO:110), wherein position 16 and 42 are substituted as follows: X₁ is any aa other than His; and X₂ is any aa other than Phe. A second IL-2 variant comprises the substitutions X₁ is Ala and X₂ is Ala (an H16A and F42A variant). A third IL-2 variant comprise the substitutions X₁ is Thr and X₂ is Ala (an H16T and F42A variant).

IL-2 variants include polypeptides having at least 90% (e.g., at least 95%, 98%, or 99%) aa sequence identity to at least 80 (e.g., at least 100, 110, 120, or 130) contiguous aas of SEQ ID NO:106, wherein the aa at position 20 is an aa other than D and the aa at position 42 is other than F. In one case, the position of D20 is substituted by Ala and the position of F42 is substituted by Ala (D20A and F42A substitutions).

IL-2 variants include polypeptides having at least 90% (e.g., at least 95%, 98%, or 99%) aa sequence identity to at least 80 (e.g., at least 100, 110, 120, or 130) contiguous aas of SEQ ID NO:106, wherein the aa at position 15 is other than E, the aa at position 20 is an aa other than D, and the aa at position 42 is other than F. In one case, the position of E15 is substituted by Ala, the position of D20 is substituted by Ala and the position of F42 is substituted by Ala (EISA, D20A, an dF42A substitutions).

IL-2 variants include polypeptides having at least 90% (e.g., at least 95%, 98%, or 99%) aa sequence identity to at least 80 (e.g., at least 100, 110, 120, or 130) contiguous aas of SEQ ID NO:106, wherein the aa at position 16 is other than H, the aa at position 20 is an aa other than D, and the aa at position 42 is other than F. In one case, the position of H16 is substituted by Ala, the position of D20 is substituted by Ala and the position of F42 is substituted by Ala (an H16A, D20A, and F42A substitution). In another case, the position H16 is substituted by Thr, the position of D20 is substituted by Ala and the position of F42 is substituted by Ala (H16T, D20A, and F42A substitutions).

IL-2 variants include polypeptides having at least 90% (e.g., at least 95%, 98%, or 99%) aa sequence identity to at least 80 (e.g., at least 100, 110, 120, or 130) contiguous aas of SEQ ID NO:106, wherein the aa at position 16 is other than H, the aa at position 42 is other than F, and the aa at position 88 is other than R. In one case, the position of H16 is substituted by Ala or Thr, the position of F42 is substituted by Ala, and the position of N88 is substituted by Arg (H16A, F42A, and N88R substitution or H16T, F42A, and N88R substitutions).

IL-2 variants include polypeptides having at least 90% (e.g., at least 95%, 98%, or 99%) aa sequence identity to at least 80 (e.g., at least 100, 110, 120, or 130) contiguous aas of SEQ ID NO:106, wherein the aa at position 16 is other than H, the aa at position 42 is other than F, and the aa at position 126 is other than Q. Such IL-2 variants include those wherein, the position of H16 is substituted by Ala or Thr, the position of F42 is substituted by Ala, and the position of Q126 is substituted by Ala (an H16A, F42A, and Q126A substitution or an H16T, F42A, and Q126A substitutions).

IL-2 variants include polypeptides having at least 90% (e.g., at least 95%, 98%, or 99%) aa sequence identity to at least 80 (e.g., at least 100, 110, 120, or 130) contiguous aas of SEQ ID NO:106, wherein the aa at position 20 is other than D, the aa at position 42 is other than F, and the aa at position 126 is other than Q. In one case, the position D20 is substituted by Ala, the position of F42 is substituted by Ala, and the position of Q126 is substituted by Ala (D20A, F42A, and Q126A substitutions).

IL-2 variants include polypeptides having at least 90% (e.g., at least 95%, 98%, or 99%) aa sequence identity to at least 80 (e.g., at least 100, 110, 120, or 130) contiguous aas of SEQ ID NO:106, wherein the aa at position 20 is other than D, the aa at position 42 is other than F, and the aa at position 45 is other than Y. In one case, the position D20 is substituted by Ala, the position of F42 is substituted by Ala, and the position of Y45 is substituted by Ala (D20A, F42A, and Y45A substitutions).

IL-2 variants include polypeptides having at least 90% (e.g., at least 95%, 98%, or 99%) aa sequence identity to at least 80 (e.g., at least 100, 110, 120, or 130) contiguous aas of SEQ ID NO:106, wherein the aa at position 16 is other than H, the aa at position 20 is other than D, the aa at position 42 is other than F, and the aa at position 45 is other than Y. Such IL-2 variants include those in which the position of H16 is substituted by Ala or Thr, the position D20 is substituted by Ala, the position of F42 is substituted by Ala, and the position of Y45 is substituted by Ala (H16A, D20A, F42A, and Y45A substitution, or H16T, D20A, F42A, and Y45A substitutions).

IL-2 variants include polypeptides having at least 90% (e.g., at least 95%, 98%, or 99%) aa sequence identity to at least 80 (e.g., at least 100, 110, 120, or 130) contiguous aas of SEQ ID NO:106, wherein the aa at position 20 is other than D, the aa at position 42 is other than F, the aa at position 45 is other than Y, and the aa at position 126 is other than Q. In one case, the position D20 is substituted by Ala, the position of F42 is substituted by Ala, the position of Y45 is substituted by Ala, and the position of Q126 is substituted by Ala (D20A, F42A, Y45A, Q126A substitutions).

IL-2 variants include polypeptides having at least 90% (e.g., at least 95%, 98%, or 99%) aa sequence identity to at least 80 (e.g., at least 100, 110, 120, or 130) contiguous aas of SEQ ID NO:106, wherein the aa at position 16 is other than H, the aa at position 20 is other than D, the aa at position 42 is other than F, the aa at position 45 is other than Y, and the aa at position 126 is other than Q. In one case, the position of H16 is substituted by Ala or Thr, the position D20 is substituted by Ala, the position of F42 is substituted by Ala, the position of Y45 is substituted by Ala, and the position of Q126 is substituted by Ala (H16A, D20A, F42A, Y45A, and Q126A substitutions or H16T, D20A, F42A, Y45A, and Q126A substitutions).

c. Fas Ligand (FasL) and its Variants

In some cases, the MOD present in a T-Cell-MP is a Fas Ligand (FasL). FasL is a homomeric type-II transmembrane protein in the tumor necrosis factor (TNF) family FasL signals by trimerization of the Fas receptor in a target cell, which forms a death-inducing complex leading to apoptosis of the target cell. Soluble FasL results from matrix metalloproteinase-7 (MMP-7) cleavage of membrane-bound FasL at a conserved site.

In an embodiment, a wt. Homo sapiens FasL protein has the sequence MQQPFNYPYP QIYWVDSSAS SPWAPPGTVL PCPTSVPRRP GQRRPPPPPP PPPLPPPPPP PPLPPLPLPP LKKRGNHSTG LCLLVMFFMV LVALVGLGLG MFQLFHLQKE LAELRESTSQ MHTASSLEKQ IGHPSPPPEK KELRKVAHLT GKSNSRSMPL EWEDTYGIVL LSGVKYKKGG LVINETGLYF VYSKVYFRGQ SCNNLPLSHK VYMRNSKYPQ DLVMMEGKMM SYCTTGQMWA RSSYLGAVFN LTSADHLYVN VSELSLVNFE ESQTFFGLYK L, (SEQ ID NO:111), NCBI Ref. Seq. NP_000630.1, UniProtKB—P48023 where residues 1-80 are cytoplasmic, 810102 are the transmembrane domain and aas 103-281 are extracellular (ectodomain).

A suitable FasL polypeptide comprises all or part of the ectodomain of FasL: QLFHLQKE LAELRESTSQ MHTASSLEKQ IGHPSPPPEK KELRKVAHLT GKSNSRSMPL EWEDTYGIVL LSGVKYKKGG LVINETGLYF VYSKVYFRGQ SCNNLPLSHK VYMRNSKYPQ DLVMMEGKMM SYCTTGQMWA RSSYLGAVFN LTSADHLYVN VSELSLVNFE ESQTFFGLYK L (SEQ ID NO:112).

A Fas receptor can have the sequence MLGIWTLLPL VLTSVARLSS KSVNAQVTDI NSKGLELRKT VTTVETQNLE GLHHDGQFCH KPCPPGERKA RDCTVNGDEP DCVPCQEGKE YTDKAHFSSK CRRCRLCDEG HGLEVEINCT RTQNTKCRCK PNFFCNSTVC EHCDPCTKCE HGIIKECTLT SNTKCKEEGS RSNLGWLCLL LLPIPLIVWV KRKEVQKTCR KHRKENQGSH ESPTLNPETV AINLSDVDLS KYITTIAGVM TLSQVKGFVR KNGVNEAKID EIKNDNVQDT AEQKVQLLRN WHQLHGKKEA YDTLIKDLKK ANLCTLAEKI QTIILKDITS DSENSNFRNE IQSLV, (SEQ ID NO:113) NCBI Reference Sequence: NP_000034.1, UniProtKB—P25445, where aas 26-173 form the ectodomain (extracellular domain), aas 174-190 form the transmembrane domain, and 191-335 the cytoplasmic domain. The ectodomain may be used to determine binding affinity with FasL.

In some cases, a variant FasL polypeptide (e.g., comprising a variant of SEQ ID NO:112) exhibits reduced binding affinity to a mature Fas receptor sequence (e.g., a FasL receptor comprising all or part of the polypeptides set forth in SEQ ID NO:113, such as its ectodomain), compared to the binding affinity of an FasL polypeptide comprising the aa sequence set forth in SEQ ID NO:112. For example, in some cases, a variant FasL polypeptide (e.g., comprising a variant of SEQ ID NO:112) binds an Fas receptor (e.g., comprising all or part of the polypeptides set forth in SEQ ID NOs: 102, such as its ectodomains), with a binding affinity that is at least 10% less, at least 20% less, at least 30% less, at least 40% less, at least 50% less, at least 60% less, at least 70% less, at least 80% less, at least 90% less, at least 95% less, or more than 95% less, than the binding affinity of an FasL polypeptide comprising the aa sequence set forth in SEQ ID NO:111 or 112.

In some cases, a variant FasL polypeptide (e.g., comprising a variant of SEQ ID NO:112) has a binding affinity for an Fas receptor (e.g., comprising all or part of the polypeptides set forth in SEQ ID NO:113, such as its ectodomain), that is from 1 nM to 1 mM (e.g., from 1 nM to 10 nM, from 10 nM to 100 nM, from 100 nM to 1 μM, from 1 μM to 10 μM, from 10 μM to 100 μM, or from 100 μM to 1 mM). As another example, in some cases, a variant FasL polypeptide (e.g., comprising a variant of SEQ ID NO:101) has a binding affinity for a mature Fas receptor (e.g., comprising all or part of the polypeptides set forth in SEQ ID NO:113, such as its ectodomains), that is from about 100 nM to about 200 nM, from about 200 nM to about 400 nM, from about 400 nM to about 600 nM, from about 600 nM to about 800 nM, from about 800 nM to about 1 μM, from about 1 μM to about 5 μM, from about 5 μM to about 10 μM, from about 10 μM to about 20 μM, from about 20 μM to about 40 μM, from about 40 μM to about 75 μM, or from about 75 μM to about 100 μM.

In some cases, a variant FasL polypeptide (e.g., comprising a variant of SEQ ID NO:112) has a single aa substitution compared to the FasL polypeptide sequence set forth in SEQ ID NO:112). In some cases, a variant FasL polypeptide (e.g., comprising a variant of SEQ ID NO:112)) has from 2 aa to 10 aa substitutions compared to the FasL polypeptide sequence set forth in SEQ ID NO:112). In some cases, a variant FasL polypeptide has 2 aa substitutions compared to the FasL polypeptide sequence set forth in SEQ ID NO:112). In some cases, a variant FasL polypeptide has 3 aa or 4 aa substitutions compared to the FasL polypeptide sequence set forth in SEQ ID NO:112). In some cases, a variant FasL polypeptide has 5 aa substitutions compared to the FasL polypeptide sequence set forth in SEQ ID NO:112). In some cases, a variant FasL polypeptide has 6 aa or 7 aa substitutions compared to the FasL polypeptide sequence set forth in SEQ ID NO:112). In some cases, a variant FasL polypeptide has 8aa, 9 aa, or 10 aa substitutions compared to the FasL polypeptide sequence set forth in SEQ ID NO:112).

Suitable variant FasL polypeptide sequences include polypeptide sequences with at least 80%, at least 85%, at least 90%, at least 95%, at least 98%, or at least 99% aa sequence identity to at least 140 contiguous aa (e.g., at least 150, at least 160, at least 170, or at least 175 contiguous aa) of SEQ ID NO:112 (e.g., which have at least one aa substitution, deletion or insertion). FasL variants include polypeptides having at least 90% (e.g., at least 95%, 98%, or 99%) aa sequence identity to at least 80 (e.g., at least 100, 110, 120, or 130) contiguous aas of SEQ ID NO:112), and bear one or more aa substitutions from aa 1 to aa 50. Such Fas L variants may comprise the substitutions of at least one, at least two, or at least three aas with an Ala or Gly.

FasL variants include polypeptides having at least 90% (e.g., at least 95%, 98%, or 99%) aa sequence identity to at least 80 (e.g., at least 100, 110, 120, or 130) contiguous aas of SEQ ID NO:112), and bear one or more aa substitutions from aa 51 to 100. Such Fas L variants may comprise the substitutions of at least one, at least two, or at least three aas with an Ala or Gly.

FasL variants include polypeptides having at least 90% (e.g., at least 95%, 98%, or 99%) aa sequence identity to at least 80 (e.g., at least 100, 110, 120, or 130) contiguous aas of SEQ ID NO:112), and bear one or more aa substitutions from aa 101 to 150. Such Fas L variants may comprise the substitutions of at least one, at least two, or at least three aas with an Ala or Gly.

FasL variants include polypeptides having at least 90% (e.g., at least 95%, 98%, or 99%) aa sequence identity to at least 80 (e.g., at least 100, 110, 120, or 130) contiguous aas of SEQ ID NO:112) and bear one or more aa substitutions from aa 151 to 170. Such Fas L variants may comprise the substitutions of at least one, at least two, or at least three aas with an Ala or Gly. Independent of, or in addition to, any Ala or Gly substitutions, any of the above-mentioned Fas L variants may comprise the substitutions of at least one, at least two, or at least three positively charged aas with an Asp (D) or Glu (E), and/or at least one, at least two, or at least three negatively charged aas with Arg (R) or Lys (K) residues.

d. PD-L1 and its Variants

As one non-limiting example, a wt. MOD or variant MOD present in a T-Cell-MP is a PD-L1 or variant PD-L1 polypeptide. Wild-type PD-L1 binds to PD1 and CD80 (also known as B7-1). A wt. human PD-L1 polypeptide can comprise the following aa sequence: MRIFAVFIFM TYWHLLNAFT VTVPKDLYVV EYGSNMTIEC KFPVEKQLDL AALIVYWEME DKNIIQFVHG EEDLKVQHSS YRQRARLLKD QLSLGNAALQ ITDVKLQDAG VYRCMISYGG ADYKRITVKV NAPYNKINQR ILVVDPVTSE HELTCQAEGY PKAEVIWTSS DHQVLSGKTT TTNSKREEKL FNVTSTLRIN TTTNEIFYCT FRRLDPEENH TAELVIPGNI LNVSIKICLT LSPST (SEQ ID NO:114); where aas 1-18 form the signal sequence, aas 19-127 form the Ig-like V-type or IgV domain, and 133-225 for the Ig-like C2 type domain.

A wt. human PD-L1 ectodomain can comprise the following aa sequence: FT VTVPKDLYVV EYGSNMTIEC KFPVEKQLDL AALIVYWEME DKNIIQFVHG EEDLKVQHSS YRQRARLLKD QLSLGNAALQ ITDVKLQDAG VYRCMISYGG ADYKRITVKV NAPYNKINQR ILVVDPVTSE HELTCQAEGY PKAEVIWTSS DHQVLSGKTT TTNSKREEKL FNVTSTLRIN TTTNEIFYCT FRRLDPEENH TAELVIPGNI LNVSIKI (SEQ ID NO:115); where aas 1-109 form the Ig-like V-type or “IgV” domain, and aas 115-207 for the Ig-like C2 type domain.

A wt. PD-L1 IgV domain, suitable for use as a MOD may comprise aas 18-127 or aas 19-127 of SEQ D No. 114, and a carboxyl terminal stabilization sequences, such as for instance the last seven aas (bolded and italicized) of the sequence: A FTVTVPKDLY VVEYGSNMTI ECKFPVEKQL DLAALIVYWE MEDKNIIQFV HGEEDLKTQH SSYRQRARLL KDQLSLGNAA [symbol] ITDVKLQD AGVYRCMISY GGADYKRITV KVNAPY [symbol] SEQ ID NO:116. Where the carboxyl stabilizing sequence comprises a histidine (e.g., a histidine approximately 5 residues to the C-terminal side of the Tyr (Y) appearing as aa 117 of SEQ ID NO:116) to about aa 122, the histidine may form a stabilizing electrostatic bond with the backbone amide at aas 82 and 83 (bolded and italicized in SEQ ID NO:116 (Q107 and L106 of SEQ ID NO:114). As an alternative, a stabilizing disulfide bond may be formed by substituting one of aas 82 or 83) (Q107 and L106 of SEQ ID NO:114) and one of aa residues 121, 122, or 123 (equivalent to aa positions 139-141 of SEQ ID NO:114).

A wt. PD-1 polypeptide can comprise the following aa sequence: PGWFLDSPDR PWNPPTFSPA LLVVTEGDNA TFTCSFSNTS ESFVLNWYRM SPSNQTDKLA AFPEDRSQPG QDCRFRVTQL PNGRDFHMSV VRARRNDSGT YLCGAISLAP KAQIKESLRA ELRVTERRAE VPTAHPSPSP RPAGQFQTLV VGVVGGLLGS LVLLVWVLAV ICSRAARGTI GARRTGQPLK EDPSAVPVFS VDYGELDFQW REKTPEPPVP CVPEQTEYAT IVFPSGMGTS SPARRGSADG PRSAQPLRPE DGHCSWPL (SEQ ID NO:117).

In some cases, a variant PD-L1 polypeptide (e.g. a variant of SEQ ID NO:115 or PD-L1's IgV domain) exhibits reduced binding affinity to PD-1 (e.g., a PD-1 polypeptide comprising the aa sequence set forth in SEQ ID NO:117), compared to the binding affinity of a PD-L1 polypeptide comprising the aa sequence set forth in SEQ ID NO:114 or SEQ ID NO:115. For example, in some cases, a variant PD-L1 polypeptide binds PD-1 (e.g., a PD-1 polypeptide comprising the aa sequence set forth in SEQ ID NO:117) with a binding affinity that is at least 10% less, at least 20% less, at least 30% less, at least 40% less, at least 50% less, at least 60% less, at least 70% less, at least 80% less, at least 90% less, at least 95% less, or more than 95% less than the binding affinity of a PD-L1 polypeptide comprising the aa sequence set forth in SEQ ID NO:114 or SEQ ID NO:115.

In some cases, a variant PD-L1 polypeptide (e.g. a variant of SEQ ID NO:115 or its IgV domain) has a binding affinity to PD-1 (e.g. of SEQ ID NO:117) that is from 1 nM to 1 mM (e.g., from 1 nM to 10 nM, from 10 nM to 100 nM, from 100 nM to 1 μM, from 1 μM to 10 μM, from 10 μM to 100 μM, or from 100 μM to 1 mM). As another example, in some cases, a variant PD-L1 polypeptide (e.g. a variant of SEQ ID NO:115) has a binding affinity for PD1 (e.g., a PD1 polypeptide comprising the aa sequence set forth in SEQ ID NO:117) that is from about 100 nM to about 200 nM, from about 200 nM to about 400 nM, from about 400 nM to about 600 nM, from about 600 nM to about 800 nM, from about 800 nM to about 1 μM, from about 1 μM to about 5 μM, from about 5 μM to about 10 μM, from about 10 μM to about 20 μM, from about 20 μM to about 40 μM, from about 40 μM to about 75 μM, or from about 75 μM to about 100 μM.

A number of aa substitutions may be made in the PD-L1 ectodomain sequences used as MODs, including substitutions to sequences having greater than 90% (95%, 98% or 99%) sequence identity to at least 85 contiguous aas (e.g., at least 90, at least 95, at least 100, or at least 105 contiguous aas) of any one of SEQ ID NO:114, SEQ ID NO:115, aas 19-127 (the IgV domain) of SEQ ID NO:114, and SEQ ID NO:116. The substitutions may include disulfide bond substitution pair D103C and G33C, or the pair V104 and S34C (based on SEQ ID NO:114). The substitutions also include salt bridge forming substitution pair Q107D and K62R or the pair Q107D and S80R (based on SEQ ID NO:114). In addition, the substitutions include the Pi stacking substitutions M36Y or M36F (based on SEQ ID NO:114). A PD-L1 MOD sequence may comprise a sequence having at least 85 contiguous aas (e.g., at least 90, at least 95, at least 100, or at least 105 contiguous aas) of SEQ ID NO:115, and at least one (e.g., at least two or at least three) disulfide, salt bridge, and/or Pi stacking substitution. A PD-L1 MOD sequence may comprise a sequence having at least 85 contiguous aas (e.g., at least 90, at least 95, at least 100, or at least 105 contiguous aas) of aas 19-127 (the IgV domain) of SEQ ID NO:114, and at least one (e.g., at least two or at least three) disulfide, salt bridge, and/or Pi stacking substitution. A PD-L1 MOD sequence may comprise a sequence having at least 85 contiguous aas (e.g., at least 90, at least 95, at least 100, or at least 105 contiguous aas) of aas SEQ ID NO:116, and at least one (e.g., at least two or at least three) disulfide, salt bridge, and/or Pi stacking substitution.

In some cases, a variant PD-L1 polypeptide has a single aa substitution compared to the PD-L1 aa sequence set forth in SEQ ID NO:114, SEQ ID NO:115 or PD-L1's IgV domain. In some cases, a variant PD-L1 polypeptide has from 2 aa to 10 aa substitutions compared to the PD-L1 aa sequence set forth in SEQ ID NO:114, SEQ ID NO:115 or PD-L1's IgV domain. In some cases, a variant PD-L1 polypeptide has 2 aa substitutions compared to the PD-L1 aa sequence set forth in SEQ ID NO:114, SEQ ID NO:115 or PD-L1's IgV domain. In some cases, a variant PD-L1 polypeptide has 3 aa or 4 aa substitutions compared to the PD-L1 aa sequence set forth in SEQ ID NO:114, SEQ ID NO:115 or PD-L1's IgV domain. In some cases, a variant PD-L1 polypeptide has 5 aa or 6 aa substitutions compared to the PD-L1 aa sequence set forth in SEQ ID NO:114, SEQ ID NO:115 or PD-L1's IgV domain. In some cases, a variant PD-L1 polypeptide has 7 aa or 8 aa substitutions compared to the PD-L1 aa sequence set forth in SEQ ID NO:114, SEQ ID NO:115 or PD-L1's IgV domain. In some cases, a variant PD-L1 polypeptide has 9 aa or 10 aa substitutions compared to the PD-L1 aa sequence set forth in SEQ ID NO:114, SEQ ID NO:115 or PD-L1's IgV domain.

Suitable variant PD-L1 polypeptide sequences include polypeptide sequences having at least 80%, at least 85%, at least 90%, at least 95%, at least 98%, or at least 99% aa sequence identity to at least 170 contiguous aa (e.g., at least 180, 190 or 200 contiguous aa) of SEQ ID NO:115 (e.g. which have at least one aa insertion, deletion or substitution). Suitable variant PD-L1 IgV polypeptide sequences include polypeptide sequences having at least 80%, at least 85%, at least 90%, at least 95%, at least 98%, or at least 99% aa sequence identity to at least 70 contiguous aa (e.g., at least 80, 90, 100 or 105 contiguous aas) of aas 1-109 of SEQ ID NO:115 (e.g. which have at least one aa insertion, deletion or substitution).

Variant PD-L1 polypeptide sequences include polypeptide sequences having at least 90% (e.g., at least 95%, 98%, or 99%) aa sequence identity to at least 80 (e.g., at least 90, 100, or 109, 110, 120, 150, 180, 190, 200, 210, or 219) contiguous aas of SEQ ID NO:115, comprising a substitution of one or more (two or more, or all three) of the Asp at aa 8, Ile at aa 36, and/or the Glu at aa 54.

Variant PD-L1 polypeptide sequences include polypeptide sequences having at least 90% (e.g., at least 95%, 98%, or 99%) aa sequence identity to at least 80 (e.g., at least 90, 100, or 109, 110, 120, 150, 180, 190, 200, 210, or 219) contiguous aas of SEQ ID NO:115, wherein the aa at position 8 is an aa other than D. In one case, the position of D8 is substituted by Ala. In another such embodiment the position of D8 is substituted by Arg.

Variant PD-L1 polypeptide sequences include polypeptide sequences having at least 90% (e.g., at least 95%, 98%, or 99%) aa sequence identity to at least 80 (e.g., at least 90, 100, or 109, 110, 120, 150, 180, 190, 200, 210, or 219) contiguous aas of SEQ ID NO:115, wherein the aa at position 36 is an aa other than I. In one case, the position of 136 is substituted by Ala. In another such embodiment, the position of 136 is substituted by Asp.

Variant PD-L1 polypeptide sequences also include polypeptide sequences having at least 90% (e.g., at least 95%, 98%, or 99%) aa sequence identity to at least 80 (e.g., at least 90, 100, or 109, 110, 120, 150, 180, 190, 200, 210, or 219) contiguous aas of SEQ ID NO:115, wherein the aa at position 54 is an aa other than E. In one case, the position of E 54 is substituted by Ala. In another such embodiment E54 is substituted by Arg.

e. CD80 and its Variants

In some cases, a variant MOD present in a T-Cell-MP is a variant CD80 polypeptide. Wild-type CD80 binds to CD28.

A wt. aa sequence of the ectodomain of human CD80 can be as follows: VIHVTK EVKEVATLSC GHNVSVEELA QTRIYWQKEK KMVLTMMSGD MNIWPEYKNR TIFDITNNLS IVILALRPSD EGTYECVVLK YEKDAFKREH LAEVTLSVKA DFPTPSISDF EIPTSNIRRI ICSTSGGFPE PHLSWLENGE ELNAINTTVS QDPETELYAV SSKLDFNMTT NHSFMCLIKY GHLRVNQTFN WNTTKQEHFP DN (SEQ ID NO:118). See NCBI Reference Sequence: NP_005182.1. The aa sequence of the IgV domain of a wt. human CD80 can be as follows: VIHVTK EVKEVATLSC GHNVSVEELA QTRIYWQKEK KMVLTMMSGD MNIWPEYKNR TIFDITNNLS IVILALRPSD EGTYECVVLK YEKDAFKREH LAEVTLSV, (SEQ ID NO:119), which is aas 1-104 of SEQ ID NO:118.

A wt. CD28 aa sequence can be as follows: MLRLLLALNL FPSIQVTGNK ILVKQSPMLV AYDNAVNLSC KYSYNLFSRE FRASLHKGLD SAVEVCVVYG NYSQQLQVYS KTGFNCDGKL GNESVTFYLQ NLYVNQTDIY FCKIEVMYPP PYLDNEKSNG TIIHVKGKHL CPSPLFPGPS KPFWVLVVVG GVLACYSLLV TVAFIIFWVR SKRSRLLHSD YMNMTPRRPG PTRKHYQPYA PPRDFAAYRS (SEQ ID NO:120).

A wt. CD28 aa sequence can be as follows: MLRLLLALNL FPSIQVTGNK ILVKQSPMLV AYDNAVNLSW KHLCPSPLFP GPSKPFWVLV VVGGVLACYS LLVTVAFIIF WVRSKRSRLL HSDYMNMTPR RPGPTRKHYQ PYAPPRDFAA YRS (SEQ ID NO:121)

A wt. CD28 aa sequence can be as follows: MLRLLLALNL FPSIQVTGKH LCPSPLFPGP SKPFWVLVVV GGVLACYSLL VTVAFIIFWV RSKRSRLLHS DYMNMTPRRP GPTRKHYQPY APPRDFAAYR S (SEQ ID NO:122).

In some cases, a variant CD80 polypeptide exhibits reduced binding affinity to CD28, compared to the binding affinity of a CD80 polypeptide comprising the aa sequence set forth in SEQ ID NO:118, or the IgV domain sequence SEQ ID NO:119, for CD28. For example, in some cases, a variant CD80 polypeptide binds CD28 with a binding affinity that is at least 10% less, at least 20% less, at least 30% less, at least 40% less, at least 50% less, at least 60% less, at least 70% less, at least 80% less, at least 90% less, at least 95% less, or more than 95% less, than the binding affinity of a CD80 polypeptide comprising the aa sequence set forth in SEQ ID NO:118 for CD28 (e.g., a CD28 polypeptide comprising the aa sequence set forth in one of SEQ ID NO:120, SEQ ID NO:121, or SEQ ID NO:122).

In some cases, a variant CD80 polypeptide has a binding affinity to CD28 that is from 100 nM to 100 μM. As another example, in some cases, a variant CD80 polypeptide of the present disclosure has a binding affinity for CD28 (e.g., a CD28 polypeptide comprising the aa sequence set forth in SEQ ID NO:120, SEQ ID NO:121, or SEQ ID NO:122) that is from about 100 nM to about 200 nM, from about 200 nM to about 400 nM, from about 400 nM to about 600 nM, from about 600 nM to about 800 nM, from about 800 nM to about 1 μM, from about 1 μM to about 5 μM, from about 5 μM to about 10 μM, from about 10 μM to about 20 μM, from about 20 μM to about 40 μM, from about 40 μM to about 75 μM, or from about 75 μM to about 100 μM.

In some cases, a variant CD80 polypeptide has a single aa substitution compared to the CD80 aa sequence set forth in SEQ ID NO:118 or 119. In some cases, a variant CD80 polypeptide has from 1 to 10 aa substitutions compared to the CD80 aa sequence set forth in SEQ ID NO:118 or 119. In some cases, a variant CD80 polypeptide has 1 aa substitution compared to the CD80 aa sequence set forth in SEQ ID NO:118 or 119. In some cases, a variant CD80 polypeptide has 2 aa substitutions compared to the CD80 aa sequence set forth in SEQ ID NO:118 or 119. In some cases, a variant CD80 polypeptide has 3 aa substitutions compared to the CD80 aa sequence set forth in SEQ ID NO:118. In some cases, a variant CD80 polypeptide has 4 aa substitutions compared to the CD80 aa sequence set forth in SEQ ID NO:118 or 119. In some cases, a variant CD80 polypeptide has 5 aa substitutions compared to the CD80 aa sequence set forth in SEQ ID NO:118 or 119. In some cases, a variant CD80 polypeptide has 6 or 7 aa substitutions compared to the CD80 aa sequence set forth in SEQ ID NO:118 or 119. In some cases, a variant CD80 polypeptide has 8, 9, or 10 aa substitutions compared to the CD80 aa sequence set forth in SEQ ID NO:118 or 119.

Some CD80 ectodomain variants with at least one aa substitution include those having at least 90%, at least 95%, at least 98%, or at least 99% aa sequence identity to SEQ ID NO:118 or the IgV domain sequence SEQ ID NO:119.

Some suitable CD80 ectodomain variants include polypeptides having at least 90%, at least 95%, at least 98%, or at least 99% aa sequence identity to SEQ ID NO:118 or the IgV domain sequence SEQ ID NO:119, and which have at least one (e.g., at least two, or at least three) aa substitution in that sequence from about residue 19 through about residue 67 including those that follow.

CD80 ectodomain variants include polypeptides having at least 90% (e.g., at least 95%, 98%, or 99%) aa sequence identity to at least 80 (e.g., at least 90, 100, or 104, 120, 150, 180, 200, or 208) contiguous aas of SEQ ID NO:118 or the IgV domain sequence SEQ ID NO:119, wherein the aa at position 19 is an aa other than N. In one case, the position of N19 is substituted by Ala. In another such embodiment N19 is substituted by Arg.

CD80 ectodomain variants include polypeptides having at least 90% (e.g., at least 95%, 98%, or 99%) aa sequence identity to at least 80 (e.g., at least 90, 100, or 104, 120, 150, 180, 200, or 208) contiguous aas of SEQ ID NO:118 or the IgV domain sequence SEQ ID NO:119, wherein the aa at position 25 is an aa other than L. In one case, the position of L25 is substituted by Ala.

CD80 ectodomain variants include polypeptides having at least 90% (e.g., at least 95%, 98%, or 99%) aa sequence identity to at least 80 (e.g., at least 90, 100, or 104, 120, 150, 180, 200, or 208) contiguous aas of SEQ ID NO:118 or the IgV domain sequence SEQ ID NO:119, wherein the aa at position 31 is an aa other than Y. In one case, the position of Y31 is substituted by Ala.

CD80 ectodomain variants include polypeptides having at least 90% (e.g., at least 95%, 98%, or 99%) aa sequence identity to at least 80 (e.g., at least 90, 100, or 104, 120, 150, 180, 200, or 208) contiguous aas of SEQ ID NO:118 or the IgV domain sequence SEQ ID NO:119, wherein the aa at position 33 is an aa other than Q. In one case, the position of Q33 is substituted by Ala.

CD80 ectodomain variants include polypeptides having at least 90% (e.g., at least 95%, 98%, or 99%) aa sequence identity to at least 80 (e.g., at least 90, 100, or 104, 120, 150, 180, 200, or 208) contiguous aas of SEQ ID NO:118 or the IgV domain sequence SEQ ID NO:119, wherein the aa at position 38 is an aa other than M. In one such embodiment, the position of M38 is substituted by Ala.

CD80 ectodomain variants include polypeptides having at least 90% (e.g., at least 95%, 98%, or 99%) aa sequence identity to at least 80 (e.g., at least 90, 100, or 104, 120, 150, 180, 200, or 208) contiguous aas of SEQ ID NO:118 or the IgV domain sequence SEQ ID NO:119, wherein the aa at position 39 is an aa other than V. In one case, the position of V39 is substituted by Ala.

CD80 ectodomain variants include polypeptides having at least 90% (e.g., at least 95%, 98%, or 99%) aa sequence identity to at least 80 (e.g., at least 90, 100, or 104, 120, 150, 180, 200, or 208) contiguous aas of SEQ ID NO:118 or the IgV domain sequence SEQ ID NO:119, wherein the aa at position 49 is an aa other than I. In one case, the position of 149 is substituted Ala.

CD80 ectodomain variants include polypeptides having at least 90% (e.g., at least 95%, 98%, or 99%) aa sequence identity to at least 80 (e.g., at least 90, 100, or 104, 120, 150, 180, 200, or 208) contiguous aas of SEQ ID NO:118 or the IgV domain sequence SEQ ID NO:119, wherein the aa at position 53 is an aa other than Y. In one case, the position of Y53 is substituted by Ala.

CD80 ectodomain variants include polypeptides having at least 90% (e.g., at least 95%, 98%, or 99%) aa sequence identity to at least 80 (e.g., at least 90, 100, or 104, 120, 150, 180, 200, or 208) contiguous aas of SEQ ID NO:118 or the IgV domain sequence SEQ ID NO:119, wherein the aa at position 60 is an aa other than D. In one case, the position of D60 is substituted by Ala.

CD80 ectodomain variants include polypeptides having at least 90% (e.g., at least 95%, 98%, or 99%) aa sequence identity to at least 80 (e.g., at least 90, 100, or 104, 120, 150, 180, 200, or 208) contiguous aas of SEQ ID NO:118 or the IgV domain sequence SEQ ID NO:119, wherein the aa at position 63 is an aa other than N. In one case, the position of N63 is substituted by Ala.

CD80 ectodomain variants include polypeptides having at least 90% (e.g., at least 95%, 98%, or 99%) aa sequence identity to at least 80 (e.g., at least 90, 100, or 104, 120, 150, 180, 200, or 208) contiguous aas of SEQ ID NO:118 or the IgV domain sequence SEQ ID NO:119, wherein the aa at position 67 is an aa other than I. In one case, the position of 167 is substituted by Ala.

Some suitable CD80 ectodomain variants include polypeptides having at least 90%, at least 95%, at least 98%, or at least 99%, aa sequence identity to at least 80 (e.g., at least 90, 100, or 104, 120, 150, 180, 200, or 208) contiguous aas of SEQ ID NO:118 or the IgV domain sequence SEQ ID NO:119, and which have at least one (e.g., at least two, or at least three) aa substitution in that sequence from about residue 86 through about residue 118, including those that follow.

CD80 ectodomain variants include polypeptides having at least 90% (e.g., at least 95%, 98%, or 99%) aa sequence identity to at least 80 (e.g., at least 90, 100, or 104, 120, 150, 180, 200, or 208) contiguous aas of SEQ ID NO:118 or the IgV domain sequence SEQ ID NO:119, wherein the aa at position 86 is an aa other than K. In one case, the position of K86 is substituted by Ala.

CD80 ectodomain variants include polypeptides having at least 90% (e.g., at least 95%, 98%, or 99%) aa sequence identity to at least 80 (e.g., at least 90, 100, or 104, 120, 150, 180, 200, or 208) contiguous aas of SEQ ID NO:118 or the IgV domain sequence SEQ ID NO:119, wherein the aa at position 119 is an aa other than F. In one case, the position of F119 is substituted by Ala. CD80 ectodomain variants include polypeptides having at least 90% (e.g., at least 95%, 98%, or 99%) aa sequence identity to at least 80 (e.g., at least 90, 100, or 104, 120, 150, 180, 200, or 208) contiguous aas of SEQ ID NO:118 or the IgV domain sequence SEQ ID NO:119, wherein the aa at position 118 is an aa other than P. In one case, the position of P118 is substituted by Ala.

Some suitable CD80 ectodomain variants include polypeptides having at least 90%, at least 95%, at least 98%, or at least 99%, aa sequence identity to at least 80 (e.g., at least 90, 100, or 104, 120, 150, 180, 200, or 208) contiguous aas of SEQ ID NO:118 or the IgV domain sequence SEQ ID NO:119, and which have at least one (e.g., at least two, or at least three) aa substitution in that sequence from about residue 156 through about residue 158, include those that follow.

CD80 ectodomain variants include polypeptides having at least 90% (e.g., at least 95%, 98%, or 99%) aa sequence identity to at least 80 (e.g., at least 90, 100, or 104, 120, 150, 180, 200, or 208) contiguous aas of SEQ ID NO:118 or the IgV domain sequence SEQ ID NO:119, wherein the aa at position 156 is an aa other than S. In one case, the position of aa156 is substituted by Ala.

CD80 ectodomain variants include polypeptides having at least 90% (e.g., at least 95%, 98%, or 99%) aa sequence identity to at least 80 (e.g., at least 90, 100, or 104, 120, 150, 180, 200, or 208) contiguous aas of SEQ ID NO:118 or the IgV domain sequence SEQ ID NO:119, wherein the aa at position 157 is an aa other than Q. In one case, the position of aa157 is substituted by Ala.

CD80 ectodomain variants include polypeptides having at least 90% (e.g., at least 95%, 98%, or 99%) aa sequence identity to at least 80 (e.g., at least 90, 100, or 104, 120, 150, 180, 200, or 208) contiguous aas of SEQ ID NO:118 or the IgV domain sequence SEQ ID NO:119, wherein the aa at position 158 is an aa other than D. In one case, the position of aa158 is substituted by Ala.

CD80 variants include polypeptides that comprises an aa sequence having at least 80% (e.g., at least 85%, at least 90%, at least 95%, at least 98%, at least 99%, or 100%) aa sequence identity to at least 80 (e.g., 90, 100, 110, 120, 130 or 133) contiguous aas of SEQ ID NO:118 or the IgV domain sequence SEQ ID NO:119 and comprise at least one of the CD80 sequence variations set forth in the Table of CD80 Variant MODs.

f. CD86 and its Variants

In some cases, a variant MOD present in a T-Cell-MP is a variant CD86 polypeptide. Wild-type CD86 binds to CD28.

The aa sequence of the full ectodomain of a wt. human CD86 can be as follows:

(SEQ ID NO: 123)
APLKIQAYFNETADLPCQFANSQNQSLSELVVFWQDQENLVLNEVYLGK
EKFDSVHSKYMNRTSFDSDSWTLRLHNLQIKDKGLYQCIIHHKKPTGMI
RIHQMNSELSVLANFSQPEIVPISNITENVYINLTCSSIHGYPEPKKMS
VLLRTKNSTIEYDGIMQKSQDNVTELYDVSISLSVSFPDVTSNMTIFCI
LETDKTRLLSSPFSIELEDPQPPPDHIP.

The aa sequence of the IgV domain of a wt. human CD86 can be as follows:

(SEQ ID NO: 124)
APLKIQAYFNETADLPCQFANSQNQSLSELVVFWQDQENLVLNEVYLGK
EKFDSVHSKYMNRTSFDSDSWTLRLHNLQIKDKGLYQCIIHHKKPTGMI
RIHQMNSELSVL.

In some cases, a variant CD86 polypeptide exhibits reduced binding affinity to CD28, compared to the binding affinity of a CD86 polypeptide comprising the aa sequence set forth in SEQ ID NO:123 or SEQ ID NO: 124 for CD28. For example, in some cases, a variant CD86 polypeptide binds CD28 with a binding affinity that is at least 10% less, at least 20% less, at least 30% less, at least 40% less, at least 50% less, at least 60% less, at least 70% less, at least 80% less, at least 90% less, at least 95% less, or more than 95% less, than the binding affinity of a CD86 polypeptide comprising the aa sequence set forth in SEQ ID NO:123 or SEQ ID NO:124 for CD28 (e.g., a CD28 polypeptide comprising the aa sequence set forth in one of SEQ ID NO:120, SEQ ID NO:121, or SEQ ID NO:122).

In some cases, a variant CD86 polypeptide has a binding affinity to CD28 that is from 100 nM to 100 μM. As another example, in some cases, a variant CD86 polypeptide of the present disclosure has a binding affinity for CD28 (e.g., a CD28 polypeptide comprising the aa sequence set forth in one of SEQ ID NO:120, SEQ ID NO:121, or SEQ ID NO:122) that is from about 100 nM to about 200 nM, from about 200 nM to about 400 nM, from about 400 nM to about 600 nM, from about 600 nM to about 800 nM, from about 800 nM to about 1 μM, from about 1 μM to about 5 μM, from about 5 μM to about 10 μM, from about 10 μM to about 20 μM, from about 20 μM to about 40 μM, from about 40 μM to about 75 μM, or from about 75 μM to about 100 μM.

In some cases, a variant CD86 polypeptide has a single aa substitution compared to the CD86 aa sequence set forth in SEQ ID NO:123 or 124. In some cases, a variant CD86 polypeptide has from 2 to 10 aa substitutions compared to the CD86 aa sequence set forth in SEQ ID NO:123. In some cases, a variant CD86 polypeptide has 2 aa substitutions compared to the CD86 aa sequence set forth in SEQ ID NO:123 or 124. In some cases, a variant CD86 polypeptide has 3 aa substitutions compared to the CD86 aa sequence set forth in SEQ ID NO:123 or 124. In some cases, a variant CD86 polypeptide has 4 aa substitutions compared to the CD86 aa sequence set forth in SEQ ID NO:123 or 124. In some cases, a variant CD86 polypeptide has 5 aa substitutions compared to the CD86 aa sequence set forth in SEQ ID NO:123 or 124. In some cases, a variant CD86 polypeptide has 6 or 7 aa substitutions compared to the CD86 aa sequence set forth in SEQ ID NO:123 or 124. In some cases, a variant CD86 polypeptide has 8, 9, or 10 aa substitutions compared to the CD86 aa sequence set forth in SEQ ID NO:123 or 124.

Some CD86 ectodomain variants with at least one aa substitution include those having at least 90%, at least 95%, at least 98%, or at least 99% aa sequence identity to at least 80 (e.g., at least 90, 100, or 109, 120, 150, 180, 200, or 224) contiguous aas of SEQ ID NO:123 or the IgV domain sequence SEQ ID NO:124. Suitable CD86 ectodomain variants include polypeptides having at least 90%, at least 95%, at least 98%, or at least 99% aa sequence identity to SEQ ID NO 8123 or 124, and which have at least one (e.g., at least two, or at least three) aa substitution in that sequence from about residue 33 through about residue 110, including those that follow.

CD86 ectodomain variants include polypeptides having at least 90% (e.g., at least 95%, 98%, or 99%) aa sequence identity to at least 80 (e.g., at least 90, 100, or 109, 120, 150, 180, 200, or 224) contiguous aas of SEQ ID NO:123 or 124, wherein the aa at position 33 is an aa other than F. In one case, the position of F33 is substituted by Ala. CD86 ectodomain variants include polypeptides having at least 90% (e.g., at least 95%, 98%, or 99%) aa sequence identity to at least 80 (e.g., at least 90, 100, or 109, 120, 150, 180, 200, or 224) contiguous aas of SEQ ID NO:123 or 124, wherein the aa at position 35 is an aa other than Q. In one case, the position of Q35 is substituted by Ala. CD86 ectodomain variants include polypeptides having at least 90% (e.g., at least 95%, 98%, or 99%) aa sequence identity to at least 80 (e.g., at least 90, 100, or 109, 120, 150, 180, 200, or 224) contiguous aas of SEQ ID NO:123 or 124, wherein the aa at position 41 is an aa other than V. In one case, the position of V41 is substituted by Ala. CD86 ectodomain variants include polypeptides having at least 90% (e.g., at least 95%, 98%, or 99%) aa sequence identity to at least 80 (e.g., at least 90, 100, or 109, 120, 150, 180, 200, or 224) contiguous aas of SEQ ID NO:123 or 124, wherein the aa at position 59 is an aa other than Y. In one case, the position of Y59 is substituted by Ala. CD86 ectodomain variants include polypeptides having at least 90% (e.g., at least 95%, 98%, or 99%) aa sequence identity to at least 80 (e.g., at least 90, 100, or 109, 120, 150, 180, 200, or 224) contiguous aas of SEQ ID NO:123 or 124, wherein the aa at position 61 is an aa other than N. In one case, the position of N61 is substituted by Ala. CD86 ectodomain variants include polypeptides having at least 90% (e.g., at least 95%, 98%, or 99%) aa sequence identity to at least 80 (e.g., at least 90, 100, or 109, 120, 150, 180, 200, or 224) contiguous aas of SEQ ID NO:123 or 124, wherein the aa at position 66 is an aa other than D. In one case, the position of D66 is substituted by Ala. CD86 ectodomain variants include polypeptides having at least 90% (e.g., at least 95%, 98%, or 99%) aa sequence identity to at least 80 (e.g., at least 90, 100, or 109, 120, 150, 180, 200, or 224) contiguous aas of SEQ ID NO:123 or 124, wherein the aa at position 70 is an aa other than W. In one case, the position of W70 is substituted by Ala. CD86 ectodomain variants include polypeptides having at least 90% (e.g., at least 95%, 98%, or 99%) aa sequence identity to at least 80 (e.g., at least 90, 100, or 109, 120, 150, 180, 200, or 224) contiguous aas of SEQ ID NO:123 or 124, wherein the aa at position 72 is an aa other than L. In one case, the position of L72 is substituted by Ala. CD86 ectodomain variants include polypeptides having at least 90% (e.g., at least 95%, 98%, or 99%) aa sequence identity to at least 80 (e.g., at least 90, 100, or 109, 120, 150, 180, 200, or 224) contiguous aas of SEQ ID NO:123 or 124, wherein the aa at position 91 is an aa other than H. In one case, the position of H91 is substituted by Ala. CD86 ectodomain variants include polypeptides having at least 90% (e.g., at least 95%, 98%, or 99%) aa sequence identity to at least 80 (e.g., at least 90, 100, or 109, 120, 150, 180, 200, or 224) contiguous aas of SEQ ID NO:123 or 124, wherein the aa at position 110 is an aa other than L. In one case, the position of L110 is substituted by Ala.

CD86 ectodomain variants include polypeptides having at least 90% (e.g., at least 95%, 98%, or 99%) aa sequence identity to at least 80 (e.g., at least 90, 100, or 109, 120, 150, 180, 200, or 224) contiguous aas of SEQ ID NO:123 or 124, wherein N61 is an aa other than Asn, and wherein H91 is an aa other than His. In an embodiment, the positions of both Asn 61 and His 91 are substituted by Ala (N61A, and H91A substitutions). CD86 ectodomain variants include polypeptides having at least 90% (e.g., at least 95%, 98%, or 99%) aa sequence identity to at least 80 (e.g., at least 90, 100, or 109, 120, 150, 180, 200, or 224) contiguous aas of SEQ ID NO:123 or 124, wherein D66 is an aa other than Asp, and wherein H91 is an aa other than His. In an embodiment, the positions of both Asp66 and His 91 are substituted by Ala (D66A and H91A substitutions). CD86 ectodomain variants include polypeptides having at least 90% (e.g., at least 95%, 98%, or 99%) aa sequence identity to at least 80 (e.g., at least 90, 100, or 109, 120, 150, 180, 200, or 224) contiguous aas of SEQ ID NO:123 or 124, wherein N61 is an aa other than Asn, D66 is an aa other than Asp, and wherein H91 is an aa other than His. In one case, the positions of Asn 61, Asp 66 and His 91 are substituted by Ala (N61A, D66A and H91A substitutions).

g. 4-1BBL and its Variants

In some cases, a variant MOD present in a T-Cell-MP is a variant 4-1BBL polypeptide. Wild-type 4-1BBL binds to 4-1BB (CD137).

A wt. 4-1BBL aa sequence can be as follows: MEYASDASLD PEAPWPPAPR ARACRVLPWA LVAGLLLLLL LAAACAVFLA CPWAVSGARA SPGSAASPRL REGPELSPDD PAGLLDLRQG MFAQLVAQNV LLIDGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:125). NCBI Reference Sequence: NP_003802.1, where aas 29-49 are a transmembrane region,

In some cases, a variant 4-1BBL polypeptide is a variant of the tumor necrosis factor (TNF) homology domain (THD) of human 4-1BBL. A wt. aa sequence of the THD of human 4-1BBL can comprise, e.g., one of SEQ ID NOs:126-128, as follows:

(SEQ ID NO: 126)
PAGLLDLRQG MFAQLVAQNV LLIDGPLSWY SDPGLAGVSL
TGGLSYKEDT KELVVAKAGV YYVFFQLELR RVVAGEGSGS
VSLALHLQPL RSAAGAAALA LTVDLPPASS EARNSAFGFQ
GRLLHLSAGQ RLGVHLHTEA RARHAWQLTQ GATVLGLFRV
TPEIPAGLPS PRSE;
(SEQ ID NO: 127)
D PAGLLDLRQG MFAQLVAQNV LLIDGPLSWY SDPGLAGVSL
TGGLSYKEDT KELVVAKAGV YYVFFQLELR RVVAGEGSGS
VSLALHLQPL RSAAGAAALA LTVDLPPASS EARNSAFGFQ
GRLLHLSAGQ RLGVHLHTEA RARHAWQLTQ GATVLGLFRV
TPEIPAGLPS PRSE;
and
(SEQ ID NO: 128)
D PAGLLDLRQG MFAQLVAQNV LLIDGPLSWY SDPGLAGVSL
TGGLSYKEDT KELVVAKAGV YYVFFQLELR RVVAGEGSGS
VSLALHLQPL RSAAGAAALA LTVDLPPASS EARNSAFGFQ
GRLLHLSAGQ RLGVHLHTEA RARHAWQLTQ GATVLGLFRV
TPEIPA.

A wt. 4-1BB aa sequence can be as follows: MGNSCYNIVA TLLLVLNFER TRSLQDPCSN CPAGTFCDNN RNQICSPCPP NSFSSAGGQR TCDICRQCKG VFRTRKECSS TSNAECDCTP GFHCLGAGCS MCEQDCKQGQ ELTKKGCKDC CFGTFNDQKR GICRPWTNCS LDGKSVLVNG TKERDVVCGP SPADLSPGAS SVTPPAPARE PGHSPQIISF FLALTSTALL FLLFFLTLRF SVVKRGRKKL LYIFKQPFMR PVQTTQEEDG CSCRFPEEEE GGCEL (SEQ ID NO:129).

In some cases, a variant 4-1BBL polypeptide exhibits reduced binding affinity to 4-1BB, compared to the binding affinity of a 4-1BBL polypeptide comprising the aa sequence set forth in one of SEQ ID NOs: 126-128. For example, in some cases, a variant 4-1BBL polypeptide of the present disclosure binds 4-1BB with a binding affinity that is at least 10% less, at least 20% less, at least 30% less, at least 40% less, at least 50% less, at least 60% less, at least 70% less, at least 80% less, at least 90% less, at least 95% less, or more than 95% less, than the binding affinity of a 4-1BBL polypeptide comprising the aa sequence set forth in one of SEQ ID NOs:125-128 for a 4-1BB polypeptide (e.g., a 4-1BB polypeptide comprising the aa sequence set forth in SEQ ID NO:129), when assayed under the same conditions.

In some cases, a variant 4-1BBL polypeptide has a binding affinity to 4-1BB that is from 100 nM to 100 μM. As another example, in some cases, a variant 4-1BBL polypeptide has a binding affinity for 4-1BB (e.g., a 4-1BB polypeptide comprising the aa sequence set forth in SEQ ID NO:129) that is from about 100 nM to about 200 nM, from about 200 nM to about 400 nM, from about 400 nM to about 600 nM, from about 600 nM to about 800 nM, from about 800 nM to about 1 μM, from about 1 μM to about 5 μM, from about 5 μM to about 10 μM, from about 10 μM to about 20 μM, from about 20 μM to about 40 μM, from about 40 μM to about 75 μM, or from about 75 μM to about 100 μM.

In some cases, a variant 4-1BBL polypeptide has a single aa substitution compared to the 4-1BBL aa sequence set forth in one of SEQ ID NOs: 126-128. In some cases, a variant 4-1BBL polypeptide has from 2 to 10 aa substitutions compared to the 4-1BBL aa sequence set forth in one of SEQ ID NOs:126-128. In some cases, a variant 4-1BBL polypeptide has 2 aa substitutions compared to the 4-1BBL aa sequence set forth in one of SEQ ID NOs:125-128. In some cases, a variant 4-1BBL polypeptide has 3 aa substitutions compared to the 4-1BBL aa sequence set forth in one of SEQ ID NOs:126-128. In some cases, a variant 4-1BBL polypeptide has 4 aa substitutions compared to the 4-1BBL aa sequence set forth in one of SEQ ID NOs:126-128. In some cases, a variant 4-1BBL polypeptide has 5 aa substitutions compared to the 4-1BBL aa sequence set forth in one of SEQ ID NOs:126-128. In some cases, a variant 4-1BBL polypeptide has 6 or 7 aa substitutions compared to the 4-1BBL aa sequence set forth in one of SEQ ID NOs:125-128. In some cases, a variant 4-1BBL polypeptide has 7, 8 or 9 aa substitutions compared to the 4-1BBL aa sequence set forth in one of SEQ ID NOs: 126-128.

Some 4-1BBL variants with at least one aa substitution include those having at least 90%, at least 95%, at least 98%, or at least 99% aa sequence identity to at least 140 (e.g., at least 160, 175, 180, or 181) contiguous aas of SEQ ID NO:126.

Suitable 4-1BBL variants include polypeptides having at least 90%, at least 95%, at least 98%, or at least 99% aa sequence identity to SEQ ID NO:126, and which have at least one aa substitution in that sequence from residue 11 through residue 30. Some 4-1BB1 variants with substitutions from residue 11 through residue 30 include polypeptides having at least 90% (e.g., at least 95%, 98%, or 99%) aa sequence identity to at least 140 (e.g., at least 160, 175, 180, or 181) contiguous aas of SEQ ID NO:126 wherein: the aa at position 11 is an aa other than M, the aa at position 12 is an aa other than F, the aa at position 14 is an aa other than Q, the aa at position 15 is an aa other than L, the aa at position 16 is an aa other than V, the aa at position 18 is an aa other than Q, the aa at position 19 is an aa other than N, the aa at position 20 is an aa other than V, the aa at position 21 is an aa other than L, the aa at position 22 is an aa other than L, the aa at position 23 is an aa other than I, the aa at position 24 is an aa other than D, the aa at position 25 is an aa other than G, the aa at position 26 is an aa other than P, the aa at position 27 is an aa other than L, the aa at position 28 is an aa other than S, the aa at position 29 is an aa other than W., or the aa at position 30 is an aa other than Y. 4-1BBL variants include polypeptides having at least 90% (e.g., at least 95%, 98%, or 99%) aa sequence identity to at least 140 (e.g., at least 160, 175, 180, or 181) contiguous aas of SEQ ID NO:126 where one of aas 11, 12, 14, 15, 16, 18, 19, 20, 21, 22, 23, 24, 25, 26, 27, 28, 29, or 30 are substituted by an Ala.

Suitable 4-1BBL variants include polypeptides having at least 90%, at least 95%, at least 98%, or at least 99% aa sequence identity to SEQ ID NO:126, and which have at least one aa substitution in that sequence from residue 31 through residue 50. Some 4-1BB1 variants with substitutions from residue 31 through residue 50 include polypeptides having at least 90% (e.g., at least 95%, 98%, or 99%) aa sequence identity to at least 140 (e.g., at least 160, 175, 180, or 181) contiguous aas of SEQ ID NO:126 wherein: the aa at position 31 is an aa other than S, the aa at position 32 is an aa other than D, the aa at position 33 is an aa other than P, the aa at position 34 is an aa other than G, the aa at position 35 is an aa other than L. the aa at position 37 is an aa other than G, the aa at position 38 is an aa other than V. the aa at position 39 is an aa other than S, the aa at position 40 is an aa other than L. the aa at position 41 is an aa other than T, the aa at position 42 is an aa other than G, the aa at position 43 is an aa other than G, the aa at position 44 is an aa other than L, the aa at position 45 is an aa other than S, the aa at position 46 is an aa other than Y, the aa at position 47 is an aa other than K, the aa at position 48 is an aa other than E, the aa at position 49 is an aa other than D, or the aa at position 50 is an aa other than T.

Suitable 4-1BBL variants include polypeptides having at least 90%, at least 95%, at least 98%, at least 99% aa sequence identity to SEQ ID NO:126, and which have at least one aa substitution in that sequence from residue 51 through residue 78. Some 4-1BB1 variants with substitutions from reside 51 through residue 78 include those that follow. 4-1BBL variants include polypeptides having at least 90% (e.g., at least 95%, 98%, or 99%) aa sequence identity to at least 140 (e.g., at least 160, 175, 180, or 181) contiguous aas of SEQ ID NO:126 where one of aas 31, 32, 33, 34, 35, 37, 38, 39, 40, 41, 42, 43, 44, 45, 46, 47, 48, 49, or 50 are substituted by an Ala.

Suitable 4-1BBL variants include polypeptides having at least 90%, at least 95%, at least 98%, or at least 99% aa sequence identity to SEQ ID NO:126, and which have at least one aa substitution in that sequence from residue 51 through residue 100. Some 4-1BB1 variants with substitutions from residue 51 through residue 100 include polypeptides having at least 90% (e.g., at least 95%, 98%, or 99%) aa sequence identity to at least 140 (e.g., at least 160, 175, 180, or 181) contiguous aas of SEQ ID NO:126 wherein: the aa at position 51 is an aa other than K, the aa at position 52 is an aa other than E, the aa at position 65 is an aa other than F, the aa at position 66 is an aa other than Q, the aa at position 67 is an aa other than, the aa at position 68 is an aa other than E, the aa at position 69 is an aa other than L, the aa at position 70 is an aa other than R, the aa at position 71 is an aa other than R, the aa at position 72 is an aa other than V, the aa at position 73 is an aa other than V, the aa at position 75 is an aa other than G, the aa at position 76 is an aa other than E, the aa at position 77 is an aa other than G, or the aa at position 78 is an aa other than S. 4-1BBL variants include polypeptides having at least 90% (e.g., at least 95%, 98%, or 99%) aa sequence identity to at least 140 (e.g., at least 160, 175, 180, or 181) contiguous aas of SEQ ID NO:126 where one of aas 51, 52, 65, 66, 67, 68, 69 70, 71, 72, 73, 74, 76, 77, or 78 are substituted by an Ala.

Suitable 4-1BBL variants include polypeptides having at least 90%, at least 95%, at least 98%, or at least 99% aa sequence identity to SEQ ID NO:126, and which have at least one aa substitution in that sequence from residue 103 through residue 117. Some 4-1BB1 variants with substitutions from residue 103 through residue 117 include polypeptides having at least 90% (e.g., at least 95%, 98%, or 99%) aa sequence identity to at least 140 (e.g., at least 160, 175, 180, or 181) contiguous aas of SEQ ID NO:126 wherein: the aa at position 103 is an aa other than V, the aa at position 104 is an aa other than D, the aa at position 105 is an aa other than L, the aa at position 106 is an aa other than P, the aa at position 109 is an aa other than S, the aa at position 110 is an aa other than S, the aa at position 111 is an aa other than E, the aa at position 113 is an aa other than R, the aa at position 125 is an aa other than N, or the aa at position 115 is an aa other than S. In one case, the position of 5115 is substituted by Ala, or the aa at position 117 is an aa other than F.

4-1BBL variants include polypeptides having at least 90% (e.g., at least 95%, 98%, or 99%) aa sequence identity to at least 140 (e.g., at least 160, 175, 180, or 181) contiguous aas of SEQ ID NO:126 where one of aas 103, 104, 105, 106, 109, 110, 111, 113, 114, 115, or 117 are substituted by an Ala.

Suitable 4-1BBL variants include polypeptides having at least 90%, at least 95%, at least 98%, or at least 99% aa sequence identity to SEQ ID NO:126, and which have at least one aa substitution in that sequence from residue 130 through residue 154. Some 4-1BB1 variants with substitutions from residue 130 through residue 154 include polypeptides having at least 90% (e.g., at least 95%, 98%, or 99%) aa sequence identity to at least 140 (e.g., at least 160, 175, 180, or 181) contiguous aas of SEQ ID NO:126 wherein: the aa at position 130 is an aa other than Q, the aa at position 131 is an aa other than R, the aa at position 132 is an aa other than L, the aa at position 133 is an aa other than G, the aa at position 134 is an aa other than V, the aa at position 135 is an aa other than H, the aa at position 136 is an aa other than L, the aa at position 137 is an aa other than H, the aa at position 138 is an aa other than T, the aa at position 139 is an aa other than E, the aa at position 141 is an aa other than R, the aa at position 143 is an aa other than R, the aa at position 144 is an aa other than H, the aa at position 146 is an aa other than W, the aa at position 147 is an aa other than Q, the aa at position 148 is an aa other than L, the aa at position 149 is an aa other than T, the aa at position 150 is an aa other than Q, the aa at position 151 is an aa other than G, the aa at position 153 is an aa other than T, or the aa at position 154 is an aa other than V. 4-1BBL variants include polypeptides having at least 90% (e.g., at least 95%, 98%, or 99%) aa sequence identity to at least 140 (e.g., at least 160, 175, 180, or 181) contiguous aas of SEQ ID NO:126 where one of aas 130, 131, 132, 133, 134, 135, 136, 137, 138, 139, 141, 143, 144, 146, 147, 148, 149, 150, 151, 153, or 154 are substituted by an Ala.

4-1BBL variants include polypeptides having at least 90% (e.g., at least 95%, at least 98%, or at least 99%) aa sequence identity to at least 140 (e.g., at least 160, 175, 180, or 181) contiguous aas of SEQ ID NO:126 and comprise a substitution of K47 (K127 of SEQ ID NO:125) with an aa other than Lys. In an embodiment, K47 is substituted with an Ala to form a K47A substitutions (a K127A substitution in SEQ ID NO:126). In an embodiment, the positions of both Asp66 and His 91 are substituted by Ala (e.g., D66A and H91A substitutions in SEQ ID NO:126).

h. Anti-CD28

In some cases, antibodies or antibody sequences directed against CD28 (e.g., an anti-CD28 antibody, an anti-body fragment binding CD28, or an scFv, nanobody, or diabody binding to CD28) may be employed as a MOD in a T-Cell-MP. The ability of anti-CD28 antibodies to act as a superagonist, agonist, or antagonist of CD28 activity has been described. See e.g., Poirier et al., (2012) Amer. J. of Transplantation, “CD28-Specific Immunomodulating Antibodies: What Can Be Learned From Experimental Models?” 12:1682-1690. Of particular interest are anti-CD28 antibodies that act as an agonist or superagonist.

Anti-CD28 antibodies or anti-CD28 sequences may be included in T-Cell-MPs in the absence of any other MOD sequences. Alternatively, antibodies or antibody sequences directed against CD28 by be incorporated into a T-Cell-MP along with one or more additional MODs, or variant MODs. In an embodiment, A T-Cell-MP comprises one or more (e.g., two) anti-CD28 antibody or anti-CD28 sequences along with one or more (e.g., two) 4-1BBL MODs or variant MODs, such as those described above. In an embodiment, A T-Cell-MP comprises one or more (e.g., two) anti-CD28 antibody or anti-CD28 sequences along with one or more (e.g., two) IL-2 MODs or variant IL-2 MODs, such as those described above. For example, the substitutions in the variant IL-2 MOD may include H16A or H16T along with an F42A or F42T substitution. By way of example, a T-Cell-MP may comprise one or more (e.g., two) anti-CD28 antibody or anti-CD28 sequences (e.g., an anti-CD28 scFv) along with one or more variant IL-2 MODs comprising H16A and/or F42A substitutions.

In some cases, an anti-CD28 antibody suitable for inclusion in a T-Cell-MP comprises: a) VL CDR1, VL CDR2, and VL CDR3 present in a light chain variable region (VL) comprising the following amino acid sequence: QWYQQKPGQPPKLLIFAASNVESGVPARFSGSGSGTNFSLNIHPVDEDDVA MYFCQQSRKVPYTFGGGTKLEIKR (SEQ ID NO:559); and b) VH CDR1, CDR2, and CDR3 present in a heavy chain variable region (VH) comprising the following amino acid sequence: QVKLQQSGPGL VTPSQSLSITCTVSGFSLSDYGVHWVRQSPGQGLEWLGVIWAGGGTNYNSALMSRKSISKDNSK SQVFLKMNSLQADDTAVYYCARDKGYSYYYSMDYWGQGTTVTVSS (SEQ ID NO:560). In some cases, the V_{H} and V_{L} CDRs are as defined by Kabat (see, e.g., the CDR Table, above; and Kabat 1991). In some cases, the V_{H} and V_{L} CDRs are as defined by Chothia (see, e.g., the CDR Table, above; and Chothia 1987). In some cases, the VH CDRs are: DYGVH (VH CDR1) (SEQ ID NO:561); VIWAGGGT NYNSALMS (VH CDR2) (SEQ ID NO:562); and DKGYSYYYSMDY (VH CDR3) (SEQ ID NO:563).

In some cases, an anti-CD28 antibody suitable for inclusion in a T-Cell-MP comprises: a) a VL region comprising an amino acid sequence having at least 90%, at least 95%, at least 98%, at least 99%, or 100%, amino acid sequence identity to the following amino acid sequence: QWYQQKPGQPPKLLIF AASNVESGVPARFSGSGSGTNFSLNIHPVDEDDVAMYFCQQSRKVPYTFGGGTKLEIKR (SEQ ID NO:559); and b) a VH region comprising an amino acid sequence having at least 90%, at least 95%, at least 98%, at least 99%, or 100%, amino acid sequence identity to the following amino acid sequence:

(SEQ ID NO: 560)
QVKLQQSGPGLVTPSQSLSITCTVSGFSLSDYGVHWVRQSPGQGLEWLG
VIWAGGGTNYNSALMSRKSISKDNSKSQVFLKMNSLQADDTAVYYCARD
KGYSYYYSMDYWGQGTTVTVSS.

In some cases, an anti-CD28 antibody suitable for inclusion in a T-Cell-MP is a scFv comprising, in order from N-terminus to C-terminus: a) a VL region comprising an amino acid sequence having at least 90%, at least 95%, at least 98%, at least 99%, or 100%, amino acid sequence identity to the following amino acid sequence: QWYQQKPGQPPKLLIFAASNVESGVPARFSGSGSGTNFSLNIHPV DEDDVAMYFCQQSRKVPYTFGGGTKLEIKR (SEQ ID NO:559); b) a peptide linker; and c) a VH region comprising an amino acid sequence having at least 90%, at least 95%, at least 98%, at least 99%, or 100%, amino acid sequence identity to the following amino acid sequence: QVKLQQSGPGLVTPSQ SLSITCTVSGFSLSDYGVHWVRQSPGQGLEWLGVIWAGGGTNYNSALMSRKSISKDNSKSQVFL KMNSLQADDTAVYYCARDKGYSYYYSMDYWGQGTTVTVSS (SEQ ID NO:560). In some cases, the peptide linker comprises the amino acid sequence (GGGGS)n, where n is an integer from 1 to 10 (e.g., where n is 1, 2, 3, 4, 5, 6, 7, 8, 9, or 10). In some cases, the peptide linker comprises the amino acid sequence GGGGSGGGGSGGGGS (SEQ ID NO:538) and has a length of 15 amino acids.

In some cases, an anti-CD28 antibody suitable for inclusion in a T-Cell-MP is a scFv comprising, in order from N-terminus to C-terminus: a) a VH region comprising an amino acid sequence having at least 90%, at least 95%, at least 98%, at least 99%, or 100%, amino acid sequence identity to the following amino acid sequence: QVKLQQSGPGLVTPSQSLSITCTVSGFSLSDYGVHWVRQSPGQG LEWLGVIWAGGGTNYNSALMSRKSISKDNSKSQVFLKMNSLQADDTAVYYCARDKGYSYYYS MDYWGQGTTVTVSS (SEQ ID NO:560); b) a peptide linker; and c) a VL region comprising an amino acid sequence having at least 90%, at least 95%, at least 98%, at least 99%, or 100%, amino acid sequence identity to the following amino acid sequence: QWYQQKPGQPPKLLIFAASNVESGVPARF SGSGSGTNFSLNIHPVDEDDVAMYFCQQSRKVPYTFGGGTKLEIKR (SEQ ID NO:559). In some cases, the peptide linker comprises the amino acid sequence (GGGGS)n, where n is an integer from 1 to 10 (e.g., where n is 1, 2, 3, 4, 5, 6, 7, 8, 9, or 10). In some cases, the peptide linker comprises the amino acid sequence GGGGSGGGGSGGGGS (SEQ ID NO:538) and has a length of 15 amino acids.

### 6 Linkers

T-Cell-MPs (and their T-Cell-MP-epitope conjugates) can include one or more independently selected linker polypeptide sequences interposed between, for example, any one or more of:
i) two MOD polypeptides located on the N-terminal side of the β2M polypeptide sequence (referred to as an L1 linker or position);
(ii) between a MOD and a β2M polypeptide sequence (referred to as an L2 linker or position);
(iii) between a β2M polypeptide sequence and a MHC-H polypeptide sequence (referred to as an L3 linker or position);
(iv) between a MHC-H polypeptide sequence and a scaffold polypeptide sequence (referred to as an L4 linker or position);
(iv) at the carboxyl end of the scaffold or between a scaffold polypeptide sequence and a MOD polypeptide sequence placed carboxy terminal to it (referred to as an L5 linker or position); or
(vi) between two MOD polypeptide sequences placed on the carboxy side of the scaffold (referred to as an L6 linker or position).

### See, e.g., FIG. 5.

Chemical conjugation sites for coupling epitopes (e.g., peptide epitopes) may be incorporated into linkers (e.g., L1-L6 linkers) including the L3 between the MHC-H and β2M polypeptide sequences. Accordingly, chemical conjugation sites including, but not limited to: sulfatase, sortase, transglutaminase, selenocysteine, non-natural amino acids, and naturally occurring proteinogenic amino acids (e.g., cysteine residues) etc. may be incorporated into linkers, including the L3 linker. Polypeptide linkers placed at either the N- or C-termini provide locations to couple additional polypeptides (e.g., histidine tags), payloads and the like, and to protect the polypeptide from exo-proteases.

Linkers may also be utilized between the peptide epitope and any reactive chemical moiety (group) used to couple the peptide epitope to the chemical conjugation site of an unconjugated T-Cell-MP (see e.g., FIG. 10). Linkers utilized between epitope (e.g., peptide epitope) and a reactive chemical moiety may be peptide/polypeptide linkers, and/or other chemical linkers (e.g., non-peptide linkers in the form of homo or hetero bifunctional linkers that comprise an alkyl group as a spacer, see e.g., FIG. 10 at entries d and e).

Suitable polypeptide linkers (also referred to as “spacers”) can be readily selected and can be of any of a number of suitable lengths, such as from 1 aa to 50 aa, from 1aa to 5 aa, from 1 aa to 15 aa, from 2 aa to 15 aa, from 2 aa to 25 aa, from 3 aa to 12 aa, from 4 aa to 10 aa, from 4 aa to 35 aa, from 5 aa to 35 aa, from 5 aa to 10 aa, from 5 aa to 20 aa, from 6 aa to 25 aa, from 7 aa to 35 aa, from 8aa to 40 aa, from 9 aa to 45 aa, from 10 to 15 aa, from 10 aa to 50 aa, from 15 to 20 aa, from 20 to 40 aa, or from 40 to 50 aa. Suitable polypeptide linkers in the range from 10 to 50 aas in length may be from 10 to 20, from 10 to 25, from 15 to 25, from 20 to 30, from 25 to 35, from 25 to 50 30 to 35, from 35 to 45, or from 40 to 50). In embodiments, a suitable linker can be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 aa in length. A polypeptide linker may have a length of from 15 aa to 50 aa, e.g., from 20 to 35, from 25 to 30, from 25 to 45, from 30 to 35, from 35 to 40, from 40 to 45, or from 45 to 50 aa in length.

Polypeptide linkers in the T-Cell-MP may include, for example, polypeptides that comprise, consist essentially of, or consists of: i) Gly and/or Ser; ii) Ala and Ser; iii) Gly, Ala, and Ser; iv) Gly, Ser, and Cys (e.g., a single Cys residue); v) Ala, Ser, and Cys (e.g., a single Cys residue); and vi) Gly, Ala, Ser, and Cys (e.g., a single Cys residue). Exemplary linkers may comprise glycine polymers, glycine-serine polymers, glycine-alanine polymers; alanine-serine polymers (including, for example polymers comprising the sequences GA, AG, AS, SA, GS, GSGGS (SEQ ID NO:130) or GGGS (SEQ ID NO:131), any of which may be repeated from 1 to 10 times (e.g., repeated 1, 2, 3, 4, 5, 6, 7, 8, 9, or 10 times); and other flexible linkers known in the art. Glycine and glycine-serine polymers can both be used as both Gly and Ser are relatively unstructured and therefore can serve as a neutral tether between components. Glycine polymers access significantly more phi-psi space than even alanine polymers, and are much less restricted than residues with longer side chains (see Scheraga, Rev. Computational Chem. 11173-142 (1992)). Exemplary linkers may also comprise an aa sequence comprising, but not limited to, GGSG (SEQ ID NO:132), GGSGG (SEQ ID NO:133), GSGSG (SEQ ID NO:134), GSGGG (SEQ ID NO:135), GGGSG (SEQ ID NO:136), GSSSG (SEQ ID NO:137), any of which may be repeated from 1 to 15 times (e.g., repeated 1, 2, 3, 4, 5, 6, 7, 8, 9, 10, 11, 12, 13, 14, or 15 times), or combinations thereof, and the like. Linkers can also comprise the sequence Gly(Ser)₄ (SEQ ID NO:138) or (Gly)₄Ser (SEQ ID NO:139), either of which may be repeated from 1 to 10 times (e.g., 1, 2, 3, 4, 5, 6, 7, 8, 9, or 10 times). In an embodiment, the linker comprises the X1-X2-X3-X₄-X5 where X1-X5 are selected from glycine and serine, and one of which may be a leucine, cysteine, methionine or alanine (SEQ ID NO:140). In one embodiment the linker comprises the aa sequence AAAGG (SEQ ID NO:532), which may be repeated from 1 to 10 times.

In some cases, a linker polypeptide, present in a T-Cell-MP includes a cysteine residue that can form a disulfide bond with a cysteine residue present in another T-Cell-MP or act as a chemical conjugation site for the coupling of an epitope (e.g., via reaction with a maleimide). In some cases, for example, the linker comprises Gly, Ser and a single Cys, such as in the aa sequence GCGGS(G₄S) (SEQ ID NO:141) where the G₄S unit may be repeated from 1 to 10 times (e.g., repeated 1, 2, 3, 4, 5, 6, 7, 8, 9, or 10 times), G [symbol] GASGGGGSGGGGS (SEQ ID NO:142), G [symbol] GGSGGGGSGGGGSGGGGS (SEQ ID NO:143) or G [symbol] GGSGGGGSGGGGS (SEQ ID NO:144).

A linker may comprise the aa sequence (GGGGS) (SEQ ID NO:139, also be represented as Gly₄ Ser or G₄S), which may be repeated from 1 to 10 times (e.g., 1, 2, 3, 4, 5, 6, 7, 8, 9, or 10 times). In some embodiments a linker comprising G₄S repeats has one glycine or serine residue replaced by a leucine or methionine. A first T-Cell-MP comprising a Gly₄ Ser containing linker polypeptide that includes a cysteine residue may, when duplexed with a second T-Cell-MP, form a disulfide bond with a cysteine residue present in the second T-Cell-MP of the duplex T-Cell-MP. Such cysteine residues present in linkers (particularly the L3 linker) may also be utilized as a chemical conjugation site for the attachment of an epitope (e.g., a peptide epitope), such as by reaction with a maleimide functionality that is part of, or indirectly connected by a linker to, the epitope. In some cases, for example, the linker comprises the aa sequence GCGGS(G₄S) (SEQ ID NO:141) where the G₄S unit may be repeated from 1 to 10 times (e.g., repeated 1, 2, 3, 4, 5, 6, 7, 8, 9, or 10 times), G [symbol] GASGGGGSGGGGS (SEQ ID NO:142), the sequence G [symbol] GGSGGGGSGGGGSGGGGS (SEQ ID NO:143) or the sequence GCGGSGGGGSGGGGS (SEQ ID NO:144). Non-peptide linkers that may be used to covalently attach epitopes, targeting sequences and/or payloads (e.g., a drug or labeling agent) to a T-Cell-MP (including its peptide linkers) may take a variety of forms, including, but not limited to, alkyl, poly(ethylene glycol), disulfide groups, thioether groups, acid labile groups, photolabile groups, peptidase labile groups, and esterase labile groups. The non-peptide linkers (or “crosslinkers”) may also be, for example, homobifunctional or heterobifunctional linkers that comprise reactive end groups such as N-hydroxysuccinimide esters, maleimide, iodoacetate esters, and the like. Examples of suitable cross-linkers include: N-succinimidyl-[(N-maleimidopropionamido)-tetraethyleneglycol]ester (NHS-PEG4-maleimide); N-succinimidyl 4-(2-pyridyldithio)butanoate (SPDB); N-succinimidyl 4-(2-pyridyldithio)2-sulfobutanoate (sulfo-SPDB); N-succinimidyl 4-(2-pyridyldithio) pentanoate (SPP); N-succinimidyl-4-(N-maleimidomethyl)-cyclohexane-1-carboxy-(6-amidocaproate) (LC-SMCC); K-maleimidoundecanoic acid N-succinimidyl ester (KMUA); γ-maleimide butyric acid N-succinimidyl ester (GMBS); ε-maleimidocaproic acid N-hydroxysuccinimide ester (EMCS); m-maleimide benzoyl-N-hydroxysuccinimide ester (MBS); N-(α-maleimidoacetoxy)-succinimide ester (AMAS); succinimidyl-6-(β-maleimidopropionamide)hexanoate (SMPH); N-succinimidyl 4-(p-maleimidophenyl)butyrate (SMPB); N-(p-maleimidophenyl)isocyanate (PMPI); N-succinimidyl 4(2-pyridylthio)pentanoate (SPP); N-succinimidyl(4-iodo-acetyl)aminobenzoate (SIAB); 6-maleimidocaproyl (MC); maleimidopropanoyl (MP); p-aminobenzyloxycarbonyl (PAB); N-succinimidyl 4-(maleimidomethyl)cyclohexanecarboxylate (SMCC); N-succinimidyl-4-(N-maleimidomethyl)-cyclohexane-1-carboxy-(6-amidocaproate), a “long chain” analog of SMCC (LC-SMCC); 3-maleimidopropanoic acid N-succinimidyl ester (BMPS); N-succinimidyl iodoacetate (SIA); N-succinimidyl bromoacetate (SBA); and N-succinimidyl 3-(bromoacetamido)propionate (SBAP).

### 7 Additional Polypeptide Sequences

A polypeptide chain of a T-Cell-MP can include one or more polypeptides in addition to those described above. Suitable additional polypeptides include epitope tags, affinity domains, and fluorescent protein sequences (e.g., green fluorescent protein). The one or more additional polypeptide(s) can be included as part of a polypeptide translated by cell or cell-free system at the N-terminus of a polypeptide chain of a multimeric polypeptide, at the C-terminus of a polypeptide chain of a multimeric polypeptide, or internally within a polypeptide chain of a multimeric polypeptide.

a. Epitope Tags and Affinity Domains

Suitable epitope tags include, but are not limited to, hemagglutinin (HA; e.g., YPYDVPDYA (SEQ ID NO:145)); c-myc (e.g., EQKLISEEDL; SEQ ID NO:146)), and the like.

Affinity domains include peptide sequences that can interact with a binding partner, e.g., such as one immobilized on a solid support, useful for identification or purification. DNA sequences encoding multiple consecutive single amino acids, such as histidine, when fused to the expressed protein, may be used for one-step purification of the recombinant protein by high affinity binding to a resin column, such as nickel SEPHAROSE®. Exemplary affinity domains include His5 (HHHHH) (SEQ ID NO:147), HisX6 (HHHHHH) (SEQ ID NO:148), C-myc (EQKLISEEDL) (SEQ ID NO:146), Flag (DYKDDDDK) (SEQ ID NO:149, StrepTag (WSHPQFEK) (SEQ ID NO:150), hemagglutinin (e.g., HA Tag (YPYDVPDYA) (SEQ ID NO:145)), glutathione-S-transferase (GST), thioredoxin, cellulose binding domain, RYIRS (SEQ ID NO:151), Phe-His-His-Thr (SEQ ID NO:152), chitin binding domain, S-peptide, T7 peptide, SH2 domain, C-end RNA tag, WEAAAREACCRECCARA (SEQ ID NO:153), metal binding domains (e.g., zinc binding domains or calcium binding domains such as those from calcium-binding proteins such as calmodulin, troponin C, calcineurin B, myosin light chain, recoverin, S-modulin, visinin, VILIP, neurocalcin, hippocalcin, frequenin, caltractin, calpain large-subunit, 5100 proteins, parvalbumin, calbindin D9K, calbindin D28K, and calretinin), inteins, biotin, streptavidin, MyoD, Id, leucine zipper sequences, and maltose binding protein.

b. Targeting Sequences

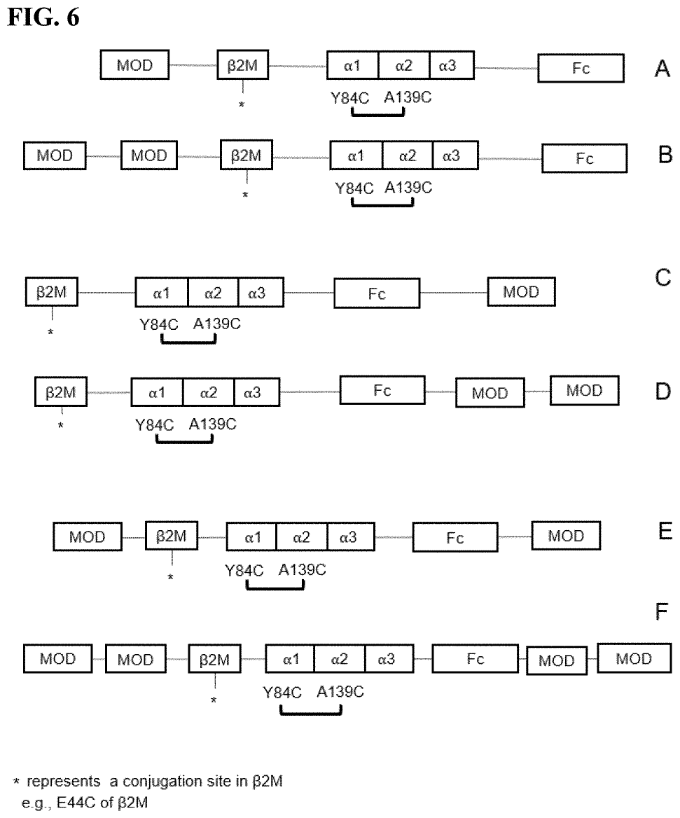

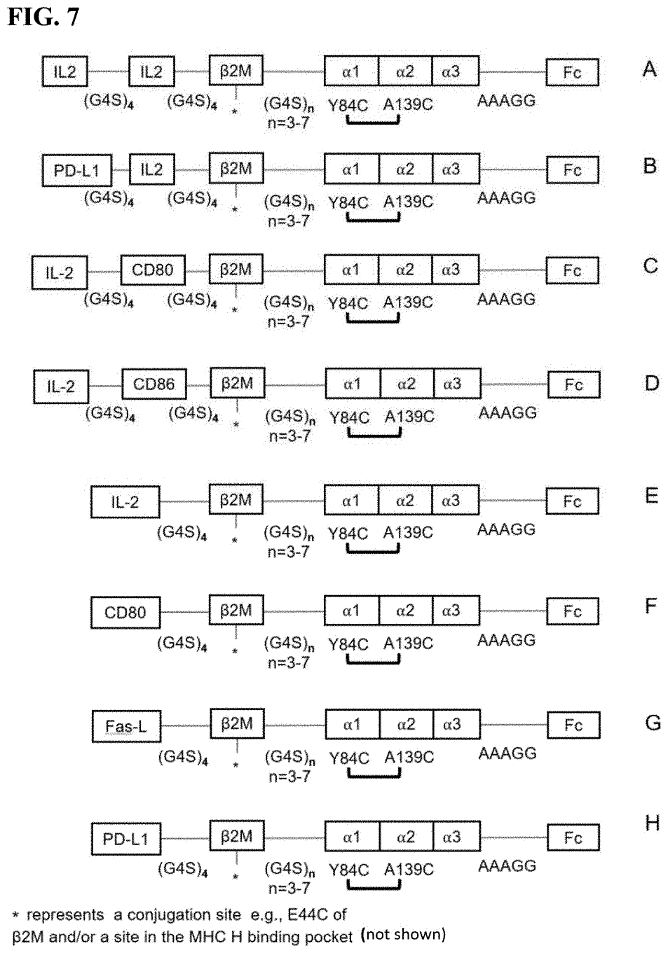

T-Cell-MPs of the present disclosure may include one or more targeting polypeptide sequence(s) or “targeting sequence(s).” Targeting sequences may be located anywhere within the T-Cell-MP polypeptide, for example within, at, or near the carboxyl terminal end of a scaffold peptide (e.g., translated with the scaffold in place of a C-terminal MOD in FIG. 5 or 6 or attached to an L5 linker). Alternatively, a targeting sequence, such as an antibody antigen-binding fragment (Fab), may be covalently or non-covalently attached to a T-Cell-MP. Covalent attachment of a targeting sequence may be made at a chemical conjugation site (e.g., a chemical conjugation site in a scaffold polypeptide), where the targeting sequence effectively becomes a payload-like molecule attached to the T-Cell-MP. Targeting sequences may also be non-covalently bound to a T-Cell-MP (e.g., a T-Cell-MP having a biotin labeled scaffold may be non-covalently attached to an avidin labeled targeting antibody or Fab directed to a cancer antigen). A bispecific antibody (e.g., a bispecific IgG or humanized antibody) having a first antigen binding site directed to a part of the T-Cell-MP (e.g., the scaffold) may also be employed to non-covalently attach a T-Cell-MP to a targeting sequence (the second bispecific antibody binding site) directed to a target (e.g., a cancer antigen). Targeting sequences serve to bind or “localize” T-Cell-MPs to cells and/or tissues displaying the protein (or other molecule) to which the targeting sequence binds. A targeting sequence may be an antibody or antigen binding fragment thereof. A targeting sequence may also be a single-chain T cell receptor (scTCR).

(i) Targets

A targeting sequence present in a T-Cell-MP of the present disclosure may target an antigen of an infecting organism and/or infected cell. In one instance, a targeting sequence may be an antibody or a polypeptide encompassing antigen binding fragment of an antibody. Targeting sequences may, for example, be directed to proteins/epitopes of infectious agents, such as viruses, bacteria, fungi, protozoans, and helminths, including those proteins/epitopes of infectious agents that are expressed on cell surfaces. By way of example, cells infected with HPV may express E6 or E7 proteins or portions thereof to which the targeting sequence may be directed. A targeting sequence may also be a Cancer Targeting Polypeptide, or “CTP” that is specific for a cancer associated antigen (“CAA”), such as an antigen associated with a non-solid cancer (e.g., a leukemia) and/or solid tumor-associated antigen. In one instance, the targeting sequence is specific for a cancer-associated peptide/HLA (pHLA) complex on the surface of a cancer cell, where the peptide can be a cancer-associated peptide (e.g., a peptide fragment of a cancer-associated antigen). T-Cell-MPs of the present disclosure can be targeted to cancer cells using targeting sequences that bind a CAA that is present on a cancer cell or presented as a peptide in the context of an HLA protein

(a) Cancer Associated Antigens (CAAs)

CAAs that can be targeted with a CTP present in a T-Cell-MP or a higher order T-Cell-MP complex, such as a duplex T-Cell-MP, of the present disclosure include, e.g., NY-ESO (New York Esophageal Squamous Cell Carcinoma 1), MART-1 (melanoma antigen recognized by T cells 1, also known as Melan-A), HPV (human papilloma virus) E6, BCMA (B-cell maturation antigen), CD123, CD133, CD171, CD19, CD20, CD22, CD30, CD33, CEA (carcinoembryonic antigen), EGFR (epidermal growth factor receptor), EGFRvIII (epidermal growth factor receptor variant III), EpCAM (epithelial cell adhesion molecule), EphA2 (ephrin type-A receptor 2), disialoganglioside GD2, GPC3 (glypican-3), HER2, IL13Ralpha2 (Interleukin 13 receptor subunit alpha-2), LeY (a difucosylated type 2 blood group-related antigen), melanoma-associated antigen (also known as melanoma-associated antigen gene product or MAGE) A3 (MAGE A3), melanoma glycoprotein, mesothelin, MUC1 (mucin 1), MUC16 (mucin 16), myelin, NKG2D (Natural Killer Group 2D) ligands, PSMA (prostate-specific membrane antigen), and ROR1 (type I receptor tyrosine kinase like orphan receptor).

CAAs that can be targeted with a CTP present in a T-Cell-MP also include, but are not limited to, 17-1A-antigen, alpha-fetoprotein (AFP), alpha-actinin-4, A3, antigen specific for A33 antibody, ART-4, B7, Ba 733, BAGE, bcl-2, bcl-6, BCMA, BrE3-antigen, CA125, CAMEL, CAP-1, carbonic anhydrase IX (CAIX), CASP-8/m, CCL19, CCL21, CD1, CD1a, CD2, CD3, CD4, CD5, CD8, CD11A, CD14, CD15, CD16, CD18, CD19, CD20, CD21, CD22, CD23, CD25, CD28, CD29, CD30, CD32b, CD33, CD37, CD38, CD40, CD40L, CD44, CD45, CD46, CD52, CD54, CD55, CD59, CD64, CD66a-e, CD67, CD70, CD70L, CD74, CD79a, CD79b, CD80, CD83, CD95, CD123, CD126, CD132, CD133, CD138, CD147, CD154, CD171, CDC27, CDK-4/m, CDKN2A, CEA, CEACAM5, CEACAM6, claudin (e.g., claudin-1, claudin-10, claudin-18 (e.g., claudin-18, isoform 2)), complement factors (such as C3, C3a, C3b, C5a and C5), colon-specific antigen-p (CSAp), c-Met, CTLA-4, CXCR4, CXCR7, CXCL12, DAM, Dickkopf-related protein (DKK), ED-B fibronectin, epidermal growth factor receptor (EGFR), EGFRvIII, EGP-1 (TROP-2), EGP-2, ELF2-M, Ep-CAM, EphA2, EphA3, fibroblast activation protein (FAP), fibroblast growth factor (FGF), Flt-1, Flt-3, folate binding protein, folate receptor, G250 antigen, gangliosides (such as GC2, GD3 and GM2), GAGE, GD2, gp100, GPC3, GRO-13, HLA-DR, HM1.24, human chorionic gonadotropin (HCG) and its subunits, HER2, HER3, HMGB-1, hypoxia inducible factor (HIF-1), HIF-1a, HSP70-2M, HST-2, Ia, IFN-gamma, IFN-alpha, IFN-beta, IFN-X, IL-4R, IL-6R, IL-13R, IL13Ralpha2, IL-15R, IL-17R, IL-18R, IL-2, IL-6, IL-8, IL-12, IL-15, IL-17, IL-18, IL-23, IL-25, ILGF, ILGF-1R, insulin-like growth factor-1 (IGF-1), IGF-1R, integrin αVβ3, integrin α5β1, KC4-antigen, killer-cell immunoglobulin-like receptor (KIR), Kras, KS-1-antigen, KS1-4, LDR/FUT, Legamma, macrophage migration inhibitory factor (MIF), MAGE, MAGE-3, MART-1, MART-2, mCRP, MCP-1, melanoma glycoprotein, mesothelin, MIP-1A, MIP-1B, MIF, mucins (such as MUC1, MUC2, MUC3, MUC4, MUC5ac, MUC13, MUC16, MUM-1/2 and MUM-3), NCA66, NCA95, NCA90, Nectin-4, NY-ESO-1, PAM4 antigen, pancreatic cancer mucin, PD-1, PD-L1, PD-1 receptor, placental growth factor, p53, PLAGL2, prostatic acid phosphatase (PAP), PSA, PRAME, PSMA, P1GF, RSS, RANTES, SAGE, 5100, survivin, survivin-2B, T101, TAC, TAG-72, tenascin, Thomson-Friedenreich antigens, Tn antigen, TNF-alpha, tumor necrosis antigens, TRAG-3, TRAIL receptors, vascular endothelial growth factor (VEGF), VEGF receptor (VEGFR) and WT-1.

A CAA targeted with a CTP of a T-Cell-MP may be an antigen associated with a hematological cancer. Examples of such antigens include, but are not limited to, BCMA, C5, CD19, CD20, CD22, CD25, CD30, CD33, CD38, CD40, CD45, CD52, CD56, CD66, CD74, CD79a, CD79b, CD80, CD138, CTLA-4, CXCR4, DKK, EphA3, GM2, HLA-DR beta, integrin αVβ3, IGF-R1, IL6, KIR, PD-1, PD-L1, TRAILR1, TRAILR2, transferrin receptor, and VEGF. In some cases, the CAA is an antigen expressed by malignant B cells, such as CD19, CD20, CD22, CD25, CD38, CD40, CD45, CD74, CD80, CTLA-4, IGF-R1, IL6, PD-1, TRAILR2, or VEGF.

A CAA targeted with a CTP of a T-Cell-MP may be associated with a solid tumor. Examples of such antigens include, but are not limited to, CAIX, cadherins, CEA, c-MET, CTLA-4, EGFR family members, EpCAM, EphA3, FAP, folate-binding protein, FR-alpha, gangliosides (such as GC2, GD3 and GM2), HER2, HER3, IGF-1R, integrin αVβ3, integrin α5β1, Legamma, Liv1, mesothelin, mucins, NaPi2b, PD-1, PD-L1, PD-1 receptor, pgA33, PSMA, RANKL, ROR1, TAG-72, tenascin, TRAILR1, TRAILR2, VEGF, VEGFR, and others listed above.

(b) Peptide/HLA Complexes

In some cases, a CTP of a T-Cell-MP or a higher order T-Cell-MP complex, such as a duplex T-Cell-MP, targets a peptide/HLA (pHLA) complex on the surface of a cancer cell, where the peptide is a cancer-associated peptide (e.g., a peptide fragment of a cancer-associated antigen). Cancer-associated peptide antigens are known in the art. In some cases, a cancer-associated peptide is bound to a HLA complex comprising an HLA-A*0201 heavy chain and a β2M polypeptide.

In some cases, the CAA peptide epitope present in the pHLA on the surface of a cancer cell is bound to an HLA complex comprising an HLA heavy chain such as HLA-A*0101, A*0201, A*0301, A*1101, A*2301, A*2402, A*2407, A*3303, and/or A*3401. In some cases, the peptide epitope present in the pHLA on the surface of a cancer cell is bound to an HLA complex comprising an HLA heavy chain such as HLA-B*0702, B*0801, B*1502, B*3802, B*4001, B*4601, and/or B*5301. In some cases, the peptide epitope present in the pHLA on the surface of a cancer cell is bound to an HLA complex comprising an HLA heavy chain such as C*0102, C*0303, C*0304, C*0401, C*0602, C*0701, C*702, C*0801, and/or C*1502.

In some cases, a CAA peptide is a peptide of from about 4 aas (aa) to about 20 aa (e.g., 4 aa to 5 aa, 6 aa to 8 aa, 9aa to 11 aa, 12 aa to 16 aa, or 16 aa to 20 aa) in length of any one of the following cancer-associated antigens: a CD28 polypeptide, a MUC1 polypeptide, an LMP2 polypeptide, an epidermal growth factor receptor (EGFR) vIII polypeptide, a HER-2/neu polypeptide, a melanoma antigen family A, 3 (MAGE A3) polypeptide, a p53 polypeptide, a mutant p53 polypeptide, an NY-ESO-1 polypeptide, a folate hydrolase (prostate-specific membrane antigen; PSMA) polypeptide, a carcinoembryonic antigen (CEA) polypeptide, a claudin polypeptide (e.g., claudin-1, claudin-10, claudin-18 (e.g., claudin-18, isoform 2)), a Nectin-4 polypeptide, a melanoma antigen recognized by T cells (melanA/MART1) polypeptide, a Ras polypeptide, a gp100 polypeptide, a proteinase3 (PR1) polypeptide, a bcr-abl polypeptide, a tyrosinase polypeptide, a survivin polypeptide, a prostate specific antigen (PSA) polypeptide, an hTERT polypeptide, a sarcoma translocation breakpoints polypeptide, a synovial sarcoma X (SSX) breakpoint polypeptide, an EphA2 polypeptide, an acid phosphatase, prostate (PAP) polypeptide, a melanoma inhibitor of apoptosis (ML-IAP) polypeptide, an epithelial cell adhesion molecule (EpCAM) polypeptide, an ERG (TMPRSS2 ETS fusion) polypeptide, a NA17 polypeptide, a paired-box-3 (PAX3) polypeptide, an anaplastic lymphoma kinase (ALK) polypeptide, an androgen receptor polypeptide, a cyclin B1 polypeptide, an N-myc proto-oncogene (MYCN) polypeptide, a Ras homolog gene family member C (RhoC) polypeptide, a tyrosinase-related protein-2 (TRP-2) polypeptide, a mesothelin polypeptide, a prostate stem cell antigen (PSCA) polypeptide, a melanoma associated antigen-1 (MAGE A1) polypeptide, a cytochrome P450 1B1 (CYP1B1) polypeptide, a placenta-specific protein 1 (PLAC1) polypeptide, a BORIS polypeptide (also known as CCCTC-binding factor or CTCF), an ETV6-AML polypeptide, a breast cancer antigen NY-BR-1 polypeptide (also referred to as ankyrin repeat domain-containing protein 30A), a regulator of G-protein signaling (RGSS) polypeptide, a squamous cell carcinoma antigen recognized by T cells (SART3) polypeptide, a carbonic anhydrase IX polypeptide, a paired box-5 (PAX5) polypeptide, an OY-TES1 (testis antigen; also known as acrosin binding protein) polypeptide, a sperm protein 17 polypeptide, a lymphocyte cell-specific protein-tyrosine kinase (LCK) polypeptide, a high molecular weight melanoma associated antigen (HMW-MAA), an A-kinase anchoring protein-4 (AKAP-4), a synovial sarcoma X breakpoint 2 (SSX2) polypeptide, an X antigen family member 1 (XAGE1) polypeptide, a B7 homolog 3 (B7H3; also known as CD276) polypeptide, a legumain polypeptide (LGMN1; also known as asparaginyl endopeptidase), a tyrosine kinase with Ig and EGF homology domains-2 (Tie-2; also known as angiopoietin-1 receptor) polypeptide, a P antigen family member 4 (PAGE4) polypeptide, a vascular endothelial growth factor receptor 2 (VEGF2) polypeptide, a MAD-CT-1 polypeptide, a fibroblast activation protein (FAP) polypeptide, a platelet derived growth factor receptor beta (PDGFI3) polypeptide, a melanoma cancer testis antigen-2 (MAD-CT-2) polypeptide, a Fos-related antigen-1 (FOSL) polypeptide; a human papilloma virus (HPV) antigen; an alpha-feto protein (AFP) antigen; and a Wilms tumor-1 (WT1) antigen.

For example, a CTP present in a T-Cell-MP may bind to: a) a WT-1 peptide bound to an HLA complex comprising an HLA heavy chain (e.g., an HLA-A*0201 heavy chain or an HLA-A*2402 heavy chain) and a β2M polypeptide; b) an HPV peptide bound to an HLA complex comprising a class I HLA heavy chain and a β2M polypeptide; c) a mesothelin peptide bound to an HLA complex comprising a class I HLA heavy chain and a β2M polypeptide; d) a Her2 peptide bound to an HLA complex comprising a class I HLA heavy chain and a β2M polypeptide; or e) a BCMA peptide bound to an HLA complex comprising a class I HLA heavy chain and a β2M polypeptide.

A CAA peptide may comprise from about 4 aas (aa) to about 20 aa (e.g., 4 aa, 5 aa, 6 aa, 7 aa, 8 aa, 9 aa, 10 aa, 11 aa, 12 aa, 13 aa, 14 aa, 15 aa, 16 aa, 17 aa, 18 aa, 19 aa, or 20 aa) in length of a mesothelin polypeptide having at least 90%, at least 95%, at least 98%, at least 99%, or 100%, aa sequence identity to the following mesothelin aa sequence: LAGE TGQEAAPLDG VLANPPNISS LSPRQLLGFP CAEVSGLSTE RVRELAVALA QKNVKLSTEQ LRCLAHRLSE PPEDLDALPL DLLLFLNPDA FSGPQACTRF FSRITKANVD LLPRGAPERQ RLLPAALACW GVRGSLLSEA DVRALGGLAC DLPGRFVAES AEVLLPRLVS CPGPLDQDQQ EAARAALQGG GPPYGPPSTW SVSTMDALRG LLPVLGQPII RSIPQGIVAA WRQRSSRDPS WRQPERTILR PRFRREVEKT ACPSGKKARE IDESLIFYKK WELEACVDAA LLATQMDRVN AIPFTYEQLD VLKHKLDELY PQGYPESVIQ HLGYLFLKMS PEDIRKWNVT SLETLKALLE VNKGHEMSPQ VATLIDRFVK GRGQLDKDTL DTLTAFYPGY LCSLSPEELS SVPPSSIWAV RPQDLDTCDP RQLDVLYPKA RLAFQNMNGS EYFVKIQSFL GGAPTEDLKA LSQQNVSMDL ATFMKLRTDA VLPLTVAEVQ KLLGPHVEGL KAEERHRPVR DWILRQRQDD LDTLGLGLQG GIPNGYLVLD LSMQEALSGT PCLLGPGPVL TVLALLLAST LA (SEQ ID NO:154). For example, a mesothelin peptide present in a pHLA complex can be: i) KLLGPHVEGL (SEQ ID NO:155); ii) AFYPGYLCSL (SEQ ID NO:156), which can bind HLA-A*2402/β2M; iii) VLPLTVAEV (SEQ ID NO:157); iv) ELAVALAQK (SEQ ID NO:158); v) ALQGGGPPY (SEQ ID NO:159); vi) FYPGYLCSL (SEQ ID NO:160); vii) LYPKARLAF (SEQ ID NO:161); viii) LLFLLFSLGWVGPSR (SEQ ID NO:162); ix) VNKGHEMSPQAPRRP (SEQ ID NO:163); x) FMKLRTDAVLPLTVA (SEQ ID NO:164); or xi) DAALLATQMD (SEQ ID NO:165).

A CAA peptide may comprise from about 4 aas (aa) to about 20 aa (e.g., 4 aa, 5 aa, 6 aa, 7 aa, 8 aa, 9 aa, 10 aa, 11 aa, 12 aa, 13 aa, 14 aa, 15 aa, 16 aa, 17 aa, 18 aa, 19 aa, or 20 aa) in length of a mesothelin polypeptide having at least 90%, at least 95%, at least 98%, at least 99%, or 100%, aa sequence identity to the following Her2 (receptor tyrosine-protein kinase erbB2) aa sequence:

(SEQ ID NO: 166)
MELAALCRWG LLLALLPPGA ASTQVCTGTD MKLRLPASPE
THLDMLRHLY QGCQVVQGNL ELTYLPTNAS LSFLQDIQEV
QGYVLIAHNQ VRQVPLQRLR IVRGTQLFED NYALAVLDNG
DPLNNTTPVT GASPGGLREL QLRSLTEILK GGVLIQRNPQ
LCYQDTILWK DIFHKNNQLA LTLIDTNRSR ACHPCSPMCK
GSRCWGESSE DCQSLTRTVC AGGCARCKGP LPTDCCHEQC
AAGCTGPKHS DCLACLHFNH SGICELHCPA LVTYNTDTFE
SMPNPEGRYT FGASCVTACP YNYLSTDVGS CTLVCPLHNQ
EVTAEDGTQR CEKCSKPCAR VCYGLGMEHL REVRAVTSAN
IQEFAGCKKI FGSLAFLPES FDGDPASNTA PLQPEQLQVF
ETLEEITGYL YISAWPDSLP DLSVFQNLQV IRGRILHNGA
YSLTLQGLGI SWLGLRSLRE LGSGLALIHH NTHLCFVHTV
PWDQLFRNPH QALLHTANRP EDECVGEGLA CHQLCARGHC
WGPGPTQCVN CSQFLRGQEC VEECRVLQGL PREYVNARHC
LPCHPECQPQ NGSVTCFGPE ADQCVACAHY KDPPFCVARC
PSGVKPDLSY MPIWKFPDEE GACQPCPINC THSCVDLDDK
GCPAEQRASP LTSIISAVVG ILLVVVLGVV FGILIKRRQQ
KIRKYTMRRL LQETELVEPL TPSGAMPNQA QMRILKETEL
RKVKVLGSGA FGTVYKGIWI PDGENVKIPV AIKVLRENTS
PKANKEILDE AYVMAGVGSP YVSRLLGICL TSTVQLVTQL
MPYGCLLDHV RENRGRLGSQ DLLNWCMQIA KGMSYLEDVR
LVHRDLAARN VLVKSPNHVK ITDFGLARLL DIDETEYHAD
GGKVPIKWMA LESILRRRFT HQSDVWSYGV TVWELMTFGA
KPYDGIPARE IPDLLEKGER LPQPPICTID VYMIMVKCWM
IDSECRPRFR ELVSEFSRMA RDPQRFVVIQ NEDLGPASPL
DSTFYRSLLE DDDMGDLVDA EEYLVPQQGF FCPDPAPGAG
GMVHHRHRSS STRNM.

A CAA peptide may comprise a peptide from about 4 aas (aa) to about 20 aa (e.g., 4 aa, 5 aa, 6 aa, 7 aa, 8 aa, 9 aa, 10 aa, 11 aa, 12 aa, 13 aa, 14 aa, 15 aa, 16 aa, 17 aa, 18 aa, 19 aa, or 20 aa) in length of a B-cell maturation protein (BCMP) polypeptide having at least 90%, at least 95%, at least 98%, at least 99%, or 100%, aa sequence identity to a portion of the following BCMA aa sequence:

(SEQ ID NO: 167)
MLQMAGQCSQ NEYFDSLLHA CIPCQLRCSS NTPPLTCQRY
CNASVTNSVK GTNAILWTCL GLSLIISLAV FVLMFLLRKI
SSEPLKDEFK NTGSGLLGMA NIDLEKSRTG DEIILPRGLE
YTVEECTCED CIKSKPKVDS DHCFPLPAME EGATILVTTK
TNDYCKSLPA ALSATEIEKS ISAR.

A CAA peptide may comprise a peptide from about 4 aas (aa) to about 20 aa (e.g., 4 aa, 5 aa, 6 aa, 7 aa, 8 aa, 9 aa, 10 aa, 11 aa, 12 aa, 13 aa, 14 aa, 15 aa, 16 aa, 17 aa, 18 aa, 19 aa, or 20 aa) in length of a mesothelin polypeptide having at least 90%, at least 95%, at least 98%, at least 99%, or 100%, aa sequence identity to the following WT-1 aa sequence: MDFLLLQDPA STCVPEPASQ HTLRSGPGCL QQPEQQGVRD PGGIWAKLGA AEASAERLQG RRSRGASGSE PQQMGSDVRD LNALLPAVPS LGGGGGCALP VSGAAQWAPV LDFAPPGASA YGSLGGPAPP PAPPPPPPPP PHSFIKQEPS WGGAEPHEEQ CLSAFTVHFS GQFTGTAGAC RYGPFGPPPP SQASSGQARM FPNAPYLPSC LESQPAIRNQ GYSTVTFDGT PSYGHTPSHH AAQFPNHSFK HEDPMGQQGS LGEQQYSVPP PVYGCHTPTD SCTGSQALLL RTPYSSDNLY QMTSQLECMT WNQMNLGATL KGHSTGYESD NHTTPILCGA QYRIHTHGVF RGIQDVRRVP GVAPTLVRSA SETSEKRPFM CAYPGCNKRY FKLSHLQMHS RKHTGEKPYQ CDFKDCERRF SRSDQLKRHQ RRHTGVKPFQ CKTCQRKFSR SDHLKTHTRT HTGEKPFSCR WPSCQKKFAR SDELVRHHNM HQRNMTKLQL AL (SEQ ID NO:168).

Non-limiting examples of WT-1 peptides include RMFPNAPYL (SEQ ID NO:397), CMTWNQMN (SEQ ID NO:403), CYTWNQMNL (SEQ ID NO:400), CMTWNQMNLGATLKG (SEQ ID NO:361), WNQMNLGATLKGVAA (SEQ ID NO:362), CMTWNYMNLGATLKG (SEQ ID NO:363), WNYMNLGATLKGVAA (SEQ ID NO:364), MTWNQMNLGATLKGV (SEQ ID NO:534), TWNQMNLGATLKGVA (SEQ ID NO:366), CMTWNLMNLGATLKG (SEQ ID NO:367), MTWNLMNLGATLKGV (SEQ ID NO:368), TWNLMNLGATLKGVA (SEQ ID NO:369), WNLMNLGATLKGVAA (SEQ ID NO:370), MNLGATLK (SEQ ID NO:371), MTWNYMNLGATLKGV (SEQ ID NO:372), TWNYMNLGATLKGVA (SEQ ID NO:373), CMTWNQMNLGATLKGVA (SEQ ID NO:374), CMTWNLMNLGATLKGVA (SEQ ID NO:375), CMTWNYMNLGATLKGVA (SEQ ID NO:376), GYLRNPTAC (SEQ ID NO:377), GALRNPTAL (SEQ ID NO:378), YALRNPTAC (SEQ ID NO:379), GLLRNPTAC (SEQ ID NO:380), RYRPHPGAL (SEQ ID NO:381), YQRPHPGAL (SEQ ID NO:382), RLRPHPGAL (SEQ ID NO:383), RIRPHPGAL (SEQ ID NO:384), QFPNHSFKHEDPMGQ (SEQ ID NO:385), HSFKHEDPY (SEQ ID NO:386), QFPNHSFKHEDPM (SEQ ID NO:387), QFPNHSFKHEDPY (SEQ ID NO:388), KRPFMCAYPGCNK (SEQ ID NO:389), KRPFMCAYPGCYK (SEQ ID NO:390), FMCAYPGCY (SEQ ID NO:391), FMCAYPGCK (SEQ ID NO:392), KRPFMCAYPGCNKRY (SEQ ID NO:393), SEKRPFMCAY PGCNK (SEQ ID NO:394), KRPFMCAYPGCYKRY (SEQ ID NO:395), NLMNLGATL (SEQ ID NO:359), NYMNLGATL (SEQ ID NO:360), and those WT-1 peptides recited in section I.A.8.d.i.(b).

In some cases, a CAA peptide is a peptide of from about 4 aas (aa) to about 20 aa (e.g., 4 aa, 5 aa, 6 aa, 7 aa, 8 aa, 9 aa, 10 aa, 11 aa, 12 aa, 13 aa, 14 aa, 15 aa, 16 aa, 17 aa, 18 aa, 19 aa, or 20 aa) in length of an HPV polypeptide having at least 90%, at least 95%, at least 98%, at least 99%, or 100%, aa sequence identity to a human papilloma virus (HPV) peptide. An HPV peptide can be a peptide of an HPV E6 polypeptide or an HPV E7 polypeptide. The HPV epitope can be an epitope of HPV of any of a variety of genotypes, including, e.g., HPV16, HPV18, HPV31, HPV33, HPV35, HPV39, HPV45, HPV51, HPV52, HPV56, HPV58, HPV59, HPV68, HPV73, or HPV82. Non-limiting examples of HPV peptides include: E6 18-26 (KLPQLCTEL; SEQ ID NO:274); E6 26-34 (LQTTIHDII; SEQ ID NO:404); E6 49-57 (VYDFAFRDL; SEQ ID NO:405); E6 52-60 (FAFRDLCIV; SEQ ID NO:406); E6 75-83 (KFYSKISEY; SEQ ID NO:407); E6 80-88 (ISEYRHYCY; SEQ ID NO:408); E7 7-15 (TLHEYMLDL; SEQ ID NO:409); E7 11-19 (YMLDLQPET; SEQ ID NO:276); E7 44-52 (QAEPDRAHY; SEQ ID NO:410); E7 49-57 (RAHYNIVTF (SEQ ID NO:411); E7 61-69 (CDSTLRLCV; SEQ ID NO:412); and E7 67-76 (LCVQSTHVDI; SEQ ID NO:413); E7 82-90 (LLMGTLGIV; SEQ ID NO:414); E7 86-93 (TLGIVCPI; SEQ ID NO:277); E7 92-93 (LLMGTLGIVCPI; SEQ ID NO:415); and those HPV peptides in section I.A.8.d.i.(c).

In some cases, a CAA peptide is a peptide of a claudin polypeptide having at least 90%, at least 95%, at least 98%, at least 99%, or 100%, amino acid sequence identity to the following claudin-18 (isoform 2) (CLDN 18.2) amino acid sequence: MAVTACQGLG FVVSLIGIAG IIAATCMDQW STQDLYNNPV TAVFNYQGLW RSCVRESSGF TECRGYFTLL GLPAMLQAVR ALMIVGIVLG AIGLLVSIFA LKCIRIGSME DSAKANMTLT SGIMFIVSGL CAIAGVSVFA NMLVTNFWMS TANMYTGMGG MVQTVQTRYT FGAALFVGWV AGGLTLIGGV MMCIACRGLA PEETNYKAVS YHASGHSVAY KPGGFKASTG FGSNTKNKKI YDGGARTEDE VQSYPSKHDY V (SEQ ID NO:169). In some cases, a cancer-associated peptide is a peptide of a claudin polypeptide having the amino acid sequence TEDEVQSYPSKHDYV (SEQ ID NO:170) (and having a length of about 15 amino acids) or EVQSYPSKHDYV (SEQ ID NO:171) (and having a length of about 12 amino acids.

In some cases, a CAA peptide is a peptide of a trophoblast cell-surface antigen-2 (Trop-2) polypeptide. Trop-2 (also known as epithelial glycoprotein-1, gastrointestinal tumor-associated antigen GA733-1, membrane component chromosome 1 surface marker-1, and tumor-associated calcium signal transducer-2) is a transmembrane glycoprotein that is upregulated in numerous cancer types, and is the protein product of the TACSTD2 gene. In some cases, a cancer-associated peptide is a peptide of a TROP-2 polypeptide having at least 90%, at least 95%, at least 98%, at least 99%, or 100%, amino acid sequence identity to the following TROP-2 amino acid sequence: QDNCTCPTNK MTVCSPDGPG GRCQCRALGS GMAVDCSTLT SKCLLLKARM SAPKNARTLV RPSEHALVDN DGLYDPDCDP EGRFKARQCN QTSVCWCVNS VGVRRTDKGD LSLRCDELVR THHILIDLRH RPTAGAFNHS DLDAELRRLF RERYRLHPKF VAAVHYEQPT IQIELRQNTS QKAAGDVDIG DAAYYFERDI KGESLFQGRG GLDLRVRGEP LQVERTLIYY LDEIPPKFSM KRLTAGLIAV IVVVVVALVA GMAVLVITNR RKSGKYKKVE IKELGELRKE PSL (SEQ ID NO:535).

(ii) Antibodies

As noted above, in some cases, a CTP present in a T-Cell-MP or a higher order T-Cell-MP complex, such as a duplex T-Cell-MP, of the present disclosure is an antibody or an antigen binding fragment thereof. In some cases, the CTP is an antibody that is specific for a CAA. In some cases, the CTP is an antibody specific for a peptide on the surface of an infected cell (e.g., viral, bacterial, or mycoplasma). In some cases, the CTP is an antibody specific for a peptide/HLA complex on the surface of a cancer cell, where the peptide can be a cancer-associated peptide (e.g., a peptide fragment of a cancer-associated antigen).

There are five major classes of immunoglobulins: IgA, IgD, IgE, IgG, and IgM, and several of these classes can be further divided into subclasses (isotypes), e.g., IgG1, IgG2, IgG3, IgG4, IgA, and IgA2. The subclasses can be further divided into types, e.g., IgG2a and IgG2b.

The term “humanized immunoglobulin” as used herein refers to an immunoglobulin comprising portions of immunoglobulins of different origin, wherein at least one portion comprises amino acid sequences of human origin. Chimeric or CDR-grafted single chain antibodies are also encompassed by the term humanized immunoglobulin.

The terms “antibodies” and “immunoglobulin” include antibodies or immunoglobulins of any isotype, fragments of antibodies that retain specific binding to antigen, including, but not limited to, Fab, F(ab′)₂, Fv, scFv, and Fd fragments, chimeric antibodies, humanized antibodies, single-chain antibodies (scAb), single domain antibodies (dAb), single domain heavy chain antibodies, a single domain light chain antibodies, nanobodies, bi-specific antibodies, multi-specific antibodies, and fusion proteins comprising an antigen-binding (also referred to herein as antigen binding) portion of an antibody and a non-antibody protein.

The term “nanobody” (Nb), as used herein, refers to the smallest antigen binding fragment or single variable domain (V_{HH}) derived from naturally occurring heavy chain antibody and is known to the person skilled in the art. They are derived from heavy chain only antibodies, seen in camelids (Hamers-Casterman et al. (1993) Nature 363:446; Desmyter et al. (1996) Nature Structural Biol. 3:803; and Desmyter et al. (2015) Curr. Opin. Struct. Biol. 32:1).

“Fv” is the minimum antibody fragment that contains a complete antigen-recognition and -binding site. This region consists of a dimer of one heavy- and one light-chain variable domain in tight, non-covalent association.

“Single-chain Fv” or “sFv” or “scFv” antibody fragments comprise the V_{H} and V_{L} domains of antibody, wherein these domains are present in a single polypeptide chain. In some embodiments, the Fv polypeptide further comprises a polypeptide linker between the VH and VL domains, which enables the sFv to form the desired structure for antigen binding. For a review of sFv, see Pluckthun in The Pharmacology of Monoclonal Antibodies, vol. 113, Rosenburg and Moore eds., Springer-Verlag, New York, pp. 269-315 (1994).

The term “diabodies” refers to small antibody fragments with two antigen-binding sites, which fragments comprise a heavy-chain variable domain (VH) connected to a light-chain variable domain (VL) in the same polypeptide chain (V_{H}-V_{L}). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al. (1993) Proc. Natl. Acad. Sci. USA 90:6444-6448.

As used herein, the term “CDR” or “complementarity determining region” is intended to mean the non-contiguous antigen combining sites found within the variable region of both heavy and light chain polypeptides. CDRs have been described by Kabat et al (1977) J. Biol. Chem. 252:6609; Kabat et al., U.S. Dept. of Health and Human Services, “Sequences of proteins of immunological interest” (1991) (also referred to herein as Kabat 1991); by Chothia et al. (1987) J. Mol. Biol. 196:901 (also referred to herein as Chothia 1987); and MacCallum et al. (1996) J. Mol. Biol. 262:732, where the definitions include overlapping or subsets of amino acid residues when compared against each other. Nevertheless, application of either definition to refer to a CDR of an antibody or grafted antibodies or variants thereof is intended to be within the scope of the term as defined and used herein. The amino acid residues, which encompass the CDRs, as defined by each of the above cited references are set forth in the CDR-table below as a comparison.

### CDR Table

| | Kabat¹ | Chothia² | MacCallum³ |
|---|---|---|---|
| V_{H} CDR-1 | 31-35 | 26-32 | 30-35 |
| V_{H} CDR-2 | 50-65 | 53-55 | 47-58 |
| V_{H} CDR-3 | 95-102 | 96-101 | 93-101 |
| V_{L} CDR-1 | 24-34 | 26-32 | 30-36 |
| V_{L} CDR-2 | 50-56 | 50-52 | 46-55 |
| V_{L} CDR-3 | 89-97 | 91-96 | 89-96 |

¹Residue numbering follows the nomenclature of Kabat et al., 1991, supra
²Residue numbering follows the nomenclature of Chothia et al., supra
³Residue numbering follows the nomenclature of MacCallum et al., supra

As used herein, the terms “CDR-L1”, “CDR-L2”, and “CDR-L3” refer, respectively, to the first, second, and third CDRs in a light chain variable region. The terms “CDR-L1”, “CDR-L2”, and “CDR-L3” may be used interchangeably with “VL CDR1,” “VL CDR2,” and “VL CDR3,” respectively. As used herein, the terms “CDR-H1”, “CDR-H2”, and “CDR-H3” refer, respectively, to the first, second, and third CDRs in a heavy chain variable region. The terms “CDR-H1”, “CDR-H2”, and “CDR-H3” may be used interchangeably with “VH CDR1,” “VH CDR2,” and “VH CDR3,” respectively. As used herein, the terms “CDR-1”, “CDR-2”, and “CDR-3” refer, respectively, to the first, second and third CDRs of either chain's variable region.

Non-limiting examples of CAA-targeted antibodies (or antigen binding fragments thereof) that can be included in a T-Cell-MP include, but are not limited to, abituzumab (anti-CD51), LL1 (anti-CD74), LL2 or RFB4 (anti-CD22), veltuzumab (hA20, anti-CD20), rituxumab (anti-CD20), obinutuzumab (GA101, anti-CD20), daratumumab (anti-CD38), lambrolizumab (anti-PD-1 receptor), nivolumab (anti-PD-1 receptor), ipilimumab (anti-CTLA-4), RS7 (anti-TROP-2), PAM4 or KC4 (both anti-mucin), MN-14 (anti-CEA), MN-15 or MN-3 (anti-CEACAM6), Mu-9 (anti-colon-specific antigen-p), Immu 31 (anti-alpha-fetoprotein), R1 (anti-IGF-1R), A19 (anti-CD19), TAG-72 (e.g., CC49), Tn, J591 or HuJ591 (anti-PSMA), AB-PG1-XG1-026 (anti-PSMA dimer), D2/B (anti-PSMA), G250 (anti-carbonic anhydrase IX), L243 (anti-HLA-DR) alemtuzumab (anti-CD52), oportuzumab (anti-EpCAM), bevacizumab (anti-VEGF), cetuximab (anti-EGFR), gemtuzumab (anti-CD33), ibritumomab tiuxetan (anti-CD20); panitumumab (anti-EGFR); tositumomab (anti-CD20); PAM4 (also known as clivatuzumab; anti-mucin), trastuzumab (anti-HER2), pertuzumab (anti-HER2), polatuzumab (anti-CD79b), and anetumab (anti-mesothelin).

In some cases, a CAA-targeted antibody (or antigen binding fragments thereof) that can be included in a T-Cell-MP is a single-chain antibody. In some cases, a CAA-targeted antibody (or antigen binding fragments thereof) that can be included in a T-Cell-MP is a scFv. In some cases, the tumor-targeting polypeptide is a nanobody (also referred to as a single domain antibody (sdAb)). In some cases, the tumor-targeting polypeptide is a heavy chain nanobody. In some cases, the tumor-targeting polypeptide is a light chain nanobody.

VH and VL aa sequences of various tumor antigen-binding antibodies are known in the art, as are the light chain and heavy chain CDRs of such antibodies. See, e.g., Ling et al. (2018) Frontiers Immunol. 9:469; WO 2005/012493; US 2019/0119375; US 2013/0066055. The following are non-limiting examples of tumor antigen-binding antibodies.

(a) Anti-Her2

An anti-Her2 antibody (or antigen binding fragments thereof) useful as a CTP may comprise: a) a light chain comprising an aa sequence having at least 90%, at least 95%, at least 98%, at least 99%, or 100%, aa sequence identity to the following aa sequence: DIQMTQSPSSLSASVGDRVTITCRASQDVN TAVAWYQQKPGKAPKLLIYSASFLYSGVPSRFSGSRSGTDFTLTISSLQPEDFATYYCQQHYTTPP TFGQGTKVEIKRTVAAPSVFIFPPSDEQLKSGTASVVCLLNNFYPREAKVQWKVDNALQSGNSQ ESVTEQDSKDSTYSLSSTLTLSKADYEKHKVYACEVTHQGLSSPVTKSFNRGEC (SEQ ID NO:172); and b) a heavy chain comprising an aa sequence having at least 90%, at least 95%, at least 98%, at least 99%, or 100%, aa sequence identity to the following aa sequence: EVQLVESGGGLVQPGGSLR LSCAASGFNIKDTYIHWVRQAPGKGLEWVARIYPTNGYTRYADSVKGRFTISADTSKNTAYLQM NSLRAEDTAVYYCSRWGGDGFYAMDYWGQGTLVTVSSASTKGPSVFPLAPSSKSTSGGTAALG CLVKDYFPEPVTVSWNSGALTSGVHTFPAVLQSSGLYSLSSVVTVPSSSLGTQTYICNVNHKPSN TKVDKKVEPKSCDKTHTCPPCPAPELLGGPSVFLFPPKPKDTLMISRTPEVTCVVVDVSHEDPEV KFNWYVDGVEVHNAKTKPREEQYNSTYRVVSVLTVLHQDWLNGKEYKCKVSNKALPAPIEKTI SKAKGQPREPQVYTLPPSREEMTKNQVSLTCLVKGFYPSDIAVEWESNGQPENNYKTTPPVLDS DGSFFLYSKLTVDKSRWQQGNVFSCSVMHEALHNHYTQKSLSLSPGK (SEQ ID NO:173).

An anti-Her2 antibody (or antigen binding fragments thereof) may comprise a light chain variable region (VL) present in the light chain aa sequence provided above; and a heavy chain variable region (VH) present in the heavy chain aa sequence provided above. For example, an anti-Her2 antibody can comprise: a) a VL comprising an aa sequence having at least 90%, at least 95%, at least 98%, at least 99%, or 100%, aa sequence identity to the aa sequence: DIQMTQSPSS LSASVGDRVT ITCRASQDVN TAVAWYQQKP GKAPKLLIYS ASFLYSGVPS RFSGSRSGTD FTLTISSLQP EDFATYYCQQ HYTTPPTFGQ GTKVEIK (SEQ ID NO:174); and b) a VH comprising an aa sequence having at least 90%, at least 95%, at least 98%, at least 99%, or 100%, aa sequence identity to the aa sequence: EVQLVESGGG LVQPGGSLR LSCAASGFNI KDTYIHWVRQ APGKGLEWVA RIYPTNGYTR YADSVKGRFT ISADTSKNTA YLQMNSLRAE DTAVYYCSRW GGDGFYAMDY WGQGTLVTVS S (SEQ ID NO:175). An anti-Her2 antibody may comprise, in order from N-terminus to C-terminus: a) a VH comprising an aa sequence having at least 90%, at least 95%, at least 98%, at least 99%, or 100%, aa sequence identity to the aa sequence: EVQLVESGGG LVQPGGSLRL SCAASGFNIK DTYIHWVRQA PGKGLEWVAR IYPTNGYTRY ADSVKGRFTI SADTSKNTAY LQMNSLRAED TAVYYCSRWG GDGFYAMDYW GQGTLVTVSS (SEQ ID NO:176); b) a linker; and c) a VL comprising an aa sequence having at least 90%, at least 95%, at least 98%, at least 99%, or 100%, aa sequence identity to the aa sequence: DIQMTQSPSS LSASVGDRVT ITCRASQDVN TAVAWYQQKP GKAPKLLIYS ASFLYSGVPS RFSGSRSGTD FTLTISSLQP EDFATYYCQQ HYTTPPTFGQ GTKVEIK (SEQ ID NO:177). Suitable linker sequences described elsewhere herein and include, e.g., (GGGGS) (SEQ ID NO:139), which may be repeated from 1 to 10 times (e.g., 1, 2, 3, 4, 5, 6, 7, 8, 9, or 10 times).

In some cases, an anti-Her2 antibody (or antigen binding fragments thereof) comprises VL CDR1, VL CDR2, and VL CDR3 present in the light chain aa sequence provided above; and VH CDR1, CDR2, and CDR3 present in the heavy chain aa sequence provided above. In some cases, the VH and VL CDRs are as defined by Kabat et al., J. Biol. Chem. 252:6609-6616 (1977); Kabat et al., U.S. Dept. of Health and Human Services, “Sequences of proteins of immunological interest” (1991) (also referred to herein as Kabat 1991. In some cases, the VH and VL CDRs are as defined by Chothia et al., J. Mol. Biol. 196:901-917 (1987) (also referred to herein as Chothia 1987). For example, an anti-Her2 antibody (or antigen binding fragments thereof) can comprise a VL CDR1 having the aa sequence RASQDVNTAVA (SEQ ID NO:179); a VL CDR2 having the aa sequence SASFLY (SEQ ID NO:180); a VL CDR3 having the aa sequence QQHYTTPP (SEQ ID NO:181); a VH CDR1 having the aa sequence GFNIKDTY (SEQ ID NO182); a VH CDR2 having the aa sequence IYPTNGYT (SEQ ID NO:183); and a VH CDR3 having the aa sequence SRWGGDGFYAMDY (SEQ ID NO:184).

In some cases, an anti-Her2 antibody (or antigen binding fragments thereof) is a scFv antibody. For example, an anti-Her2 scFv can comprise an aa sequence having at least 90%, at least 95%, at least 98%, at least 99%, or 100%, aa sequence identity to the following aa sequence: EVQLVESGGGLVQPG GSLRLSCAASGFNIKDTYIHWVRQAPGKGLEWVARIYPTNGYTRYADSVKGRFTISADTSKNTA YLQMNSLRAEDTAVYYCSRWGGDGFYAMDYWGQGTLVTVSSGGGGSGGGGSGGGGSDIQMT QSPSSLSASVGDRVTITCRASQDVNTAVAWYQQKPGKAPKLLIYSASFLYSGVPSRFSGSRSGTD FTLTISSLQPEDFATYYCQQHYTTPPTFGQGTKVEIK (SEQ ID NO:185).

As another example, in some cases, an anti-Her2 antibody (or antigen binding fragments thereof) comprises: a) a light chain variable region (VL) comprising an aa sequence having at least 90%, at least 95%, at least 98%, at least 99%, or 100%, aa sequence identity to the following aa sequence: DIQMTQSPSSLSASVGDRVTITCKASQDVSIGVAWYQQKPGKAPKL LIYSASYRYTGVP-SRFSGSGSGTDFTLTISSLQPEDFATYYCQQYYIYPYTFGQGTKVEIKRTVAAPSVFIFPPSDEQLK SGTASVVCLLNNFYPREAKVQWKVDNALQSGNSQESVTEQDSKDSTYSLSSTLTLSKADYEKHK VYACEVTHQGLSSPVTKSFNRGEC (SEQ ID NO:186); and b) a heavy chain variable region (VH) comprising an aa sequence having at least 90%, at least 95%, at least 98%, at least 99%, or 100%, aa sequence identity to the following aa sequence: EVQLVESGGGLVQPGGSLRLSCAASGFTFTDYT MDWVRQAPGKGLEWVADVNPNSGGSIYNQRFKGRFTLSVDRSKNTLYLQMNSLRAEDTAVYY CARNLGPSFYFDYWGQGTLVTVSSASTKGPSVFPLAPSSKSTSGGTAALGCLVKDYFPEPVTVS WNSGALTSGVHTFPAVLQSSGLYSLSSVVTVPSSSLGTQTYICNVNHKPSNTKVDKKVEPKSCDK THTCPPCPAPELLGGPSVFLFPPKPKDTLMISRTPEVTCVVVDVSHEDPEVKFNWYVDGVEVHNA KTKPREEQYNSTYRVVSVLTVLHQDWLNGKEYKCKVSNKALPAPIEKTISKAKGQPREPQVYTL PPSREEMTKNQVSLTCLVKGFYPSDIAVEWESNGQPENNYKTTPPVLDSDGSFFLYSKLTVDKSR WQQGNVFSCSVMHEALHNHYTQKSLSLSPG (SEQ ID NO:187).

In some cases, an anti-Her2 antibody (or antigen binding fragments thereof) useful as a CTP comprises a VL present in the light chain aa sequence provided above; and a VH present in the heavy chain aa sequence provided above. For example, an anti-Her2 antibody can comprise: a) a VL comprising an aa sequence having at least 90%, at least 95%, at least 98%, at least 99%, or 100%, aa sequence identity to the aa sequence: DIQMTQSPSSLSASVGDRVTITCKAS QDVSIGVAWYQQKP GKAPKLLIYSASYRYTGVPSRFSGSGSGTDFTLTISSLQPEDFATYYCQQYYIYPYTFGQGTKVEI K (SEQ ID NO:188); and b) a VH comprising an aa sequence having at least 90%, at least 95%, at least 98%, at least 99%, or 100%, aa sequence identity to the aa sequence: EVQLVESGGGLVQPGGSLRL-SCAASGFTFTDYTMDWVRQAPGKGLEWVADVNPNSGGSIYNQRFKGRFTLSVDRSKNTLYLQM NSLRAEDTAVYYCARNLGPSFYFDYWGQGTLVTVSS (SEQ ID NO:189).

In some cases, an anti-Her2 antibody (or antigen binding fragments thereof) used as a CTP comprises VL CDR1, VL CDR2, and VL CDR3 present in the light chain aa sequence provided above; and VH CDR1, CDR2, and CDR3 present in the heavy chain aa sequence provided above. In some cases, the VH and VL CDRs are as defined by Kabat (see, e.g., Kabat 1991). In some cases, the VH and VL CDRs are as defined by Chothia (see, e.g., Chothia 1987). For example, an anti-HER2 antibody can comprise a VL CDR1 having the aa sequence KASQDVSIGVA (SEQ ID NO:190); a VL CDR2 having the aa sequence SASYRY (SEQ ID NO:191); a VL CDR3 having the aa sequence QQYYIYPY (SEQ ID NO:192); a VH CDR1 having the aa sequence GFTFTDYTMD (SEQ ID NO:193); a VH CDR2 having the aa sequence ADVNPNSGGSIYNQRFKG (SEQ ID NO:194); and a VH CDR3 having the aa sequence ARNLGPSFYFDY (SEQ ID NO:195).

In some cases, an anti-Her2 antibody (or antigen binding fragments thereof) is a scFv. For example, in some cases, an anti-Her2 scFv comprises an aa sequence having at least 90%, at least 95%, at least 98%, at least 99%, or 100%, aa sequence identity to the following aa sequence:

(SEQ ID NO: 196)
EVQLVESGGGLVQPGGSLRLSCAASGFNIKDTYIHWVRQAPGKGLEWVA
RIYPTNGYTRYADSVKGRFTISADTSKNTAYLQMNSLRAEDTAVYYCSR
WGGDGFYAMDYWGQGTLVTVSSGGGGSGGGGSGGGGSDIQMTQSPSSLS
ASVGDRVTITCRASQDVNTAVAWYQQKPGKAPKLLIYSASFLYSGVPSR
FSGSRSGTDFTLTISSLQPEDFATYYCQQHYTTPPTFGQGTKVEIK.

(b) Anti-CD19

Anti-CD19 antibodies (and antigen binding fragments thereof) useful as a CTP are known in the art; and the VH and VL, or the VH and VL CDRs, of any anti-CD19 antibody can be used in a T-Cell-MP. See e.g., WO 2005/012493.

An anti-CD19 antibody (or antigen binding fragments thereof) may include a VL CDR1 comprising the aa sequence KASQSVDYDGDSYLN (SEQ ID NO:197); a VL CDR2 comprising the aa sequence DASNLVS (SEQ ID NO:198); and a VL CDR3 comprising the aa sequence QQSTEDPWT (SEQ ID NO:199). An anti-CD19 antibody (or antigen binding fragments thereof) may include a VH CDR1 comprising the aa sequence SYWMN (SEQ ID NO:200); a VH CDR2 comprising the aa sequence QIWPGDGDTNYNGKFKG (SEQ ID NO:201); and a VH CDR3 comprising the aa sequence RETTTVGRYYYAMDY (SEQ ID NO:202). An anti-CD19 antibody may include a VL CDR1 comprising the aa sequence KASQSVDYDGDSYLN (SEQ ID NO:197); a VL CDR2 comprising the aa sequence DASNLVS (SEQ ID NO:198); a VL CDR3 comprising the aa sequence QQSTEDPWT (SEQ ID NO:199); a VH CDR1 comprising the aa sequence SYWMN (SEQ ID NO:200); a VH CDR2 comprising the aa sequence QIWPGDGDTNYNGKFKG (SEQ ID NO:201); and a VH CDR3 comprising the aa sequence RETTTVGRYYYAMDY (SEQ ID NO:202).

An anti-CD19 antibody (or antigen binding fragments thereof) may be a scFv. For example, an anti-CD19 scFv may comprises an aa sequence having at least 90%, at least 95%, at least 98%, at least 99%, or 100%, aa sequence identity to the following aa sequence: DIQLTQSPAS LAVSLGQRAT ISCKASQSVD YDGDSYLNWY QQIPGQPPKL LIYDASNLVS GIPPRFSGSG SGTDFTLNIH PVEKVDAATY HCQQSTEDPW TFGGGTKLEI KGGGGSGGGG SGGGGSQVQL QQSGAELVRP GSSVKISCKA SGYAFSSYWM NWVKQRPGQG LEWIGQIWPG DGDTNYNGKF KGKATLTADE SSSTAYMQLS SLASEDSAVY FCARRETTTV GRYYYAMDYW GQGTTVTVS (SEQ ID NO:203).

(c) Anti-Mesothelin

Anti-mesothelin antibodies (or antigen binding fragments thereof) useful as a CTP are known in the art; and the VH and VL, or the VH and VL CDRs, of any anti-mesothelin antibody can be used in a T-Cell-MP as targeting sequences. See, e.g., U.S. 2019/0000944; WO 2009/045957; WO 2014/031476; U.S. Pat. No. 8,460,660; US 2013/0066055; and WO 2009/068204.

An anti-mesothelin antibody (or antigen binding fragments thereof) may comprise: a) a light chain comprising an aa sequence having at least 90%, at least 95%, at least 98%, at least 99%, or 100%, aa sequence identity to the following aa sequence: DIALTQPASV SGSPGQSITI SCTGTSSDIG GYNSVSWYQQ HPGKAPKLMI YGVNNRPSGV SNRFSGSKSG NTASLTISGL QAEDEADYYC SSYDIESATP VFGGGTKLTV LGQPKAAPSV TLFPPSSEEL QANKATLVCL ISDFYPGAVT VAWKGDSSPV KAGVETTTPS KQSNNKYAAS SYLSLTPEQW KSHRSYSCQVT HEGSTVEKTV APTESS (SEQ ID NO:204); and b) a heavy chain comprising an aa sequence having at least 90%, at least 95%, at least 98%, at least 99%, or 100%, aa sequence identity to the following aa sequence:

(SEQ ID NO: 205)
QVELVQSGAE VKKPGESLKI SCKGSGYSFT SYWIGWVRQA
PGKGLEWMGI IDPGDSRTRY SPSFQGQVTI SADKSISTAY
LQWSSLKASD TAMYYCARGQ LYGGTYMDGW GQGTLVTVSS
ASTKGPSVFP LAPSSKSTSG GTAALGCLVK DYFPEPVTVS
WNSGALTSG VHTFPAVLQS SGLYSLSSV VTVPSSSLGT
QTYICNVNHK PSNTKVDKKV EPKSCDKTHT CPPCPAPELL
GGPSVFLFPP KPKDTLMISR TPEVTCVVVD VSHEDPEVK
FNWYVDGVEV HNAKTKPRE EQYNSTYRVV SVLTVLHQDW
LNGKEYKCKV SNKALPAPIE KTISKAKGQ PREPQVYTL
PPSRDELTKN QVSLTCLVKG FYPSDIAVE WESNGQPEN
NYKTTPPVL DSDGSFFLYS KLTVDKSRWQQ GNVFSCSVMH
EALHNHYTQ KSLSLSPGK.

An anti-mesothelin antibody (or antigen binding fragments thereof) may comprise a VL present in the light chain aa sequence provided above; and a VH present in the heavy chain aa sequence provided above. For example, an anti-mesothelin antibody can comprise: a) a VL comprising an aa sequence having at least 90%, at least 95%, at least 98%, at least 99%, or 100%, aa sequence identity to the aa sequence: DIALTQPASVSGSPGQSITISCTGTSSDIGGYNSVSWYQQHPGKAPKLMI-YGVNNRPSGVSNRFSGSKSGNTASLTISGLQAEDEADYYCSSYDIESATPVFGGGTK (SEQ ID NO:206); and b) a VH comprising an aa sequence having at least 90%, at least 95%, at least 98%, at least 99%, or 100%, aa sequence identity to the aa sequence: QVELVQSGAE VKKPGESLKI SCKGSGYSFT SYWIGWVRQ APGKGLEWMG IIDPGDSRTR YSPSFQGQV TISADKSIST AYLQWSSLK ASDTAMYYCA RGQLYGGTYM DGWGQGTLV TVSS (SEQ ID NO:207).

An anti-mesothelin antibody (or antigen binding fragments thereof) may comprise a VL CDR1, VL CDR2, and VL CDR3 present in the light chain aa sequence provided above; and VH CDR1, CDR2, and CDR3 present in the heavy chain aa sequence provided above. The VH and VL CDRs may be as defined by Kabat (see, e.g., Kabat 1991). In some cases, the VH and VL CDRs are as defined by Chothia (see, e.g., Chothia 1987). For example, an anti-mesothelin antibody (or antigen binding fragments thereof) can comprise a VL CDR1 having the aa sequence TGTSSDIGGYNSVS (SEQ ID NO:208); a VL CDR2 having the aa sequence LMIYGVNNRPS (SEQ ID NO:209); a VL CDR3 having the aa sequence SSYDIESATP (SEQ ID NO:210); a VH CDR1 having the aa sequence GYSFTSYWIG (SEQ ID NO:211); a VH CDR2 having the aa sequence WMGIIDPGDSRTRYSP (SEQ ID NO:212); and a VH CDR3 having the aa sequence GQLYGGTYMDG (SEQ ID NO:213). An anti-mesothelin antibody can be a scFv. As one non-limiting example, an antimesothelin scFv can comprise the following aa sequence: QVQLQQSGAE VKKPGASVKV SCKASGYT FTGYYMHWVR QAPGQGLEWM GRINPNSGGT NYAQKFQGRV TMTRDTSIST AYMELSRLRS EDTAVYYCAR GRYYGMDVWG QGTMVTVSSG GGGSGGGGSG GGGSGGGGSE IVLTQSPATL SLSPGERATI SC [symbol] WYQQRPG QAPRLLIY [symbol] GIPPR FSGSGSGTDF TLTISSLEPE DFAAYYC [symbol] GQG TKVDIK (SEQ ID NO:214), where VH CDR1, CDR2, and CDR3 are underlined; and VL CDR1, CDR2, and CDR3 are bolded and underlined.

As one non-limiting example, an anti-mesothelin scFv can comprise the following aa sequence: QVQLVQSGAEV KKPGASVKVS CKASGYTFTG YYMHWVRQAP GQGLEWMGWI NPNSGGTNYA QKFQGRVTMT RDTSISTAYM ELSRLRSDDT AVYYCARDLR RTVVTPRAYY GMDVWGQGTT VTVSSGGGGS GGGGSGGGGS GGGGSDIQLT QSPSTLSASV GDRVTITC [symbol] [symbol] W YQQKAGKAPK LLIY [symbol] GVPSRFSGS GSGTDFSFTI SSLQPEDIAT YYC [symbol] TFGQGTKVE IK (SEQ ID NO:215), where VH CDR1, CDR2, and CDR3 are underlined; and VL CDR1, CDR2, and CDR3 are bolded and underlined.

In some cases, an anti-mesothelin antibody suitable for inclusion in a T-Cell-MP as a targeting sequence comprises: a) VL CDR1, VL CDR2, and VL CDR3 present in a light chain variable region (VL) comprising the following amino acid sequence: EIVLTQSPGTLSLSPGERATLSCRASQSVSSSYLAW YQQKPGQAPRLLIYGASSRATGIPDRFSGSGSGTDFTLTISRLEPEDFAVYYCQQYGSSPIFTFGPG TKVDIK (SEQ ID NO:536); and b) VH CDR1, CDR2, and CDR3 present in a heavy chain variable region (VH) comprising the following amino acid sequence: QMQLVESGGGVVQPGRSLRLSCTASG FTFSNNGMHWVRQAPGKGLEWVAVIWFDGMNKFYVDSVKGRFTISRDNSKNTLYLEMNSLRA EDTAIYYCAREGDGSGIYYYYGMDVWGQGTTVTVSS (SEQ ID NO:537). In some cases, the VH and VL CDRs are as defined by Kabat (see, e.g., the CDR Table above; and Kabat 1991). In some cases, the VH and VL CDRs are as defined by Chothia (see, e.g., the CDR Table above; and Chothia 1987).

In some cases, an anti-mesothelin antibody suitable for inclusion in a T-Cell-MP as a targeting sequence comprises: a) a VL region comprising an amino acid sequence having at least 90%, at least 95%, at least 98%, at least 99%, or 100%, amino acid sequence identity to the following amino acid sequence: EIVLTQSPGTLSLSPGERATLSCRASQSVSSSYLAWYQQKPGQAPRLLIYGASSRAT GIPDRFSGSGSGTDFTLTISRLEPEDFAVYYCQQYGSSPIFTFGPGTKVDIK (SEQ ID NO:536); and b) a VH region comprising an amino acid sequence having at least 90%, at least 95%, at least 98%, at least 99%, or 100%, amino acid sequence identity to the following amino acid sequence: QMQLVESGG GVVQPGRSLRLSCTASGFTFSNNGMHWVRQAPGKGLEWVAVIWFDGMNKFYVDSVKGRFTISR DNSKNTLYLEMNSLRAEDTAIYYCAREGDGSGIYYYYGMDVWGQGTTVTVSS (SEQ ID NO:537).

In some cases, an anti-mesothelin antibody suitable for inclusion in a T-Cell-MP as a targeting sequence is a scFv comprising, in order from N-terminus to C-terminus: a) a VH region comprising an amino acid sequence having at least 90%, at least 95%, at least 98%, at least 99%, or 100%, amino acid sequence identity to the following amino acid sequence: QMQLVESGGGVVQPGRSLRLSCTASGFTF SNNGMHWVRQAPGKGLEWVAVIWFDGMNKFYVDSVKGRFTISRDNSKNTLYLEMNSLRAEDT AIYYCAREGDGSGIYYYYGMDVWGQGTTVTVSS (SEQ ID NO:537); b) a peptide linker; and c) a VL region comprising an amino acid sequence having at least 90%, at least 95%, at least 98%, at least 99%, or 100%, amino acid sequence identity to the following amino acid sequence: EIVLTQSPGTLSLSPGERATLSCRASQSVSSSYLAWYQQKPGQAPRLLIYGASSRATGIPDRFSGSG SGTDFTLTISRLEPEDFAVYYCQQYGSSPIFTFGPGTKVDIK (SEQ ID NO:536). In some cases, the peptide linker comprises the amino acid sequence (GGGGS)n, where n is an integer from 1 to 10 (e.g., where n is 1, 2, 3, 4, 5, 6, 7, 8, 9, or 10). In some cases, the peptide linker comprises the amino acid sequence GGGGSGGGGSGGGGS (SEQ ID NO:538) and has a length of 15 amino acids.

In some cases, an anti-mesothelin antibody suitable for inclusion in a T-Cell-MP as a targeting sequence is a scFv comprising, in order from N-terminus to C-terminus: a) a VL region comprising an amino acid sequence having at least 90%, at least 95%, at least 98%, at least 99%, or 100%, amino acid sequence identity to the following amino acid sequence: EIVLTQSPGTLSLSPGERATLSCRASQSVSS SYLAWYQQKPGQAPRLLIYGASSRATGIPDRFSGSGSGTDFTLTISRLEPEDFAVYYCQQYGSSPI FTFGPGTKVDIK (SEQ ID NO:536); b) a peptide linker; and c) a VH region comprising an amino acid sequence having at least 90%, at least 95%, at least 98%, at least 99%, or 100%, amino acid sequence identity to the following amino acid sequence: QMQLVESGGGVVQPGRSLRLSCTASGFTFSNNGM HWVRQAPGKGLEWVAVIWFDGMNKFYVDSVKGRFTISRDNSKNTLYLEMNSLRAEDTAIYYC AREGDGSGIYYYYGMDVWGQGTTVTVSS (SEQ ID NO:537). In some cases, the peptide linker comprises the amino acid sequence (GGGGS)n, where n is an integer from 1 to 10 (e.g., where n is 1, 2, 3, 4, 5, 6, 7, 8, 9, or 10). In some cases, the peptide linker comprises the amino acid sequence GGGGSGGGGSGGGGS (SEQ ID NO:538) and has a length of 15 amino acids.

In some cases, an anti-mesothelin antibody suitable for inclusion in a T-Cell-MP as a targeting sequence comprises: a) VL CDR1, VL CDR2, and VL CDR3 present in a light chain variable region (VL) comprising the following amino acid sequence: DIELTQSPAIMSASPGEKVTMTCSASSSVSYMHWYQQKSGTSPKRWIYDTSKLASGVPGRFSGSG SGNSYSLTISSVEAEDDATYYCQQWSKHPLTFGSGTKVEIK (SEQ ID NO:539); and b) VH CDR1, CDR2, and CDR3 present in a heavy chain variable region (VH) comprising the following amino acid sequence: QVQLQQSGPELEKPGASVKISCKASGYSFTGYTMNWVKQSHGKSLEWIGLITPYNG ASSYNQKFRGKATLTVDKSSSTAYMDLLSLTSEDSAVYFCARGGYDGRGFDYWGSGTPVTVSS (SEQ ID NO:540). In some cases, the VH and VL CDRs are as defined by Kabat (see, e.g., the CDR Table above; and Kabat 1991). In some cases, the VH and VL CDRs are as defined by Chothia (see, e.g., the CDR Table above; and Chothia 1987).

In some cases, an anti-mesothelin antibody suitable for inclusion in a T-Cell-MP as a targeting sequence comprises: a) a VL region comprising an amino acid sequence having at least 90%, at least 95%, at least 98%, at least 99%, or 100%, amino acid sequence identity to the following amino acid sequence: DIELTQSPAIMSASPGEKVTMTCSASSSVSYMHWYQQKSGTSPKRWIYDTSKLASG VPGRFSGSGSGNSYSLTISSVEAEDDATYYCQQWSKHPLTFGSGTKVEIK (SEQ ID NO:539); and b) a VH region comprising an amino acid sequence having at least 90%, at least 95%, at least 98%, at least 99%, or 100%, amino acid sequence identity to the following amino acid sequence: QVQLQQSGPE LEKPGASVKISCKASGYSFTGYTMNWVKQSHGKSLEWIGLITPYNGASSYNQKFRGKATLTVDK SSSTAYMDLLSLTSEDSAVYFCARGGYDGRGFDYWGSGTPVTVSS (SEQ ID NO:540).

In some cases, an anti-mesothelin antibody suitable for inclusion in a T-Cell-MP as a targeting sequence is a scFv comprising, in order from N-terminus to C-terminus: a) a VL region comprising an amino acid sequence having at least 90%, at least 95%, at least 98%, at least 99%, or 100%, amino acid sequence identity to the following amino acid sequence: DIELTQSPAIMSASPGEKVTMTCSASSSVSYMHWYQQKSGTSPKRWIYDTSKLASGVPGRFSGSG SGNSYSLTISSVEAEDDATYYCQQWSKHPLTFGSGTKVEIK (SEQ ID NO:539); b) a peptide linker; and c) a VH region comprising an amino acid sequence having at least 90%, at least 95%, at least 98%, at least 99%, or 100%, amino acid sequence identity to the following amino acid sequence: QVQLQQSGPELEKPGASVKISCKASGYSFTGYTMNWVKQSHGKSLEWIGLITPYNGASSYNQKF RGKATLTVDKSSSTAYMDLLSLTSEDSAVYFCARGGYDGRGFDYWGSGTPVTVSS (SEQ ID NO:540). In some cases, the peptide linker comprises the amino acid sequence (GGGGS)n, where n is an integer from 1 to 10 (e.g., where n is 1, 2, 3, 4, 5, 6, 7, 8, 9, or 10). In some cases, the peptide linker comprises the amino acid sequence GGGGSGGGGSGGGGS (SEQ ID NO:538) and has a length of 15 amino acids.

In some cases, an anti-mesothelin antibody suitable for inclusion in a T-Cell-MP as a targeting sequence is a scFv comprising, in order from N-terminus to C-terminus: a) a VH region comprising an amino acid sequence having at least 90%, at least 95%, at least 98%, at least 99%, or 100%, amino acid sequence identity to the following amino acid sequence: QVQLQQSGPELEKPGASVKISCKASGYSFTGYTMNWVKQSHGKSLEWIGLITPYNGASSYNQKF RGKATLTVDKSSSTAYMDLLSLTSEDSAVYFCARGGYDGRGFDYWGSGTPVTVSS (SEQ ID NO:540); b) a peptide linker; and c) a VL region comprising an amino acid sequence having at least 90%, at least 95%, at least 98%, at least 99%, or 100%, amino acid sequence identity to the following amino acid sequence: DIELTQSPAIMSASPGEKVTMTCSASSSVSYMHWYQQKSGTSPKRWIYDTSKLASGVPGRFSGSG SGNSYSLTISSVEAEDDATYYCQQWSKHPLTFGSGTKVEIK (SEQ ID NO:539). In some cases, the peptide linker comprises the amino acid sequence (GGGGS)n, where n is an integer from 1 to 10 (e.g., where n is 1, 2, 3, 4, 5, 6, 7, 8, 9, or 10). In some cases, the peptide linker comprises the amino acid sequence GGGGSGGGGSGGGGS (SEQ ID NO:538) and has a length of 15 amino acids.

In some cases, an anti-mesothelin antibody suitable for inclusion in a T-Cell-MP as a targeting sequence comprises: a) VL CDR1, VL CDR2, and VL CDR3 present in a light chain variable region (VL) comprising the following amino acid sequence: DIALTQPASVSGSPGQSITISCTGTSSDIGGYNSVSWYQQHPGKAPKLMIYGVNNRPSGVSNRFSG SKSGNTASLTISGLQAEDEADYYCSSYDIESATPVFGGGTKLTVLG (SEQ ID NO:541); and b) VH CDR1, CDR2, and CDR3 present in a heavy chain variable region (VH) comprising the following amino acid sequence: QVELVQSGAEVKKPGESLKISCKGSGYSFTSYWIGWVRQAPGKGLEWMGIIDPG DSRTRYSPSFQGQVTISADKSISTAYLQWSSLKASDTAMYYCARGQLYGGTYMDGWGQGTLVT VSS (SEQ ID NO:542). In some cases, the VH and VL CDRs are as defined by Kabat (see, e.g., the CDR Table, above; and Kabat 1991). In some cases, the VH and VL CDRs are as defined by Chothia (see, e.g., the CDR Table, above; and Chothia 1987).

In some cases, an anti-mesothelin antibody suitable for inclusion in a T-Cell-MP as a targeting sequence comprises: a) a VL region comprising an amino acid sequence having at least 90%, at least 95%, at least 98%, at least 99%, or 100%, amino acid sequence identity to the following amino acid sequence: DIALTQPASVSGSPGQSITISCTGTSSDIGGYNSVSWYQQHPGKAPKLMIYGVNNRPSGVSNRFSG SKSGNTASLTISGLQAEDEADYYCSSYDIESATPVFGGGTKLTVLG (SEQ ID NO:541); and b) a VH region comprising an amino acid sequence having at least 90%, at least 95%, at least 98%, at least 99%, or 100%, amino acid sequence identity to the following amino acid sequence:

(SEQ ID NO: 542)
QVELVQSGAEVKKPGESLKISCKGSGYSFTSYWIGWVRQAPGKGLEWMG
IIDPGDSRTRYSPSFQGQVTISADKSISTAYLQWSSLKASDTAMYYCAR
GQLYGGTYMDGWGQGTLVTVSS.

An anti-mesothelin antibody suitable for inclusion in a T-Cell-MP as a targeting sequence is a scFv that may comprise, in order from N-terminus to C-terminus: a) a VL region comprising an amino acid sequence having at least 90%, at least 95%, at least 98%, at least 99%, or 100%, amino acid sequence identity to the following amino acid sequence: DIALTQPASVSGSPGQSITISCTGTSSDIGGYNSVSWYQQHPGKAPKLMIYGVNNRPSGVSNRFSG SKSGNTASLTISGLQAEDEADYYCSSYDIESATPVFGGGTKLTVLG (SEQ ID NO:541); b) a peptide linker; and c) a VH region comprising an amino acid sequence having at least 90%, at least 95%, at least 98%, at least 99%, or 100%, amino acid sequence identity to the following amino acid sequence: QVELVQSGAEVKKPGESLKISCKGSGYSFTSYWIGWVRQAPGKGLEWMGIIDPGDSRTRYSPSFQ GQVTISADKSISTAYLQWSSLKASDTAMYYCARGQLYGGTYMDGWGQGTLVTVSS (SEQ ID NO:542). In some cases, the peptide linker comprises the amino acid sequence (GGGGS)n, where n is an integer from 1 to 10 (e.g., where n is 1, 2, 3, 4, 5, 6, 7, 8, 9, or 10). In some cases, the peptide linker comprises the amino acid sequence GGGGSGGGGSGGGGS (SEQ ID NO:538) and has a length of 15 amino acids.

An anti-mesothelin antibody suitable for inclusion in a T-Cell-MP as a targeting sequence is a scFv that may comprise in order from N-terminus to C-terminus: a) a VH region comprising an amino acid sequence having at least 90%, at least 95%, at least 98%, at least 99%, or 100%, amino acid sequence identity to the following amino acid sequence: QVELVQSGAEVKKPGESLKISCKGSGYSFTSYWIGWVRQAPGKGLEWMGIIDPGDSRTRYSPSFQ GQVTISADKSISTAYLQWSSLKASDTAMYYCARGQLYGGTYMDGWGQGTLVTVSS (SEQ ID NO:542); b) a peptide linker; and c) a VL region comprising an amino acid sequence having at least 90%, at least 95%, at least 98%, at least 99%, or 100%, amino acid sequence identity to the following amino acid sequence: DIALTQPASVSGSPGQSITISCTGTSSDIGGYNSVSWYQQHPGKAPKLMIYGVNNR PSGVSNRFSGSKSGNTASLTISGLQAEDEADYYCSSYDIESATPVFGGGTKLTVLG (SEQ ID NO:541). In some cases, the peptide linker comprises the amino acid sequence (GGGGS)n, where n is an integer from 1 to 10 (e.g., where n is 1, 2, 3, 4, 5, 6, 7, 8, 9, or 10). In some cases, the peptide linker comprises the amino acid sequence GGGGSGGGGSGGGGS (SEQ ID NO:538) and has a length of 15 amino acids.

In some cases, an anti-mesothelin antibody suitable for inclusion in a T-Cell-MP comprises: a) VL CDR1, VL CDR2, and VL CDR3 present in a light chain variable region (VL) comprising the following amino acid sequence: DIQMTQSPSSLSASVGDRVTITCSASSSVSYMHWYQQKSGKAPKLLIYDTSKLASGVPSRFSGSGS GTDFTLTISSLQPEDFATYYCQQWSKHPLTFGQGTKLEIK (SEQ ID NO:543); and b) VH CDR1, CDR2, and CDR3 present in a heavy chain variable region (VH) comprising the following amino acid sequence: QVQLVQSGAEVKKPGASVKVSCKASGYSFTGYTMNWVRQAPGQGLEWMGLITPYN GASSYNQKFRGKATMTVDTSTSTVYMELSSLRSEDTAVYYCARGGYDGRGFDYWGQGTLVTV SS (SEQ ID NO:544). In some cases, the VH and VL CDRs are as defined by Kabat (see, e.g., the CDR Table above; and Kabat 1991). In some cases, the VH and VL CDRs are as defined by Chothia (see, e.g., the CDR Table above; and Chothia 1987).

In some cases, an anti-mesothelin antibody suitable for inclusion in a T-Cell-MP comprises: a) a VL region comprising an amino acid sequence having at least 90%, at least 95%, at least 98%, at least 99%, or 100%, amino acid sequence identity to the following amino acid sequence: DIQMTQSPSSLSA SVGDRVTITCSASSSVSYMHWYQQKSGKAPKLLIYDTSKLASGVPSRFSGSGSGTDFTLT ISSLQPEDFATYYCQQWSKHPLTFGQGTKLEIK (SEQ ID NO:543); and b) a VH region comprising an amino acid sequence having at least 90%, at least 95%, at least 98%, at least 99%, or 100%, amino acid sequence identity to the following amino acid sequence: QVQLVQSGAEVKKPGA SVKVSCKASGYSFTGYTMNWVRQAPGQGLEWMGLITPYNGASSYNQKFRGKATMTV DTSTSTVYMELSSLRSEDTAVYYCARGGYDGRGFDYWGQGTLVTVSS (SEQ ID NO:544).

In some cases, an anti-mesothelin antibody suitable for inclusion in a T-Cell-MP is a scFv comprising, in order from N-terminus to C-terminus: a) a VL region comprising an amino acid sequence having at least 90%, at least 95%, at least 98%, at least 99%, or 100%, amino acid sequence identity to the following amino acid sequence: DIQMTQSPSSLSASVGDRVTITCSASSSVSYMHWYQQKSGKAPKLLIYDTSKLASGVPSR FSGSGSGTDFTLTISSLQPEDFATYYCQQWSKHPLTFGQGTKLEIK (SEQ ID NO:543); b) a peptide linker; and c) a VH region comprising an amino acid sequence having at least 90%, at least 95%, at least 98%, at least 99%, or 100%, amino acid sequence identity to the following amino acid sequence: QVQLVQSGAEVKKPGASVKVSCKASGYSFTGYTMNWVRQAPGQGLEWMGLITPYNG ASSYNQKFRGKATMTVDTSTSTVYMELSSLRSEDTAVYYCARGGYDGRGFDYWGQGT LVTVSS (SEQ ID NO:544). In some cases, the peptide linker comprises the amino acid sequence (GGGGS)n, where n is an integer from 1 to 10 (e.g., where n is 1, 2, 3, 4, 5, 6, 7, 8, 9, or 10). In some cases, the peptide linker comprises the amino acid sequence GGGGSGGGGSGGGGS (SEQ ID NO:538) and has a length of 15 amino acids.

In some cases, an anti-mesothelin antibody suitable for inclusion in a T-Cell-MP is a scFv comprising, in order from N-terminus to C-terminus: a) a VH region comprising an amino acid sequence having at least 90%, at least 95%, at least 98%, at least 99%, or 100%, amino acid sequence identity to the following amino acid sequence: QVQLVQSGAEVKKPGASVKVSCKASGYSFTGYTMNWVRQA PGQGLEWMGLITPYNGASSYNQKFRGKATMTVDTSTSTVYMELSSLRSEDTAVYYCARGGYDG RGFDYWGQGTLVTVSS (SEQ ID NO:544); b) a peptide linker; and c) a VL region comprising an amino acid sequence having at least 90%, at least 95%, at least 98%, at least 99%, or 100%, amino acid sequence identity to the following amino acid sequence: DIQMTQSPSSLSASVGDRVTITCSASSSV SYMHWYQQKSGKAPKLLIYDTSKLASGVPSRFSGSGSGTDFTLTISSLQPEDFATYYCQQWSKHP LTFGQGTKLEIK (SEQ ID NO:543). In some cases, the peptide linker comprises the amino acid sequence (GGGGS)n, where n is an integer from 1 to 10 (e.g., where n is 1, 2, 3, 4, 5, 6, 7, 8, 9, or 10). In some cases, the peptide linker comprises the amino acid sequence GGGGSGGGGSGGGGS (SEQ ID NO:538) and has a length of 15 amino acids.

(d) Anti-TROP-2

Trophoblast cell surface antigen 2 (Trop-2) (also known as epithelial glycoprotein-1, gastrointestinal tumor-associated antigen GA733-1, membrane component chromosome 1 surface marker-1, and tumor-associated calcium signal transducer-2) is a transmembrane glycoprotein that is upregulated in numerous cancer types, and is the protein product of the TACSTD2 gene.

In some cases, the CTP of a T-Cell-MP is an anti-TROP-2 scFv or an anti-TROP-2 nanobody comprising VH and VL CDRs present in any one of the amino acid sequences set forth in FIG. 23A-23D. In some cases, the TTP is an anti-TROP-2 scFv comprising an amino acid sequence as set forth in any one of FIG. 23A-23D.

Anti-TROP-2 antibodies are known in the art; and the VH and VL, or the VH and VL CDRs, of any anti-TROP-2 antibody can be used in a T-Cell-MP of the present disclosure as a tarting sequence. See, e.g., U.S. Pat. No. 7,238,785). In some cases, an anti-TROP-2 antibody comprises: i) light chain CDR sequences CDR1 (KASQDVSIAVA; SEQ ID NO:545); CDR2 (SASYRYT; SEQ ID NO:546); and CDR3 (QQHYITPLT; SEQ ID NO:547); and ii) heavy chain CDR sequences CDR1 (NYGMN; SEQ ID NO:548); CDR2 (WINTYTGEPTYTDDFKG; SEQ ID NO:549); and CDR3 (GGFGSSYWYFDV; SEQ ID NO:550).

In some cases, an anti-TROP-2 antibody comprises: i) heavy chain CDR sequences CDR1 (TAGMQ; SEQ ID NO:551); CDR2 (WINTHSGVPKYAEDFKG (SEQ ID NO:552); and CDR3 (SGFGSSYWYFDV; SEQ ID NO:553); and ii) light chain CDR sequences CDR1 (KASQDVSTAVA; SEQ ID NO:554); CDR2 (SASYRYT; SEQ ID NO:546); and CDR3 (QQHYITPLT; SEQ ID NO:547).

In some cases, an anti-TROP2 antibody suitable for inclusion in a T-Cell-MP comprises: a) VL CDR1, VL CDR2, and VL CDR3 present in a light chain variable region (VL) comprising the following amino acid sequence: DIQLTQSPSSLSASVGDRVSITCKASQDVSIAVAWYQQKPGKAPKLLIYSASYRYTGVPDRFSGSG SGTDFTLTISSLQPEDFAVYYCQQHYITPLTFGAGTKVEIK (SEQ ID NO:555); and b) VH CDR1, CDR2, and CDR3 present in a heavy chain variable region (VH) comprising the following amino acid sequence: QVQLQQSGSELKKPGASVKVSCKASGYTFTNYGMNWVKQAPGQGLKWMGWINTY TGEPTYTDDFKGRFAFSLDTSVSTAYLQISSLKADDTAVYFCARGGFGSSYWYFDVWGQGSLVT VSS (SEQ ID NO:556). In some cases, the V_{H} and V_{L} CDRs are as defined by Kabat (see, e.g., the CDR Table, above; and Kabat 1991). In some cases, the V_{H} and V_{L} CDRs are as defined by Chothia (see, e.g., the CDR Table above; and Chothia 1987).

In some cases, an anti-TROP-2 antibody suitable for inclusion in a T-Cell-MP comprises: a) a VL region comprising an amino acid sequence having at least 90%, at least 95%, at least 98%, at least 99%, or 100%, amino acid sequence identity to the following amino acid sequence: DIQLTQSPSSLSASVGD RVSITCKASQDVSIAVAWYQQKPGKAPKLLIYSASYRYTGVPDRFSGSGSGTDFTLTISSLQPEDF AVYYCQQHYITPLTFGAGTKVEIK (SEQ ID NO:555); and b) a VH region comprising an amino acid sequence having at least 90%, at least 95%, at least 98%, at least 99%, or 100%, amino acid sequence identity to the following amino acid sequence: QVQLQQSGSELKKPGASVKVSCKASGYTFTNYGM NWVKQAPGQGLKWMGWINTYTGEPTYTDDFKGRFAFSLDTSVSTAYLQISSLKADDTAVYFCA RGGFGSSYWYFDVWGQGSLVTVSS (SEQ ID NO:556).

In some cases, an anti-TROP-2 antibody suitable for inclusion in a T-Cell-MP is a scFv comprising, in order from N-terminus to C-terminus: a) a VL region comprising an amino acid sequence having at least 90%, at least 95%, at least 98%, at least 99%, or 100%, amino acid sequence identity to the following amino acid sequence: DIQLTQSPSSLSASVGDRVSITCKASQDVSIAVAWYQQKPGK APKLLIYSASYRYTGVPDRFSGSGSGTDFTLTISSLQPEDFAVYYCQQHYITPLTFGAGTKVEIK (SEQ ID NO:555); b) a peptide linker; and c) a VH region comprising an amino acid sequence having at least 90%, at least 95%, at least 98%, at least 99%, or 100%, amino acid sequence identity to the following amino acid sequence: QVQLQQSGSELKKPGASVKVSCKASGYTFTNYGMNWVKQAPGQ GLKWMGWINTYTGEPTYTDDFKGRFAFSLDTSVSTAYLQISSLKADDTAVYFCARGGFGSSYW YFDVWGQGSLVTVSS (SEQ ID NO:556). In some cases, the peptide linker comprises the amino acid sequence (GGGGS)n, where n is an integer from 1 to 10 (e.g., where n is 1, 2, 3, 4, 5, 6, 7, 8, 9, or 10). In some cases, the peptide linker comprises the amino acid sequence GGGGSGGGGSGGGGS (SEQ ID NO:538) and has a length of 15 amino acids.

In some cases, an anti-TROP-2 antibody suitable for inclusion in a T-Cell-MP is a scFv comprising, in order from N-terminus to C-terminus: a) a VH region comprising an amino acid sequence having at least 90%, at least 95%, at least 98%, at least 99%, or 100%, amino acid sequence identity to the following amino acid sequence: QVQLQQSGSELKKPGASVKVSCKASGYTFTNYGMNWVKQ APGQGLKWMGWINTYTGEPTYTDDFKGRFAFSLDTSVSTAYLQISSLKADDTAVYFCARGGFGS SYWYFDVWGQGSLVTVSS (SEQ ID NO:556); b) a peptide linker; and c) a VL region comprising an amino acid sequence having at least 90%, at least 95%, at least 98%, at least 99%, or 100%, amino acid sequence identity to the following amino acid sequence: DIQLTQSPSSLSASVGDRVSITCKASQDVSIAVAWYQQKPGKAPKLLIYSASYRYTGVPDRFSGSG SGTDFTLTISSLQPEDFAVYYCQQHYITPLTFGAGTKVEIK (SEQ ID NO:555). In some cases, the peptide linker comprises the amino acid sequence (GGGGS)n, where n is an integer from 1 to 10 (e.g., where n is 1, 2, 3, 4, 5, 6, 7, 8, 9, or 10). In some cases, the peptide linker comprises the amino acid sequence GGGGSGGGGSGGGGS (SEQ ID NO:538) and has a length of 15 amino acids.

In some cases, an anti-TROP2 antibody suitable for inclusion in T-Cell-MP comprises: a) VL CDR1, VL CDR2, and VL CDR3 present in a light chain variable region (VL) comprising the following amino acid sequence: DIQMTQSPSSLSASVGDRVTITCKASQDVSTAVAWYQQKPGKAPKLLIYSASYRYTGVPSRFSGS GSGTDFTLTISSLQPEDFAVYYCQQHYITPLTFGQGTKLEIK (SEQ ID NO:557); and b) VH CDR1, CDR2, and CDR3 present in a heavy chain variable region (VH) comprising the following amino acid sequence: QVQLVQSGAEVKKPGASVKVSCKASGYTFTTAGMQWVRQAPGQGLEWMGWINTHSGVPKYA EDFKGRVTISADTSTSTAYLQLSSLKSEDTAVYYCARSGFGSSYWYFDVWGQGTLVTVSS (SEQ ID NO:558). In some cases, the V_{H} and V_{L} CDRs are as defined by Kabat (see, e.g., the CDR Table, above; and Kabat 1991). In some cases, the V_{H} and V_{L} CDRs are as defined by Chothia (see, e.g., the CDR Table, above; and Chothia 1987).

In some cases, an anti-TROP-2 antibody suitable for inclusion in a T-Cell-MP comprises: a) a VL region comprising an amino acid sequence having at least 90%, at least 95%, at least 98%, at least 99%, or 100%, amino acid sequence identity to the following amino acid sequence: DIQMTQSPSSLSASVGDRVTITCKASQDVSTAVAWYQQKPGKAPKLLIYSASYRYTGVPSRFSGS GSGTDFTLTISSLQPEDFAVYYCQQHYITPLTFGQGTKLEIK (SEQ ID NO:557); and b) a VH region comprising an amino acid sequence having at least 90%, at least 95%, at least 98%, at least 99%, or 100%, amino acid sequence identity to the following amino acid sequence:

(SEQ ID NO: 558)
QVQLVQSGAEVKKPGASVKVSCKASGYTFTTAGMQWVRQAPGQGLEWMG
WINTHSGVPKYAEDFKGRVTISADTSTSTAYLQLSSLKSEDTAVYYCAR
SGFGSSYWYFDVWGQGTLVTVSS.

In some cases, an anti-TROP-2 antibody suitable for inclusion in a T-Cell-MP is a scFv comprising, in order from N-terminus to C-terminus: a) a VL region comprising an amino acid sequence having at least 90%, at least 95%, at least 98%, at least 99%, or 100%, amino acid sequence identity to the following amino acid sequence: DIQMTQSPSSLSASVGDRVTITCKASQDVSTAVAWYQQKPGKAPKLLIYSASYRYTGVPSRFSGS GSGTDFTLTISSLQPEDFAVYYCQQHYITPLTFGQGTKLEIK (SEQ ID NO:557); b) a peptide linker; and c) a VH region comprising an amino acid sequence having at least 90%, at least 95%, at least 98%, at least 99%, or 100%, amino acid sequence identity to the following amino acid sequence: QVQLVQSGAEVKKPGASVKVSCKASGYTFTTAGMQWVRQAPGQGLEWMGWINTHSGVPKYA EDFKGRVTISADTSTSTAYLQLSSLKSEDTAVYYCARSGFGSSYWYFDVWGQGTLVTVSS (SEQ ID NO:558). In some cases, the peptide linker comprises the amino acid sequence (GGGGS)n, where n is an integer from 1 to 10 (e.g., where n is 1, 2, 3, 4, 5, 6, 7, 8, 9, or 10). In some cases, the peptide linker comprises the amino acid sequence GGGGSGGGGSGGGGS (SEQ ID NO:538) and has a length of 15 amino acids.

In some cases, an anti-TROP-2 antibody suitable for inclusion in a T-Cell-MP is a scFv comprising, in order from N-terminus to C-terminus: a) a VH region comprising an amino acid sequence having at least 90%, at least 95%, at least 98%, at least 99%, or 100%, amino acid sequence identity to the following amino acid sequence: QVQLVQSGAEVKKPGASVKVSCKASGYTFTTAGMQWVRQAPGQGLEWMGWINTHSGVPKYA EDFKGRVTISADTSTSTAYLQLSSLKSEDTAVYYCARSGFGSSYWYFDVWGQGTLVTVSS (SEQ ID NO:558); b) a peptide linker; and c) a VL region comprising an amino acid sequence having at least 90%, at least 95%, at least 98%, at least 99%, or 100%, amino acid sequence identity to the following amino acid sequence: DIQMTQSPSSLSASVGDRVTITCKASQDVSTAVAWYQQKPGKAPKLLIYSASYRYTGVPSRFSGS GSGTDFTLTISSLQPEDFAVYYCQQHYITPLTFGQGTKLEIK (SEQ ID NO:557). In some cases, the peptide linker comprises the amino acid sequence (GGGGS)n, where n is an integer from 1 to 10 (e.g., where n is 1, 2, 3, 4, 5, 6, 7, 8, 9, or 10). In some cases, the peptide linker comprises the amino acid sequence GGGGSGGGGSGGGGS (SEQ ID NO:538) and has a length of 15 amino acids.

(e) Anti-BCMA

Anti-BCMA (B-cell maturation antigen) antibodies (or antigen binding fragments thereof) are known in the art; and the VH and VL, or the VH and VL CDRs, of any anti-BCMA antibody can be used in targeting a T-Cell-MP. See, e.g., WO 2014/089335; and US 2019/0153061.

An anti-BCMA antibody (or antigen binding fragments thereof) may comprise: a) a light chain comprising an aa sequence having at least 90%, at least 95%, at least 98%, at least 99%, or 100%, aa sequence identity to the following aa sequence: QSVLTQPPSA SGTPGQRVTI SCSGSSSNIGSNTVNWYQQL PGTAPKLLIF NYHQRPSGVP DRFSGSKSGS SASLAISGLQ SEDEADYYCA AWDDSLNGWV FGGGTKLTVL GQPKAAPSVT LFPPSSEELQ ANKATLVCLI SDFYPGAVTV AWKADSSPVK AGVETTTPDS KQSNNKYAAS SYLSLTPEQW KSHRSYSCQV THEGSTVEKT VAPTECS (SEQ ID NO:216); and b) a heavy chain comprising an aa sequence having at least 90%, at least 95%, at least 98%, at least 99%, or 100%, aa sequence identity to the following aa sequence: EVQLVESGGG LVKPGGSLRL SCAASGFTFG DYALSWFRQ APGKGLEWVG VSRSKAYGG TTDYAASVKG RFTISRDDS KSTAYLQMNS LKTEDTAVY YCASSGYSSG WTPFDYWGQG TLVTVSSAST KGPSVFPLAP SSKSTSGGTA ALGCLVKDYF PEPVTVSWNS GALTSGVHTF PAVLQSSGLY SLSSVVTVPS SSLGTQTYIC NVNHKPSNTK VDKKVEPKSC DKTHTCPPCP APELLGGPSV FLFPPKPKDT LMISRTPEVT CVVVDVSHED PEVKFNWYVD GVEVHNAKTK PREEQYNSTY RVVSVLTVLH QDWLNGKEYK CKVSNKALPA PIEKTISKAK GQPREPQVYT LPPSREEMTK NQVSLTCLVK GFYPSDIAVE WESNGQPENN YKTTPPVLDS DGSFFLYSKL TVDKSRWQQG NVFSCSVMHE ALHNHYTQKS LSLSPGK (SEQ ID NO:217).

An anti-BCMA (or antigen binding fragments thereof) may comprise a VL present in the light chain aa sequence provided above; and a VH present in the heavy chain aa sequence provided above. For example, an anti-BCMA antibody can comprise: a) a VL comprising an aa sequence having at least 90%, at least 95%, at least 98%, at least 99%, or 100%, aa sequence identity to the aa sequence: QSVLTQPPSA SGTPGQRVTI SCSGSSSNIG SNTVNWYQQL PGTAPKLLIF NYHQRPSGVP DRFSGSKSGS SASLAISGLQ SEDEADYYCA AWDDSLNGWV FGGGTKLTVL G (SEQ ID NO:218); and b) a VH comprising an aa sequence having at least 90%, at least 95%, at least 98%, at least 99%, or 100%, aa sequence identity to the aa sequence: EVQLVESGGG LVKPGGSLR LSCAASGFTF GDYALSWFRQ APGKGLEWVG VSRSKAYGGT TDYAASVKGR FTISRDDSKST AYLQMNSLKT EDTAVYYCAS SGYSSGWTPF DYWGQGTLVT VSSASTKGPSV (SEQ ID NO:219).

In some cases, an anti-BCMA antibody (or antigen binding fragments thereof) comprises VL CDR1, VL CDR2, and VL CDR3 present in the light chain aa sequence provided above; and VH CDR1, CDR2, and CDR3 present in the heavy chain aa sequence provided above. In some cases, the VH and VL CDRs are as defined by Kabat (see, e.g., Kabat 1991). In some cases, the VH and VL CDRs are as defined by Chothia (see, e.g., Chothia 1987).

For example, an anti-BCMA antibody (or antigen binding fragments thereof) can comprise a VL CDR1 having the aa sequence SSNIGSNT (SEQ ID NO:220), a VL CDR2 having the aa sequence NYH, a VL CDR3 having the aa sequence AAWDDSLNGWV (SEQ ID NO:221)), a VH CDR1 having the aa sequence GFTFGDYA (SEQ ID NO:222), a VH CDR2 having the aa sequence SRSKAYGGTT (SEQ ID NO:223), and a VH CDR3 having the aa sequence ASSGYSSGWTPFDY (SEQ ID NO:224).

An anti-BCMA antibody can be a scFv. As one non-limiting example, an anti-BCMA scFv can comprise the following aa sequence: QVQLVQSGAE VKKPGSSVKV SCKASGGTFS NYWMHWVRQA PGQGLEWMGA TYRGHSDTYY NQKFKGRVTI TADKSTSTAY MELSSLRSED TAVYYCARGA IYNGYDVLDN WGQGTLVTVS SGGGGSGGGG SDIQMTQSPS SLSASVGDRVT ITCSASQDIS NYLNWYQQKP GKAPKLLIYY TSNLHSGVPS RFSGSGSGT DFTLTISSLQP EDFATYYCQQ YRKLPWTFGQG TKLEIKR (SEQ ID NO:225), or the sequence:

(SEQ ID NO: 564)
QVQLVQSGAEVKKPGSSVKVSCKASGGTFSNYWMHWVRQAPGQGLEWMGAT
YRGHSDTYYNQKFKGRVTITADKSTSTAYMELSSLRSEDTAVYYCARGAIY
NGYDVLDNWGQGTLVTVSSGGGGSGGGGSGGGGSGGGGSDIQMTQSPSSLS
ASVGDRVTITCSASQDISNYLNWYQQKPGKAPKLLIYYTSNLHSGVPSRFS
GSGSGTDFTLTISSLQPEDFATYYCQQYRKLPWTFGQGTKLEIKR.

As another example, an anti-BCMA scFv can comprise the following aa sequence:

(SEQ ID NO: 226)
DIQMTQSPSS LSASVGDRVT ITCSASQDIS NYLNWYQQKP
GKAPKLLIYY TSNLHSGVPS RFSGSGSGTD FTLTISSLQP
EDFATYYCQQ YRKLPWTFGQ GTKLEIKRGG GGSGGGGSGG
GGSGGGGSQV QLVQSGAEVK KPGSSVKVSC KASGGTFSNY
WMHWVRQAPG QGLEWMGA TYRGHSDTYY NQKFKGRVTI
TADKSTSTAY MELSSLRSED TAVYYCARGA IYNGYDVLDN
WGQGTLVTVS S.

In some cases, an anti-BCMA antibody can comprise a VL CDR1 having the amino acid sequence SASQDISNYLN (SEQ ID NO:565); a VL CDR2 having the amino acid sequence YTSNLHS (SEQ ID NO:566); a VL CDR3 having the amino acid sequence QQYRKLPWT (SEQ ID NO:567); a VH CDR1 having the amino acid sequence NYWMH (SEQ ID NO:568); a VH CDR2 having the amino acid sequence ATYRGHSDTYYNQKFKG (SEQ ID NO:569); and a VH CDR3 having the amino acid sequence GAIYNGYDVLDN (SEQ ID NO:570).

In some cases, an anti-BCMA antibody comprises: a) a light chain comprising an amino acid sequence having at least 90%, at least 95%, at least 98%, at least 99%, or 100%, amino acid sequence identity to the following amino acid sequence:

(SEQ ID NO: 571)
DIQMTQSPSSLSASVGDRVTITCSASQDISNYLNWYQQKPGKAPKLLIYYT
SNLHSGVPSRFSGSGSGTDFTLTISSLQPEDFATYYCQQYRKLPWTFGQGT
KLEIKR.

In some cases, an anti-BCMA antibody comprises: a) a heavy chain comprising an amino acid sequence having at least 90%, at least 95%, at least 98%, at least 99%, or 100%, amino acid sequence identity to the following amino acid sequence:

(SEQ ID NO: 572)
QVQLVQSGAEVKKPGSSVKVSCKASGGTFSNYWMHWVRQAPGQGLEWMGAT
YRGHSDTYYNQKFKGRVTITADKSTSTAYMELSSLRSEDTAVYYCARGAIY
DGYDVLDNWGQGTLVTVSS.

In some cases, an anti-BCMA antibody (e.g., an antibody referred to in the literature as belantamab) comprises a light chain comprising the amino acid sequence: DIQMTQSPSSLSASVGDRVTITCSASQDISNYLNWYQQKPGKAPKLLIYYTSNLHSGVPSRFSGSG SGTDFTLTISSLQPEDFATYYCQQYRKLPWTFGQGTKLEIKR (SEQ ID NO:571); and a heavy chain comprising the amino acid sequence:

(SEQ ID NO: 572)
QVQLVQSGAEVKKPGSSVKVSCKASGGTFSNYWMHWVRQAPGQGLEWMGAT
YRGHSDTYYNQKFKGRVTITADKSTSTAYMELSSLRSEDTAVYYCARGAIY
DGYDVLDNWGQGTLVTVSS.

In some cases, the anti-BCMA antibody has a cancer chemotherapeutic agent linked to the antibody. For example, in some cases, the anti-BCMA antibody is GSK2857916 (belantamab-mafodotin), where monomethyl auristatin F (MMAF) is linked via a maleimidocaproyl linker to the anti-BCMA antibody belantamab.

(f) Anti-MUC1

In some cases, a targeting sequence present in a T-Cell-MP of the present disclosure is an antibody specific for MUC1. For example, a targeting sequence can be specific for a MUC1 polypeptide present on a cancer cell. In some cases, the targeting sequence is specific for the cleaved form of MUC1; see, e.g., Fessler et al. (2009) Breast Cancer Res. Treat. 118:113. In some cases, the targeting sequence is an antibody specific for a glycosylated MUC1 peptide; see, e.g., Naito et al. (2017) ACS Omega 2:7493; and U.S. Pat. No. 10,017,580.

As one non-limiting example, a targeting sequence can be a single-chain Fv specific for MUC1. See, e.g., Singh et al. (2007) Mol. Cancer Ther. 6:562; Thie et al. (2011) PLoSOne 6:e15921; Imai et al. (2004) Leukemia 18:676; Posey et al. (2016) Immunity 44:1444; EP3130607; EP3164418; WO 2002/044217; and US 2018/0112007. In some cases, a targeting sequence is a scFv specific for the MUC1 peptide VTSAPDTRPAPGSTAPPAHG (SEQ ID NO:227). In some cases, a targeting sequence is a scFv specific for the MUC1 peptide: SNIKFRPGSVVVQLTLAFREGTINVHDVETQFNQYKTEAASRY (SEQ ID NO:228). In some cases, a targeting sequence is a scFv specific for the MUC1 peptide SVVVQLTLAFREGTINVHDVETQFNQ YKTEAASRY (SEQ ID NO:229). In some cases, a targeting sequence is a scFv specific for the MUC1 peptide LAFREGTINVHDVETQFNQY (SEQ ID NO:230). In some cases, a targeting sequence is a scFv specific for the MUC1 peptide SNIKFRPGSVVVQLTLAAFREGTIN (SEQ ID NO:231).

As an example, an anti-MUC1 antibody can comprise: a VH CDR1 having the amino acid sequence RYGMS (SEQ ID NO:232); a VH CDR2 having the amino acid sequence TISGGGTYIYYPDSVKG (SEQ ID NO:233); a VH CDR3 having the amino acid sequence DNYGRNYDYGMDY (SEQ ID NO:234); a VL CDR1 having the amino acid sequence SATSSVSYIH (SEQ ID NO:235); a VL CDR2 having the amino acid sequence STSNLAS (SEQ ID NO:236); and a VL CDR3 having the amino acid sequence QQRSSSPFT (SEQ ID NO:237). See, e.g., US 2018/0112007.

As another example, an anti-MUC1 antibody can comprise a VH CDR1 having the amino acid sequence GYAMS (SEQ ID NO:238); a VH CDR2 having the amino acid sequence TISSGGTYIYYPD SVKG (SEQ ID NO:239); a VH CDR3 having the amino acid sequence LGGDNYYEYFDV (SEQ ID NO:240); a VL CDR1 having the amino acid sequence RASKSVSTSGYSYMH (SEQ ID NO:241); a VL CDR2 having the amino acid sequence LASNLES (SEQ ID NO:242); and a VL CDR3 having the amino acid sequence QHSRELPFT (SEQ ID NO:243). See, e.g., US 2018/0112007.

As another example, an anti-MUC1 antibody can comprise a VH CDR1 having the amino acid sequence DYAMN (SEQ ID NO:244); a VH CDR2 having the amino acid sequence VISTFSGNINFN QKFKG (SEQ ID NO:245); a VH CDR3 having the amino acid sequence SDYYGPYFDY (SEQ ID NO:246); a VL CDR1 having the amino acid sequence RSSQTIVHSNGNTYLE (SEQ ID NO:247); a VL CDR2 having the amino acid sequence KVSNRFS (SEQ ID NO:248); and a VL CDR3 having the amino acid sequence FQGSHVPFT (SEQ ID NO:249). See, e.g., US 2018/0112007.

As another example, an anti-MUC1 antibody can comprise a VH CDR1 having the amino acid sequence GYAMS (SEQ ID NO:238); a VH CDR2 having the amino acid sequence TISSGGTYIYYPDSVKG (SEQ ID NO:239); a VH CDR3 having the amino acid sequence LGGDNYYEY (SEQ ID NO:250); a VL CDR1 having the amino acid sequence TASKSVSTSGYSYMH (SEQ ID NO:251); a VL CDR2 having the amino acid sequence LVSNLES (SEQ ID NO:252); and a VL CDR3 having the amino acid sequence QHIRELTRSE (SEQ ID NO:253). See, e.g., US 2018/0112007.

(g) Anti-MUC16

In some cases, a targeting sequence present in a T-Cell-MP of the present disclosure is an antibody specific for MUC16 (also known as CA125). See, e.g., Yin et al. (2002) Int. J. Cancer 98:737. For example, a targeting sequence can be specific for a MUC16 polypeptide present on a cancer cell. See, e.g., US 2018/0118848; and US 2018/0112008. In some cases, a MUC16-specific targeting sequence is a scFv. In some cases, a MUC16-specific targeting sequence is a nanobody.

As one example, an anti-MUC16 antibody can comprise a VH CDR1 having the amino acid sequence GFTFSNYY (SEQ ID NO:254); a VH CDR2 having the amino acid sequence ISGRGSTI (SEQ ID NO:255); a VH CDR3 having the amino acid sequence VKDRGGYSPY (SEQ ID NO:256); a VL CDR1 having the amino acid sequence QSISTY (SEQ ID NO:257); a VL CDR2 having the amino acid sequence TAS; and a VL CDR3 having the amino acid sequence QQSYSTPPIT (SEQ ID NO:258). See, e.g., US 2018/0118848.

(h) Anti-Claudin-18.2

In some cases, a targeting sequence present in a T-Cell-MP of the present disclosure is an antibody specific for claudin-18 isoform 2 (“claudin-18.2”). See, e.g., WO 2013/167259. In some cases, a claudin-18.2-specific targeting sequence is a scFv. In some cases, a claudin-18.2-specific targeting sequence is a nanobody. In some cases, a CTP present in a T-Cell-MP of the present disclosure is an antibody specific for TEDEVQSYPSKHDYV (SEQ ID NO:170) or EVQSYPSKHDYV (SEQ ID NO:171).

As one example, an anti-claudin-18.2 antibody can comprise a VH CDR1 having the amino acid sequence GYTFTDYS (SEQ ID NO:259); a VH CDR2 having the amino acid sequence INTETGVP (SEQ ID NO:260); a VH CDR3 having the amino acid sequence ARRTGFDY (SEQ ID NO:261); a VL CDR1 having the amino acid sequence KNLLHSDGITY (SEQ ID NO:262); a VL CDR2 having the amino acid sequence RVS; and a VL CDR3 having the amino acid sequence VQVLELPFT (SEQ ID NO:263).

As another example, an anti-claudin-s antibody can comprise a VH CDR1 having the amino acid sequence GFTFSSYA (SEQ ID NO:264); a VH CDR2 having the amino acid sequence ISDGGSYS (SEQ ID NO:265); a VH CDR3 having the amino acid sequence ARDSYYDNSYVRDY (SEQ ID NO:266); a VL CDR1 having the amino acid sequence QDINTF (SEQ ID NO:267); a VL CDR2 having the amino acid sequence RTN; and a VL CDR3 having the amino acid sequence LQYDEFPLT (SEQ ID NO:268).

(iii) Single-Chain T Cell Receptors

In some cases, a CTP present in a T-Cell-MP is a scTCR. A CTP can be a scTCR specific for a peptide/HLA complex on the surface of a cancer cell, where the peptide can be a cancer-associated peptide (e.g., a peptide fragment of a cancer-associated antigen) Amino acid sequences of scTCRs specific for cancer-associated peptides bound to an HLA complex are known in the art. See, e.g., US 2019/0135914; US 2019/0062398; and US 2018/0371049.

A scTCR includes an alpha chain variable region (Vα) and a beta chain variable region (Vβ) covalently linked through a suitable peptide linker sequence. For example, the Vα can be covalently linked to the Vβ through a suitable peptide linker (L) sequence fused to the C-terminus of the Vα and the N-terminus of the Vβ. A scTCR can have the structure Vα-L-Vβ. A scTCR can have the structure Vβ-L-Vα. A scTCR can also comprise a constant domain (also referred to as constant region). In some cases, a scTCR comprises, in order from N-terminus to C-terminus: i) a TCR α chain variable domain polypeptide; ii) a peptide linker; iii) a TCR R chain variable domain polypeptide; and iv) a TCR R chain constant region extracellular domain polypeptide. In some cases, a scTCR comprises, in order from N-terminus to C-terminus: i) a TCR R chain variable domain polypeptide; ii) a peptide linker; iii) a TCR α chain variable domain polypeptide; and iv) a TCR α chain constant region extracellular domain polypeptide.

Amino acid sequences of scTCRs specific for peptide/HLA complexes, where the peptide is a cancer-associated peptide, are known in the art. See, e.g., US 2019/0135914; US 2019/0062398; US 2018/0371049; US 2019/0144563; and US 2019/0119350. For example, a scTCR can be specific for an NY-ESO epitope such as an SLLMWITQC (SEQ ID NO:178) peptide bound to an HLA complex comprising an HLA-A*0201 heavy chain and a β2M polypeptide. As an example, such a scTCR can comprise: i) a TCR α chain variable region comprising an aa sequence having at least 90%, at least 95%, at least 98%, at least 99%, or 100%, aa sequence identity to the aa sequence: MQEVTQIPAA LSVPEGENLV LNCSFTDSA IYNLQWFRQD PGKGLTSLLL IQSSQREQTS GRLNASLDKS SGRSTLYIAA SQPGDSATYL CAVRPTSGGS YIPTFGRGTS LIVHPY (SEQ ID NO:269), where aa 20 can be V or A; aa 51 can be Q, P, S, T, or M; aa 52 can be S, P, F, or G, aa 53 can be S, W, H, or T; aa 94 can be P, H, or A; aa 95 can be T, L, M, A, Q, Y, E, I, F, V, N, G, S, D, or R; aa 96 can be S, L, T, Y, I, Q, V, E, A, W, R, G, H, D, or K; aa 97 can be G, D, N, V, S, T, or A; aa 98 can be G, P, H, S, T, W, or A; aa 99 can be S, T, Y, D, H, V, N, E, G, Q, K, A, I, or R; aa 100 can be Y, F, M, or D; aa 101 can be I, P, T, or M; and aa 103 can be T or A; and ii) a TCR R chain variable region comprising an aa sequence having at least 90%, at least 95%, at least 98%, at least 99%, or 100%, aa sequence identity to the aa sequence: MGVTQTPKFQVLKT GQSMTLQCAQDMNHEYMSWYRQDPGMGLRLIHYSVGAGITDQGEVPNGYNVSRSTTEDFPLRL LSAAPSQTSVYFCASSYVGNTGELFFGEGSR LTVL (SEQ ID NO:270), where aa 18 can be M or V; aa 50 can be G, V, or I; aa 52 can be G or Q; aa 53 can be I, T, or M; aa 55 can be D or R; aa 56 can be Q or R; aa 70 can be T or I; aa 94 can be Y, N, or F; aa 95 can be V or L; and aa 97 can be N, G, or D. For example, in some cases, a scTCR can comprise: i) a TCR α chain variable region comprising the aa sequence: MQEVTQIPAA LSVPEGENL VLNCSFTDS AIYNLQWFRQ DPGKGLTSL LLIMSHQREQ TSGRLNASLD KSSGRSTLYI AASQPGDSAT YLCAVRPTSG GSYIPTFGRG TSLIVHPY (SEQ ID NO:271); and a TCR R chain variable region comprising the aa sequence:

(SEQ ID NO: 272)
MGVTQTPKFQVLKTGQSMTLQCAQDMNHEYMSWYRQDPGMGLRLIHYSVSA
GITDQGEVPNGYNVSRSTTEDFPLRLLSAAPSQTSVYFCASSYVGNTGELF
FGEGSR LTVL.

As another example, a scTCR can be specific for an HPV peptide epitope (e.g., an HPV peptide of the aa sequence YIIFVYIPL (HPV 16 E563-71; SEQ ID NO:273), KLPQLCTEL (HPV 16 E611-19; SEQ ID NO:274), TIHEIILECV (HPV 16 E6; SEQ ID NO:275), YMLDLQPET (HPV 16 E711-19; SEQ ID NO:276), TLGIVCPI (HPV 16 E786-93; SEQ ID NO:277), KCIDFYSRI (HPV 18 E667-75; SEQ ID NO:278), or FQQLFLNTL (HPV 18 E786-94; SEQ ID NO:279)) bound to an HLA complex comprising an HL heavy chain and a β2M polypeptide. As an example, such a scTCR can comprise: i) a TCR α chain variable region comprising an aa sequence having at least 90%, at least 95%, at least 98%, at least 99%, or 100%, aa sequence identity to the aa sequence: METLLGLLILQ LQLQWVSSKQ EVTQIPAALS VPEGENLVLN CSFTDSAIYN LQWFRQDPG KGLTSLLLIQ SSQREQTSGR LNASLDKSSG RSTLYIAASQ PGDSATYLCA VRETSGSRLT FGEGTQLTVN PD (SEQ ID NO:280); and ii) a TCR (3 chain variable region comprising an aa sequence having at least 90%, at least 95%, at least 98%, at least 99%, or 100%, aa sequence identity to the aa sequence: MGIRLLCRVA FCFLAVGLVD VKVTQSSRYL VKRTGEKVFL ECVQDMDHEN MFWYRQDPGL GLRLIYFSYD VKMKEKGDIP EGYSVSREKK ERFSLILESA STNQTSMYLC ASSFWGRSTD TQYFGPGTRL TVL (SEQ ID NO:281).

8 Epitopes and their Assessment

An unconjugated T-Cell-MP of the present disclosure may be conjugated at a chemical conjugation site to a variety of molecules that present an antigenic determinate to form a T-Cell-MP-epitope conjugate. The molecules presenting an epitope that may be conjugated to an unconjugated T-Cell-MP include those presenting non-peptide epitopes (e.g., carbohydrate epitopes), and peptide epitopes, phosphopeptide epitopes, glycosylated peptide (glycopeptide) epitopes, carbohydrate, and lipopeptide epitopes (e.g., peptides modified with fatty acids, isoprenoids, sterols, phospholipids, or glycosylphosphatidyl inositol); collectively referred to as an “epitope” or “epitopes”. The epitope presenting sequence of the peptide, phospho-peptide, lipopeptide, or glycopeptide) present in a T-Cell-MP-epitope conjugate can be a peptide of from 4 to 25 contiguous aas (e.g., 4 aa, 5 aa, 6 aa, 7 aa, 8 aa, 9 aa, 10 aa, 11 aa, 12 aa, 13 aa, 14 aa, 15 aa, 16 aa, 17 aa, 18 aa, 19 aa, 20 aa, 21 aa, 22 aa, 23 aa, 24 aa, or 25 aa, or from 7 aa to 25 aa, from 7 aa to 12 aa, from 7 aa to 25 aa, from 10 aa to 15 aa, from 15 aa to 20 aa, or from 20 aa to 25 aa).

Epitopes of a T-Cell-MP-epitope conjugate are not part of the T-Cell-MP as translated from mRNA, but, as indicated above, are added to a T-Cell-MP at a chemical conjugation site. Selection of candidate MHC allele and peptide (e.g., phosphopeptide, lipopeptides or glycopeptide) epitope combinations for effective presentation to a TCR by a T-Cell-MP-epitope conjugate can be accomplished using any of a number of well-known methods to determine if the free peptide has affinity for the specific HLA allele used to construct the T-Cell-MP in which it will be presented as part of the epitope conjugate.

It is possible to determine if the peptide in combination with the specific heavy chain allele and β2M can affect the T-Cell in the desired manner (e.g., induction of proliferation, anergy, or apoptosis). Applicable methods include binding assays and T cell activation assays including BLI assays utilized for assessing binding affinity of T-Cell-MPs with wt. and variant MODs discussed above. The epitope (e.g., peptide epitope) that will be used to prepare a T-Cell-MP-epitope conjugate of the present disclosure may bind to a T cell receptor (TCR) on a T cell with an affinity of at least 100 μM (e.g., at least 10 μM, at least 1 μM, at least 100 nM, at least 10 nM, or at least 1 nM). In some cases, the epitope binds to a TCR on a T cell with an affinity of from about 10 M to about 10⁻⁵ M, from about 10⁻⁵ M to about 10⁻⁶ M, from about 10⁻⁶ M to about 10 M, from about 10 M to about 10 M, or from about 10 M to about 10⁻⁹ M. Expressed another way, in some cases, the epitope present in a T-Cell-MP binds to a TCR on a T cell with an affinity of from about 1 nM to about to about 10 nM, from about 10 nM to about 100 nM, from about 0.1 μM to about 1 μM, from about 1 μM to about 10 μM, from about 10 μM to about 25 μM, from about 25 μM to about 50 μM, from about 50 μM to about 75 μM, or from about 75 μM to about 100 μM.

a. Cell-Based Binding Assays

As one example, cell-based peptide-induced stabilization assays can be used to determine if a candidate peptide binds an HLA class I allele intended for use in a T-Cell-MP-epitope conjugate. The binding assay can be used in the selection of peptides for incorporation into a T-Cell-MP-epitope conjugate using the intended allele. In this assay, a peptide of interest is allowed to bind to a TAP-deficient cell, i.e., a cell that has defective transporter associated with antigen processing (TAP) machinery, and consequently, few surface class I molecules. Such cells include, e.g., the human T2 cell line (T2 (174×CEM.T2; American Type Culture Collection (ATCC) No. CRL-1992)). Henderson et al. (1992) Science 255:1264. Without efficient TAP-mediated transport of cytosolic peptides into the endoplasmic reticulum, assembled class I complexes are structurally unstable, and retained only transiently at the cell surface. However, when T2 cells are incubated with an exogenous peptide capable of binding class I, surface peptide-HLA class I complexes are stabilized and can be detected by flow cytometry with, e.g., a pan anti-class I monoclonal antibody, or directly where the peptide is fluorescently labeled. The stabilization and resultant increased life-span of peptide-HLA complexes on the cell surface by the addition of a peptide validates their identity. Accordingly, binding of candidate peptides for presentation by various Class I HLA heavy chain alleles can be tested by genetically modifying the T2 or similar TAP deficient cells to express the HLA H allele of interest.

In a non-limiting example of use of a T2 assay to assess peptide binding to HLA A*0201, T2 cells are washed in cell culture medium, and suspended at 10⁶ cells/ml. Peptides of interest are prepared in cell culture medium and serially diluted providing concentrations of 200 μM, 100 μM, 20 μM and 2 μM. The cells are mixed 1:1 with each peptide dilution to give a final volume of 200 μL and final peptide concentrations of 100 μM, 50 μM, 10 μM and 1 μM. A HLA A*0201 binding peptide, GILGFVFTL (SEQ ID NO:282), and a non-HLA A*0201-restricted peptide, HPVGEADYF (HLA-B*3501; SEQ ID NO:283), are included as positive and negative controls, respectively. The cell/peptide mixtures are kept at 37° C. in 5% CO₂ for ten minutes; then incubated at room temperature overnight. Cells are then incubated for 2 hours at 37° C. and stained with a fluorescently-labeled anti-human HLA antibody. The cells are washed twice with phosphate-buffered saline and analyzed using flow cytometry. The average mean fluorescence intensity (MFI) of the anti-HLA antibody staining is used to measure the strength of binding.

Labeled (e.g., a radio or fluorescently labeled payload) T-Cell-MP-epitope conjugates including MOD-less T-Cell-MP-epitope conjugates, particularly in the form higher order complexes (e.g., duplexes, tetramers or pentamers) may be used in vitro to establish epitope specific binding between a T-Cell-MP-epitope conjugate and a T cell. T cell binding by T-MP-epitope conjugates and/or MOD-less T-Cell-epitope conjugates is not, however, limited to in vitro applications. Binding, particularly by higher order complexes of T-Cell-MP-epitope conjugates may be conducted in vivo or ex vivo to, for example, track epitope specific T cell movement and localization. The use of MOD-less molecules is advantageous as it limits the potential interference due to interactions between a MOD on a T-Cell-MP-epitope conjugate and Co-MOD on cells that are not of interest. In such in vivo or ex vivo binding assessments a labeled (e.g., fluorescent or radio labeled) T-Cell-MP-epitope conjugate, which may be MOD-less, is administered to a subject in vivo, or contacted with a tissue ex vivo. Once the T-Cell-MP-epitope conjugate binds a T-cell in the subject or tissue it will effectively label the T cell which may circulate or be localized as evidenced by the localization of the label. Accordingly, such labeled T-Cell-MP-epitope conjugates, including their MOD-less variants, find use both in research and as companion diagnostics. The label permits evaluation of epitope specific binding between the T-Cell-MP-epitope conjugate and target T cells and tracking of epitope specific T cells to determine of their fate. The label also permits a determination of the localization of the T-Cell-MP-epitope conjugate in vivo and/or ex vivo, which may be used to determine if a T-Cell-MP-epitope conjugate is localized to a tissue, including tissues to which a medical treatment is desired (e.g., tumor tissue).

b. Biochemical Binding Assays

MHC Class I complexes comprising a β2M polypeptide complexed with an HLA heavy chain polypeptide of a specific allele intended for use in construction of a T-Cell-MP can be tested for binding to a peptide of interest in a cell-free in vitro assay system. For example, a labeled reference peptide (e.g., fluorescently labeled) is allowed to bind the MHC-class I complex to form a MHC-reference peptide complex. The ability of a test peptide of interest to displace the labeled reference peptide from the complex is tested. The relative binding affinity is calculated as the amount of test peptide needed to displace the bound reference peptide. See, e.g., van der Burg et al. (1995) Human Immunol. 44:189.

As another example, a peptide of interest can be incubated with a MHC Class I complex (containing an HLA heavy chain peptide and β2M) and the stabilization of the MHC complex by bound peptide can be measured in an immunoassay format. The ability of a peptide of interest to stabilize the MHC complex is compared to that of a control peptide presenting a known T cell epitope. Detection of stabilization is based on the presence or absence of the native conformation of the MHC complex bound to the peptide using an anti-HLA antibody. See, e.g., Westrop et al. (2009) J. Immunol. Methods 341:76; Steinitz et al. (2012) Blood 119:4073; and U.S. Pat. No. 9,205,144.

c. T Cell Activation Assays

Whether a given peptide binds a MHC Class I complex (comprising an HLA heavy chain and a β2M polypeptide), and, when bound to the HLA complex, can effectively present an epitope to a TCR, can be determined by assessing T cell response to the peptide-HLA complex. T cell responses that can be measured include, e.g., interferon-gamma (IFNγ) production, cytotoxic activity, and the like.

(i) ELISPOT Assays

Suitable T cell activation assays include, e.g., an enzyme linked immunospot (ELISPOT) assay where production of a product by target cells (e.g., IFNγ production by target CD8+T) is measured following contact of the target with an antigen-presenting cell (APC) that presents a peptide of interest complexed with a class I MHC (e.g., HLA). Antibody to the target cell produced factor (e.g., IFNγ) is immobilized on wells of a multi-well plate. APCs are added to the wells, and the plates are incubated for a period of time with a peptide of interest, such that the peptide binds HLA class I on the surface of the APCs. CD8+ T cells specific for the peptide are added to the wells, and the plate is incubated for about 24 hours. The wells are then washed, and any released factor (e.g., IFNγ) bound to the immobilized antibody is detected using a detectably labeled antibody. A colorimetric assay can be used. For example, where IFNγ release is measured, a detectably labeled anti-IFNγ antibody can be a biotin-labeled anti-IFNγ antibody, which can be detected using, e.g., streptavidin conjugated to alkaline phosphatase, with a BCIP/NBT (5-bromo-4-chloro-3-indolyl phosphate/nitro blue tetrazolium) solution added, to develop the assay. The presence of IFNγ-secreting T cells is identified by colored spots. Negative controls include APCs not contacted with the peptide. APCs expressing various HLA heavy chain alleles can be used to determine whether a peptide of interest effectively binds to a HLA class I molecule comprising a particular HLA H chain.

(ii) Cytotoxicity Assays

Whether a given epitope (e.g., peptide) binds to a particular MHC class I heavy chain allele complexed with β2M, and, when bound, can effectively present an epitope to a TCR, can also be determined using a cytotoxicity assay. A cytotoxicity assay involves incubation of a target cell with a cytotoxic CD8⁺ T cell. The target cell displays on its surface a MHC class I complex comprising β2M, and the epitope and MHC heavy chain allele combination to be tested. The target cells can be radioactively labeled, e.g., with ⁵¹Cr. If the target cell effectively presents the epitope to a TCR on the cytotoxic CD8⁺ T cell, it induces cytotoxic activity by the CD8⁺ T cell toward the target cell, which is determined by measuring release of ⁵¹Cr from the lysed target cell. Specific cytotoxicity can be calculated as the amount of cytotoxic activity in the presence of the peptide minus the amount of cytotoxic activity in the absence of the peptide.

(iii) Detection of Antigen-Specific T Cells with Peptide-HLA Tetramers

As another example, multimers (e.g., dimers, tetramers, or pentamers) of peptide-MHC complexes are generated with a label or tag (e.g., fluorescent or heavy metal tags). The multimers can then be used to identify and quantify specific T cells via flow cytometry (FACS) or mass cytometry (CyTOF). Detection of epitope-specific T cells provides direct evidence that the peptide-bound HLA molecule is capable of binding to a specific TCR on a subset of antigen-specific T cells. See, e.g., Klenerman et al. (2002) Nature Reviews Immunol. 2:263.

d. Epitopes

An epitope present in a T-Cell-MP-epitope conjugate may be bound in an epitope-specific manner by a T cell (i.e., the epitope is specifically bound by an epitope-specific T cell whose TCR recognizes the peptide). An epitope-specific T cell binds an epitope having a reference aa sequence in the context of a specific MHC-H allele polypeptide/β2M complex, but does not substantially bind an epitope that differs from the reference aa sequence presented in the same context. For example, an epitope-specific T cell may bind an epitope in the context of a specific MHC-H allele polypeptide/β2M complex having a reference aa sequence, and may bind an epitope that differs from the reference aa sequence presented in the same context, if at all, with an affinity that is less than 10⁻⁻⁶ M, less than 10⁻⁵ M, or less than 10⁻⁴ M. An epitope-specific T cell may bind an epitope (e.g., a peptide presenting an epitope of interest) for which it is specific with an affinity of at least 10⁻⁷ M, at least 10⁻⁸ M, at least 10⁻⁹ M, or at least 10⁻¹⁰ M.

In some cases, the peptide epitope present in a T-Cell-MP-epitope conjugate presents an epitope-specific to an HLA-A, -B, -C, -E, —F or -G allele. In an embodiment, the peptide epitope present in a T-Cell-MP presents an epitope restricted to HLA-A*0101, A*0201, A*0301, A*1101, A*2301, A*2402, A*2407, A*3303, and/or A*3401. In an embodiment, the peptide epitope present in a T-Cell-MP presents an epitope restricted to HLA-B*0702, B*0801, B*1502, B*3802, B*4001, B*4601, and/or B*5301. In an embodiment, the peptide epitope present in a T-Cell-MP presents an epitope restricted to C*0102, C*0303, C*0304, C*0401, C*0602, C*0701, C*702, C*0801, and/or C*1502.

Among the epitopes that may be bound and presented to a TCR by a T-Cell-MP with a class I MHC-H and a β2M polypeptide sequence are cancer antigens, and antigens from infectious agents (e.g., viral or bacterial agents). Where T Cell dysregulation (e.g., CD8+ T cell dysregulation) resulting in over reaction to allergens, the epitopes that may be presented include the epitopes of self-antigens (self-epitopes) and allergens. For example, an allergen may be selected from protein or non-protein components of: nuts (e.g., tree and/or peanuts), glutens, pollens, eggs (e.g. chicken, Gallus domesticus eggs), shellfish, soy, fish, and insect venoms (e.g., bee and/or wasp venom antigens). Similarly, where dysregulation of CD8+T reg cells and self-reactive CD8+ effector T cells result in autoimmune diseases the epitope presented may be from a protein associated with, for example, multiple sclerosis, Rasmussen's encephalitis, paraneoplastic syndromes. Celiac disease, systemic sclerosis (SSc), type 1 diabetes (T1D), Grave's disease (GD), systemic lupus erythematosus (SLE), aplastic anemia (AA), or vitiligo.

(i) Epitopes Present in Cancers—Cancer-Associated Antigens (“CAAs”)

Suitable epitopes for inclusion in a T-Cell-MP-epitope conjugate or higher order complex of T-Cell-MP-epitope conjugates include, but are not limited to, epitopes present in cancer-associated antigens. Cancer-associated antigens are known in the art; see, e.g., Cheever et al. (2009) Clin. Cancer Res. 15:5323. Cancer-associated antigens include, but are not limited to, α-folate receptor; carbonic anhydrase IX (CAIX); CD19; CD20; CD22; CD30; CD33; CD44v7/8; carcinoembryonic antigen (CEA); epithelial glycoprotein-2 (EGP-2); epithelial glycoprotein-40 (EGP-40); folate binding protein (FBP); fetal acetylcholine receptor; ganglioside antigen GD2; Her2/neu; IL-13R-a2; kappa light chain; LeY; L1 cell adhesion molecule; melanoma-associated antigen (MAGE); MAGE-A1; mesothelin; MUC1; NKG2D ligands; oncofetal antigen (h5T4); prostate stem cell antigen (PSCA); prostate-specific membrane antigen (PSMA); tumor-associate glycoprotein-72 (TAG-72); vascular endothelial growth factor receptor-2 (VEGF-R2) (see, e.g., Vigneron et al. (2013) Cancer Immunity 13:15; and Vigneron (2015) BioMed Res. Int'l Article ID 948501); and epidermal growth factor receptor (EGFR) vIII polypeptide (see, e.g., Wong et al. (1992) Proc. Natl. Acad. Sci. USA 89:2965; and Miao et al. (2014) PLoSOne 9:e94281).

In some cases, a suitable peptide epitope for incorporation into a T-Cell-MP-epitope conjugate is a peptide fragment of from about 4 aas (aa) to about 20 aa (e.g., 4 aa, 5 aa, 6 aa, 7 aa, 8 aa, 9 aa, 10 aa, 11 aa, 12 aa, 13 aa, 14 aa, 15 aa, 16 aa, 17 aa, 18 aa, 19 aa, or 20 aa) in length of a MUC1 polypeptide, an LMP2 polypeptide, an epidermal growth factor receptor (EGFR) vIII polypeptide, a HER-2/neu polypeptide, a melanoma antigen (e.g., MAGE A3) polypeptide, a p53 polypeptide, a mutant p53 polypeptide, an NY-ESO-1 polypeptide, a folate hydrolase (prostate-specific membrane antigen; PSMA) polypeptide, a CEA polypeptide, a melanoma antigen recognized by T cells (melanA/MART1) polypeptide, a Ras polypeptide (including a KRAS polypeptide), a gp100 polypeptide, a proteinase3 (PR1) polypeptide, a bcr-abl polypeptide, a tyrosinase polypeptide, a survivin polypeptide, a prostate specific antigen (PSA) polypeptide, an hTERT polypeptide, a sarcoma translocation breakpoints polypeptide, a synovial sarcoma X (SSX) breakpoint polypeptide, an EphA2 polypeptide, an acid phosphatase, prostate (PAP) polypeptide, a melanoma inhibitor of apoptosis (ML-IAP) polypeptide, an alpha-fetoprotein (AFP) polypeptide, an epithelial cell adhesion molecule (EpCAM) polypeptide, an ERG (TMPRSS2 ETS fusion) polypeptide, a NA17 polypeptide, a paired-box-3 (PAX3) polypeptide, an anaplastic lymphoma kinase (ALK) polypeptide, an androgen receptor polypeptide, a cyclin B1 polypeptide, an N-myc proto-oncogene (MYCN) polypeptide, a Ras homolog gene family member C (RhoC) polypeptide, a tyrosinase-related protein-2 (TRP-2) polypeptide, a mesothelin polypeptide, a prostate stem cell antigen (PSCA) polypeptide, a melanoma associated antigen-1 (MAGE A1) polypeptide, a cytochrome P450 1B1 (CYP1B1) polypeptide, a placenta-specific protein 1 (PLAC1) polypeptide, a BORIS polypeptide (also known as CCCTC-binding factor or CTCF), an ETV6-AML polypeptide, a breast cancer antigen NY-BR-1 polypeptide (also referred to as ankyrin repeat domain-containing protein 30A), a regulator of G-protein signaling (RGSS) polypeptide, a squamous cell carcinoma antigen recognized by T cells (SART3) polypeptide, a carbonic anhydrase IX polypeptide, a paired box-5 (PAX5) polypeptide, an OY-TES1 (testis antigen; also known as acrosin binding protein) polypeptide, a sperm protein 17 polypeptide, a lymphocyte cell-specific protein-tyrosine kinase (LCK) polypeptide, a high molecular weight melanoma associated antigen (HMW-MAA), an A-kinase anchoring protein-4 (AKAP-4), a synovial sarcoma X breakpoint 2 (SSX2) polypeptide, an X antigen family member 1 (XAGE1) polypeptide, a B7 homolog 3 (B7H3; also known as CD276) polypeptide, a legumain polypeptide (LGMN1; also known as asparaginyl endopeptidase), a tyrosine kinase with Ig and EGF homology domains-2 (Tie-2; also known as angiopoietin-1 receptor) polypeptide, a P antigen family member 4 (PAGE4) polypeptide, a vascular endothelial growth factor receptor 2 (VEGF2) polypeptide, a MAD-CT-1 polypeptide, a fibroblast activation protein (FAP) polypeptide, a platelet derived growth factor receptor beta (PDGFI3) polypeptide, a MAD-CT-2 polypeptide, a Fos-related antigen-1 (FOSL) polypeptide or a claudin (e.g. claudin 18.2) polypeptide. In some cases, a human papilloma virus (HPV) antigen is specifically excluded. In some cases, an alpha-feto protein (AFP) antigen is specifically excluded. In some cases, a Wilms tumor-1 (WT1) antigen is specifically excluded.

Amino acid sequences of cancer-associated antigens that may be incorporated into a T-Cell-MP-epitope conjugate are known in the art; see, e.g., MUC1 (GenBank CAA56734); LMP2 (GenBank CAA47024); EGFRvIII (GenBank NP_001333870); HER-2/neu (GenBank AAI67147); MAGE-A3 (GenBank AAH11744); p53 (GenBank BAC16799); NY-ESO-1 (GenBank CAA05908); PSMA (GenBank AAH25672); CEA (GenBank AAA51967); melan/MART1 (GenBank NP_005502); Ras (GenBank NP_001123914); gp100 (GenBank AAC60634); bcr-abl (GenBank AAB60388); tyrosinase (GenBank AAB60319); survivin (GenBank AAC51660); PSA (GenBank CAD54617); hTERT (GenBank BAC11010); SSX (GenBank NP_001265620); Eph2A (GenBank NP_004422); PAP (GenBank AAH16344); ML-IAP (GenBank AAH14475); EpCAM (GenBank NP_002345); ERG (TMPRSS2 ETS fusion) (GenBank ACA81385); PAX3 (GenBank AAI01301); ALK (GenBank NP_004295); androgen receptor (GenBank NP_000035); cyclin B1 (GenBank CA099273); MYCN (GenBank NP_001280157); RhoC (GenBank AAH52808); TRP-2 (GenBank AAC60627); mesothelin (GenBank AAH09272); PSCA (GenBank AAH65183); MAGE A1 (GenBank NP_004979); CYP1B1 (GenBank AAM50512); PLAC1 (GenBank AAG22596); BORIS (GenBank NP_001255969); ETV6 (GenBank NP_001978); NY-BR1 (GenBank NP_443723); SART3 (GenBank NP_055521); carbonic anhydrase IX (GenBank EAW58359); PAX5 (GenBank NP_057953); OY-TES1 (GenBank NP_115878); sperm protein 17 (GenBank AAK20878); LCK (GenBank NP_001036236); HMW-MAA (GenBank NP_001888); AKAP-4 (GenBank NP_003877); SSX2 (GenBank CAA60111); XAGE1 (GenBank NP_001091073; XP_001125834; XP_001125856; and XP_001125872); B7H3 (GenBank NP_001019907; XP_947368; XP_950958; XP_950960; XP_950962; XP_950963; XP_950965; and XP_950967); LGMN1 (GenBank NP_001008530); TIE-2 (GenBank NP_000450); PAGE4 (GenBank NP_001305806); VEGFR2 (GenBank NP_002244); MAD-CT-1 (GenBank NP_005893 NP_056215); FAP (GenBank NP_004451); PDGFI3 (GenBank NP_002600); MAD-CT-2 (GenBank NP_001138574); and FOSL (GenBank NP_005429). These polypeptides are also discussed in, e.g., Cheever et al. (2009) Clin. Cancer Res. 15:5323, and references cited therein; Wagner et al. (2003) J. Cell. Sci. 116:1653; Matsui et al. (1990) Oncogene 5:249; Zhang et al. (1996) Nature 383:168.

(a) Alpha Feto Protein (AFP)

T-Cell-MP-epitope conjugates, or their higher order complexes (e.g., duplexes), may comprise a peptide presenting an epitope of alpha-feto protein (AFP), which has been associated with hepatocellular carcinoma, pancreatic cancer, stomach cancer, colorectal cancer, hepatoblastoma, and an ovarian yolk sac tumors. The AFP epitope may be presented in the context of a Class I MHC polypeptide sequence that may have a) an aa sequence having at least 95% aa (e.g., at least 97%, 98%, or 99%) sequence identity to the HLA-A*0101, HLA-A*0201, HLA-A*1101, HLA-A*2301, HLA-A*2402, HLA-A*2407, HLA-A*3303, or HLA-A*3401; b) an aa sequence having at least 95% aa (e.g., at least 97%, 98%, or 99%) sequence identity to the HLA-B*0702, HLA-B*0801, HLA-B*1502, HLA-B*3802, HLA-B*4001, HLA-B*4601, or HLA-B*5301; or c) an aa sequence having at least 95% aa (e.g., at least 97%, 98%, or 99%) sequence identity to the HLA-C*0102, HLA-C*0303, HLA-C*0304, HLA-C*0401, HLA-C*0602, HLA-C*0701, HLA-C*0702, HLA-C*0801, or HLA-C*1502 depicted in FIGS. 3A-3G.

AFP peptides that may be included in a T-Cell-MP-epitope conjugate include, but are not limited to, AITRKMAAT (SEQ ID NO:284); AYTKKAPQL (SEQ ID NO:285); LLNQHACAV (SEQ ID NO:286); KLVLDVAHV (SEQ ID NO:287); FMNKFIYEI (SEQ ID NO:288); SIPLFQVPE (SEQ ID NO:289); LLNFTESRT (SEQ ID NO:290); FVQEATYKF (SEQ ID NO:291); ATYKEVSKM (SEQ ID NO:292); KEVSKMVKD (SEQ ID NO:293); RHNCFLAHK (SEQ ID NO:294); ATAATCCQL (SEQ ID NO:295); YIQESQALA (SEQ ID NO:296); QLTSSELMAI (SEQ ID NO:297); KLSQKFTKV (SEQ ID NO:298); KELRESSLL (SEQ ID NO:299); SLVVDETYV (SEQ ID NO:300); ILLWAARYD (SEQ ID NO:301); KIIPSCCKA (SEQ ID NO:302); CRGDVLDCL (SEQ ID NO:303); QQDTLSNKI (SEQ ID NO:304); TMKQEFLINL (SEQ ID NO:305); NLVKQKPQI (SEQ ID NO:306); AVIADFSGL (SEQ ID NO:307); LLACGEGAA (SEQ ID NO:308); LACGEGAAD (SEQ ID NO:309); KAPQLTSSEL (SEQ ID NO:310); YICSQQDTL (SEQ ID NO:311); TECCKLTTL (SEQ ID NO:312); CTAEISLADL (SEQ ID NO:313); VTKELRESSL (SEQ ID NO:314); IMSYICSQQD (SEQ ID NO:315); TRTFQAITV (SEQ ID NO:316); FQKLGEYYL (SEQ ID NO:317); RVAKGYQEL (SEQ ID NO:318); SYQCTAEISL (SEQ ID NO:319); KQEFLINLV (SEQ ID NO:320); MKWVESIFL (SEQ ID NO:321); PVNPGVGQC (SEQ ID NO:322); AADIIIGHL (SEQ ID NO:323); QVPEPVTSC (SEQ ID NO:324); TTLERGQCII (SEQ ID NO:325); KMAATAATC (SEQ ID NO:326); QAQGVALQTM (SEQ ID NO:327); FQAITVTKL (SEQ ID NO:328); LLEKCFQTE (SEQ ID NO:329); VAYTKKAPQ (SEQ ID NO:330); KYIQESQAL (SEQ ID NO:331); GVALQTMKQ (SEQ ID NO:332); GQEQEVCFA (SEQ ID NO:333); SEEGRHNCFL (SEQ ID NO:334); RHPFLYAPTI (SEQ ID NO:335); TEIQKLVLDV (SEQ ID NO:336); RRHPQLAVSV (SEQ ID NO:337); GEYYLQNAFL (SEQ ID NO:338); NRRPCFSSLV (SEQ ID NO:339); LQTMKQEFLI (SEQ ID NO:340); IADFSGLLEK (SEQ ID NO:341); GLLEKCCQGQ (SEQ ID NO:342); TLSNKITEC (SEQ ID NO:343); LQDGEKIMSY (SEQ ID NO:344); GLFQKLGBY (SEQ ID NO:345); NEYGIASILD (SEQ ID NO:346); KMVKDALTAI (SEQ ID NO:347); FLASFVHEY (SEQ ID NO:348); AQFVQEATY (SEQ ID NO:349); EYSRRHPQL (SEQ ID NO:350); AYEEDRETF (SEQ ID NO:351; SYANRRPCF (SEQ ID NO:352); CFAEEGQKL (SEQ ID NO:353); RSCGLFQKL (SEQ ID NO:354); IFLIFLLNF (SEQ ID NO:355); KPEGLSPNL (SEQ ID NO:356); and GLSPNLNRFL (SEQ ID NO:357).

In some cases, the AFP peptide present in a T-Cell-MP-epitope conjugate presents an HLA-A*2402-restricted epitope. Non-limiting examples of AFP peptides that present an HLA-A*2402-restricted epitope include: KYIQESQAL (SEQ ID NO:331); EYYLQNAFL (SEQ ID NO:358); AYTKKAPQL (SEQ ID NO:285); EYSRRHPQL (SEQ ID NO:350); RSCGLFQKL (SEQ ID NO:354) and AYEEDRETF (SEQ ID NO:351).

In some cases, the AFP peptide present in a T-Cell-MP-epitope conjugate of the present disclosure is KYIQESQAL (SEQ ID NO:331). In some cases, the AFP peptide present in a T-Cell-MP-epitope conjugate of the present disclosure is EYYLQNAFL (SEQ ID NO:358). In some cases, the AFP peptide present in T-Cell-MP-epitope conjugate of the present disclosure is AYTKKAPQL (SEQ ID NO:285). In some cases, the AFP peptide present in a T-Cell-MP-epitope conjugate of the present disclosure is EYSRRHPQL (SEQ ID NO:350). In some cases, the AFP peptide present in a T-Cell-MP-epitope conjugate of the present disclosure is RSCGLFQKL (SEQ ID NO:354).

In some cases, the AFP peptide present in a T-Cell-MP of the present disclosure presents an HLA-A*0201-restricted epitope. Non-limiting examples of AFP peptides that present an HLA-A*0201-restricted epitope are: FMNKFIYEI (SEQ ID NO:288); and GLSPNLNRFL (SEQ ID NO:357).

(b) Wilms Tumor Antigen (WT-1)

T-Cell-MP-epitope conjugates and their higher order complexes (e.g., duplexes) may comprise a peptide presenting an epitope of Wilms Tumor-1 protein, which has been associated with myeloid leukemia, myeloma, ovarian cancer, pancreatic cancer, non-small cell lung cancer, colorectal cancer, breast cancer, Wilms tumor, mesothelioma, soft tissue sarcoma, neuroblastoma, and nephroblastoma. The WT-1 epitope may be presented in the context of a class I MHC presenting sequence. The WT-1 epitope may be presented in the context of a class I MHC presenting complex having at least 95% (e.g., at least 97%, 98%, or 99%) aa sequence identity to the HLA-A*0101, HLA-A*0201, HLA-A*1101, HLA-A*2301, HLA-A*2402, HLA-A*2407, HLA-A*3303, or HLA-A*3401; b) an aa sequence having at least 95% aa sequence identity to the HLA-B*0702, HLA-B*0801, HLA-B*1502, HLA-B*3802, HLA-B*4001, HLA-B*4601, or HLA-B*5301; or c) an aa sequence having at least 95% aa sequence identity to the HLA-C*0102, HLA-C*0303, HLA-C*0304, HLA-C*0401, HLA-C*0602, HLA-C*0701, HLA-C*0702, HLA-C*0801, or HLA-C*1502 depicted in FIGS. 3A-3G.

WT-1 peptides that may be included in a T-Cell-MP-epitope conjugate include, but are not limited to, NLMNLGATL (SEQ ID NO:359), NYMNLGATL (SEQ ID NO:360), CMTWNQMNLGATLKG (SEQ ID NO:361), WNQMNLGATLKGVAA (SEQ ID NO:362), CMTWNYMNLGATLKG (SEQ ID NO:363), WNYMNLGATLKGVAA (SEQ ID NO:364), TWNQMNLGATLKGV (SEQ ID NO:365), TWNQMNLGATLKGVA (SEQ ID NO:366), CMTWNLMNLGATLKG (SEQ ID NO:367), MTWNLMNLGATLKGV (SEQ ID NO:368), TWNLMNLGATLKGVA (SEQ ID NO:369), WNLMNLGATLKGVAA (SEQ ID NO:370), MNLGATLK (SEQ ID NO:371), MTWNYMNLGATLKGV SEQ ID NO:372), TWNYMNLGATLKGVA (SEQ ID NO:373), CMTWNQMNLGATLKGVA (SEQ ID NO:374), CMTWNLMNLGATLKGVA (SEQ ID NO:375), CMTWNYMNLGATLKGVA (SEQ ID NO:376), GYLRNPTAC (SEQ ID NO:377), GALRNPTAL (SEQ ID NO:378), YALRNPTAC (SEQ ID NO:379), GLLRNPTAC (SEQ ID NO:380), RYRPHPGAL (SEQ ID NO:381), YQRPHPGAL (SEQ ID NO:382), RLRPHPGAL (SEQ ID NO:383), RIRPHPGAL (SEQ ID NO:384), QFPNHSFKHEDPMGQ (SEQ ID NO:385), HSFKHEDPY (SEQ ID NO:386), QFPNHSFKHEDPM (SEQ ID NO:387), QFPNHSFKHEDPY (SEQ ID NO:388), KRPFMCAYPGCNK (SEQ ID NO:389), KRPFMCAYPGCYK (SEQ ID NO:390), FMCAYPGCY (SEQ ID NO:391), FMCAYPGCK (SEQ ID NO:392), KRPFMCAYPGCNKRY (SEQ ID NO:393), SEKRPFMCAYPGCNK (SEQ ID NO:394), KRPFMCAYPGCYKRY (SEQ ID NO:395), VLDFAPPGA (SEQ ID NO:396); RMFPNAPYL (SEQ ID NO:397); YMFPNAPYL (SEQ ID NO:398); SLGEQQYSV (SEQ ID NO:399); CYTWNQMNL (SEQ ID NO:400); CMTWNQMNL (SEQ ID NO:401); and NQMNLGATL (SEQ ID NO:402).

In some cases, the WT-1 peptide present in a T-Cell-MP-epitope conjugate presents an HLA-A*2402-restricted epitope. WT-1 peptides that present an HLA-A*2402-restricted epitope include, e.g., CMTWNQMN (SEQ ID NO:403); NYMNLGATL (SEQ ID NO:360) (WT-1 239-247; Q240Y); CYTWNQMNL (SEQ ID NO:400) (WT-1 235-243); CMTWNQMNL (SEQ ID NO:401) (WT-1 235-243); NQMNLGATL (SEQ ID NO:402) (WT-1 239-247); and NLMNLGATL (SEQ ID NO:359) (WT-1239-247; Q240L).

In some cases, the WT-1 peptide present in a T-Cell-MP-epitope conjugate presents an HLA-A*0201-restricted epitope. WT-1 peptides that present an HLA-A*0201-restricted epitope include, e.g., VLDFAPPGA (SEQ ID NO:396) (WT-1 37-45); RMFPNAPYL (SEQ ID NO:397) (WT-1 126-134); YMFPNAPYL (SEQ ID NO:398) (WT-1 126-134; R126Y); SLGEQQYSV (SEQ ID NO:399) (WT-1 187-195); and NLMNLGATL (SEQ ID NO:359) (WT-1 239-247; Q240L).

(c) Human Papilloma Virus I (HPV)

T-Cell-MP-epitope conjugates and their higher order complexes (e.g., duplexes) may comprise a peptide presenting an epitope of a human papilloma virus (HPV), which has been associated with cervical cancer, prostate cancer, or ovarian cancer. HPV epitopes may be presented in the context of a class I MHC presenting sequence. The HPV epitope may be presented in the context of a class I MHC presenting complex having at least 95% aa sequence identity to the HLA-A*0101, HLA-A*0201, HLA-A*1101, HLA-A*2301, HLA-A*2402, HLA-A*2407, HLA-A*3303, or HLA-A*3401; b) an aa sequence having at least 95% (e.g., at least 97%, 98%, or 99%) aa sequence identity to the HLA-B*0702, HLA-B*0801, HLA-B*1502, HLA-B*3802, HLA-B*4001, HLA-B*4601, or HLA-B*5301; or c) an aa sequence having at least 95% aa sequence identity to the HLA-C*0102, HLA-C*0303, HLA-C*0304, HLA-C*0401, HLA-C*0602, HLA-C*0701, HLA-C*0702, HLA-C*0801, or HLA-C*1502 depicted in FIGS. 3A-3G. HPV is also an infectious agent and its epitopes may be employed to alter the immune response to HPV for prophylaxis or treatment of an infection.

HPV peptide epitopes include, but are not limited to, those from the E6 and E7 gene products: E6 18-26 (KLPQLCTEL; SEQ ID NO:274); E6 26-34 (LQTTIHDII; SEQ ID NO:404); E6 49-57 (VYDFAFRDL; SEQ ID NO:405); E6 52-60 (FAFRDLCIV; SEQ ID NO:406); E6 75-83 (KFYSKISEY; SEQ ID NO:407); E6 80-88 (ISEYRHYCY; SEQ ID NO:408); E7 7-15 (TLHEYMLDL; SEQ ID NO:409); E7 11-19 (YMLDLQPET; SEQ ID NO:276); E7 44-52 (QAEPDRAHY; SEQ ID NO:410); E7 49-57 (RAHYNIVTF (SEQ ID NO:411); E7 61-69 (CDSTLRLCV; SEQ ID NO:412); E7 67-76 (LCVQSTHVDI; SEQ ID NO:413); E7 82-90 (LLMGTLGIV; SEQ ID NO:414); E7 86-93 (TLGIVCPI; SEQ ID NO:277); or E7 92-93 (LLMGTLGIVCPI; SEQ ID NO:415).

In some cases, the epitope is HPV16E7/82-90 (LLMGTLGIV; SEQ ID NO:414). In some cases, the epitope is HPV16E7/86-93 (TLGIVCPI; SEQ ID NO:277). In some cases, the epitope is HPV16E7/11-20 (YMLDLQPETT; SEQ ID NO:416). In some cases, the epitope is HPV16E7/11-19 (YMLDLQPET; SEQ ID NO:276). See, e.g., Ressing et al. ((1995) J. Immunol. 154:5934) for additional suitable HPV epitopes.

HPV peptides suitable for inclusion into a T-Cell-MP-epitope conjugate include HPV E6 peptides that binds HLA-A24 (e.g., is an HLA-A2401-restricted epitope). Non-limiting examples of such peptides include: VYDFAFRDL (SEQ ID NO:405); CYSLYGTTL (SEQ ID NO:417); EYRHYCYSL (SEQ ID NO:418); KLPQLCTEL (SEQ ID NO:274); DPQERPRKL (SEQ ID NO:419); HYCYSLYGT (SEQ ID NO:420); DFAFRDLCI (SEQ ID NO:421); LYGTTLEQQY (SEQ ID NO:422); HYCYSLYGTT (SEQ ID NO:423); EVYDFAFRDL (SEQ ID NO:424); EYRHYCYSLY (SEQ ID NO:425); VYDFAFRDLC (SEQ ID NO:426); YCYSIYGTTL (SEQ ID NO:427); VYCKTVLEL (SEQ ID NO:428); VYGDTLEKL (SEQ ID NO:429); and LTNTGLYNLL (SEQ ID NO:430).

In some cases, an HPV peptide suitable for inclusion into a T-Cell-MP-epitope conjugate is selected from the group consisting of: DLQPETTDL (SEQ ID NO:431); TLHEYMLDL (SEQ ID NO:409); TPTLHEYML (SEQ ID NO:432); RAHYNIVTF (SEQ ID NO:411); GTLGIVCPI (SEQ ID NO:433); EPDRAHYNI (SEQ ID NO:434); QLFLNTLSF (SEQ ID NO:435); FQQLFLNTL (SEQ ID NO:279); and AFQQLFLNTL (SEQ ID NO:436).

In some cases, a suitable HPV peptide presents an HLA-A*2401-restricted epitope. Nonlimiting examples of HPV peptides presenting an HLA-A*2401-restricted epitope are: VYDFAFRDL (SEQ ID NO:405); RAHYNIVTF (SEQ ID NO:411); CDSTLRLCV (SEQ ID NO:412); and LCVQSTHVDI (SEQ ID NO:413). In some cases, an HPV peptide suitable for inclusion in a T-Cell-MP of the present disclosure is VYDFAFRDL (SEQ ID NO:405). In some cases, an HPV peptide suitable for inclusion in a T-Cell-MP-epitope conjugate of the present disclosure is RAHYNIVTF (SEQ ID NO:411). In some cases, an HPV peptide suitable for inclusion in a T-Cell-MP of the present disclosure is CDSTLRLCV (SEQ ID NO:412). In some cases, an HPV peptide suitable for inclusion in a T-Cell-MP of the present disclosure is LCVQSTHVDI (SEQ ID NO:413).

(d) Hepatitis B Virus (HBV)

T-Cell-MP-epitope conjugates and their higher order complexes (e.g., duplexes) may comprise a peptide presenting an epitope of a hepatitis B virus (HBV), which has been associated with hepatocellular carcinoma. HBV epitopes may be presented in the context of a class I MHC presenting complex. The class I MHC may be a) an aa sequence having at least 95% a sequence identity to the HLA-A*0101, HLA-A*0201, HLA-A*1101, HLA-A*2301, HLA-A*2402, HLA-A*2407, HLA-A*3303, or HLA-A*3401; b) an aa sequence having at least 95% aa sequence identity to the HLA-B*0702, HLA-B*0801, HLA-B*1502, HLA-B*3802, HLA-B*4001, HLA-B*4601, or HLA-B*5301; or c) an aa sequence having at least 95% aa sequence identity to the HLA-C*0102, HLA-C*0303, HLA-C*0304, HLA-C*0401, HLA-C*0602, HLA-C*0701, HLA-C*0702, HLA-C*0801, or HLA-C*1502 depicted in FIGS. 3A-3G. HBV is also an infectious agent and its epitopes may be employed to alter the immune response to HBV for prophylaxis or treatment of an infection.

HBV peptides suitable for inclusion into a T-Cell-MP-Epitope conjugate include, but are not limited to, FLPSDFFPSV (SEQ ID NO:437), GLSRYVARLG (SEQ ID NO:438), KLHLYSHPI (SEQ ID NO:439), FLLSLGIHL (SEQ ID NO:440), ALMPLYACI (SEQ ID NO:441), SLYADSPSV (SEQ ID NO:442), STLPETTVV (SEQ ID NO:443), LIMPARFYPK (SEQ ID NO:444), AIMPARFYPK (SEQ ID NO:445), YVNVNMGLK (SEQ ID NO:446), MQWNSTALHQALQDP (SEQ ID NO:447), LLDPRVRGL (SEQ ID NO:448), SILSKTGDPV (SEQ ID NO:449), VLQAGFFLL (SEQ ID NO:450), FLLTRILTI (SEQ ID NO:451), FLGGTPVCL (SEQ ID NO:452), LLCLIFLLV (SEQ ID NO:453), LVLLDYQGML (SEQ ID NO:454), LLDYQGMLPV (SEQ ID NO:455), IPIPSSWAF (SEQ ID NO:456), WLSLLVPFV (SEQ ID NO:457), GLSPTVWLSV (SEQ ID NO:458), SIVSPFIPLL (SEQ ID NO:459), ILSPFLPLL (SEQ ID NO:460), ATVELLSFLPSDFFPSV (SEQ ID NO:461), LPSDFFPSV (SEQ ID NO:462), CLTFGRETV (SEQ ID NO:463), VLEYLVSFGV (SEQ ID NO:464), EYLVSFGVW (SEQ ID NO:465), ILSTLPETTV (SEQ ID NO:466), STLPETTVVRR (SEQ ID NO:467), NVSIPWTHK (SEQ ID NO:468), KVGNFTGLY (SEQ ID NO:469), GLYSSTVPV (SEQ ID NO:470), TLWKAGILYK (SEQ ID NO:471), TPARVTGGVF (SEQ ID NO:472), LVVDFSQFSR (SEQ ID NO:473), GLSRYVARL (SEQ ID NO:474), SIACSVVRR (SEQ ID NO:475), YMDDVVLGA (SEQ ID NO:476), PLGFFPDH (SEQ ID NO:477), QAFTFSPTYK (SEQ ID NO:478), KYTSFPWLL (SEQ ID NO:479), ILRGTSFVYV (SEQ ID NO:480), HLSLRGLFV (SEQ ID NO:481), VLHKRTLGL (SEQ ID NO:482), GLSAMSTTDL (SEQ ID NO:483), CLFKDWEEL (SEQ ID NO:484), and VLGGCRHKL (SEQ ID NO:485).

(ii) Infectious Agents

Suitable epitopes from infectious agents that may be included in a T-Cell-MP-epitope conjugate or its or higher order complexes (e.g., duplexes) T-Cell-MPs include, but are not limited to, epitopes present in an infectious virus, bacterium, fungus, protozoan, or helminth disease causing agents, e.g., an epitope presented by a virus-encoded polypeptide.

Examples of viral infectious disease agents include, e.g., Adenoviruses, Adeno-associated virus, Alphaviruses (Togaviruses), Eastern equine encephalitis virus, Eastern equine encephalomyelitis virus, Venezuelan equine encephalomyelitis vaccine strain TC-83, Western equine encephalomyelitis virus, Arenaviruses, Lymphocytic choriomeningitis virus (non-neurotropic strains), Tacaribe virus complex, Bunyaviruses, Bunyamwera virus, Rift Valley fever virus vaccine strain MP-12, Chikungunya virus, Calciviruses, Coronaviruses, Cowpox virus, Flaviviruses (Togaviruses)-Group B Arboviruses, Dengue virus serotypes 1, 2, 3, and 4, Yellow fever virus vaccine strain 17D, Hepatitis A, B, C, D, and E viruses, the Cytomegalovirus, Epstein Barr virus, Eastern Equine encephalitis virus, Herpes simplex types 1 and 2, Herpes zoster, Human herpesvirus types 6 and 7, hepatitis C virus (HVC), hepatitis B virus (HBV), Influenza viruses types A, B, and C, Papovaviruses, Newcastle disease virus, Measles virus, Mumps virus, Parainfluenza viruses types 1, 2, 3, and 4, polyomaviruses (JC virus, BK virus), Respiratory syncytial virus, Human parvovirus (B 19), Coxsackie viruses types A and B, Echoviruses, Polioviruses, Rhinoviruses, Alastrim (Variola minor virus), Smallpox (Variola major virus), Whitepox Reoviruses, Coltivirus, human Rotavirus, and Orbivirus (Colorado tick fever virus), Rabies virus, Vesicular stomatitis virus, Rubivirus (rubella), Semliki Forest virus, St. Louis encephalitis virus, Venezuelan equine encephalitis virus, Venezuelan equine encephalomyelitis virus, Arenaviruses (a.k.a. South American Hemorrhagic Fever virus), Flexal, Lymphocytic choriomeningitis virus (LCM) (neurotropic strains), Hantaviruses including Hantaan virus, Rift Valley fever virus, Japanese encephalitis virus, Yellow fever virus, Monkeypox virus, Human immunodeficiency virus (HIV) types 1 and 2, Human T cell lymphotropic virus (HTLV) types 1 and 2, Simian immunodeficiency virus (SIV), Vesicular stomatitis virus, Guanarito virus, Lassa fever virus, Junin virus, Machupo virus, Sabia, Crimean-Congo hemorrhagic fever virus, Ebola viruses, Marburg virus, Tick-borne encephalitis virus complex (flavi) including Central European tick-borne encephalitis, Far Eastern tick-borne encephalitis, Hanzalova, Hypr, Kumlinge, Kyasanur Forest disease, Omsk hemorrhagic fever, and Russian Spring Summer encephalitis viruses, Herpesvirus simiae (Herpes B or Monkey B virus), Cercopithecine herpesvirus 1 (Herpes B virus), Equine morbillivirus (Hendra and Hendra-like viruses), Nipah virus, Variola major virus (Smallpox virus), Variola minor virus (Alastrim), African swine fever virus, African horse sickness virus, Akabane virus, Avian influenza virus (highly pathogenic), Blue tongue virus, Camel pox virus, Classical swine fever virus, Cowdria ruminantium (heartwater), Foot and mouth disease virus, Goat pox virus, Japanese encephalitis virus, Lumpy skin disease virus, Malignant catarrhal fever virus, Menangle virus, Newcastle disease virus (VVND), Vesicular stomatitis virus (exotic), and Zika virus. Antigens encoded by such viruses are known in the art; a peptide epitope suitable for use in a T-Cell-MP-epitope conjugate of the present disclosure can include a peptide from any known viral antigen.

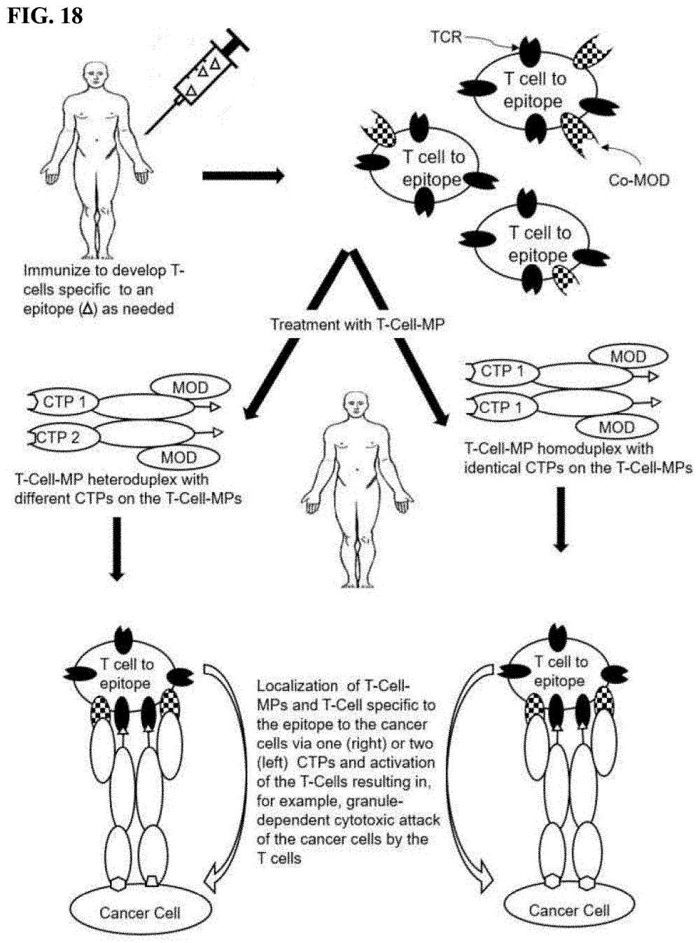

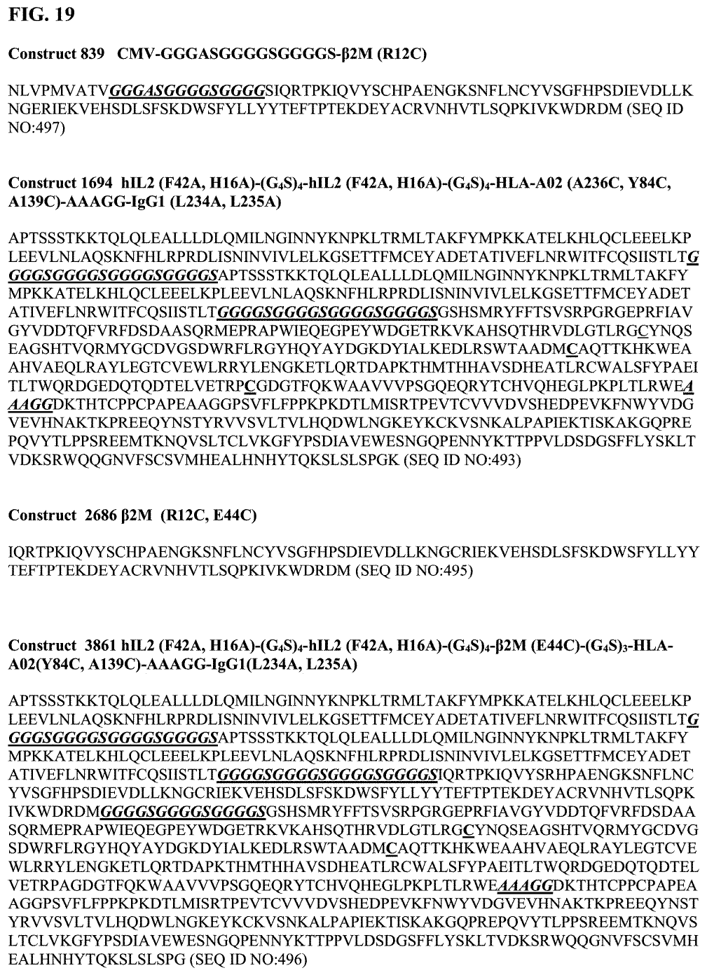

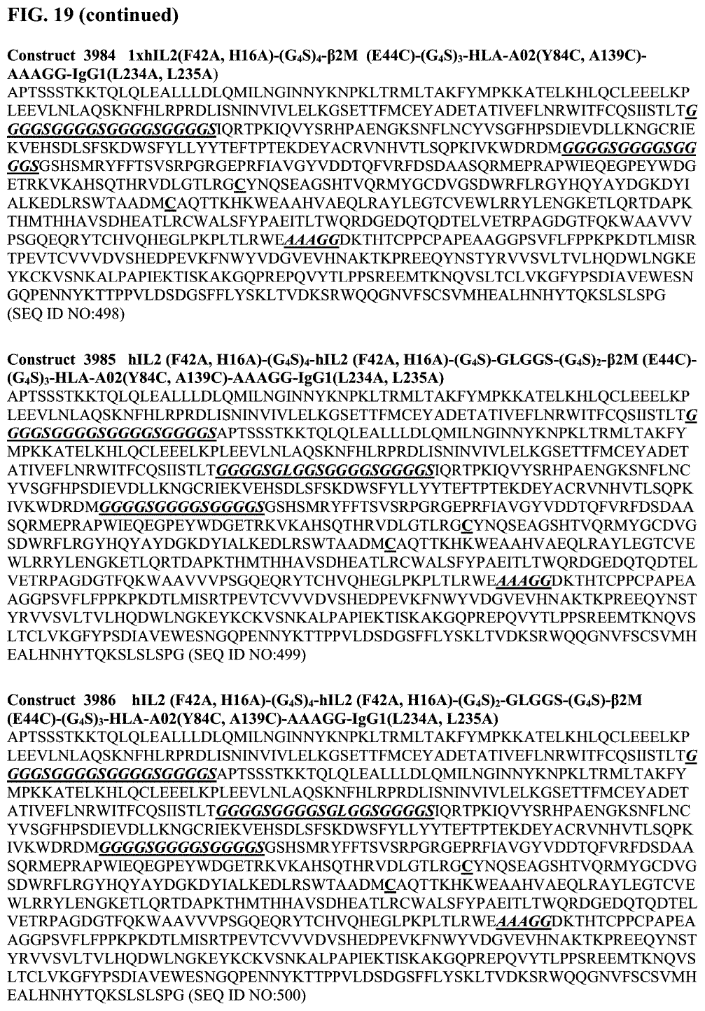

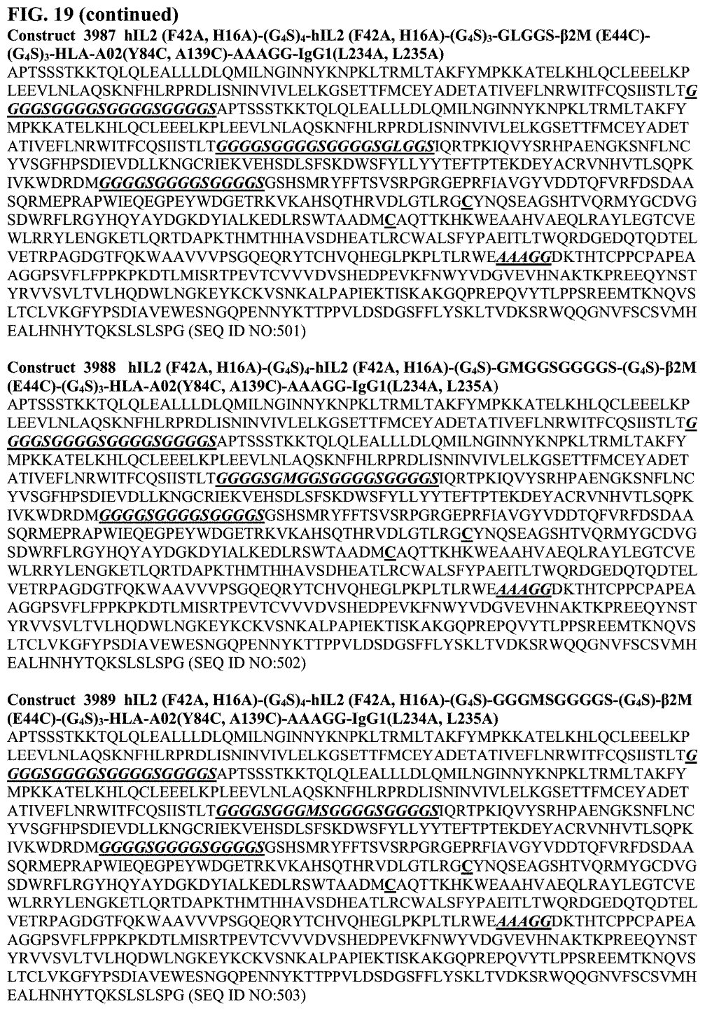

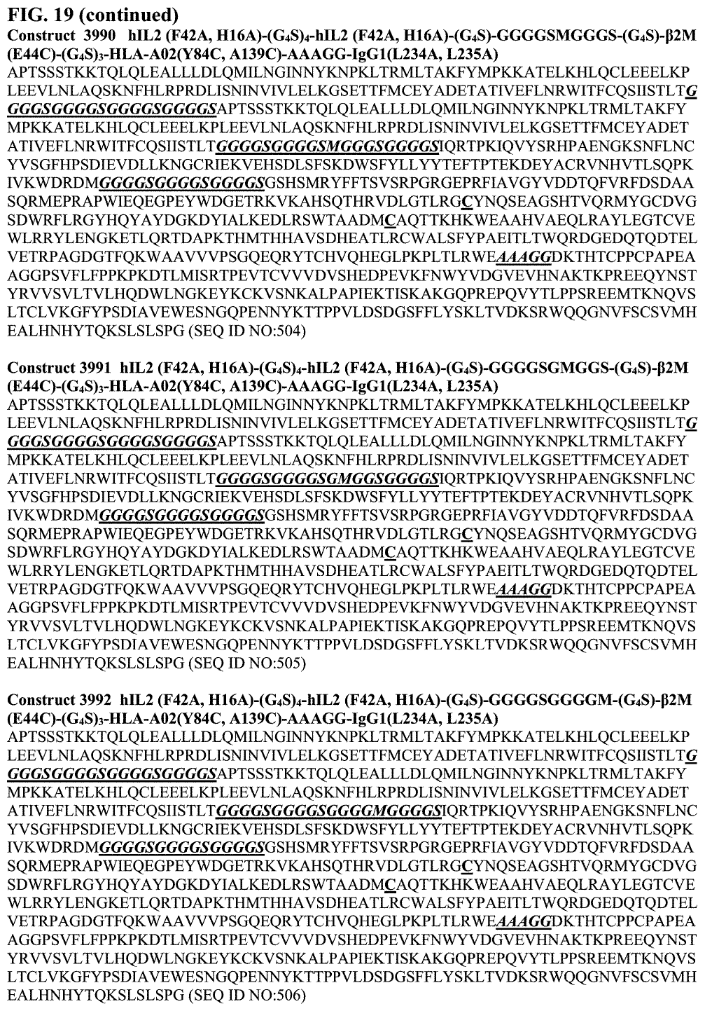

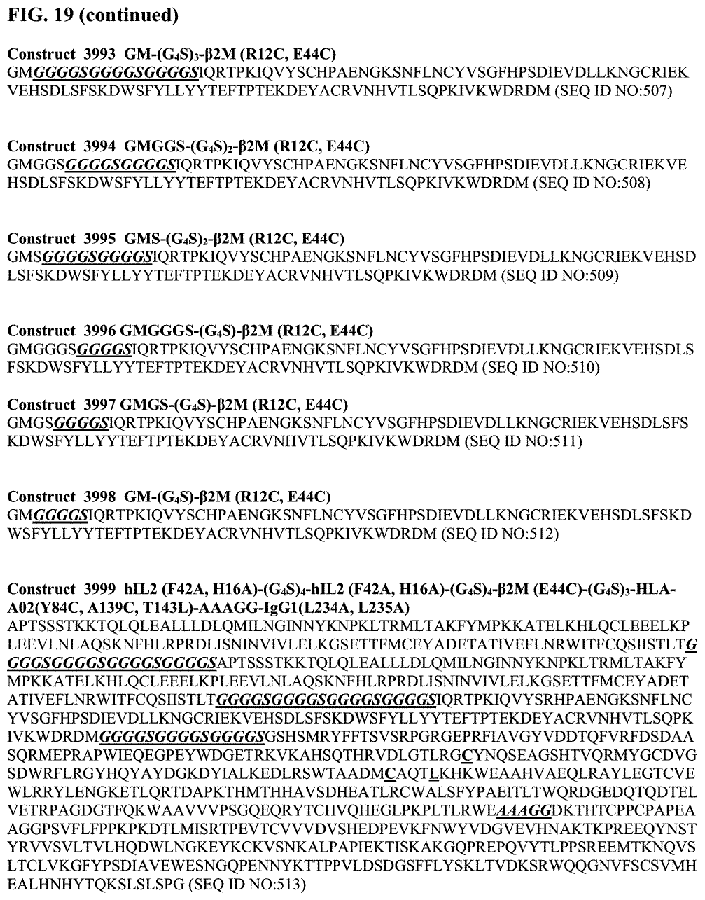

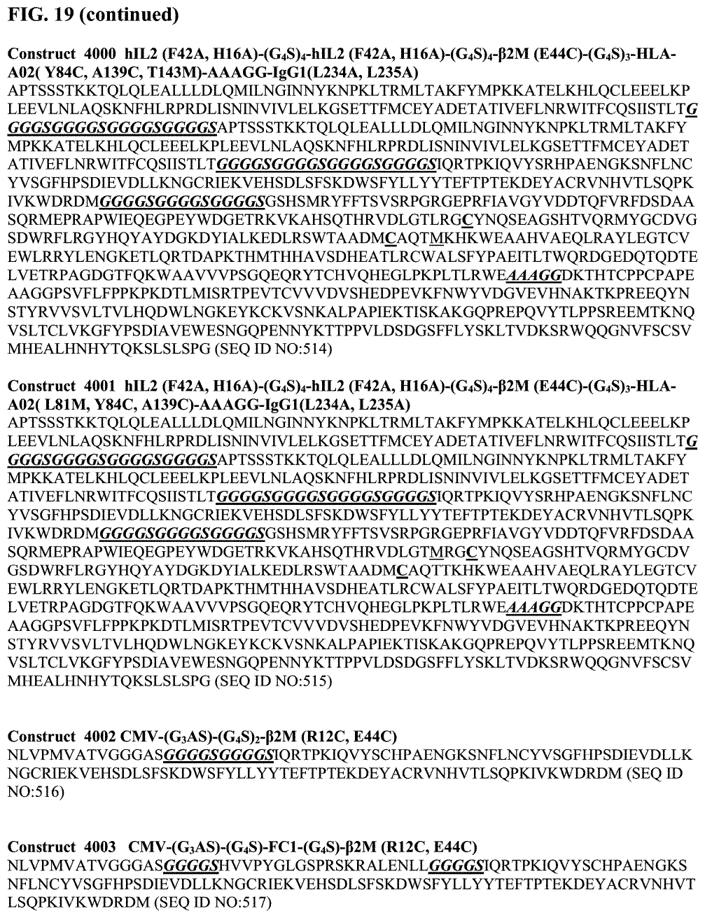

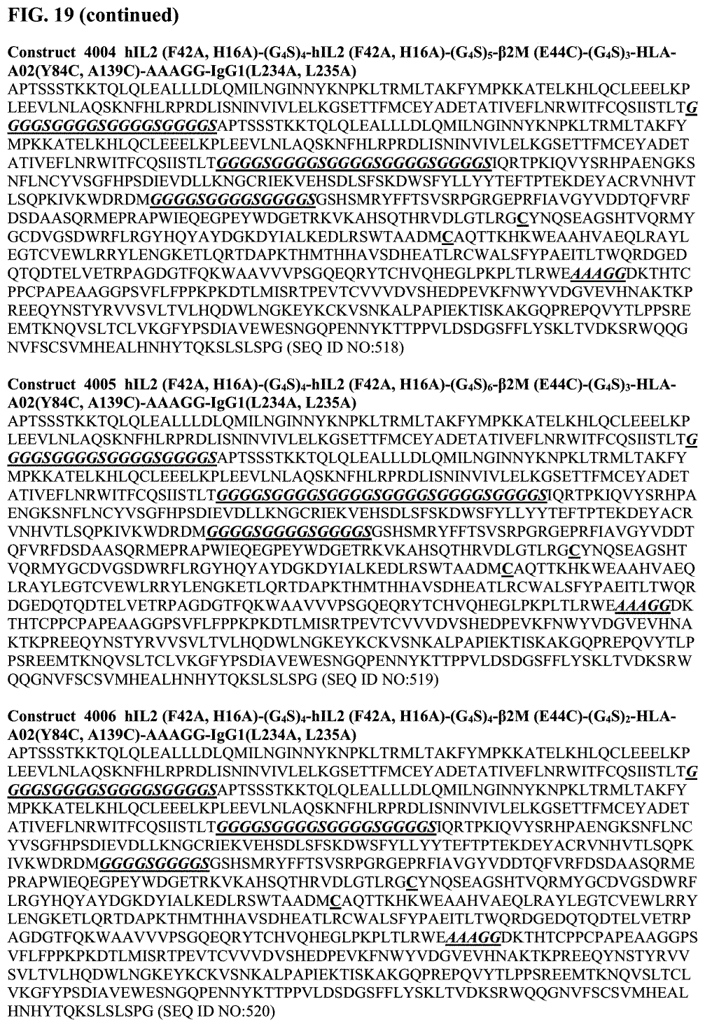

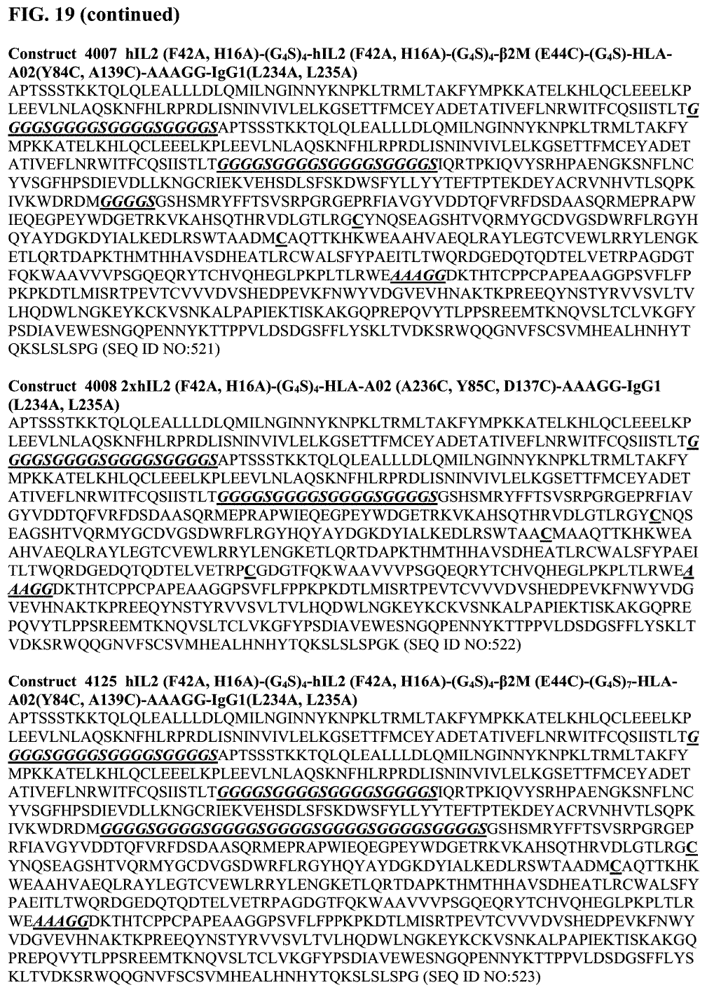

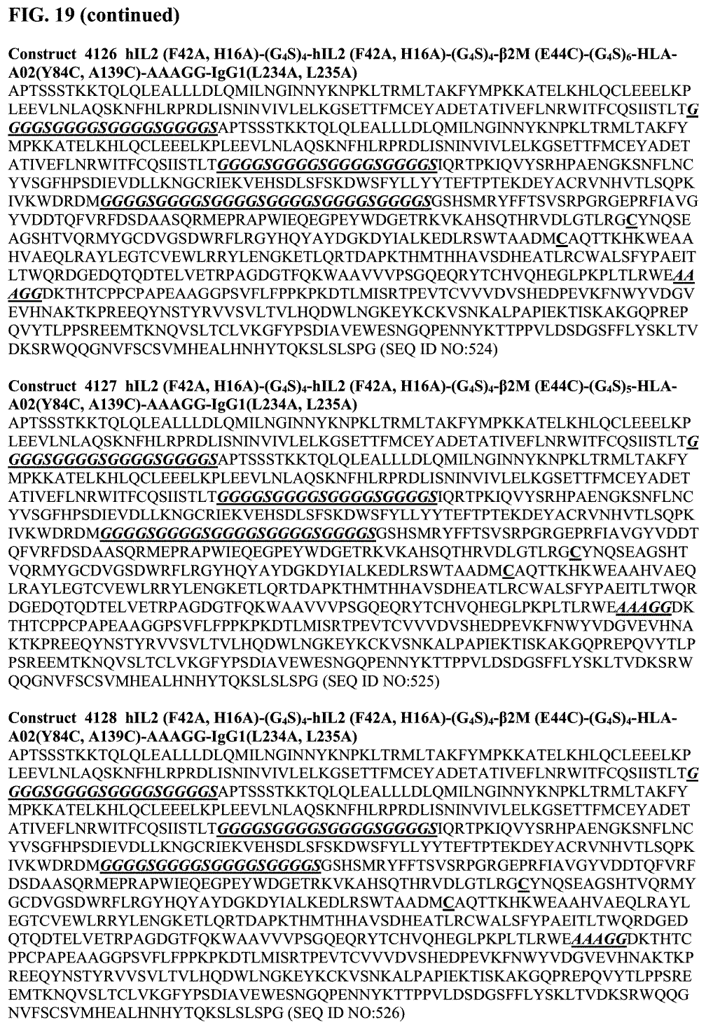

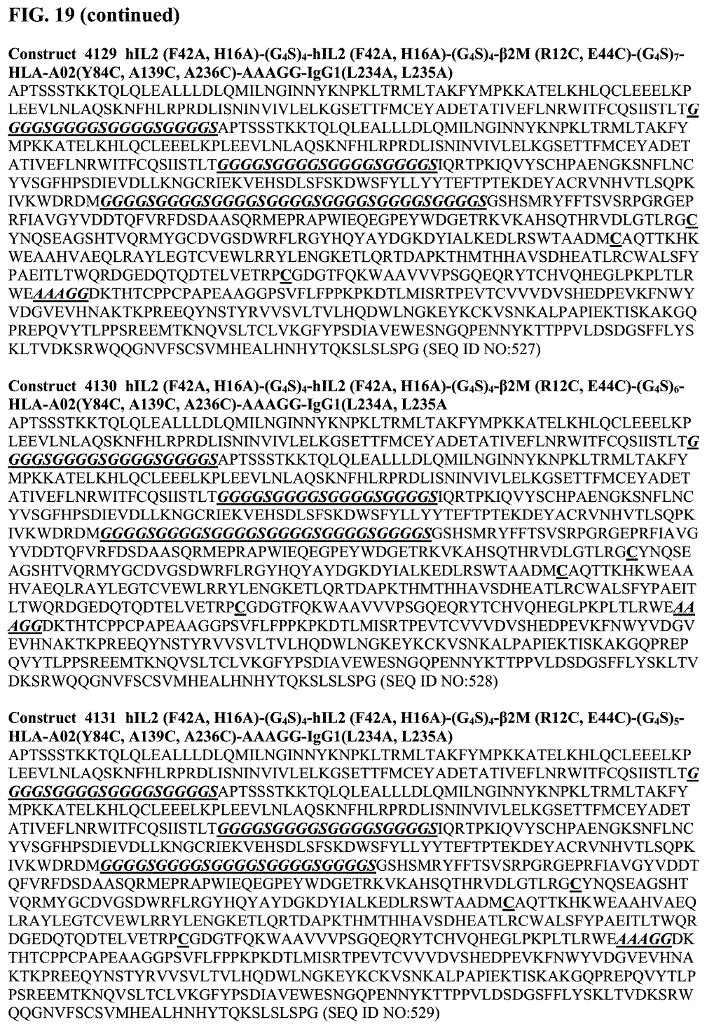

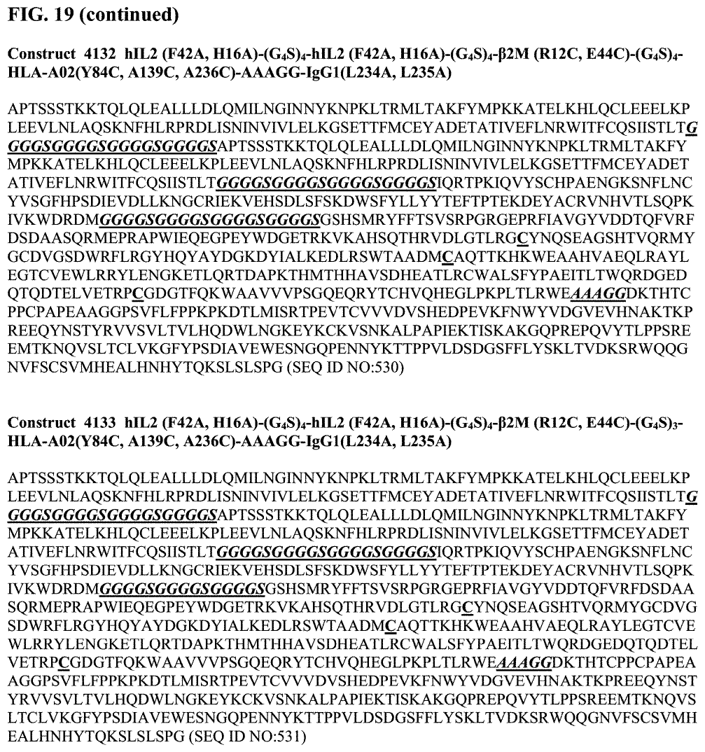

In embodiments where the T-Cell-MP-epitope conjugate includes a targeting sequence (e.g., a targeting sequence directed against a cancer-associated polypeptide) that directs the T-Cell-MP-epitope conjugate to a predetermined target cell or tissue (e.g., a cancerous cell or tissue), the epitope is advantageously one that binds T cells that already are present in the patient, e.g., resulting from exposure to a foreign agent such as a virus or bacteria, or from vaccination. For example, the epitope can be an epitope present in a viral antigen encoded by a virus that infects a majority of the human population, e.g., cytomegalovirus (CMV), Epstein-Barr virus (EBV), human papilloma virus, adenovirus, and the like, or for which a majority of the human population has immunity through vaccination, e.g., tetanus, or for which a patient has been specifically vaccinated, e.g., with a CMV, tetanus or HPV vaccine, prior to treatment with the T-Cell-MP-epitope conjugate. The result is that the T cells present in the patient are effectively redirected from taking action against cells presenting the epitope to taking action against target cells or tissues recognized by the targeting sequence. See e.g., FIG. 18.

(a) CMV Peptide Epitopes

As mentioned above, in some cases a T-Cell-MP-epitope conjugate of the present disclosure comprises a CMV peptide epitope, i.e., a peptide that when in a MHC/peptide complex (e.g., an HLA/peptide complex), presents a CMV epitope (i.e., an epitope present in a CMV antigen) to a T cell. As with other peptide epitopes of this disclosure, a CMV peptide epitope has a length of at least 4 amino acids, e.g., from 4 amino acids to about 25 amino acids (e.g., 4 amino acids (aa), 5 aa, 6 aa, 7 aa, 8 aa, 9 aa, 10 aa, 11 aa, 12 aa, 13 aa, 14 aa, 15 aa, 16 aa, 17 aa, 18 aa, 19 aa, 20 aa, 21 aa, 22 aa, 23 aa, 24 aa, or 25 aa, including within a range of from 4 to 20 aa., from 6 to 18 aa., from 8 to 15 aa. from 8 to 12 aa., from 5 to 10 aa., from 10 to 15 aa., from 15 to 20 aa., from 10 to 20 aa., or from 15 to 25 aa. in length).

A given CMV epitope-specific T cell binds an epitope having a reference amino acid sequence of a given CMV epitope, but does not substantially bind an epitope that differs from the reference amino acid sequence. For example, a given CMV epitope-specific T cell binds a CMV epitope having a reference amino acid sequence, and binds an epitope that differs from the reference amino acid sequence, if at all, with an affinity that is less than 10-6 M, less than 10-5 M, or less than 10-4 M. A given CMV epitope-specific T cell can bind an epitope for which it is specific with an affinity of at least 10-7 M, at least 10-8 M, at least 10-9 M, or at least 10-10 M.

In some cases, a CMV peptide epitope present in a T-Cell-MP-epitope conjugate of the present disclosure is a peptide from CMV pp65. In some cases, a CMV peptide epitope present in a T-Cell-MP-epitope conjugate of the present disclosure is a peptide from CMV gB (glycoprotein B).

For example, in some cases, a CMV peptide epitope present in a T-Cell-MP-epitope conjugate of the present disclosure is a peptide of a CMV polypeptide having a length of at least 4 amino acids, e.g., from 4 amino acids to about 25 amino acids (e.g., 4 amino acids (aa), 5 aa, 6 aa, 7 aa, 8 aa, 9 aa, 10 aa, 11 aa, 12 aa, 13 aa, 14 aa, 15 aa, 16 aa, 17 aa, 18 aa, 19 aa, 20 aa, 21 aa, 22 aa, 23 aa, 24 aa, or 25 aa, including within a range of from 4 to 20 aa., from 6 to 18 aa., from 8 to 15 aa. from 8 to 12 aa., from 5 to 10 aa., from 10 to 15 aa., from 15 to 20 aa., from 10 to 20 aa., or from 15 to 25 aa. in length), and comprising an amino acid sequence having at least 80%, at least 85%, at least 90%, at least 95%, at least 98%, at least 99%, or 100%, amino acid sequence identity to the following CMV pp65 amino acid sequence:

(SEQ ID NO: 486)
MESRGRRCPE MISVLGPISG HVLKAVFSRG DTPVLPHETR
LLQTGIHVRV SQPSLILVSQ YTPDSTPCHR GDNQLQVQHT
YFTGSEVENV SVNVHNPTGR SICPSQEPMS IYVYALPLKM
LNIPSINVHH YPSAAERKHR HLPVADAVIH ASGKQMWQAR
LTVSGLAWTR QQNQWKEPDV YYTSAFVFPT KDVALRHVVC
AHELVCSMEN TRATKMQVIG DQYVKVYLES FCEDVPSGKL
FMHVTLGSDV EEDLTMTRNP QPFMRPHERN GFTVLCPKNM
IIKPGKISHI MLDVAFTSHE HFGLLCPKSI PGLSISGNLL
MNGQQIFLEV QAIRETVELR QYDPVAALFF FDIDLLLQRG
PQYSEHPTFT SQYRIQGKLE YRHTWDRHDE GAAQGDDDVW
TSGSDSDEEL VTTERKTPRV TGGGAMAGAS TSAGRKRKSA
SSATACTSGV MTRGRLKAES TVAPEEDTDE DSDNEIHNPA
VFTWPPWQAG ILARNLVPMV ATVQGQNLKY QEFFWDANDI
YRIFAELEGV WQPAAQPKRR RHRQDALPGP CIASTPKKHR G.

As one non-limiting example, a CMV peptide epitope present in a T-Cell-MP-epitope conjugate of the present disclosure has the amino acid sequence NLVPMVATV (SEQ ID NO:487) and has a length of 9 amino acids.

In some cases, a CMV peptide epitope present in a T-Cell-MP-epitope conjugate of the present disclosure is a peptide having a length of at least 4 amino acids, e.g., from 4 amino acids to about 25 amino acids (e.g., 4 amino acids (aa), 5 aa, 6 aa, 7 aa, 8 aa, 9 aa, 10 aa, 11 aa, 12 aa, 13 aa, 14 aa, 15 aa, 16 aa, 17 aa, 18 aa, 19 aa, 20 aa, 21 aa, 22 aa, 23 aa, 24 aa, or 25 aa, including within a range of from 4 to 20 aa., from 6 to 18 aa., from 8 to 15 aa. from 8 to 12 aa., from 5 to 10 aa., from 10 to 15 aa., from 15 to 20 aa., from 10 to 20 aa., or from 15 to 25 aa. in length) of a CMV polypeptide comprising an amino acid sequence having at least 80%, at least 85%, at least 90%, at least 95%, at least 98%, at least 99%, or 100%, amino acid sequence identity to the following CMV gB amino acid sequence:

(SEQ ID NO: 488)
MESRIWCLVVCVNLCIVCLGAAVSSSSTSHATSSTHNGSHTSRTTSAQTRS
VYSQHVTSSEAVSHRANETIYNTTLKYGDVVGVNTTKYPYRVCSMAQGTDL
IRFERNIICTSMKPINEDLDEGIMVVYKRNIVAHTFKVRVYQKVLTFRRSY
AYIYTTYLLGSNTEYVAPPMWEIHHINKFAQCYSSYSRVIGGTVFVAYHRD
SYENKTMQLIPDDYSNTHSTRYVTVKDQWHSRGSTWLYRETCNLNCMLTIT
TARSKYPYHFFATSTGDVVYISPFYNGTNRNASYFGENADKFFIFPNYTIV
SDFGRPNAAPETHRLVAFLERADSVISWDIQDEKNVTCQLTFWEASERTIR
SEAEDSYHFSSAKMTATFLSKKQEVNMSDSALDCVRDEAINKLQQIFNTSY
NQTYEKYGNVSVFETSGGLVVFWQGIKQKSLVELERLANRSSLNITHRTRR
STSDNNTTHLSSMESVHNLVYAQLQFTYDTLRGYINRALAQIAEAWCVDQR
RTLEVFKELSKINPSAILSAIYNKPIAARFMGDVLGLASCVTINQTSVKVL
RDMNVKESPGRCYSRPVVIFNFANSSYVQYGQLGEDNEILLGNHRTEECQL
PSLKIFIAGNSAYEYVDYLFKRMIDLSSISTVDSMIALDIDPLENTDFRVL
ELYSQKELRSSNVFDLEEIMREFNSYKQRVKYVEDKVVDPLPPYLKGLDDL
MSGLGAAGKAVGVAIGAVGGAVASVVEGVATFLKNPFGAFTIILVAIAVVI
ITYLIYTRQRRLCTQPLQNLFPYLVSADGTTVTSGSTKDTSLQAPPSYEES
VYNSGRKGPGPPSSDASTAAPPYTNEQAYQMLLALARLDAEQRAQQNGTDS
LDGQTGTQDKGQKPNLLDRLRHRKNGYRHLKDSDEEENV.

In some cases, the CMV epitope present in a T-Cell-MP-epitope conjugate of the present disclosure presents an epitope specific to an HLA-A, -B, -C, -E, -F, or -G allele. In some cases, the peptide epitope present in a T-Cell-MP-epitope conjugate presents an epitope restricted to HLA-A*0101, A*0201, A*0301, A*1101, A*2301, A*2402, A*2407, A*3303, and/or A*3401. In some cases, the CMV epitope present in a T-Cell-MP-epitope conjugate of the present disclosure presents an epitope restricted to HLA-B*0702, B*0801, B*1502, B*3802, B*4001, B*4601, and/or B*5301. In some cases, the CMV epitope present in a T-Cell-MP-epitope conjugate of the present disclosure presents an epitope restricted to C*0102, C*0303, C*0304, C*0401, C*0602, C*0701, C*702, C*0801, and/or C*1502. As one example, in some cases, a T-Cell-MP-epitope conjugate of the present disclosure comprises: a) a CMV peptide epitope having amino acid sequence NLVPMVATV (SEQ ID NO:487) and having a length of 9 amino acids; b) an HLA-A*0201 class I heavy chain polypeptide; and c) a β2M polypeptide.

In some cases, a T-Cell-MP-epitope conjugate of the present disclosure comprises, as the cancer-targeting polypeptide (CTP), a scFv or a nanobody specific for a Her2 polypeptide present on the surface of a cancer cell; and comprises, as the epitope a CMV peptide epitope. In some cases, the CMV peptide is a peptide of a CMV pp65 polypeptide. In some cases, the CMV peptide epitope is a peptide of a CMV gB polypeptide. In some cases, the CMV peptide epitope has the amino acid sequence NLVPMVATV (SEQ ID NO:487) and has a length of 9 amino acids.

In some cases, a T-Cell-MP-epitope conjugate of the present disclosure comprises, as the CTP, a scFv or a nanobody specific for a MUC1 polypeptide present on the surface of a cancer cell; and comprises, as the epitope a CMV peptide epitope. In some cases, the CMV peptide epitope is a peptide of a CMV pp65 polypeptide. In some cases, the CMV peptide is a peptide of a CMV gB polypeptide. In some cases, the CMV peptide has the amino acid sequence NLVPMVATV (SEQ ID NO:487) and has a length of 9 amino acids.

In some cases, a T-Cell-MP-epitope conjugate of the present disclosure comprises, as the CTP, a scFv or a nanobody specific for a WT1 polypeptide present on the surface of a cancer cell; and comprises, as the epitope a CMV peptide epitope. In some cases, the CMV peptide epitope is a peptide of a CMV pp65 polypeptide. In some cases, the CMV peptide epitope is a peptide of a CMV gB polypeptide. In some cases, the CMV peptide epitope has the amino acid sequence NLVPMVATV (SEQ ID NO:487) and has a length of 9 amino acids.

In some cases, a T-Cell-MP-epitope conjugate of the present disclosure comprises, as the CTP, a scFv or a nanobody specific for a mesothelin polypeptide present on the surface of a cancer cell; and comprises, as the epitope a CMV peptide epitope. In some cases, the CMV peptide epitope is a peptide of a CMV pp65 polypeptide. In some cases, the CMV peptide epitope is a peptide of a CMV gB polypeptide. In some cases, the CMV peptide epitope has the amino acid sequence NLVPMVATV (SEQ ID NO: 487) and has a length of 9 amino acids.

In some cases, a T-Cell-MP-epitope conjugate of the present disclosure comprises, as the CTP, a scFv or a nanobody specific for a CD19 polypeptide present on the surface of a cancer cell; and comprises, as the epitope a CMV peptide epitope. In some cases, the CMV peptide epitope is a peptide of a CMV pp65 polypeptide. In some cases, the CMV peptide epitope is a peptide of a CMV gB polypeptide. In some cases, the CMV peptide epitope has the amino acid sequence NLVPMVATV (SEQ ID NO: 487) and has a length of 9 amino acids.

In some cases, a T-Cell-MP-epitope conjugate of the present disclosure comprises, as the CTP, a scFv or a nanobody specific for a BCMA polypeptide present on the surface of a cancer cell; and comprises, as the epitope a CMV peptide epitope. In some cases, the CMV peptide epitope is a peptide of a CMV pp65 polypeptide. In some cases, the CMV peptide epitope is a peptide of a CMV gB polypeptide. In some cases, the CMV peptide epitope has the amino acid sequence NLVPMVATV (SEQ ID NO: 487) and has a length of 9 amino acids.

In some cases, a T-Cell-MP-epitope conjugate of the present disclosure comprises, as the CTP, a scFv or a nanobody specific for a MUC16 polypeptide present on the surface of a cancer cell; and comprises, as the epitope a CMV peptide epitope. In some cases, the CMV peptide epitope is a peptide of a CMV pp65 polypeptide. In some cases, the CMV peptide epitope is a peptide of a CMV gB polypeptide. In some cases, the CMV peptide epitope has the amino acid sequence NLVPMVATV (SEQ ID NO: 487) and has a length of 9 amino acids.

### 9 Payloads—Drug And Other Conjugates

A polypeptide chain of a T-Cell-MP can comprise an attached payload such as a therapeutic (e.g., a small molecule drug or therapeutic) a label (e.g., a fluorescent label or radio label), or other biologically active agent that is linked (e.g., covalently attached) to the polypeptide chain at a chemical conjugation site. For example, where a T-Cell-MP comprises an Fc polypeptide, the Fc polypeptide may comprise a covalently linked payload molecule that treats a cancer, infectious disease, or an autoimmune disease, or is an agent that relieves a symptom of such diseases.

A payload can be linked directly or indirectly to a chemical conjugation site that is part of the polypeptide chain of a T-Cell-MP of the present disclosure (e.g., to scaffold such as an Ig Fc polypeptide). Direct linkage can involve linkage directly to an aa side chain. Indirect linkage can be linkage via a cross-linker, such as a bifunctional cross cross-linker. A payload can be linked to a T-Cell-MP by any acceptable chemical linkage including, but not limited to a thioether bond, an amide bond, a carbamate bond, a disulfide bond, or an ether bond formed by reaction with a crosslinking agent.

Crosslinkers (crosslinking agents) include cleavable cross-linkers and non-cleavable cross-linkers may be used to link payloads and/or targeting sequences to a T-Cell-MP polypeptide. The crosslinkers may comprise reactive NHS, maleimide, iodoacetate, bromoacetate and/or carboxyl groups. In some cases, the cross-linker is a protease-cleavable cross-linker. Suitable cross-linkers may include, for example, peptides (e.g., from 2 to 10 aas in length; e.g., 2, 3, 4, 5, 6, 7, 8, 9, or 10 aas in length), alkyl chains, poly(ethylene glycol), disulfide groups, thioether groups, acid labile groups, photolabile groups, peptidase labile groups, and esterase labile groups. Non-limiting example of suitable cross-linkers are: N-succinimidyl-[(N-maleimidopropionamido)-tetraethyleneglycol] ester (NHS-PEG4-maleimide); N-succinimidyl 4-(2-pyridyldithio)butanoate (SPDB); N-succinimidyl 4-(2-pyridyldithio)2-sulfobutanoate (sulfo-SPDB); N-succinimidyl 4-(2-pyridyldithio) pentanoate (SPP); N-succinimidyl-4-(N-maleimidomethyl)-cyclohexane-1-carboxy-(6-amidocaproate) (LC-SMCC); κ-maleimidoundecanoic acid N-succinimidyl ester (KMUA); γ-maleimide butyric acid N-succinimidyl ester (GMBS); ε-maleimidocaproic acid N-hydroxysuccinimide ester (EMCS); m-maleimide benzoyl-N-hydroxysuccinimide ester (MBS); N-(α-maleimidoacetoxy)-succinimide ester (AMAS); succinimidyl-6-(β-maleimidopropionamide)hexanoate (SMPH); N-succinimidyl 4-(p-maleimidophenyl)butyrate (SMPB); N-(p-maleimidophenyl)isocyanate (PMPI); N-succinimidyl 4(2-pyridylthio)pentanoate (SPP); N-succinimidyl(4-iodo-acetyl)aminobenzoate (SIAB); 6-maleimidocaproyl (MC); maleimidopropanoyl (MP); p-aminobenzyloxycarbonyl (PAB); N-succinimidyl 4-(maleimidomethyl)cyclohexanecarboxylate (SMCC); N-succinimidyl-4-(N-maleimidomethyl)-cyclohexane-1-carboxy-(6-amidocaproate), a “long chain” analog of SMCC (LC-SMCC); 3-maleimidopropanoic acid N-succinimidyl ester (BMPS); N-succinimidyl iodoacetate (SIA); N-succinimidyl bromoacetate (SBA); and N-succinimidyl 3-(bromoacetamido)propionate (SBAP).

T-Cell-MP-payload conjugates may be formed by reaction of a T-Cell-MP polypeptide (e.g., an Ig Fc polypeptide of a T-Cell-MP) with a crosslinking reagent to introduce 1-10 reactive groups. The polypeptide is then reacted with the molecule to be conjugated (e.g., a thiol-containing payload drug, label or agent) to produce a T-Cell-MP-payload conjugate. For example, where a T-Cell-MP of the present disclosure comprises an Ig Fc polypeptide, the conjugate can be of the form (A)-(L)-(C), where (A) is the polypeptide chain comprising the Ig Fc polypeptide; where (L), if present, is a cross-linker; and where (C) is a payload. (L), if present, links (A) to (C). In some cases, the T-Cell-MP includes an Ig Fc polypeptide sequence that comprises one or more (e.g., 2, 3, 4, 5, or more than 5) molecules of a payload. Introducing payloads into a T-Cell-MP using an excess of crosslinking agents can result in multiple molecules of payload being incorporated into the T-Cell-MP.

Suitable payloads (e.g., drugs) include virtually any small molecule (e.g., less than 2,000 Daltons in molecular weight) approved by the U.S. Food and Drug Administration, and/or listed in the 2020 U.S. Pharmacopeia or National Formulary. In an embodiment, those drugs are less than 1,000 molecular weight. Suitable drugs include antibiotics, chemotherapeutic (antineoplastic), anti-fungal, or anti-helminth agents and the like (e.g., sulfasalazine, azathioprine, cyclophosphamide, leflunomide; methotrexate, antimalarials, D-penicillamine, cyclosporine). Suitable chemotherapeutics may be alkylating agents, cytoskeletal disruptors (taxanes), epothilone, histone deacetylase inhibitors, topoisomerase I inhibitors, topoisomerase II inhibitors, kinase inhibitors, nucleotide analog or precursor analogs, peptide antineoplastic antibiotics (e.g. bleomycin or actinomycin), platinum-based agents, retinoids, or vinca alkaloids. Suitable drugs also include non-steroidal anti-inflammatory drugs and glucocorticoids, and the like. Suitable chemotherapeutics also include alkylating agents, cytoskeletal disruptors (taxanes), epothilone, histone deacetylase inhibitors, topoisomerase I inhibitors, topoisomerase II inhibitors, kinase inhibitors, nucleotide analog or precursor analogs, peptide antineoplastic antibiotics (e.g. bleomycin or actinomycin), platinum-based agents, retinoids, or vinca alkaloids.

In an embodiment, the payload is selected from the group consisting of: biologically active agents or drugs, diagnostic agents or labels, nucleotide or nucleoside analogs, nucleic acids or synthetic nucleic acids (e.g., antisense nucleic acids, small interfering RNA, double stranded (ds)DNA, single stranded (ss)DNA, ssRNA, dsRNA), toxins, liposomes (e.g., incorporating a chemotherapeutic such as 5-fluorodeoxyuridine), nanoparticles (e.g., gold or other metal bearing nucleic acids or other molecules, lipids, particles bearing nucleic acids or other molecules), and combinations thereof.

In an embodiment, the payload is selected from biologically active agents or drugs selected independently from the group consisting of: therapeutic agents (e.g., drugs or prodrugs), chemotherapeutic agents, cytotoxic agents, antibiotics, antivirals (e.g., remdesivir), cell cycle synchronizing agents, ligands for cell surface receptor(s), immunomodulatory agents (e.g., immunosuppressants such as cyclosporine), pro-apoptotic agents, anti-angiogenic agents, cytokines, chemokines, growth factors, proteins or polypeptides, antibodies or antigen binding fragments thereof, enzymes, proenzymes, hormones and combinations thereof.

In an embodiment the payload is a label, selected independently from the group consisting of photo detectable labels (e.g., dyes, fluorescent labels, phosphorescent labels, and luminescent labels), contrast agents (e.g., iodine or barium containing materials), radiolabels, imaging agents, paramagnetic labels/imaging agents (gadolinium containing magnetic resonance imaging labels), ultrasound labels and combinations thereof. In some embodiments, the payload is a label that is or includes a radioisotope. Examples of radioisotopes or other labels include, but are not limited to, ³H, ¹¹C, ¹⁴C, ¹⁵N, ³⁵S, ¹⁸F, ³²P, ³³P, ⁶⁴Cu, ⁶⁸Ga, ⁸⁹Zr, ⁹⁰Y, ⁹⁹Tc, ¹²³I, ¹²⁴I, ¹²⁵I, ¹³¹I, ¹¹¹In, ¹³¹In, ¹⁵³Sm, ¹⁸⁶Re, ¹⁸⁸Re, ²¹¹At, and ¹⁵³Pb.

### II. Nucleic Acids

The present disclosure provides a nucleic acid comprising a nucleotide sequence encoding a T-Cell-MP or more than one T-Cell-MP (e.g., a pair of T-Cell-MPs that form an interspecific heterodimer). The individual T-Cell-MPs of heteromer (e.g., an interspecific pair forming a heteroduplex) may be encoded in separate nucleic acids. Alternatively, the T-Cell-MPs of a heteromeric T-Cell-MP (e.g., an interspecific pair) may also be encoded in a single nucleic acid. Such nucleic acids include those comprising a nucleotide sequence encoding a T-Cell-MP having chemical conjugation sites (e.g., cysteine residues) that are provided in the MHC-H, β2M or scaffold polypeptide sequences of the T-Cell-MP, or into any linker (e.g., an L3 linker) joining those polypeptide sequences.

A. Nucleic Acids Encoding Unconjugated T-Cell-MPs

The present disclosure provides nucleic acids comprising nucleotide sequences encoding an unconjugated T-Cell-MP that may form higher order complexes (e.g., duplexes). The nucleotide sequences encoding an unconjugated T-Cell-MP may be operably linked to transcriptional control elements, e.g., promoters, such as promoters that are functional in a eukaryotic cell, where the promoter can be a constitutive promoter or an inducible promoter. As noted above, in some cases, the individual unconjugated T-Cell-MPs form heteromeric complexes (e.g., a heteroduplex T-Cell-MP comprising an interspecific scaffold pair). Heteromeric unconjugated T-Cell-MPs may be encoded in a single polycistronic nucleic acid sequence. Alternatively, heteromeric T cell-MPs may be encoded in separate monocistronic nucleic acid sequences with expression driven by separate transcriptional control elements. Where separate monocistronic sequences are utilized, they may be present in a single vector or in separate vectors.

The present disclosure includes and provides for a nucleic acid sequence encoding an unconjugated T-Cell-MP polypeptide that comprises (e.g., from N-terminus to C-terminus): (i) optionally one or more MOD polypeptide sequences (e.g., two or more MOD polypeptide sequences, such as in tandem, wherein when there are two or more MOD polypeptide sequences they are optionally joined to each other by independently selected L1 linkers); (ii) an optional L2 linker polypeptide sequence joining the one or more MOD polypeptide sequences to a β2M polypeptide sequence; (iii) the β2M polypeptide sequence; (iv) an optional L3 linker polypeptide sequence (e.g., from 10-50 aa in length); (v) a class I MHC-H polypeptide sequence; (vi) an optional L4 linker polypeptide sequence; (vii) a scaffold polypeptide sequence (e.g., an immunoglobulin Fc sequence); (viii) an optional L5 linker polypeptide sequence; and (ix) optionally one or more MOD polypeptide sequence (e.g., two or more MOD polypeptide sequences, such as in tandem, wherein when there are two or more MOD polypeptide sequences they are optionally joined to each other by independently selected L6 linkers); wherein the unconjugated T cell modulatory polypeptide comprises at least one MOD polypeptide sequence (e.g., the MOD(s) of element (i) and/or (ix)); and wherein at least one of the β2M polypeptide sequence, the L3 linker polypeptide sequence, and/or the MHC-H polypeptide sequence comprises a chemical conjugation site for epitope conjugation.

The present disclosure includes and provides for a nucleic acid sequence encoding an unconjugated T-Cell-MP polypeptide that comprises from N- to C-terminus: (i) optionally one or more MOD polypeptide sequences (e.g., two or more MOD polypeptide sequences, such as in tandem, wherein when there are two or more MOD polypeptide sequences they are optionally joined to each other by independently selected L1 linkers); (ii) an optional L2 linker polypeptide sequence; (iii) a β2M polypeptide sequence; (iv) an optional L3 linker polypeptide sequence (e.g., from 10-50 aa in length); (v) a class I MHC-H polypeptide sequence; (vi) an optional L4 linker polypeptide sequence; (vii) a scaffold polypeptide sequence (e.g., an immunoglobulin Fc sequence); (viii) an optional L5 linker polypeptide sequence; and (ix) optionally one or more MOD polypeptide sequence (e.g., two or more MOD polypeptide sequences, such as in tandem, wherein when there are two or more MOD polypeptide sequences they are optionally joined to each other by independently selected L6 linkers); wherein the unconjugated T cell modulatory polypeptide comprises at least one MOD polypeptide sequence (e.g., the MOD(s) of element (i) and/or (ix)); and wherein at least one of the β2M polypeptide sequence, the L3 linker polypeptide sequence, and/or the MHC-H polypeptide sequence comprises a chemical conjugation site for epitope conjugation.

The present disclosure includes and provides for a nucleic acid sequence encoding an unconjugated T-Cell-MP polypeptide that comprises from N- to C-terminus: (i) one or more MOD polypeptide sequences (e.g., two or more MOD polypeptide sequences, such as in tandem, wherein when there are two or more MOD polypeptide sequences they are optionally joined to each other by independently selected L1 linkers); (ii) an optional L2 linker polypeptide sequence; (iii) a β2M polypeptide sequence; (iv) an optional L3 linker polypeptide sequence (e.g., from 10-50 aa in length); (v) a class I MHC-H polypeptide sequence; (vi) an optional L4 linker polypeptide sequence; (vii) a scaffold polypeptide sequence (e.g., an immunoglobulin Fc sequence); (viii) an optional L5 linker polypeptide sequence; and (ix) optionally one or more MOD polypeptide sequence (e.g., two or more MOD polypeptide sequences, such as in tandem, wherein when there are two or more MOD polypeptide sequences they are optionally joined to each other by independently selected L6 linkers); wherein the unconjugated T cell modulatory polypeptide comprises at least one MOD polypeptide sequence (e.g., the MOD(s) of element (i) and/or (ix)); and wherein at least one of the β2M polypeptide sequence, the L3 linker polypeptide sequence, and/or the MHC-H polypeptide sequence comprises a chemical conjugation site for epitope conjugation.

Suitable MHC-H, β2-microglobulin (β2M) polypeptide, and scaffold polypeptides are described above. The MHC-H polypeptide may be a HLA-A, HLA-B, HLA-C, HLA-E, HLA-F, or HLA-G heavy chain. In some cases, the MHC-H polypeptide comprises an amino acid sequence having at least 85% aa sequence identity to the amino acid sequence depicted in any one of FIGS. 3A-3H. In such an embodiment the MHC Class I heavy chain polypeptide may not include a transmembrane anchoring domain and intracellular domain (see, e.g., the MHC-H polypeptides in FIG. 3D). In some cases, the first MHC polypeptide comprises a β2-microglobulin (β2M) polypeptide; and the second MHC polypeptide comprises a MHC Class I heavy chain polypeptide. In some cases, the β2M polypeptide comprises an amino acid sequence having at least about 85% (e.g., at lease about 90%, 95%, 98%, 99%, or even 100%) aa sequence identity to a β2M amino acid sequence depicted in FIG. 4

B. Recombinant Expression Vectors

The present disclosure provides recombinant expression vectors comprising nucleic acid sequence encoding T-Cell-MPs of the present disclosure. In some cases, the recombinant expression vector is a non-viral vector. In some embodiments, the recombinant expression vector is a viral construct, e.g., a recombinant adeno-associated virus construct (see, e.g., U.S. Pat. No. 7,078,387), a recombinant adenoviral construct, a recombinant lentiviral construct, a recombinant retroviral construct, a non-integrating viral vector, etc.

Suitable expression vectors include, but are not limited to, viral vectors (e.g., viral vectors based on vaccinia virus; poliovirus; adenovirus (see, e.g., Li et al., Invest Opthalmol Vis Sci 35:2543 2549, 1994; Borras et al., Gene Ther 6:515 524, 1999; Li and Davidson, PNAS 92:7700 7704, 1995; Sakamoto et al., H Gene Ther 5:1088 1097, 1999; WO 94/12649, WO 93/03769; WO 93/19191; WO 94/28938; WO 95/11984 and WO 95/00655); adeno-associated virus (see, e.g., Ali et al., Hum Gene Ther 9:81 86, 1998, Flannery et al., PNAS 94:6916 6921, 1997; Bennett et al., Invest Opthalmol Vis Sci 38:2857 2863, 1997; Jomary et al., Gene Ther 4:683 690, 1997, Rolling et al., Hum Gene Ther 10:641 648, 1999; Ali et al., Hum Mol Genet 5:591 594, 1996; Srivastava in WO 93/09239, Samulski et al., J. Vir. (1989) 63:3822-3828; Mendelson et al., Virol. (1988) 166:154-165; and Flotte et al., PNAS (1993) 90:10613-10617); SV40; herpes simplex virus; human immunodeficiency virus (see, e.g., Miyoshi et al., PNAS 94:10319 23, 1997; Takahashi et al., J Virol 73:7812 7816, 1999); a retroviral vector (e.g., Murine Leukemia Virus, spleen necrosis virus, and vectors derived from retroviruses such as Rous Sarcoma Virus, Harvey Sarcoma Virus, avian leukosis virus, lentivirus, human immunodeficiency virus, myeloproliferative sarcoma virus, and mammary tumor virus); and the like.

Numerous suitable expression vectors are known to those of skill in the art, and many are commercially available. The following vectors are provided by way of example for eukaryotic host cells: pXT1, pSG5 (Stratagene®), pSVK3, pBPV, pMSG, and pSVLSV40 (Pharmacia). However, any other vector may be used so long as it is compatible with the host cell.

Depending on the host/vector system utilized, any of a number of suitable transcription and translation control elements, including constitutive and inducible promoters, transcription enhancer elements, transcription terminators, etc., may be used in the expression vector (see, e.g., Bitter et al. (1987), Methods in Enzymology, 153:516-544).

Non-limiting examples of suitable eukaryotic promoters (promoters functional in a eukaryotic cell) include those from cytomegalovirus (CMV) immediate early, herpes simplex virus (HSV) thymidine kinase, early and late SV40, long terminal repeats (LTRs) from retrovirus, and mouse metallothionein-I. Selection of the appropriate vector and promoter is well within the level of ordinary skill in the art. The expression vector may also contain a ribosome binding site for translation initiation and a transcription terminator. The expression vector may also include appropriate sequences for amplifying expression.

### III. Methods of Generating and Selecting T-Cell-MPs

The present disclosure provides a method of obtaining T-Cell-MPs (both unconjugated T-Cell-MPs and/or T-Cell-MP-epitope conjugates) including in duplex and other higher order aggregates, which may include one or more wt. MOD polypeptide sequences and/or one or more variant MOD polypeptide sequences that exhibit lower affinity for a Co-MOD compared to the affinity of the corresponding wt. MOD polypeptide sequence for the Co-MOD, the method comprising:
A) generating a T-Cell-MP (or a higher order complex such as a duplex) by introducing into cells or cell-free systems one or more nucleic acids encoding an unconjugated T-Cell-MP or each of the unconjugated T-Cell-MPs that make up a heteromer (e.g., a heterodimeric duplex of unconjugated T-Cell-MPs);
wherein when the T-Cell-MP comprises one or more nascent chemical conjugation sites, the nascent chemical conjugation site may be activated to produce an unconjugated T-Cell-MP with chemical conjugation site (e.g., reacting sulfatase motifs with an FGE to convert a Cys residue to a fGly residue if the cells translating the T-Cell-MP nucleic acids do not express a formylglycine generating enzyme).

The above-mentioned method of generating T-Cell-MPs may further comprise providing one or more nucleic acids encoding the unconjugated T-Cell-MP, including those specifically described in the present disclosure, which may be present in a recombinant expression vector and/or operably linked to a transcriptional control elements such as those functional in a eukaryotic cell. The method may be stopped at this point and the unconjugated T-Cell-MP (e.g., unconjugated duplex T-Cell-MP) that is unpurified (including cell lysates and unpurified media) may be obtained. Alternatively, the unconjugated T-Cell-MP may be purified using, for example, one or more of salt precipitation (e.g., ammonium sulfate), affinity chromatography, and/or size exclusion chromatography, to produce crude (less than 60% by weight), initially refined (at least 60% by weight), partly refined (at least 80% by weight), substantially refined (at least 95% by weight), partially pure or partially purified (at least 98% by weight), substantially pure or substantially purified (at least 99% by weight), essentially pure or essentially purified (at least 99.5% by weight) or purified (at least 99.8%) or highly purified (at least 99.9% by weight) of the unconjugated T-Cell-MP based on the total weight of protein present in the sample may be obtained by purification. Where a T-Cell-MP-epitope conjugate is desired, the method may be continued by reacting anywhere from a crude preparation to a highly purified preparation T-Cell-MP with an epitope presenting molecule as in step B:
B) providing an epitope (e.g., an epitope-presenting peptide) suitable for conjugation with the chemical conjugation site present in the unconjugated T-Cell-MP of step A (e.g., a hydrazinyl or hydrazinyl indole modified peptide for reaction with a formyl glycine of a sulfatase motif or a maleimide containing peptide for reaction with a cysteine residue), and contacting the epitope with the T-Cell-MP (e.g., under suitable reaction conditions) to produce a T-Cell-MP-epitope conjugate.
   The choice of how purified the unconjugated material entered into the reaction needs to be depends on a number of factors including the conjugation reaction and conditions, the potential for side reactions, and the degree to which the final epitope conjugate will need to be purified.

The T-Cell-MP-epitope conjugate (e.g., as a duplex or a higher order complex) may be purified by, for example, salt precipitation, size separation, and/or affinity chromatography, so that it is at least partly refined (at least 80% by weight of protein present in the sample), substantially refined (at least 95% by weight), partially pure or partially purified (at least 98% by weight), substantially pure or substantially purified (at least 99% by weight), essentially pure or essentially purified (at least 99.5% by weight), purified (at least 99.8%), or highly purified (at least 99.9% by weight) of the T-Cell-MP-epitope conjugate based on the total weight of protein present in the sample.

Where it is desirable for a T-Cell-MP or higher order complexes to contain a payload, the payload may be reacted with the unconjugated T-Cell-MP or the T-Cell-MP-epitope conjugate. The selectivity of the epitope and the payload for different conjugation sites may be controlled through the use of orthogonal chemistries and/or control of stoichiometry in the conjugation reactions. In embodiments, linkers (e.g., polypeptides or other bifunctional chemical linkers) may be used to attach the epitope and/or payloads to their conjugation sites. The payload may be a cytotoxic agent that is selected from, for example, maytansinoids, benzodiazepines, taxoids, CC-1065, duocarmycin, a duocarmycin analogs, calicheamicin, dolastatin, a dolastatin analogs, auristatin, tomaymycin, and leptomycin, or a pro-drug of any one of the foregoing. The payload may be a retinoid. When possible, a single purification scheme that removes reagents and other materials present from the conjugation of the epitope and attachment of the payload is employed to minimize loss of the protein.

A variety of cells and cell-free systems may be used for the preparation of unconjugated T-Cell-MPs. As discussed in the section titled “Genetically Modified Host cells,” the cells may be eukaryotic origin, and more specifically of mammalian, primate or even human origin.

The present disclosure provides a method of obtaining an unconjugated T-Cell-MP or T-Cell-MP-epitope conjugate (or their higher order complexes, such as duplexes) comprising one or more wt. MODs and/or variant MODs that exhibit reduced affinity for a Co-MOD compared to the affinity of the corresponding parental wt. MOD for the Co-MOD. Where a variant MOD having reduced affinity is desired, the method can comprise preparing a library of variant MOD polypeptides (e.g., that have at least one insertion, deletion or substitution) and selecting from the library of MOD polypeptides a plurality of members that exhibit reduced affinity for their Co-MOD (such as by BLI as described above). Once a variant MOD is selected a nucleic acid encoding the unconjugated T-Cell-MP including the variant MOD is prepared and expressed. After the unconjugated T-Cell-MP has been expressed it can be purified, and if desired conjugated to an epitope to produce the selected T-Cell-MP-epitope conjugate. The process may be repeated to prepare a library of unconjugated T-Cell-MPs or their epitope conjugates.

The present disclosure provides a method of obtaining a T-Cell-MP-epitope conjugate or its higher order complexes, such as a duplex) that exhibits selective binding to a T cell, the method comprising:
A) generating a library of T-Cell-MP-epitope conjugates (or their higher order complexes) comprising a plurality of members, wherein each member comprises a different variant MOD on the T-Cell-MP-epitope conjugate, wherein the variant MOD differs in amino acid sequence (e.g., by from 1 aa to 10 aas) from its parental wt. MOD, and wherein the T-Cell-MP-epitope conjugate library members further comprise an epitope tag or a fluorescent label), and
B) contacting a T-Cell-MP-epitope conjugate library member with a target T cell expressing on its surface: i) a Co-MOD that binds the parental wt. MOD; and ii) a TCR that binds to the epitope;
C) selecting a T-Cell-MP-epitope conjugate library member that selectively binds the target T cell relative to its binding under the same conditions to a control T cell that comprises: i) the Co-MOD that binds the parental wt. MOD; and ii) a TCR that binds to an epitope other than the epitope present in the T-Cell-MP library member (e.g., choosing the T-Cell-MP-epitope conjugate that has higher avidity or affinity for the target T cell than the control T cell such as by BLI as described above).
   A T-Cell-MP-epitope conjugate library member that is identified as selectively binding to a target T cell may be isolated from the library.

When the T-Cell-MP-epitope conjugate comprises an epitope tag or label, identifying a T-Cell-MP-epitope conjugate selective for a target T cell may comprise detecting the epitope tag or label associated with target and control T cells by using, for example, flow cytometry. While labeled T-Cell-MPs (e.g., fluorescently labeled) do not require modification to be detected, epitope tagged molecules may require contacting with an agent that renders the epitope tag visible (e.g., a fluorescent agent that binds the epitope tag). The affinity/avidity of the T-Cell-MP-epitope conjugate can be determined by measuring the agent or label associated with target and control T cells (e.g., by measuring the mean fluorescence intensity using flow cytometry) over a range of concentrations. The T-Cell-MP-epitope conjugate that binds with the highest affinity or avidity to the target T cell relative to the control T cell is understood to selectively bind to the target T cell.

MOD and Co-MOD pairs, including wt. and variant MOD and Co-MOD pairs, utilized in the methods of obtaining T-Cell-MPs and methods of obtaining a T-Cell-MP-epitope conjugate that exhibits selective binding to a T cell may be selected from: IL-2 and IL-2 receptor; 4-1BBL and 4-1BB; PD-L1 and PD-1; FasL and Fas; TGF-β and TGF-β receptor; CD80 and CD28; CD86 and CD28; OX40L and OX40; ICOS-L and ICOS; ICAM and LFA-1; JAG1 and Notch; JAG1 and CD46; CD70 and CD27; CD80 and CTLA4; and CD86 and CTLA4. Alternatively, they may be selected from IL-2 and IL-2 receptor; 4-1BBL and 4-1BB; PD-L1 and PD-1; FasL and Fas; CD80 and CD28; CD86 and CD28; CD80 and CTLA4; and CD86 and CTLA4. In some cases, the variant MODs present in a T-Cell-MP, which are independently selected, comprise from 1 to 20 aa independently selected sequence variations (e.g., 1, 2, 3, 4, 5, 6, 7, 8, 9, 10, 11, 12, 13, 14, 15, 16, 17, 18, 19, or 20 aa substitutions, deletions, or insertions) compared to the corresponding parental wt. MOD.

A T-Cell-MP (unconjugated T cell-MP or T-Cell-MP-epitope conjugate) may comprise two or more wt. and/or variant MODs. The two or more MODs may comprise the same or different amino acid sequence. The two or more MODs may be on the same T-Cell-MP (e.g., in tandem) of a T cell-MP-duplex. The first of two or more MODs may be on the first T-Cell-MP of a T-Cell-MP duplex and the second of two variant MODs may be on the second T-Cell-MP of the duplex.

### IV. Genetically Modified Host Cells

The present disclosure provides a genetically modified host cell, where the host cell is genetically modified with a nucleic acid of the present disclosure (e.g., a nucleic acid encoding an unconjugated T-Cell-MP that may be operably linked to a promoter). Where such cell express T-Cell-MPs they may be utilized in methods of generating and selecting T-Cell-MPs as discussed in the preceding section.

Suitable host cells include eukaryotic cells, such as yeast cells, insect cells, and mammalian cells. In some cases, the host cell is a cell of a mammalian cell line. Suitable mammalian cell lines include human cell lines, non-human primate cell lines, rodent (e.g., mouse, rat) cell lines, and the like. Suitable mammalian cell lines include, but are not limited to, HeLa cells (e.g., American Type Culture Collection (ATCC) No. CCL-2™), CHO cells (e.g., ATCC Nos. CRL-9618™, CCL-61TM, CRL9096), 293 cells (e.g., ATCC No. CRL-1573™), Vero cells, NIH 3T3 cells (e.g., ATCC No. CRL-1658), Huh-7 cells, BHK cells (e.g., ATCC No. CCL-10™), PC12 cells (ATCC No. CRL-1721™), COS cells, COS-7 cells (ATCC No. CRL1651), RAT1 cells, mouse L cells (ATCC No. CCLI.3), human embryonic kidney (HEK) cells (ATCC No. CRL1573), HLHepG2 cells, and the like.

In some cases, the host cell is a mammalian cell that has been genetically modified such that it does not synthesize endogenous β2M and/or such that it does not synthesize endogenous MHC Class I heavy chains (MHC-H). In addition to the foregoing, host cells expressing formylglycine generating enzyme (FGE) activity are discussed above for use with T-Cell-MPs comprising a sulfatase motif, and such cells may advantageously be modified such that they do not express at least one, if not both, of the endogenous MHC β2M and MHC-H proteins.

### V. Compositions and Formulations

The present disclosure provides compositions and formulations, including pharmaceutical compositions and formulations. Compositions may comprise: a) a T-Cell-MP and b) an excipient. Where the excipient(s) present in a composition or formulation are pharmaceutically acceptable excipients, the composition may be a pharmaceutically composition or formulation. Pharmaceutical compositions or formulations may also be sterile and/or pyrogen free. Some pharmaceutically acceptable excipients are provided below. The present disclosure also provides compositions and formulations, including pharmaceutical compositions, comprising a nucleic acid or a recombinant expression vector, where the nucleic acid or expression nucleic acid encodes all or part of a T-Cell-MP or its higher order complexes (e.g., one T-Cell-MP of a heterodimeric T-Cell-MP duplex).

A. Compositions Comprising T-Cell-MP-Epitope Conjugates

Compositions of the present disclosure may comprise, in addition to a T-Cell-MP, one or more of: a salt, e.g., NaCl, MgCl₂, CaCl₂, KCl, MgSO₄, sodium acetate, sodium lactate, etc.; a buffering agent, (e.g., a Tris buffer, N-(2-Hydroxyethyl)piperazine-N′-(2-ethanesulfonic acid) (HEPES), 2-(N-Morpholino)ethanesulfonic acid (MES), 2-(N-Morpholino)ethanesulfonic acid sodium salt (MES), 3-(N-Morpholino)propanesulfonic acid (MOPS), N-tris[Hydroxymethyl]methyl-3-aminopropanesulfonic acid (TAPS), etc.); a solubilizing agent; a detergent (surfactants), e.g., a non-ionic detergent such as Tween-20, etc.; a protease inhibitor; glycerol; and the like; any or all of which may be in the form of solvates (e.g., mixed ionic salts with water and/or organic solvents), hydrates, or the like.

A pharmaceutically acceptable compositions comprising a T-Cell-MP epitope conjugate may comprise, in addition to the T-Cell-MP, a pharmaceutically acceptable excipient, a variety of which are known in the art and need not be discussed in detail herein. Pharmaceutically acceptable compositions (e.g., injectable formulations) may be sterile and/or free of pyrogens and other materials detrimental to administration to patients or subjects (e.g., lipopolysaccharides). Pharmaceutically acceptable excipients have been amply described in a variety of publications including, for example, “Remington: The Science and Practice of Pharmacy”, 19^{th} Ed. (1995), or latest edition, Mack Publishing Co; A. Gennaro (2000) “Remington: The Science and Practice of Pharmacy,” 20th edition, Lippincott, Williams, & Wilkins; Pharmaceutical Dosage Forms and Drug Delivery Systems (1999) H. C. Ansel et al., eds 7^{th} ed., Lippincott, Williams, & Wilkins; and Handbook of Pharmaceutical Excipients (2000) A. H. Kibbe et al., eds., 3^{rd} ed. Amer. Pharmaceutical Assoc.

A subject pharmaceutical composition may be suitable for administration to a subject, e.g., will generally be sterile. For example, in some embodiments, a subject pharmaceutical composition will be suitable for administration to a human subject, e.g., where the composition is sterile and is free of detectable pyrogens and/or other toxins. A pharmaceutical composition may be suitable for use ex vivo or in vitro (ex vivo treatment of cells) where, for example, it may be contacted with cells and then subsequently removed prior to administration of the cells to a subject.

The T-Cell-MP compositions, including pharmaceutical compositions, may also comprise components, such as mannitol, lactose, starch, magnesium stearate, sodium saccharin, talcum, cellulose, glucose, sucrose, glycerol, magnesium, carbonate, and the like, any or all of which may be pharmaceutical grade.

Compositions may be in the form of aqueous or other solutions, powders, granules, tablets, pills, suppositories, capsules, suspensions, sprays, and the like. The composition may be formulated according to the various routes of administration described below.

Where a T-Cell-MP epitope conjugate of the present disclosure is administered as an injectable (e.g., subcutaneously, intraperitoneally, intramuscularly, and/or intravenously) directly into a tissue, a formulation can be provided as a ready-to-use dosage form, a non-aqueous form (e.g., a reconstitutable storage-stable powder) or an aqueous form, such as liquid composed of pharmaceutically acceptable carriers and excipients. T-Cell-MP formulations may also be provided so as to enhance serum half-life of the subject protein following administration. For example, the T-Cell-MP may be provided in a liposome formulation, prepared as a colloid, or other conventional techniques for extending serum half-life. A variety of methods are available for preparing liposomes, as described in, e.g., Szoka et al. 1980 Ann. Rev. Biophys. Bioeng. 9:467, U.S. Pat. Nos. 4,235,871, 4,501,728 and 4,837,028. The preparations may also be provided in controlled release or slow-release forms.

Other examples of formulations suitable for parenteral administration include those comprising sterile injection solutions, salts, anti-oxidants, bacteriostats, and/or solutes that render the formulation isotonic with the blood of the intended recipient. Such parenteral formulations may also include one or more independently selected suspending agents, solubilizers, thickening agents, stabilizers, and preservatives.

Formulations or pharmaceutical composition comprising a T-Cell-MP can be present in a container, e.g., a sterile container, such as a syringe. The formulations can also be presented in unit-dose or multi-dose sealed containers, such as ampules and vials, any of which may be sterile. The formulation or pharmaceutical compositions may be stored in a sterile freeze-dried (lyophilized) condition requiring only the addition of the sterile liquid excipient, for example, water, for injections, immediately prior to use. Extemporaneous injection solutions and suspensions can be prepared from sterile solutions, powders, granules, and/or tablets that comprise the T-Cell-MP.

The concentration of a T-Cell-MP in a formulation can vary widely (e.g., from less than about 0.1%, usually at or at least about 2% to as much as 20% to 50% or more by weight) and will usually be selected primarily based on fluid volumes, viscosities, and patient-based factors in accordance with the particular mode of administration selected and the patient's needs.

In some cases, a T-Cell-MP is present in a liquid composition. Thus, the present disclosure provides compositions (e.g., liquid compositions, including pharmaceutical compositions) comprising a T-Cell-MP of the present disclosure. The present disclosure also provides a composition comprising: a) a T-Cell-MP of the present disclosure; and b) saline (e.g., 0.9% or about 0.9% NaCl). In some cases, the composition is sterile. The composition may be suitable for administration to a human subject, e.g., where the composition is sterile and is free of detectable pyrogens and/or other toxins. Thus, the present disclosure provides a composition comprising: a) a T-Cell-MP-epitope conjugate; and b) saline (e.g., 0.9% or about 0.9% NaCl), where the composition is sterile and is free of detectable pyrogens and/or other toxins.

B. Compositions Comprising a Nucleic Acid or a Recombinant Expression Vector

The present disclosure provides compositions (e.g., pharmaceutical compositions) comprising a nucleic acid or a recombinant expression vector of the present disclosure (see, e.g., supra) that comprise one or more nucleic acid sequences encoding any one or more T-Cell-MP polypeptide (or each of the polypeptides of a duplex T-Cell-MP multimer such as a heterodimer). Pharmaceutically acceptable excipients are known in the art and have been amply described in a variety of publications, including, for example, A. Gennaro (2000) “Remington: The Science and Practice of Pharmacy”, 20th edition, Lippincott, Williams, & Wilkins; Pharmaceutical Dosage Forms and Drug Delivery Systems (1999) H. C. Ansel et al., eds 7^{th} ed., Lippincott, Williams, & Wilkins; and Handbook of Pharmaceutical Excipients (2000) A. H. Kibbe et al., eds., 3^{rd} ed. Amer. Pharmaceutical Assoc.

A composition of the present disclosure can include: a) one or more nucleic acids or one or more recombinant expression vectors comprising nucleotide sequences encoding a T-Cell-MP polypeptide (or all polypeptides of a T-Cell-MP) of the present disclosure; and b) one or more of: a salt, a buffer, a surfactant, an antioxidant, a hydrophilic polymer, a dextrin, a chelating agent, a suspending agent, a solubilizer, a thickening agent, a stabilizer, a bacteriostatic agent, and a preservative. Suitable buffers include, but are not limited to, (such as N,N-bis(2-hydroxyethyl)-2-aminoethanesulfonic acid (BES), bis(2-hydroxyethyl)amino-tris(hydroxymethyl)methane (BIS-Tris), N-(2-hydroxyethyl)piperazine-N′3-propanesulfonic acid (EPPS or HEPPS), glycylglycine, N-2-hydroxyehtylpiperazine-N′-2-ethanesulfonic acid (HEPES), 3-(N-morpholino)propane sulfonic acid (MOPS), piperazine-N,N′-bis(2-ethane-sulfonic acid) (PIPES), sodium bicarbonate, 3-(N-tris(hydroxymethyl)-methyl-amino)-2-hydroxy-propanesulfonic acid) TAPSO, (N-tris(hydroxymethyl)methyl-2-aminoethanesulfonic acid (TES), N-tris(hydroxymethyl)methyl-glycine (Tricine), tris(hydroxymethyl)-aminomethane (Tris), etc.). Suitable salts include, e.g., NaCl, MgCl₂, KCl, MgSO₄, etc.

A pharmaceutical formulation of the present disclosure can include a nucleic acid or recombinant expression vector of the present disclosure in an amount of from about 0.001% to about 90% (w/w). In the description of formulations, below, “subject nucleic acid or recombinant expression vector” will be understood to include a nucleic acid or recombinant expression vector of the present disclosure. For example, formulation may comprise a subject nucleic acid or subject recombinant expression vector of the present disclosure.

A subject nucleic acid or recombinant expression vector can be admixed, encapsulated, conjugated or otherwise associated with other compounds or mixtures of compounds; such compounds can include, e.g., liposomes or receptor-targeted molecules. A subject nucleic acid or recombinant expression vector can be combined in a formulation with one or more components that assist in uptake, distribution and/or absorption.

A subject nucleic acid or recombinant expression vector composition can be formulated into any of many possible dosage forms such as, but not limited to, tablets, capsules, gel capsules, liquid syrups, soft gels, suppositories, and enemas. A subject nucleic acid or recombinant expression vector composition can also be formulated as a solution or suspensions in aqueous, non-aqueous or mixed media.

A formulation comprising a subject nucleic acid or recombinant expression vector can be a liposomal formulation. As used herein, the term “liposome” includes unilamellar or multilamellar vesicles having an aqueous interior that may contain the composition (e.g., a subject nucleic acid) to be delivered. Cationic liposomes comprise positively charged lipids that can interact with negatively charged DNA molecules to form a stable complex. Liposomes that are pH sensitive or negatively charged are believed to entrap DNA rather than complex with it. Both cationic and noncationic lipids, which may form liposomes, can be used to deliver a subject nucleic acid or recombinant expression vector in vitro, ex vivo, or in vivo.

Liposomes also include “sterically stabilized” liposomes, a term which, as used herein, refers to liposomes comprising one or more specialized lipids that, when incorporated into liposomes, result in enhanced circulation lifetimes relative to liposomes lacking such specialized lipids. Examples of sterically stabilized liposomes include those comprising one or more glycolipids and those comprising lipids derivatized with one or more hydrophilic polymers (e.g., a polyethylene glycol (PEG) moiety). Liposomes and their uses are further described, for example, in U.S. Pat. No. 6,287,860.

Penetration enhancers may be included in compositions comprising a subject nucleic acid or expression vector to effect their efficient delivery of the nucleic acids. In addition to aiding the diffusion of non-lipophilic drugs such as nucleic acids across cell membranes, penetration enhancers also enhance the permeability of lipophilic drugs, such as those that may co-administered with a subject nucleic acid. Penetration enhancers may be classified as belonging to one of five broad categories, i.e., surfactants, fatty acids, bile salts, chelating agents, and non-chelating non-surfactants. Penetration enhancers and their uses are further described, for example, in U.S. Pat. No. 6,287,860.

Compositions and formulations for oral administration include powders or granules, microparticulates, nanoparticulates, suspensions or solutions in water or non-aqueous media, capsules, gel capsules, sachets, tablets, or minitablets. Thickeners, flavoring agents, diluents, emulsifiers, dispersing aids or binders may be desirable. Suitable oral formulations include those in which a subject nucleic acid is administered in conjunction with one or more penetration enhancers surfactants and chelators. Suitable surfactants include, but are not limited to, fatty acids and/or esters or salts thereof, bile acids and/or salts thereof. Suitable bile acids/salts and fatty acids and their uses are further described in U.S. Pat. No. 6,287,860. Also suitable are combinations of penetration enhancers, for example, fatty acids/salts in combination with bile acids/salts. An exemplary suitable combination is the sodium salt of lauric acid, capric acid, and UDCA. Further penetration enhancers include, but are not limited to, polyoxyethylene-9-lauryl ether, and polyoxyethylene-20-cetyl ether. Suitable penetration enhancers also include propylene glycol, dimethyl sulfoxide, triethanolamine, N,N-dimethylacetamide, N,N-dimethylformamide, 2-pyrrolidone and derivatives thereof, tetrahydrofurfuryl alcohol, and AZONE™.

### VI. Methods of Modulating Immune Responses and Treating Diseases and Disorders

T-Cell-MPs and higher order T-Cell-MP complexes (e.g., duplex T-Cell-MP) of the present disclosure are useful for modulating an activity of a T cell, and directly or indirectly modulating the activity of other cells of the immune system. The present disclosure provides methods of modulating an activity of a T cell selective for a epitope (e.g., an “epitope-specific T cell” or an “epitope selective T cell”), the methods generally involving contacting a target T cell with a T-Cell-MP-epitope conjugate or a higher order complex of T-Cell-MP-epitope conjugates (e.g., duplex T-Cell-MP-epitope conjugates) of the present disclosure. A T-Cell-MP-epitope conjugate or its higher order complexes may comprise one or more independently selected MODs that activate an epitope-specific T cell that recognizes a cancer, neoplasm, or pathogen specific antigen (e.g., a tumor, viral or bacterial antigen). In some cases, the activated T cells are cytotoxic T cells (e.g., CD8⁺ cells). Accordingly, the disclosure includes and provides for a method of treating a cancer, neoplasm (e.g., a non-malignant but inoperable tumor), or infection, the method comprising administering to an individual in need thereof an effective amount of a T-Cell-MP-epitope conjugate or a higher order complex thereof that comprises one or more independently selected MODs that activate an epitope-specific T cell that recognizes an epitope specific to the cancer, neoplasm, or pathogen (e.g., a viral or bacterial) antigen. An effective amount of such a T-Cell-MP-epitope conjugate or its higher order complex may be an amount that activates a CD8⁺ T cell specific to the conjugated epitope (e.g., increasing proliferation of the CD8⁺ T cells and/or increasing proliferation related cell signaling, increasing release of their cytotoxic agents such as granzyme, and/or inducing or enhancing release of their cytokines such as interferon γ).

A T-Cell-MP-epitope conjugate or its higher order complexes may also comprise one or more independently selected MODs that inhibit an epitope-specific T cell. Such T-Cell-MP-epitope conjugates are useful for the treatment of disease and disorders where the subject fails to make a sufficient immune response due to, for example, CD8+ T reg cell suppression as may occur in various tumors.

In addition to the foregoing, this disclosure contemplates and provides for the use of T-Cell-MPs for the delivery of MOD polypeptides. The delivery of MODs may be accomplished in epitope selective manner using a T-Cell-MP epitope conjugate, and may also be accomplished in a non-specific manner using an unconjugated T-Cell-MP. The methods of delivering MODs may be utilized in the treatment of diseases or disorders affecting mammalian subjects (e.g., human patients in need of treatment).

A. Methods of Modulating T Cell Activity

The present disclosure provides a method of selectively modulating the activity of a T cell, the method comprising contacting or administering to a subject a T-Cell-MP or a higher order complex thereof, in some instances with a payload. The contacting or administration may occur in vivo where the molecule is administered to an animal (e.g., a mammal such as a human, rat, mouse, dog, cat, pig, horse, or primate), in vitro, or ex vivo; where it may constitute all or part of a method of treating a disease or disorder as discussed further below. The T cells subject to modulation may be, for example, CD8+ T cells, a NK-T cells, and/or T reg cells. In some cases, the T cell is a CD8+ effector T cell.

The present disclosure provides a method of selectively modulating the activity of an epitope-specific T cell. The method comprises contacting the T cell with a T-Cell-MP-epitope conjugate (e.g., in duplex form) bearing an epitope recognized by the epitope-specific T-Cell. The contacting results in selectively modulating the activity of the epitope-specific T cell with the selectivity driven by the epitope and the resultant activation driven, at least in part, by the MOD polypeptide sequence of the T-Cell-MP-epitope conjugate. Contacting T cells with T-Cell-MP-epitope conjugates, or higher order T-Cell-MP complexes (e.g., duplex T-Cell-MP-epitope conjugates) can result in activation or suppression of T cells expressing a TCR specific for the conjugated epitope (an epitope-specific T cell) including induction or suppression of granule dependent and independent responses. Granule-independent responses include, but are not limited to, changes in the number or percentage of epitope-specific CD 8+ T cell (e.g., in a population of cells such as in blood, lymphatics, and/or in a target tissue), changes in the expression of Fas ligand (Fas-L, which can result in activation of caspases and target cell death through apoptosis), and cytokine/chemokine production (e.g., production and release of interferon gamma (IFN-γ). Granule-dependent effector actions include the release of granzymes, perforin, and/or granulysin. Activation of epitope-specific CD8⁺ cytotoxic T cells (e.g., CD8⁺ cytotoxic effector T cells) can result in the targeted killing of, for example, cancer cells and/or infected cells by epitope-specific T cells that recognize the epitope presented by the T-Cell-MP-epitope conjugate (or higher order complex thereof (e.g., a duplex) through granule-dependent and/or independent responses.

Contacting a T-Cell-MP-epitope conjugate or higher order complex thereof (e.g., a duplex) bearing an activating MOD, where the T-Cell-MP is conjugated to an epitope recognize by the TCR of a target T cell (an epitope specific T cell), may result in one or more of: i) proliferation of the epitope-specific T cell (e.g., CD8+ cytotoxic T cells); ii) epitope-specific induction cytotoxic activity; iii) release of one or more cytotoxic molecules (e.g., a perforin; a granzyme; a granulysin) by the epitope specific cytotoxic (e.g., CD8+) T cell. In contrast, contacting a T-Cell-MP-epitope conjugate or higher order complex thereof (e.g., a duplex) bearing an inhibitory MOD, where the T-Cell-MP is conjugated to an epitope recognize by TCR of a target T cell (an epitope specific T cell), may result in one or more of: i) suppression of proliferation and/or reduction the number of the epitope-specific T cells (e.g., CD8+ cytotoxic T cells); ii) epitope-specific suppression of a cytotoxic activity; iii) suppression the production and/or release of one or more cytotoxic molecules (e.g., a perforin; a granzyme; a granulysin) by the epitope specific cytotoxic (e.g., CD8+) T cell. Contacting a T-Cell-MP-epitope conjugate or higher order complex thereof (e.g., a duplex) conjugated to an epitope recognize by TCR of a T cell (an epitope specific T cell) and bearing an inhibitory MOD may also result in one or more of: i) epitope-specific inhibition autoreactive T cell; or ii) induction of epitope specific CD8+ T regulatory cells; and the like.

In some cases, a T-Cell-MP-epitope conjugate (or higher order complex thereof (e.g., a duplex) comprises a cancer epitope and it induces a CD8+ T cell response (e.g., a cytotoxic CD8+ T cell response to a cancer cell). In some cases, a T-Cell-MP-epitope conjugate (or higher order complex thereof (e.g., a duplex) comprises an epitope of an infectious agent, and it activates a CD8+ T cell response (e.g., a cytotoxic CD8+ T cell response) to a cell expressing an antigen of an infectious agent/pathogen.

The present disclosure provides a method of increasing the proliferation (e.g., proliferation rate) and/or the total number of CD 8+ effector T cells in an animal or tissue that are specific to the epitope presented by a T-Cell-MP epitope conjugate or higher order complex thereof (e.g., a duplex) bearing an activating MOD such as IL-2. A method of increasing T cell proliferation or numbers comprises contacting (e.g., in vitro, in vivo, or ex vivo) T cells with a T-Cell-MP-epitope conjugate or higher order a complex thereof. Contacting may occur, for example, by administering to a subject in one or more doses a T-Cell-MP-epitope conjugate). The contacting or administering may increase the number of CD8+ effector T cells having a TCR capable of binding the epitope present in the T-Cell-MP-epitope conjugate relative to the number (e.g., total number or percentage) of T cells present in a tissue (e.g., in a population of cells such as in blood, lymphatics, and/or in a target tissue such as a tumor). For example, the absolute or relative number of CD 8+ effector T cells specific to the epitope presented by the T-Cell-MP-epitope conjugate or its higher order complex (e.g., duplex) can be increased by at least 5%, at least 10%, at least 20%, at least 30%, at least 40%, at least 50%, least 75%, at least 100%, at least 2-fold, at least 2.5-fold, at least 5-fold, at least 10-fold, or more than 10-fold following one or more contacts with doses or administrations of the T-Cell-MP-epitope conjugate or a higher order complex thereof. The increase may be calculated relative the CD8+ T cell numbers present prior to the contacting or administrations, or relative to the population of T cells present in a sample (e.g., a sample of blood or tissue) that has not been contacted with the T-Cell-MP-epitope conjugate or is higher order complex.

The present disclosure provides a method of increasing granule-dependent and/or granule-independent responses of epitope-specific CD 8+ T cell comprising contacting or administering (e.g., in vitro, in vivo, or ex vivo) T cells with a T-Cell-MP-epitope conjugate or a higher order complex thereof, (e.g., with a CD80, and/or CD86 MOD). The contacting or administering may result in, for example, an increased expression of Fas ligand expression, cytokines/chemokines (e.g., IL-2, IL-4, and/or IL-5), release of interferons (e.g., IFN-γ), release of granzymes, release of perforin, and/or release of granulysin. For example, contacting a CD 8+ effector cell with a T-Cell-MP-epitope conjugate or complex thereof (e.g., a duplex) presenting epitope-specific to the effector cell can increase one or more of Fas ligand expression, interferon gamma (IFN-γ) release, granzyme release, perforin release, and/or granulysin release by at least 5%, at least 10%, at least 20%, at least 30%, at least 40%, at least 50%, least 75%, at least 100%, at least 2-fold, at least 2.5-fold, at least 5-fold, at least 10-fold, or more than 10-fold. The increase may be calculated relative the level of expression or release prior to the contacting or administrations, or relative to the population of T cells present in a sample (e.g., a sample of blood or tissue) that has not been contacted with the T-Cell-MP-epitope conjugate or a complex thereof.

Dysregulation of CD8⁺ T reg cells and self-reactive CD8⁺ effector T cells have both been associated with the pathogenesis of autoimmune diseases including, but not limited to, multiple sclerosis, Rasmussen's encephalitis, paraneoplastic syndromes, systemic sclerosis (SSc), Grave's disease (GD), systemic lupus erythematosus (SLE), aplastic anemia (AA), and vitiligo (see e.g., Pilli et al, Frontiers in Immunology, Article 652, vol. 8, June 2017; Coppieters et al, J. Exp. Med. Vol. 209 No. 1 51-60 (2012); Han et al., PNAS (USA), 110(32):13074-13078 (2013) and Pellegrino et al. PLOS ONE, https://doi.org/10.1371/journal.pone.0210839 Jan. 16, (2019). Deng et al, has reviewed the epigenetic role of CD8+ T cell in autoimmune diseases (see Deng et al, Frontiers in Immunology, Article 856, vol. 10, April 2019). CD8+ effectors may also promote autoimmune diseases via dysregulated secretion of inflammatory cytokines, skewed differentiation profiles, inappropriate apoptosis, or induction of effector T cells functions directed against target cells. In some cases, a T-Cell-MP-epitope conjugate or a higher order complex thereof (e.g., duplex) presenting a self-epitope (e.g., an antigenic determinate of a self-antigen) may reduce the activity of an autoreactive CD8+ effector T cells by direct interaction with the cell. Contacting such T-Cell-MP-epitope conjugates presenting a self-antigen and one or more independently selected inhibitory MODs (e.g., PDL1 and/or FasL) with an autoreactive CD8+ effector T cell may be employed as a means to block autoimmune disease by regulating (e.g., reducing) the release of proinflammatory molecules by such T cells and/or by eliminating self-reactive cells.

Where it is desirable to reduce the activity of epitope-specific T cells (e.g., where they are directed against a self-antigen) they may be contacted with T-Cell-MP-epitope conjugates or complexes thereof (e.g., duplexes) presenting the epitope and bearing MODs that modulate their epitope-specific response. Modulation of the cytotoxic CD8+ T cells by T-Cell-MP-epitope conjugates and their higher order complexes may result in, but is not limited to, one or more of: i) suppression of FasL expression by the T cell; ii) suppression of chemokine and/or cytokine release (e.g., IFN-γ); and/or iii) suppression of cytotoxin (e.g., a perforin; a granzyme; a granulysin) synthesis or release. The disclosure includes and provides for a method of reducing (e.g., in vivo, in vitro, or ex vivo) effector T cell activity in an epitope-specific manner, such as where the T cell is directed to a self-antigen. For example, the absolute or relative number of CD 8+ effector T cells specific to the epitope presented by the T-Cell-MP-epitope conjugate or its higher order complexes (e.g., duplex) can be decreased by at least 5%, at least 10%, at least 20%, at least 30%, at least 40%, at least 50%, or at least 75%, following one or more contacts with doses or administrations of the T-Cell-MP-epitope conjugate or a higher order complex thereof.” The method comprises contacting (such as by administering to a subject) an epitope-specific T cell with one or more doses of a T-Cell-MP-epitope conjugate or a higher order complex thereof presenting the epitope and bearing a MOD (e.g., TGF-β). The contacting or administering resulting in a reduction in one or more of: (i) FasL expression by the T cell; ii) suppression of chemokine and/or cytokine release (e.g., IFN-γ); and iii) suppression of cytotoxin (e.g., a perforin; a granzyme; a granulysin) synthesis or release by at least 5%, at least 10%, at least 20%, at least 30%, at least 40%, at least 50%, least 75%, at least 100%, at least 2-fold, at least 2.5-fold, at least 5-fold, at least 10-fold, or more than 10-fold. The change may be calculated relative the level of expression or release prior to the contacting or administrations, or relative to the population of T cells present in a sample (e.g., a sample of blood or tissue) that has not been contacted with the T-Cell-MP-epitope conjugate or a higher order complex thereof.

In other instances, a T-Cell-MP-epitope conjugate, particularly when presenting one or more IL-6 MODs, or a higher order complex thereof presenting an antigen (e.g., an epitope of a self-antigen) may interact with and increase the number or activity of CD8+ regulatory T cells (CD8+ T regs, characterized e.g., as CD8+ FOXP3+ or CD8+ FOXP3+CD25+) specific to the epitope. Various CD8⁺ T reg subsets function by, for example, secreting cytokines and chemokines, including IL-10, TGF-β, IL-16, IFN-γ and chemokine (C-C motif) ligand 4 (CCL4), and thereby suppressing the activity of effector T cells and potentially the activity of CD4+ T cells such as by the action of any of those cytokines. CD8⁺ T regs may also inhibit T cell function through cell-to-cell contact in which surface proteins such as TGF-β and cytotoxic T-lymphocyte associated protein 4 (CTLA-4) act on the T effector cell. See e.g., Yu et al. Oncol. Lett 15(6):8187-8194 (2018).

The present disclosure provides methods of increasing the number (proliferation) of epitope-specific CD8⁺ T regs directed to a self-antigen and/or the release of one or more of IL-10, TGF-β, IL-16, IFN-γ and CCL4 and thereby suppressing immune/autoimmune responses. One method of increasing the number of self-antigen specific CD8+ T regs (e.g., in a subject) comprises contacting (in vitro, ex vivo, or in vivo such as by administering to a subject) an epitope-specific T cell with one or more doses of a T-Cell-MP-epitope conjugate or a higher order complex thereof presenting the self-epitope and bearing a MOD stimulatory to CD8⁺ T reg proliferation (e.g., IL-6, see e.g., Nakagawa et al., International Immunology, Vol. 22, No. 2, pp. 129-139, (2009)), where the contacting increases proliferation of CD8+ T regs by at least 5%, at least 10%, at least 20%, at least 30%, at least 40%, at least 50%, least 75%, at least 100%, at least 2-fold, at least 2.5-fold, at least 5-fold, at least 10-fold, or more than 10-fold relative to the number of CD8⁺ T regs present in a sample (e.g., a sample of blood or tissue) that has not been contacted with the T-Cell-MP or T-Cell-MP complex.

The present disclosure also provides methods of increasing one or more of IL-10, TGF-β, IL-16, IFN-γ and CCL4 and thereby suppressing immune/autoimmune responses. The method comprising contacting (in vitro, ex vivo, or in vivo such as by administering to a subject) an epitope-specific T cell with one or more doses of a T-Cell-MP-epitope conjugate or a higher order complex thereof (e.g., a duplex) presenting the antigen and bearing a MOD stimulatory to CD8⁺ T reg cells. The contacting increases the release at least one of IL-10, TGF-β, IL-16, IFN-γ and CCL4 by at least 5%, at least 10%, at least 20%, at least 30%, at least 40%, at least 50%, least 75%, at least 100%, at least 2-fold, at least 2.5-fold, at least 5-fold, at least 10-fold, or more than 10-fold relative to the amounts prior to the contacting or relative to the population of T cells present in a sample (e.g., a sample of blood or tissue) that has not been contacted with the T-Cell-MP or T-Cell-MP complex.

Where it is desirable to eliminate epitope-specific CD8+ effectors (e.g., where they are directed against a self-antigen) they may be contacted with a T-Cell-MP-epitope conjugate or a higher order complex thereof (e.g., a duplex) presenting the epitope. The T-Cell-MP may comprise one or more MODs that lead to apoptosis and/or comprise an Ig Fc region facilitating antibody-dependent cell-mediated cytotoxicity (ADCC) and/or complement-dependent cytotoxicity (CDC) even in the absence of a MOD polypeptide sequences (e.g., a MOD-less T-Cell-MP with a wt. Ig Fc scaffold). Apoptosis may occur, for example, when the T-Cell-MP-epitope conjugate or a higher order complex thereof (e.g., a duplex) comprises both an epitope (e.g., a self-antigen) and a MOD such as FasL that induces FAS mediated apoptosis. Elimination of epitope-specific T cells may also occur as a result of antibody-dependent cell-mediated cytotoxicity (ADCC) and/or complement-dependent cytotoxicity (CDC) where the T-Cell-MP-epitope conjugate or a higher order complex thereof (e.g., a duplex) presents an epitope and comprises an immunoglobulin Fc polypeptide with wt. or enhanced ADCC and/or CDC functionality. Accordingly, this disclosure includes and provides for a method of eliminating (e.g., in vivo, in vitro, or ex vivo) effector T cells in an epitope-specific manner, such as where the T cell is directed to a self-antigen. The method comprises contacting (such as by administering to a subject in vivo, or to cells in vitro or ex vivo) an epitope-specific T cell with one or more doses of a T-Cell-MP-epitope conjugate or a higher order complex thereof (e.g., a duplex) presenting the epitope and bearing a MOD that can induce T cell apoptosis, such as TNF or Fas-L (resulting in Fas receptor (CD95) mediated cell death), and/or an Ig Fc region with ADCC or CDC activity. The contacting or administering resulting elimination of at least 5%, at least 10%, at least 20%, at least 30%, at least 40%, at least 50%, at least 75%, at least 80%, at least 85%, at least 90%, at least 95%, or 100% of the CD8+ cells specific to the epitope presented by the T-Cell-MP-epitope conjugate or a higher order complex thereof (e.g., a duplex). The change may be calculated relative the number of T cells present in a sample (e.g., a sample of blood or tissue) prior to the contacting (administration) of the T-Cell-MP-epitope conjugate or a higher order complex thereof (e.g., a duplex), or a sample that has not been contacted with the T-Cell-MP-epitope conjugate or a higher order complex thereof (e.g., a duplex).

In addition to epitope-restricted modulation of T cells, the present disclosure also provides a method of modulating the activity T cells (and other cells of the immune system) by providing immunomodulatory polypeptides in a manner unrestricted by a specific epitope. The method comprises administering to a subject or otherwise contacting cells of the immune system with an unconjugated T-Cell-MP or higher order complex thereof (e.g., a duplex T-Cell-MP) where the T-Cell-MP bears one or more MODs, or a nucleic acid encoding such an unconjugated T-Cell-MP. For example, an unconjugated T-Cell-MP bearing IL-2 and/or CD80 MODs may be utilized to effectively provide those cytokines to a subject or patient in need thereof in an epitope independent manner.

B. Methods of Selectively Delivering a MOD (Costimulatory Polypeptide)

The present disclosure provides a method of delivering a MOD (a costimulatory polypeptide) such as IL-2, 4-1BBL, CD-80, CD-86, Fas-L, PD-L1, or a reduced-affinity variant of any thereof (e.g., a PD-L1 and/or an IL-2 variant disclosed herein) to a selected T cell or a selected T cell population having a TCR specific for a given epitope. The method comprises contacting (such as by administration to a subject) a population of T cells with a T-Cell-MP-epitope conjugate or a higher order complex thereof (e.g., a duplex). The population of T cells can be a mixed population that comprises: i) the target T cell with a TCR specific to a target epitope; and ii) non-target T cells that are not specific for the target epitope (e.g., T cells that are specific for epitope(s) other than the epitope to which the epitope-specific T cell binds). The epitope-specific T cell is specific for the epitope present in and presented by the T-Cell-MP-epitope conjugate or a higher order complex thereof and binds to the peptide MHC complex provided by the T-Cell-MP epitope conjugate, thereby selectively delivering the MODs present in the T-Cell-MP-epitope conjugate to the target T cell(s). The contacting or administration may be conducted in vitro, ex vivo, or in vivo, and may constitute all or part of a method of treatment. Thus, for example, the present disclosure provides a method of delivering a costimulatory polypeptide such as PD-L1, or a reduced-affinity variant of a naturally occurring costimulatory polypeptide such as a PD-L1 variant disclosed herein, or a combination of both, selectively to a target T cell, which form part of a treatment of a disease or disorder.

By way of example, a T-Cell-MP-epitope conjugate or a higher order complex thereof (e.g., a duplex) is contacted with a population of T cells comprising: i) a target T cell(s) that is/are specific for the epitope present in the epitope conjugate; and ii) a non-target T cell(s), e.g., a T cell(s) that is specific for a second epitope(s) that is not the epitope present in the epitope conjugate. Contacting the population results in selective delivery of the MOD(s) or reduced-affinity variant MOD(s) to the target T cell. Less than 50%, less than 40%, less than 30%, less than 25%, less than 20%, less than 15%, less than 10%, less than 5%, or less than 4%, 3%, 2% or 1%, of the T-Cell-MP-epitope conjugate or higher order complex there of (e.g., duplex T-Cell-MP) may bind to non-target T cells and, as a result, the MOD(s) is/are selectively delivered to target T cell (and accordingly, substantially not delivered to the non-target T cells).

In some cases, the population of T cells to which the MOD(s) and/or variant MOD(s) is/are delivered is present in vitro or ex vivo, and a biological response (e.g., T cell activation, expansion, and/or phenotypic differentiation) of the target T cell population to the T-Cell-MP-epitope conjugate or a higher order complex thereof (e.g., a duplex) is elicited in the context of an in vitro or ex vivo setting. For example, a mixed population of T cells can be obtained from an individual and can be contacted with the T-Cell-MP-epitope conjugate or a higher order complex thereof (e.g., a duplex) in vitro or ex vivo. Such contacting can comprise single or multiple exposures of the population of T cells to a defined dose(s) and/or exposure schedule(s). In some cases, said contacting results in selectively binding/activating and/or expanding target T cells within the population of T cells, and results in generation of a population of activated and/or expanded target T cells. As an example, a mixed population of T cells can be peripheral blood mononuclear cells (PBMC). For example, PBMCs from a patient can be obtained by standard blood drawing and PBMC enrichment techniques before being exposed to 0.1-1000 nM of a T-Cell-MP-epitope conjugate or a higher order complex thereof (e.g., a duplex) under standard lymphocyte culture conditions. At time points before, during, and after exposure of the mixed T cell population at a defined dose and schedule, the abundance of target T cells in the in vitro culture can be monitored by specific peptide-MHC multimers, phenotypic markers, and/or functional activity (e.g. cytokine ELISpot assays). In some cases, upon achieving an optimal abundance and/or phenotype of antigen specific cells in vitro, all or a portion of the population of activated and/or expanded target T cells is administered to an individual (e.g., the individual from whom the mixed population of T cells was obtained as a treatment for a disease of disorder).

For example, a mixed population of T cells is obtained from an individual and is contacted with a T-Cell-MP-epitope conjugate or a higher order complex thereof (e.g., a duplex) in vitro. Such contacting, which can comprise single or multiple exposures of the T cells to a defined dose(s) and/or exposure schedule(s) in the context of in vitro cell culture, can be used to determine whether the mixed population of T cells includes T cells that are specific for the epitope presented by the T-Cell-MP-epitope conjugate or higher order complex. The presence of T cells that are specific for the epitope of the T-Cell-MP or higher order complex can be determined by assaying a sample comprising a mixed population of T cells, which population of T cells comprises T cells that are not specific for the epitope (non-target T cells) and may comprise T cells that are specific for the epitope (target T cells). Known assays can be used to detect activation and/or proliferation of the target T cells, thereby providing an ex vivo assay that can determine whether a particular T-Cell-MP-epitope conjugate or a higher order complex thereof possesses an epitope that binds to T cells present in the individual, and thus whether the epitope conjugate has potential use as a therapeutic composition for that individual. Suitable known assays for detection of activation and/or proliferation of target T cells include, e.g., flow cytometric characterization of T cell phenotype and/or antigen specificity and/or proliferation. Such an assay to detect the presence of epitope-specific T cells, e.g., a companion diagnostic, may further include additional assays (e.g. effector cytokine ELISpot assays) and/or appropriate controls (e.g. antigen-specific and antigen-nonspecific multimeric peptide-HLA staining reagents) to determine whether the T-Cell-MP-epitope conjugate or a higher order complex thereof (e.g., a duplex) is selectively binding, modulating (activating or inhibiting), and/or expanding the target T cells. Thus, for example, the present disclosure provides a method of detecting, in a mixed population of T cells obtained from an individual, the presence of a target T cell that binds an epitope of interest, the method comprising: a) contacting in vitro the mixed population of T cells with a T-Cell-MP-epitope conjugate or a higher order complex thereof (e.g., a duplex); and b) detecting modulation (activation or inhibition) and/or proliferation of T cells in response to said contacting, wherein modulation of and/or proliferation of T cells indicates the presence of the target T cell. Alternatively, or in addition, if activation and/or expansion (proliferation) of the desired T cell population is obtained using a T-Cell-MP-epitope conjugate or a higher order complex thereof (e.g., a duplex), then all or a portion of the population of T cells comprising the activated/expanded T cells can be administered back to the individual as a therapy.

In some instances, the population of T cells is in vivo in an individual. In such instances, a method of the present disclosure for selectively delivering one or more costimulatory polypeptides (e.g., IL-2 or PD-L1 or a reduced-affinity IL-2 or PD-L1) to an epitope-specific T cell comprises administering the T-Cell-MP-epitope conjugate or a higher order complex thereof (e.g., duplex) to the individual. In some instances, the epitope-specific T cell to which one or more MOD polypeptide sequences (e.g., a wild-type or reduced-affinity variant of IL-2 or PD-L1) is/are being selectively delivered is a target T cell.

C. Methods of Treatment

The present disclosure provides methods of treatment for a variety of diseases and disorders. The diseases and/or disorders that can be treated include neoplasms (e.g., non-malignant neoplasms), cancers, infections, allergies, transplant (graft) rejection, graft vs host disease, and an autoimmune diseases or disorders. The methods of treatment may comprise administering to an individual an amount of: (i) at least one T-Cell-MP (either unconjugated or as a epitope conjugate) or a higher order complex thereof (e.g., a duplex); or (ii) one or more nucleic acids or expression vectors encoding an unconjugated T-Cell-MP (which may assemble into a higher order complex). Where it is desirable to selectively modulate the activity of an epitope-specific T cell in an individual and thereby effect a method of treating a disease or condition, a T-Cell-MP-epitope conjugate or a higher order complex thereof (e.g., a duplex) may be administered to the individual. Unconjugated T-Cell-MPs or T-Cell-MP-epitope conjugates utilized in methods of treatment may comprise one or more (e.g., two or more) independently selected MOD and/or variant MOD polypeptide sequences.

Where treatment with an immunomodulatory polypeptide that is not restricted to an target epitope specific target cell population is desired, an unconjugated T-Cell-MP or a nucleic acid encoding an unconjugated T-Cell-MP may be administered to a patient or subject. One treatment method of the present disclosure comprises administering to an individual in need thereof one or more nucleic acids (e.g., a recombinant expression vectors) comprising nucleotide sequences encoding an unconjugated T-Cell-MP that may assemble into a higher order T-Cell-MP complex. Another treatment method of the present disclosure comprises administering to an individual in need thereof one or more mRNA molecules comprising nucleotide sequences encoding an unconjugated T-Cell-MPs of the present disclosure. The diseases and/or disorders that can be treated with unconjugated T-Cell-MPs include those where of the immunomodulatory action of the MOD(s) will enhance or suppress the response of cells bearing cognate co-MODs and thereby produce an immune response that alleviates disease symptoms or cures the underlying disease or disorder.

The present disclosure provides a method of selectively modulating the activity of an epitope-specific T cell in an individual, thereby effecting a treatment, the method comprising administering to the individual an effective amount of a T-Cell-MP-epitope conjugate or a higher order complex thereof (e.g., a duplex), where the administered molecule selectively modulates the activity of the epitope-specific T cell in the individual, thereby treating the disease or disorder in the individual. Thus, the present disclosure provides a treatment method comprising administering to an individual in need thereof an effective amount of a T-Cell-MP-epitope conjugate or a higher order complex thereof sufficient to effect treatment. Administering the T-Cell-MP-epitope conjugate induces an epitope-specific T cell response and may also induce an epitope-non-specific T cell response, where the ratio of the epitope-specific T cell response to the epitope-non-specific T cell response is at least 2:1. In some cases, the ratio of the epitope-specific T cell response to the epitope-non-specific T cell response is at least 5:1. In some cases, the ratio of the epitope-specific T cell response to the epitope-non-specific T cell response is at least 10:1. In some cases, the ratio of the epitope-specific T cell response to the epitope-non-specific T cell response is at least 25:1. In some cases, the ratio of the epitope-specific T cell response to the epitope-non-specific T cell response is at least 50:1. In some cases, the ratio of the epitope-specific T cell response to the epitope-non-specific T cell response is at least 100:1. In some cases, the individual is a human. In some cases, the modulating increases a cytotoxic T cell response to a cancer or infected cell, e.g., a cell expressing a virus or cancer antigen that displays the same epitope displayed by the peptide epitope present in the T-Cell-MP-epitope conjugate. As discussed below, in some cases, the administering is intravenous, subcutaneous, intramuscular, systemic, intralymphatic, distal to a treatment site, local, or at or near a treatment site the doses needed to administer an effective amount of the administered molecule are discussed herein below.

The present disclosure also includes and provides for methods of redirecting a T cell (e.g., a CD8+ effector T cell) directed to a specified epitope (e.g., a specified epitope of a CMV protein) toward a selected cell or tissue. If the patient does not have (or might not have) sufficient T cells directed to the specified epitope the method comprises an initial step of immunizing the patient to be treated with an antigen (e.g., a cytomegalovirus “CMV” protein) that induces T cells specific to the specified epitope (see FIG. 18 top section). The patient, having sufficient T cells specific to the epitope, is treated with a T-Cell-MP conjugated to the specified epitope that further comprises one or more (e.g., two or more) targeting sequences and one or more MODs. See FIG. 18 middle portion “Treatment with T-Cell-MP”. By way of example, the T-Cell-MP conjugated to the specified epitope may comprise as targeting sequences cancer targeting peptides CTP1 and/or CTP2 and a MOD (e.g., stimulatory to a cytotoxic T cell such as a wt. or variant IL-2). The T-Cell-MP-epitope conjugate bearing the targeting sequence will be localized on a target cell or tissue (e.g., a cancer cell as shown in FIG. 18 but any cell or tissue with a specific surface antigen could be targeted). Localization of the T-Cell-MP-conjugated to the specified epitope causes the localization of T-cells specific to the specified epitope, while the signal from the MOD in combination with the signal from the TCR that has now been engaged by the specified epitope presented by the T-Cell-MP, directs the T cells response (e.g., cytotoxic attack of the target cell as shown in the lower section of FIG. 18). The overall process permits the actions of a T cell to be redirected from cells or tissues expressing the specified epitope to cells or tissues that express an antigen the targeting sequence(s) recognize. Where the target is cancerous cells or tissues, it may be advantageous to use heteromeric T-Cell-MPs (e.g., heterodimers) having more than one targeting sequence directed against different antigens of the cancerous cell or tissue (e.g., CTP1 and CTP2 as in FIG. 18) to prevent the cancer's escape from the treatment by loss or mutation of the target antigen.

Any of: (i) an unconjugated T-Cell or higher order complex thereof (e.g. a duplex); (ii) one or more nucleic acids encoding an unconjugated T-Cell MP or a higher order complex thereof (e.g., a homoduplex or heteroduplex); or (iii) a T-Cell-MP-epitope conjugate or a higher order complex thereof (e.g., a homoduplex or heteroduplex) may be administered alone or with one or more additional therapeutic agents or drugs. The therapeutic agents (e.g., antibodies against check point inhibitors such as: anti-PD-1, for example Nivolumab, Cemiplimab, and Pembrolizumab; anti-PDL-1 such as Atezolizumab, Avelumab, or Durvalumab; or anti-CTLA-4, for example Ipilimumab, which, along with others, are further described below) may be administered before, during, or subsequent to T-Cell-MP administration, or the administration of nucleic acids encoding one or more unconjugated T-Cell-MP molecules. When an additional therapeutic agent or drug is administered with a composition or formulation comprising a T-Cell-MP or a higher order complex thereof (e.g., a duplex), or a nucleic acid encoding an unconjugated T-Cell-MP, the therapeutic agent or drug may be administered concurrently with any of those molecules. Alternatively, the therapeutic agents may be co-administered with the T-Cell-MP or nucleic acid as part of a single formulation or composition (e.g., a pharmaceutical composition).

Where the epitope is associated with an allergen the T-Cell-MP-epitope conjugate may be utilized in methods of treating allergic reactions. Where the epitope is associated with an infectious agent (e.g., a virus, bacterium, fungi, protozoan, or helminth), the T-Cell-MP-epitope conjugate may be utilized in methods of treatment or prophylaxis of an infection by a pathogen. Where the epitope is associated with a neoplastic or cancerous cell or tissue (e.g., an epitope of a cancer-associated antigen, neoantigen, or an antigen of a virus known to be associated with a specific cancer such as certain HPV an HBV antigens), the T-Cell-MP-epitope conjugate may be utilized in methods of treating various neoplasms or cancers.

1 Neoplasms and Cancers

Cancers (e.g., malignant neoplasms) and neoplasms (e.g., benign neoplasms or benign tumors) that can be treated with a method of the present disclosure include any neoplasm or cancer that can be targeted with a targeting sequence, including by redirection of T cell action as described above with regard to FIG. 18. Cancers that can be treated with a method of the present disclosure include carcinomas, sarcomas, melanoma, leukemias, and lymphomas. Cancers and neoplasms that can be treated with a method of the present disclosure include solid tumors. Cancers that can be treated with a method of the present disclosure include metastatic cancers.

In some cases, a T-Cell-MP-epitope conjugate or a higher order complex thereof (e.g., a duplex) comprises (i) a cancer-specific epitope (e.g. a cancer-associated antigen), and (ii) one or more independently selected activating MOD polypeptide sequences that activates an epitope-specific T cell (e.g., activating effector functions and/or proliferation). Contacting CD8+ T cells with T-Cell-MP-epitope conjugates bearing one or two IL-2 MODs (e.g., two or four IL-2 MODs bearing H16 and/or F42 substitutions, such as H16A and/or F42A substitutions, in the duplex) can result in an expansion of T cells with a TCR specific to the presented epitope. Where the T cells are cytotoxic T cells (e.g., CD8+ cells), such a T-Cell-MP-epitope conjugate or its higher order complexes may increase the number and/or activity of a CD8+ effector T cell specific for a cancer cell or pathogen infected cell expressing the epitope. Activation of CD8+ T cells can result in increased proliferation of the CD8+ T cells and/or inducing or enhancing release of chemokines and/or cytokines by CD8+ T cells. Accordingly, the disclosure provides a method of treating a cancer or infection that includes administering to an individual in need thereof an effective amount of a T-Cell-MP-epitope conjugate or a higher order complex thereof (e.g., a duplex) comprising: (i) a cancer epitope (e.g. an epitope of a cancer-associated antigen); and (ii) one or more independently selected activating MOD polypeptide sequences that activates a T cell specific for the conjugated epitope. In some instances, an effective amount of a T-Cell-MP-epitope conjugate or a higher order complex thereof (e.g., a duplex) is an amount that increases the number or activity of CD8+ effector cells.

CD8+ T regulatory cells (T regs) have been shown to suppress antitumor immunity (see e.g., Wang, R-F. Human Immunity, 69(11): 811-814 (2008)). In cases where a T-Cell-MP-epitope conjugate or a higher order complex thereof comprises an inhibitory MOD (e.g., PD-L1, FasL, and the like) and an epitope specific to a T cell reactive toward a cancer or neoplasm it may be utilize in treatments (therapeutic methods) to reduce the proliferation and/or activity of a CD8⁺ T reg (e.g., FoxP3⁺, CD8⁺ T cells) specific to the epitope presented by the T-Cell-MP epitope conjugate. Such, treatments, which enhance antitumor immunity, may be utilized alone or in combination with other therapies for the treatment of cancers and neoplasms. Accordingly, the present disclosure provides a method of treating an individual having a cancer or neoplasm in which an individual fails to make a sufficient anti-tumor immune response due at least in part to suppression of the response by CD8+ T-reg cells. The method of treating diseases or disorders involving excess CD8+ T reg activity includes administering to an individual in need thereof an effective amount of a T-Cell-MP-epitope conjugate or a higher order complex thereof (e.g., a duplex) comprising: (i) an epitope specific to the neoplasm or cancer (e.g., an antigenic determinate of a cancer-associated antigen); and (ii) one or more independently selected inhibitory MOD polypeptide sequences that inhibit a CD8+ T reg with specificity for the epitope conjugated to the T-Cell-MP. An effective amount of such a T-Cell-MP-epitope conjugate or a higher order complex thereof may also be an amount, that when administered in one or more doses, causes a reduction in proliferation, absolute number, and/or activity (e.g., release of one or more of IL-10, TGF-β, IL-16, IFN-γ, or CCL4) of CD8+ T regs with specificity for the neoplasm or cancer epitope presented by T-Cell-MP-epitope conjugate. The reduction in CD8+ T regs with specificity for the neoplasm or cancer epitope may be at least 10%, at least 15%, at least 20%, at least 25%, at least 30%, at least 40%, at least 50%, at least 60%, at least 70%, at least 80%, at least 90%, or at least 95%, compared to number of those cells prior to administration of the T-Cell-MP-epitope conjugate or a higher order complex thereof.

The doses and routes of administration required to provide an effective amount of a T-Cell-MP to effect a treatment are discussed below.

Carcinomas that can be treated by a method disclosed herein include, but are not limited to, esophageal carcinoma, hepatocellular carcinoma, basal cell carcinoma (a form of skin cancer), squamous cell carcinoma (various tissues), bladder carcinoma, including transitional cell carcinoma (a malignant neoplasm of the bladder), bronchogenic carcinoma, colon carcinoma, colorectal carcinoma, gastric carcinoma, lung carcinoma, including small cell carcinoma and non-small cell carcinoma of the lung, adrenocortical carcinoma, thyroid carcinoma, pancreatic carcinoma, breast carcinoma, ovarian carcinoma, prostate carcinoma, adenocarcinoma, sweat gland carcinoma, sebaceous gland carcinoma, papillary carcinoma, papillary adenocarcinoma, cystadenocarcinoma, medullary carcinoma, renal cell carcinoma, ductal carcinoma in situ or bile duct carcinoma, choriocarcinoma, seminoma, embryonal carcinoma, Wilm's tumor, cervical carcinoma, uterine carcinoma, testicular carcinoma, osteogenic carcinoma, epithelial carcinoma, and nasopharyngeal carcinoma.

Sarcomas that can be treated by a method disclosed herein include, but are not limited to, fibrosarcoma, myxosarcoma, liposarcoma, chondrosarcoma, chordoma, osteogenic sarcoma, osteosarcoma, angiosarcoma, endotheliosarcoma, lymphangiosarcoma, lymphangioendotheliosarcoma, synovioma, mesothelioma, Ewing's sarcoma, leiomyosarcoma, rhabdomyosarcoma, and other soft tissue sarcomas.

Other solid tumors that can be treated by a method disclosed herein include, but are not limited to, glioma, astrocytoma, medulloblastoma, craniopharyngioma, ependymoma, pinealoma, hemangioblastoma, acoustic neuroma, oligodendroglioma, menangioma, melanoma, neuroblastoma, and retinoblastoma.

Leukemias that can be amenable to therapy by a method disclosed herein include, but are not limited to, a) chronic myeloproliferative syndromes (neoplastic disorders of multipotential hematopoietic stem cells); b) acute myelogenous leukemias (neoplastic transformation of a multipotential hematopoietic stem cell or a hematopoietic cell of restricted lineage potential; c) chronic lymphocytic leukemias (CLL; clonal proliferation of immunologically immature and functionally incompetent small lymphocytes), including B-cell CLL, T cell CLL prolymphocytic leukemia, and hairy cell leukemia; and d) acute lymphoblastic leukemias (characterized by accumulation of lymphoblasts). Lymphomas that can be treated using a subject method include, but are not limited to, B-cell lymphomas (e.g., Burkitt's lymphoma); Hodgkin's lymphoma; non-Hodgkin's lymphoma, and the like.

Other cancers that can be treated according to the methods disclosed herein include atypical meningioma, islet cell carcinoma, medullary carcinoma of the thyroid, mesenchymoma, hepatocellular carcinoma, hepatoblastoma, clear cell carcinoma of the kidney, and neurofibroma mediastinum.

As noted above, in some cases, in carrying out a subject treatment method, a T-Cell-MP-epitope conjugate or a higher order complex thereof (e.g., a duplex) of the present disclosure is administered to an individual in need thereof, as the polypeptide per se.

In addition to the administration of a T-Cell-MP-epitope conjugate, methods of treating a cancer or neoplasm may further comprising administering one or more therapeutic agents that, for example, enhance CD 8+ T cell functions (e.g., effector function) and/or otherwise treat the cancer or neoplasm or alleviate its symptoms. Accordingly, an anti-TGF-β antibody such as Metelimumab (CAT192) directed against TGF-β1 and Fresolimub directed against TGF-β1 and TGF-β2, or a TGF-β trap may be administered in conjunction with a T cell-MP-epitope conjugate for treatment of a cancer or neoplasm Treatment with an anti-TGF-β antibody may be subject to the proviso that the T-Cell-MP does not comprise an aa sequence to which the antibodies or TGF-β trap bind).

Other therapeutic agents that enhances CD 8+ function that may be administered in conjunction with a T cell-MP or a higher order complex thereof (e.g., a duplex) for the treatment of a cancer or neoplasm include, but are not limited to checkpoint inhibitors (discussed below), antibodies directed against: B lymphocyte antigens (e.g., ibritumomab, tiuxetan, obinutuzumab, ofatumumab, rituximab to CD20, brentuximab vedotin directed against CD30, and alemtuzumab to CD52); EGFR (e.g., cetuximab, panitumumab, and necitumumab); VEGF (e.g., bevacizumab); VEGFR2 (e.g., ramucirumab); HER2 (e.g., pertuzumab, trastuzumab, and ado-trastuzumab); PD-1 (e.g., nivolumab and pembrolizumab targeting a check point inhibition); RANKL (e.g., denosumab); CTLA-4 (e.g., ipilimumab targeting check point inhibition); IL-6 (e.g., siltuximab); disialoganglioside (GD2), (e.g., dinutuximab) disialoganglioside (GD2); CD38 (e.g., daratumumab); SLAMF7 (Elotuzumab); both EpCAM and CD3 (e.g., catumaxomab); or both CD19 and CD3 (e.g., blinatumomab) (optionally subject to the proviso that the T-Cell-MP or duplexed T-Cell-MP does not comprise a aa sequence to which the antibodies bind).

Chemotherapeutic agents that may be administered in conjunction with a T-Cell-MP-epitope conjugate for the treatment of cancers and neoplasms include, but are not limited to, alkylating agents, cytoskeletal disruptors (e.g., taxane), epothilones, histone deacetylase inhibitors, topoisomerase I inhibitors, topoisomerase II inhibitors, kinase inhibitors, nucleotide analog or precursor analogs, peptide antineoplastic antibiotics (e.g. bleomycin or actinomycin), platinum-based agents, retinoids, or vinca alkaloids and their derivatives. The chemotherapeutic agents may be selected from the group consisting of actinomycin all-trans retinoic acid, azacytidine, azathioprine, bleomycin, bortezomib, carboplatin, capecitabine, cisplatin, chlorambucil, cyclophosphamide, cytarabine, daunorubicin, docetaxel, doxifluridine, doxorubicin, epirubicin, epothilone, etoposide, fluorouracil, gemcitabine, hydroxyurea, idarubicin, imatinib, irinotecan, mechlorethamine, mercaptopurine, methotrexate, mitoxantrone, oxaliplatin, paclitaxel, pemetrexed, teniposide, tioguanine, topotecan, valrubicin, vemurafenib, vinblastine, vincristine, and vindesine.

2. Immune Checkpoint Inhibitors

As noted above, one type of therapeutic agent that may be administered in conjunction with a T cell-MP or a higher order complex thereof (e.g., a duplex) for the treatment of a cancer or neoplasm is an immune checkpoint inhibitor. Exemplary immune checkpoint inhibitors include inhibitors that target immune checkpoint polypeptide such as CD27, CD28, CD40, CD122, CD96, CD73, CD47, OX40, GITR, CSF1R, JAK, PI3K delta, PI3K gamma, TAM, arginase, CD137 (also known as 4-1BB), ICOS, A2AR, B7-H3, B7-H4, BTLA, CTLA-4, LAG3, TIM3, VISTA, CD96, TIGIT, CD122, PD-1, PD-L1 and PD-L2. In some cases, the immune checkpoint polypeptide is a stimulatory checkpoint molecule selected from CD27, CD28, CD40, ICOS, OX40, GITR, CD122 and CD137. In some cases, the immune checkpoint polypeptide is an inhibitory checkpoint molecule selected from A2AR, B7-H3, B7-H4, BTLA, CTLA-4, IDO, KIR, LAG3, PD-1, TIM3, CD96, TIGIT and VISTA.

In some cases, the immune checkpoint inhibitor is an antibody specific for an immune checkpoint, e.g., a monoclonal antibody. The anti-immune checkpoint antibody may be a fully human, humanized, or de-immunized such that the antibody does not substantially elicit an immune response in a human. In some cases, the anti-immune checkpoint antibody inhibits binding of the immune checkpoint polypeptide to a ligand for the immune checkpoint polypeptide. In some cases, the anti-immune checkpoint antibody inhibits binding of the immune checkpoint polypeptide to a receptor for the immune checkpoint polypeptide.

Antibodies, e.g., monoclonal antibodies, that are specific for immune checkpoints and that function as immune checkpoint inhibitors, are known in the art. See, e.g., Wurz et al. (2016) Ther. Adv. Med. Oncol. 8:4; and Naidoo et al. (2015) Ann. Oncol. 26:2375. Suitable anti-immune checkpoint antibodies include, but are not limited to, nivolumab (Bristol-Myers Squibb), pembrolizumab (Merck), pidilizumab (Curetech), AMP-224 (GlaxoSmithKline/Amplimmune), MPDL3280A (Roche), MDX-1105 (Medarex, Inc./Bristol Myer Squibb), MEDI-4736 (Medimmune/AstraZeneca), arelumab (Merck Serono), ipilimumab (YERVOY, (Bristol-Myers Squibb), tremelimumab (Pfizer), pidilizumab (CureTech, Ltd.), IMP321 (Immutep S.A.), MGA271 (Macrogenics), BMS-986016 (Bristol-Meyers Squibb), lirilumab (Bristol-Myers Squibb), urelumab (Bristol-Meyers Squibb), PF-05082566 (Pfizer), IPH2101 (Innate Pharma/Bristol-Myers Squibb), MEDI-6469 (MedImmune/AZ), CP-870,893 (Genentech), Mogamulizumab (Kyowa Hakko Kirin), Varlilumab (CelIDex Therapeutics), Avelumab (EMD Serono), Galiximab (Biogen Idec), AMP-514 (Amplimmune/AZ), AUNP 12 (Aurigene and Pierre Fabre), Indoximod (NewLink Genetics), NLG-919 (NewLink Genetics), INCB024360 (Incyte) and combinations thereof. Suitable anti-LAG3 antibodies include, e.g., BMS-986016 and LAG525. Suitable anti-GITR antibodies include, e.g., TRX518, MK-4166, INCAGN01876, and MK-1248. Suitable anti-OX40 antibodies include, e.g., MEDI0562, INCAGN01949, GSK2831781, GSK-3174998, MOXR-0916, PF-04518600, and LAG525. Suitable anti-VISTA antibodies are provided in, e.g., WO 2015/097536.

A suitable dosage of an anti-immune checkpoint antibody is from about 1 mg/kg to about 2400 mg/kg per day, such as from about 1 mg/kg to about 1200 mg/kg per day, including from about 50 mg/kg to about 1200 mg/kg per day. Other representative dosages of such agents include about 5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 60 mg/kg, 70 mg/kg, 80 mg/kg, 90 mg/kg, 100 mg/kg, 125 mg/kg, 150 mg/kg, 175 mg/kg, 200 mg/kg, 250 mg/kg, 300 mg/kg, 400 mg/kg, 500 mg/kg, 600 mg/kg, 700 mg/kg, 800 mg/kg, 900 mg/kg, 1000 mg/kg, 1100 mg/kg, 1200 mg/kg, 1300 mg/kg, 1400 mg/kg, 1500 mg/kg, 1600 mg/kg, 1700 mg/kg, 1800 mg/kg, 1900 mg/kg, 2000 mg/kg, 2100 mg/kg, 2200 mg/kg, and 2300 mg/kg per day. The effective dose of the antibody may be administered as two, three, four, five, six or more sub-doses, administered separately at appropriate intervals throughout the day.

In some cases, an immune checkpoint inhibitor is an anti-PD-1 antibody. Suitable anti-PD-1 antibodies include, e.g., nivolumab, pembrolizumab (also known as MK-3475), pidilizumab, SHR-1210, PDR001, and AMP-224. In some cases, the anti-PD-1 monoclonal antibody is nivolumab, pembrolizumab or PDR001. Suitable anti-PD1 antibodies are described in U.S. Patent Publication No. 2017/0044259. For pidilizumab, see, e.g., Rosenblatt et al. (2011) J. Immunother. 34:409-18.

In some cases, the anti-PD1 antibody is pembrolizumab. In some cases, the anti-PD-1 antibody is nivolumab (also known as MDX-1106 or BMS-936558; see, e.g., Topalian et al. (2012) N. Eng. J. Med. 366:2443-2454; and U.S. Pat. No. 8,008,449). In some cases, the anti-CTLA-4 antibody is ipilimumab or tremelimumab. For tremelimumab, see, e.g., Ribas et al. (2013) J. Clin. Oncol. 31:616-22.

In some cases, the immune checkpoint inhibitor is an anti-PD-L1 monoclonal antibody. In some cases, the anti-PD-L1 monoclonal antibody is BMS-935559, MEDI4736, MPDL3280A (also known as RG7446), or MSB0010718C. In some embodiments, the anti-PD-L1 monoclonal antibody is MPDL3280A (atezolizumab) or MEDI4736 (durvalumab). For durvalumab, see, e.g., WO 2011/066389. For atezolizumab, see, e.g., U.S. Pat. No. 8,217,149.

In some cases, the anti-PD-L1 antibody is atezolizumab.

3 Autoimmunity and Allergic Reactions

In some cases, a T-Cell-MP-epitope conjugate or a higher order complex thereof (e.g., a duplex) comprises one or more independently selected inhibitory MOD polypeptide sequences that inhibits the activity of an epitope-specific T cell (e.g., inhibiting effector functions and/or proliferation including granule dependent and/or granule independent responses).

Accordingly, the present disclosure provides a method of treating a disease or disorder in an individual involving an active T cell response to an antigenic determinate (a specific epitope) that needs to be suppressed (e.g., an allergic reaction or an autoimmune disorder); where the method comprises administering to the individual an effective amount of a T-Cell-MP-epitope conjugate or a higher order complex thereof (e.g., a duplex) conjugated to an the specific epitope and comprising one or more independently selected MODs (e.g., wild-type and/or variant inhibitory MODs). Where the epitope is a determinate of a self-antigen, such a T-Cell-MP-epitope conjugate or its higher order complexes may selectively inhibit the activity of a self-reactive T cell. Accordingly, the present disclosure provides a method of treating an autoimmune disease or disorder in an individual, the method comprising administering to the individual an effective amount of a T-Cell-MP-epitope conjugate or a higher order complex thereof (e.g., a duplex), where the conjugated epitope is a self-epitope (epitope of a self-antigen), and where the T-Cell-MP-epitope conjugate or a higher order complex thereof (e.g., a duplex) comprises one or more independently selected MODs (e.g., wild-type and/or variant inhibitory MODs and/or variant MODs).

An “effective amount” of the T-Cell-MP-epitope conjugate or its higher order complexes may also be an amount that, when administered in one or more doses to an individual in need thereof, reduces the number of T cells that are specific to the conjugated epitope (e.g., epitope of a self-antigen or allergen) by at least 10%, at least 15%, at least 20%, at least 25%, at least 30%, at least 40%, at least 50%, at least 60%, at least 70%, at least 80%, at least 90%, or at least 95%, compared to number of self-reactive T cells in the individual before administration, or in the absence of administration of the T-Cell-MP-epitope conjugate or its higher order complex. An “effective amount” for treating a disease or disorder involving a response to specific epitope of an allergen (e.g., an allergic reaction) or self-antigen (e.g., an autoimmune reaction), may be an amount that, when administered in one or more doses to an individual in need thereof, reduces production of Th2 cytokines in the individual. In some cases, an “effective amount” of a T-Cell-MP-epitope conjugate or a higher order complex thereof (e.g., a duplex) for treating an unwanted immune response to an antigen (e.g., allergic response or autoimmune response) is an amount that, when administered in one or more doses to an individual in need thereof, ameliorates one or more symptoms associated with the unwanted immune response. An effective amount of a T-Cell-MP-epitope conjugate or a higher order complex thereof (e.g., a duplex) may be an amount that reduces the number of CD8+ self-reactive T cells. In some instances, an effective amount of a T-Cell-MP-epitope conjugate or a higher order complex thereof (e.g., a duplex) is an amount that increases the number of CD8+ T regs, which in turn reduces the number of CD8+ self-reactive T effector cells and/or the cytokines or cytotoxic components (e.g., a perforin; a granzyme; a granulysin) released by activated CD8+ effector cells. The doses and routes of administration required to provide an effective amount of a T-Cell-MP to effect a treatment are discussed below.

In addition to the administration of a T-Cell-MP-epitope conjugate, methods of treating an autoimmune disease or allergy may further comprising administering one or more therapeutic agents suppress inflammation and/or immune response including, but not limited to: cyclooxygenase inhibitors such as NSAIDs (e.g., Cox-1 and/or Cox-2 inhibitors such as celecoxib, diclofenac, diflunisal, etodolac, ibuprofen, indomethacin, ketoprofen, and naproxen); corticosteroids (e.g., cortisone, dexamethasone, hydrocortisone, ethamethasoneb, fludrocortisone, methylprednisolone, prednisone, prednisolone and triamcinolone); agents that block one or more actions of tumor necrosis factor alpha (e.g., an anti-TNF alpha such as golimumab, infliximab, certolizumab, adalimumab or a TNF alpha decoy receptor such as etanercept); agents that bind to the IL-1 receptor competitively with IL-1 (e.g., anakinra); agents that bind to the IL-6 receptor and inhibits IL-6 from signaling through the receptor (e.g., tocilizumab). The use of such agents is subject to the proviso that where they are antibodies, the T-Cell-MP does not comprise an aa sequence (e.g., a wt, MOD or variant MOD) to which the antibody binds and/or an aa sequence to which the agent binds).

4 Infection by Pathogenic Agents

In some cases, a T-Cell-MP-epitope conjugate or a higher order complex thereof (e.g., a duplex) comprises (i) a pathogen specific epitope (e.g. an epitope of a viral or bacterial antigen), and (ii) one or more independently selected activating MOD polypeptide sequences that activate a target T cell specific to the epitope (e.g., activating granule dependent or granule independent effector functions). Where the target T cells are cytotoxic T cells (e.g., CD8+ cells), such a T-Cell-MP-epitope conjugate or its higher order complexes may increase the number and/or activity of a CD8+ effector T cell specific for an epitope expressed by pathogen infected cell or tissue. Activation of CD8+ T cells can increase proliferation of CD8+ T cells and/or induce or enhancing release of chemokines and/or cytokines by CD8+ T cells. In some instances, the epitope-specific T cell is a T cell that is specific for a peptide, phosphopeptide, or glycopeptide epitope (such as those from a spike glycoprotein, nucleoprotein, membrane protein, replicase protein, or non-structural protein of a virus, bacteria, or other pathogen), and contacting the epitope-specific T cell with the T-Cell-MP-epitope conjugate increases cytotoxic activity of the target T cell toward a pathogen infected cell or tissue.

An epitope-specific T cell may be a T cell that is specific for a virus epitope, and contacting the epitope-specific T cell with a T-Cell-MP conjugated to the virus epitope increases the number of those epitope-specific T cells and/or their cytotoxic activity towards cells infected with the virus.

Accordingly, this disclosure provides a method of treating an individual with an infection by a pathogen that includes administering to an individual in need thereof an effective amount of a T-Cell-MP-epitope conjugate or a higher order complex thereof (e.g., a duplex) comprising: (i) a pathogen specific epitope (e.g. an epitope of a viral or bacterial antigen); and (ii) one or more independently selected activating MOD polypeptide sequences that activates a T cell specific to the pathogen specific epitope. In some instances, an effective amount of a T-Cell-MP-epitope conjugate or a higher order complex thereof (e.g., a duplex) is an amount that increases the number or activity of CD8+ effector cells directed against the pathogen specific epitope.

In addition to the administration of a T-Cell-MP-epitope conjugate, methods of treating an infection (e.g., a pathogenic infection) may further comprising administering one or more therapeutic agents separately (e.g., sequentially, such as directly preceding or following the administration of the T-Cell-MP or on alternate days or weeks from the T-Cell-MP administration) or in concurrently (simultaneously or in admixture) such as one or more antibiotic, antifungal, antiviral, and/or anti-helminth agents.

5 Transplant Rejection and Graft vs Host Disease

Patients/subjects being treated for graft vs host or transplant rejection may receive therapeutic agents in addition to the administration of a T-Cell-MP for the purposes of suppressing those immune responses. Such therapeutic agent include, but not limited to, a corticosteroid (e.g., prednisone), an anti-proliferative agent (e.g., mycophenolate) and/or a calcineurin inhibitor (e.g., cyclosporine or tacrolimus). The therapeutic agents may be administered concurrently (simultaneously or in admixture) with the T-Cell-MP or separately (e.g., sequentially, such as directly preceding or following the administration of the T-Cell-MP or on alternate days or weeks from the T-Cell-MP administration).

6 Additional Therapeutic Agents for Use in Method of Treatment

Suitable therapeutic agents or drugs that may be administered with a T-Cell-MP or higher order T-Cell-MP complex, or a nucleic acid encoding an unconjugated T-Cell-MP, include virtually any therapeutic agent. Suitable therapeutic agents or drugs include but are not limited to, small molecule therapeutics (e.g., less than 2,000 Daltons in molecular weight) approved by the U.S. Food and Drug Administration, and/or listed in the 2020 U.S. Pharmacopeia or National Formulary. In an embodiment, those therapeutic agents or drugs are less than 1,000 molecular weight. Suitable drugs include, but are not limited to, antibiotics, chemotherapeutic (antineoplastic), anti-fungal, or anti-helminth agents and the like (e.g., sulfasalazine, azathioprine, cyclophosphamide, leflunomide; methotrexate, antimalarials, D-penicillamine, cyclosporine). Suitable chemotherapeutics may be alkylating agents, cytoskeletal disruptors (taxanes), epothilones, histone deacetylase inhibitors, topoisomerase I inhibitors, topoisomerase II inhibitors, kinase inhibitors, nucleotide analog or precursor analogs, peptide antineoplastic antibiotics (e.g. bleomycin or actinomycin), platinum-based agents, retinoids, or vinca alkaloids. Suitable drugs also include non-steroidal anti-inflammatory drugs and glucocorticoids, and the like.

In an embodiment, a suitable therapeutic agent that may be administered with a T-Cell-MP-epitope conjugate, or its higher order complexes, comprises an anti-TGF-β antibody, such as Metelimumab (CAT192) directed against TGF-β1 and/or Fresolimub directed against TGF-β1 and TGF-β2, or a TGF-β trap (e.g., Cablivi® caplacizumab-yhdp). Such antibodies would, as a generality, not be administered in conjunction with a T-Cell-MP or higher order T-Cell-MP complex that comprise a sequence to which the antibodies bind such as a TGF-β1 or TGF-β2 MOD.

In an embodiment, a suitable therapeutic agent that may be administered with a T-Cell-MP or higher order T-Cell-MP complex comprises one or more antibodies directed against: B lymphocyte antigens (e.g., ibritumomab tiuxetan, obinutuzumab, ofatumumab, rituximab to CD20, brentuximab vedotin directed against CD30, and alemtuzumab to CD52); EGFR (e.g., cetuximab, panitumumab, and necitumumab); VEGF (e.g., bevacizumab); VEGFR2 (e.g., ramucirumab); HER2 (e.g., pertuzumab, trastuzumab, and ado-trastuzumab); PD-1 (e.g., nivolumab and pembrolizumab targeting a check point inhibition); RANKL (e.g., denosumab); CTLA-4 (e.g., ipilimumab targeting check point inhibition); IL-6 (e.g., siltuximab); disialoganglioside (GD2), (e.g., dinutuximab) disialoganglioside (GD2); CD38 (e.g., daratumumab); SLAMF7 (Elotuzumab); both EpCAM and CD3 (e.g., catumaxomab); or both CD19 and CD3 (blinatumomab). Such antibodies would, as a generality, not be administered in conjunction with a T-Cell-MP or higher order T-Cell-MP complex (e.g., a duplexed T-Cell-MP) that comprise a sequence to which any of the administered antibodies bind.

A suitable therapeutic agent that may be administered with a T-Cell-MP or higher order T-Cell-MP complex, particularly for the treatment of a cancer or neoplasm, may comprises one or more chemotherapeutic agents. Such chemotherapeutic agents may be selected from: alkylating agents, cytoskeletal disruptors (e.g., taxanes), epothilones, histone deacetylase inhibitors, topoisomerase I inhibitors, topoisomerase II inhibitors, kinase inhibitors, nucleotide analogs or precursor analogs, peptide antineoplastic antibiotics (e.g. bleomycin or actinomycin), platinum-based agents, retinoids, or vinca alkaloids and their derivatives. In an embodiment, the chemotherapeutic agents are selected from actinomycin all-trans retinoic acid, azacytidine, azathioprine, bleomycin, bortezomib, carboplatin, capecitabine, cisplatin, chlorambucil, cyclophosphamide, cytarabine, daunorubicin, docetaxel, doxifluridine, doxorubicin, epirubicin, epothilone, etoposide, fluorouracil, gemcitabine, hydroxyurea, idarubicin, imatinib, irinotecan, mechlorethamine, mercaptopurine, methotrexate, mitoxantrone, oxaliplatin, paclitaxel, pemetrexed, teniposide, tioguanine, topotecan, valrubicin, vemurafenib, vinblastine, vincristine, and vindesine.

When being administered to treat a bacterial, fungal, and/or helminth infection, a suitable therapeutic agent that may be administered with a T-Cell-MP or higher order T-Cell-MP complex can comprise an antibiotic, anti-fungal, and/or anti-helminth agent.

When being administered to treat an autoimmune diseases or disorders, a suitable therapeutic agent that may be administered with a T-Cell-MP or higher order T-Cell-MP complex include, but are not limited to: an NSAID, corticosteroid (e.g., prednisone), an anti-proliferative agent (e.g., mycophenolate) and/or a calcineurin inhibitor (e.g., cyclosporine or tacrolimus).

### VII. Subjects Suitable for Treatment

Subjects suitable for treatment, e.g., by selectively delivering a MOD to a T cell or by modulating their T cell activity, include those with a cancer, infectious diseases (e.g., including those with viral, bacterial, and/or mycoplasma causative agents), graft vs host disease, transplant rejection, allergic reactions, and/or autoimmune diseases.

Subjects suitable for treatment who have a cancer include, but are not limited to, individuals who have been provided other treatments for the cancer but who failed to respond to the treatment. Cancers and neoplasms that can be treated with a method of the present disclosure include, but are not limited to, those displaying any of the cancer epitopes recited herein (see, e.g., the epitopes recited in Section I) including, but not limited to, AFP, WT-1, HPV and HBV epitopes, and those cancers and neoplasms recited in the methods of treatment described herein (see, e.g., Section VI).

Subjects suitable for treatment who have an allergy include, but are not limited to, individuals who have been provided other treatments for the allergy but who failed to respond to the treatment. Allergic conditions that can be treated with a method of the present disclosure include, but are not limited to, those resulting from exposure to nuts (e.g., tree and/or peanuts), pollen, and insect venoms (e.g., bee and/or wasp venom antigens).

Subjects suitable for treatment who have an autoimmune disease include, but are not limited to, individuals who have been provided other treatments for the autoimmune disease but who failed to respond to the treatment. Autoimmune diseases that can be treated with a method of the present disclosure include, but are not limited to, Addison's disease, alopecia areata, ankylosing spondylitis, autoimmune encephalomyelitis, autoimmune hemolytic anemia, autoimmune hepatitis, autoimmune-associated infertility, autoimmune thrombocytopenic purpura, bullous pemphigoid, Celiac Disease, Crohn's disease, Goodpasture's syndrome, glomerulonephritis (e.g., crescentic glomerulonephritis, proliferative glomerulonephritis), Grave's disease, Hashimoto's thyroiditis, mixed connective tissue disease, multiple sclerosis, myasthenia gravis (MG), pemphigus (e.g., pemphigus vulgaris), pernicious anemia, polymyositis, psoriasis, psoriatic arthritis, rheumatoid arthritis, scleroderma, Sjögren's syndrome, systemic lupus erythematosus (SLE), Type 1 diabetes, vasculitis, and vitiligo.

Subjects suitable for treatment included those who may become exposed to an infectious agent (e.g., a pathogen such as a virus, bacteria, fungus, protozoan, or helminth), those with suspected exposures, and those who have an active infection. Such subjects include, but are not limited to, individuals who have been provided other treatments for the infectious disease but who failed to respond to the treatment. Infectious diseases that can be treated with a method of the present disclosure include, but are not limited to, those having an infectious agent (e.g., a virus such as HPV, HBV, and the like) recited herein (see, e.g., Infectious Agents in Section I).

Subjects suitable for treatment include individuals that have received a transplant and that have, or at risk of an immune response to implanted tissues in the form of graft vs host disease or of transplant rejection. The subjects include, but are not limited to, individuals who have been provided other treatments but who failed to respond to the treatment. The subjects may be receiving other treatments concurrent with administration of a T-Cell-MP for the purposes of suppressing immune responses to transplanted tissues including, but not limited to, a corticosteroid (e.g., prednisone), an anti-proliferative agent (e.g., mycophenolate) and/or a calcineurin inhibitor (e.g., cyclosporine or tacrolimus).

### VIII. Dosages and Routes of Administration

A. Dosages

A suitable dosage of a T-Cell-MP (e.g., a T-Cell-MP-epitope conjugate) can be determined by an attending physician, or other qualified medical personnel, based on various clinical factors. As is well known in the medical arts, dosages for any one patient depend upon many factors, including the patient's size, body surface area, age, the particular T-Cell-MP (e.g., a T-Cell-MP-epitope conjugate) to be administered, sex of the patient, time, route of administration, general health, and other drugs being administered concurrently. Those of skill will also appreciate that dose levels can vary as a function of the specific T-Cell-MP being administered, the severity of the symptoms and the susceptibility of the subject to side effects. Preferred dosages for a given compound are readily determinable by those of skill in the art by a variety of means.

A T-Cell-MP (e.g., a T-Cell-MP-epitope conjugate) may be administered in amounts between 1 ng/kg body weight and 100 mg/kg body weight per dose, e.g., from 0.01 μg to 100 mg per kg of body weight, from 0.1 μg to 10 mg per kg of body weight, from 1 μg to 50 mg per kg of body weight, from 10 μg to 20 mg per kg of body weight, from 100 μg to 15 mg per kg of body weight, from 500 μg to 10 mg per kg of body weight (e.g., from 0.1-0.5 mg per kg of body weight, 0.5-1.0 mg per kg of body weight, 1.0 to 5.0 mg per kg of body weight, 5.0 to 10.0 mg per kg of body weight, 1.0-3.0 mg per kg of body weight, 2.0-4.0 mg per kg of body weight, 3.0-5.0 mg per kg of body weight, 4.0-6.0 mg per kg of body weight, 5.0-7.0 mg per kg of body weight, 6.0-8.0 mg per kg, 7.0-9.0 mg per kg of body weight, and 8.0-10.0 mg per kg of body weight), or from 0.5 mg/kg body weight to 5 mg/kg body weight; however, doses below or above these exemplary ranges are envisioned, especially considering the aforementioned factors. If the regimen is a continuous infusion the above-mentioned doses can be utilized, or doses can be, for example, in the range of 1 μg to 10 mg per kilogram of body weight per minute. A T-Cell-MP (e.g., a T-Cell-MP-epitope conjugate) can also be administered in an amount of from about 0.1 mg/kg body weight to 50 mg/kg body weight, e.g., from about 0.1 mg/kg body weight to about 5 mg/kg body weight, from about 5 mg/kg body weight to about 10 mg/kg body weight, from about 10 mg/kg body weight to about 20 mg/kg body weight, from about 20 mg/kg body weight to about 30 mg/kg body weight, from about 30 mg/kg body weight to about 40 mg/kg body weight, or from about 40 mg/kg body weight to about 50 mg/kg body weight. Persons of ordinary skill in the art can easily estimate repetition rates for dosing based on measured residence times and concentrations of the administered agent in bodily fluids or tissues.

Following successful treatment, it may be desirable to have the patient undergo maintenance therapy to prevent the recurrence of the disease state, wherein a T-Cell-MP (e.g., a T-Cell-MP-epitope conjugate) is administered in maintenance doses, for example, ranging from 0.01 μg to 100 mg per kg of body weight, from 0.1 μg to 100 mg per kg of body weight, from 1 μg to 50 mg per kg of body weight, from 10 μg to 20 mg per kg of body weight, from 100 μg to 15 mg per kg of body weight, or from 500 μg to 10 mg per kg of body weight (e.g., from 0.1-0.5 mg per kg, 0.5-1.0 mg per kg, 1.0-3.0 mg per kg, 2.0-4.0 mg per kg, 3.0-5.0 mg per kg, 4.0-6.0 mg per kg, 5.0-7.0 mg per kg, 6.0-8.0 mg per kg, 7.0-9.0 mg per kg, and 8.0-10.0 mg per kg).

The frequency of administration of a T-Cell-MP (e.g., a T-Cell-MP-epitope conjugate) can vary depending on any of a variety of factors, e.g., severity of the symptoms, etc. For example, in some embodiments, a T-Cell-MP is administered once every two months, once per month, twice per month, once every two weeks, three times per month, once every three weeks, every other week (qow), once every week, once per week (qw), twice per week (biw), three times per week (tiw), four times per week, five times per week, six times per week, every other day (qod), daily (qd), twice a day (qid), or three times a day (tid).

The duration of administration of a T-Cell-MP (e.g., a T-Cell-MP-epitope conjugate) of the present disclosure (e.g., the period of time over which a T-Cell-MP is administered in one or more doses) can vary depending on any of a variety of factors including patient response, etc. For example, a T-Cell-MP-epitope conjugate of the present disclosure can be administered over a period of time ranging from about one day to about one week, from about two weeks to about four weeks, from about one month to about two months, from about two months to about four months, from about four months to about six months, from about six months to about eight months, from about eight months to about 1 year, from about 1 year to about 2 years, or from about 2 years to about 4 years, or more.

B. Routes of Administration

A T-Cell-MP (e.g., an unconjugated T-Cell-MP or T-Cell-MP-epitope conjugate of the present disclosure) or a nucleic acid encoding all or part of an unconjugated T-Cell-MP may be administered to an individual using any available method and route suitable for delivery, including in vivo and ex vivo methods, as well as systemic and localized routes of administration.

A T-Cell-MP of the present disclosure may be administered to a host using any available methods and routes suitable for delivery of conventional drugs, including systemic or localized routes. In general, routes of administration contemplated for use in a method of the present disclosure include, but are not necessarily limited to, enteral, parenteral, and inhalational routes. Some acceptable routes of administration include intratumoral, peritumoral, intramuscular, intralymphatic, intratracheal, intracranial, subcutaneous, intradermal, topical, intravenous, intra-arterial, rectal, nasal, oral, and other enteral and parenteral routes of administration. Routes of administration may be combined, if desired, or adjusted depending upon the T-Cell-MP administered and/or the desired effect. A T-Cell-MP can be administered in a single dose or in multiple doses.

A T-Cell-MP (e.g., a T-Cell-MP-epitope conjugate) may be administered intravenously. In some embodiments, a T-Cell-MP is administered intramuscularly. A T-Cell-MP (e.g., a T-Cell-MP-epitope conjugate) may be administered intralymphatically. A T-Cell-MP (e.g., a T-Cell-MP-epitope conjugate) may be administered locally (e.g., pulmonary administration such as in a nebulized or other aerosolized form). A T-Cell-MP (e.g., a T-Cell-MP-epitope conjugate) may be administered intracranially. A T-Cell-MP (e.g., a T-Cell-MP-epitope conjugate) may be administered subcutaneously.

Parenteral routes of administration other than inhalation administration include, but are not necessarily limited to, topical, transdermal, subcutaneous, intramuscular, intraorbital, intracapsular, intraspinal, intrasternal, intratumoral, intralymphatic, peritumoral, and intravenous routes, i.e., any route of administration other than through the alimentary canal. Parenteral administration can be carried out to effect systemic or local delivery of a T-Cell-MP. Where systemic delivery is desired, administration typically involves invasive or systemically absorbed topical or mucosal administration of pharmaceutical preparations.

### IX. Certain Aspects

While the present invention has been described with reference to the specific embodiments thereof, it should be understood by those skilled in the art that various changes may be made, and equivalents may be substituted without departing from the true spirit and scope of the invention. In addition, many modifications may be made to adapt a particular situation, material, composition of matter, process, and/or process step or steps, to the objective, spirit and scope of the present invention. All such modifications are intended to be within the scope of the claims appended hereto.
1. An unconjugated T cell modulatory polypeptide (T-Cell-MP), the polypeptide comprising (e.g., from N-terminus to C-terminus):
   (i) optionally one or more MOD polypeptide sequences (e.g., two or more MOD polypeptide sequences, such as in tandem, wherein when there are two or more MOD polypeptide sequences they are optionally joined to each other by independently selected L1 linkers);
   (ii) an optional L2 linker polypeptide sequence joining the one or more MOD polypeptide sequences to a β2M polypeptide sequence;
   (iii) the β2M polypeptide sequence;
   (iv) an optional L3 linker polypeptide sequence (e.g., from 10-50 aas in length);
   (v) a class I MHC-H polypeptide sequence;
   (vi) an optional L4 linker polypeptide sequence;
   (vii) a scaffold polypeptide sequence (e.g., an immunoglobulin Fc sequence);
   (viii) an optional L5 linker polypeptide sequence; and
   (ix) optionally one or more MOD polypeptide sequences (e.g., two or more MOD polypeptide sequences, such as in tandem, wherein when there are two or more MOD polypeptide sequences they are optionally joined to each other by independently selected L6 linkers);
wherein the unconjugated T-Cell-MP comprises at least one MOD polypeptide sequence (e.g., the MOD(s) of element (i) or (ix)); and
wherein at least one of the β2M polypeptide sequence, the L3 linker polypeptide sequence, and/or the MHC-H polypeptide sequence comprises one or more chemical conjugation sites for epitope conjugation.
2. The unconjugated T-Cell-MP of aspect 1, the polypeptide comprising from N-terminus to C-terminus:
   (i) optionally one or more MOD polypeptide sequences (e.g., two or more MOD polypeptide sequences, such as in tandem, wherein when there are two or more MOD polypeptide sequences they are optionally joined to each other by independently selected L1 linkers);
   (ii) an optional L2 linker polypeptide sequence;
   (iii) a β2M polypeptide sequence;
   (iv) an optional L3 linker polypeptide sequence (e.g., from 10-50 aas in length);
   (v) a class I MHC-H polypeptide sequence;
   (vi) an optional L4 linker polypeptide sequence;
   (vii) a scaffold polypeptide sequence (e.g., an immunoglobulin Fc sequence);
   (viii) an optional L5 linker polypeptide sequence; and
   (ix) optionally one or more MOD polypeptide sequences (e.g., two or more MOD polypeptide sequences, such as in tandem, wherein when there are two or more MOD polypeptide sequences they are optionally joined to each other by independently selected L6 linkers);
wherein the unconjugated T-Cell-MP comprises at least one MOD polypeptide sequence (e.g., the MOD(s) of element (i) or (ix)); and
wherein at least one of the β2M polypeptide sequence, the L3 linker polypeptide sequence, and/or the MHC-H polypeptide sequence comprises one or more chemical conjugation sites for epitope conjugation.
The chemical conjugation site for epitope conjugation of aspects 1 and 2 permits the covalent attachment of an epitope presenting molecule (e.g., a peptide epitope) to the T-Cell-MP such that it can be bound by the MHC-H polypeptide and presented to a TCR. It is understood that the unconjugated T-Cell-MPs of aspects 1 and 2 do not comprise a peptide epitope (either covalently attached to, or as a fusion with, the T-Cell-MP polypeptide) that can be located in the binding cleft of the MHC-H/β2M polypeptide sequences and presented to a TCR.
3. The unconjugated T-Cell-MP of aspect 1 or aspect 2, wherein the MHC-H polypeptide sequence comprises a human class I MHC-H chain polypeptide sequence selected from HLA-A, HLA-B, HLA-C, HLA-E, HLA-F, and HLA-G MHC-H polypeptide sequences having at least 85% (e.g., at least 90%, at least 95% or 98%) or 100% sequence identity to at least 200 (e.g., at least 225, at least 250, at least 260, or at least 275) contiguous aas of a MHC-H polypeptide provided in any of FIGS. 3A-3H.
4. The unconjugated T-Cell-MP of any preceding aspect, wherein the MHC-H sequence does not include the MHC-H transmembrane domain, or a portion thereof, that will anchor the T-Cell-MP in a cell membrane.
5. The unconjugated T-Cell-MP of any preceding aspect, wherein the MHC-H polypeptide sequence has at least 85% (e.g., at least 90%, at least 95% or 98%) or 100% sequence identity to at least 200 (e.g., at least 225, at least 250, at least 260, or at least 275) contiguous aas of the α1, α2, and α3 domains of a HLA-A allele.
6. The unconjugated T-Cell-MP of any of aspects 1-5, wherein the MHC-H polypeptide sequence has at least 85% (e.g., at least 90%, at least 95% or 98%) or 100% sequence identity to at least 200 (e.g., at least 225, at least 250, at least 260, or at least 275) contiguous aas of a HLA-A*0101, HLA-A*0201, HLA-A*0301, HLA-A*1101, HLA-A*2301, HLA-A*2402, HLA-A*2407, HLA-A*3303, or HLA-A*3401 polypeptide sequence provided in FIG. 3E.
7. The unconjugated T-Cell-MP of any of aspects 1-6, wherein the MHC-H polypeptide sequence has at least 85% (e.g., at least 90%, at least 95% or 98%) or 100% sequence identity to at least 200 (e.g., at least 225, at least 250, at least 260, or at least 275) contiguous aas of a HLA-A*0101, HLA-A*0201, HLA-A*1101, HLA-A*2402, HLA-A*3303, or HLA-A*3401 polypeptide sequence (e.g., as provided in FIG. 3E).
8. The unconjugated T-Cell-MP of any of aspects 1-4, wherein the MHC-H polypeptide sequence has at least 85% (e.g., at least 90%, at least 95% or 98%) or 100% sequence identity to at least 200 (e.g., at least 225, at least 250, at least 260, or at least 275) contiguous aas of the α1, α2, and α3 domains of a HLA-B allele.
9. The unconjugated T-Cell-MP of any of aspects 1-4 or 8, wherein the MHC-H polypeptide sequence has at least 85% (e.g., at least 90%, at least 95% or 98%) or 100% sequence identity to at least 200 (e.g., at least 225, at least 250, at least 260, or at least 275) contiguous aas of a HLA-B*0702, HLA-B*0801, HLA-B*1502, B27 (subtypes HLA-B*2701-2759), HLA-B*3802, HLA-B*4001, HLA-B*4601, or HLA-B*5301 polypeptide sequence (e.g., as provided in FIG. 3F).
10. The unconjugated T-Cell-MP of any of aspects 1-4 or 8, wherein the MHC-H sequence has at least 85% (e.g., at least 90%, at least 95% or 98%) or 100% sequence identity to at least 200 (e.g., at least 225, at least 250, at least 260, or at least 275) contiguous aas of HLA-B*0702.
11. The unconjugated T-Cell-MP of any of aspects 1-4, wherein the MHC-H polypeptide sequence has at least 85% (e.g., at least 90%, at least 95% or 98%) or 100% sequence identity to at least 200 (e.g., at least 225, at least 250, at least 260, or at least 275) contiguous aas of the α1, α2, and α3 domains of a HLA-C allele.
12. The unconjugated T-Cell-MP of any of aspects 1-4 or 11, wherein the MHC-H sequence has at least 85% (e.g., at least 90%, at least 95% or 98%) or 100% sequence identity to at least 200 (e.g., at least 225, at least 250, at least 260, or at least 275) contiguous aas of a HLA-C*0102, HLA-C*0303, HLA-C*0304, HLA-C*0401, HLA-C*0602, HLA-C*0701, HLA-C*0702, HLA-C*0801, or HLA-C*1502 polypeptide sequence (e.g., as provided in FIG. 3G).
13. The unconjugated T-Cell-MP of any of aspects 1-4 or 11, wherein the MHC-H polypeptide sequence has at least 85% (e.g., at least 90%, at least 95% or 98%) or 100% sequence identity to at least 200 (e.g., at least 225, at least 250, at least 260, or at least 275) contiguous aas of HLA-C*0701.
14. The unconjugated T-Cell-MP of any of aspects 1-4, wherein the MHC-H polypeptide sequence has at least 85% (e.g., at least 90%, at least 95% or 98%) or 100% sequence identity to at least 200 (e.g., at least 225, at least 250, at least 260, or at least 275) contiguous aas of the α1, α2, and α3 domains of a HLA-E allele.
15. The unconjugated T-Cell-MP of any of aspects 1-4 or 14, wherein the MHC-H polypeptide sequence has at least 85% (e.g., at least 90%, at least 95% or 98%) or 100% sequence identity to at least 200 (e.g., at least 225, at least 250, at least 260, or at least 275) contiguous aas of a HLA-E*0101, HLA-E*01:03, HLA-E*01:04, HLA-E*01:05, HLA-E*01:06, HLA-E*01:07, HLA-E*01:09, or HLA-E*01:10 polypeptide sequence (e.g., as provided in FIG. 3H).
16. The unconjugated T-Cell-MP of any of aspects 1-4 or 14, wherein the MHC-H polypeptide sequence has at least 85% (e.g., at least 90%, at least 95% or 98%) or 100% sequence identity to at least 200 (e.g., at least 225, at least 250, at least 260, or at least 275) contiguous aas of the HLA-E allele consensus sequence:

GSHSLKYFHT SVSRPGRGEP RFISVGYVDD TQFVRFDNDA
ASPRMVPRAP WMEQEGSEYW DRETRSARDT AQIFRVNLRT
LRG [symbol] YNQSX1A GSHTLQWMHG CELGPDX2RFL RGYEQFAYDG
KDYLTLNEDL RSWTAVDT [symbol] A QISEQKSNDA SEAEHQX3X4YL
EDTCVEWLHK YLEKGKETLL HLEPPKTHVT HHPISDHEAT
LRCWALGFYP AEITLTWQQD GEGHTQDTEL VETRP [symbol] GDGT
FQKWAAVVVP SGEEX5RYTCH VQHEGLX6EPV TLRWKPASQP
TIPI,

wherein X1=K or E, X2=R or G, X3=R or G, X4=A or V, X5=Q or P, and X6=P or S. (SEQ ID NO:58)
17. The unconjugated T-Cell-MP of any of aspects 1-4, wherein the MHC-H polypeptide sequence has at least 85% (e.g., at least 90%, at least 95% or 98%) or 100% sequence identity to at least 200 (e.g., at least 225, at least 250, at least 260, or at least 275) contiguous aas of the α1, α2, and α3 domains of a HLA-F allele.
18. The unconjugated T-Cell-MP of any of aspects 1-4 or 17, wherein the MHC-H polypeptide sequence has at least 85% (e.g., at least 90%, at least 95% or 98%) or 100% sequence identity to at least 200 (e.g., at least 225, at least 250, at least 260, or at least 275) contiguous aas of a HLA-F*0101 (HLA-F*01:01:01:01), HLA-F*01:02, HLA-F*01:03 (HLA-F*01:03:01:01), HLA-F*01:04, HLA-F*01:05, or HLA-F*01:06. polypeptide sequence (e.g., as provided in FIG. 3H).
19. The unconjugated T-Cell-MP of any of aspects 1-4 or 17, wherein the MHC-H polypeptide sequence has at least 85% (e.g., at least 90%, at least 95% or 98%) or 100% sequence identity to at least 200 (e.g., at least 225, at least 250, at least 260, or at least 275) contiguous aas of the HLA-F allele consensus sequence:

GSHSLRX1FST AVSRPGRGEP RYIAVEYVDD TQFLRFDSDA
AIPRMEPREX2 WVEQEGPQYW EWTTGYAKAN AQTDRVALRN
LLRRYNQSEA GSHTLQGMNG CDMGPDGRLL RGYHQHAYDG
KDYISLNEDL RSWTAADTVA QITQRFYEAE EYAEEFRTYL
EGECLELLRR YLENGKETLQ RADPPKAHVA HHPISDHEAT
LRCWALGFYP AEITLTWQRD GEEQTQDTEL VETRPAGDGT
FQKWAAVVVP X3GEEQRYTCH VQHEGLPQPL ILRWEQSX4QP TIPI,

wherein X1=Y or F; X2=P or Q; X3=S or P; and X4=P or L. (SEQ ID NO:59)
20. The unconjugated T-Cell-MP of any of aspects 1-4, wherein the MHC-H polypeptide sequence has at least 85% (e.g., at least 90%, at least 95% or 98%) or 100% sequence identity to at least 200 (e.g., at least 225, at least 250, at least 260, or at least 275) contiguous aas of the α1, α2, and α3 domains of a HLA-G allele.
21. The unconjugated T-Cell-MP of any of aspects 1-4 or 20, wherein the MHC-H polypeptide sequence has at least 85% (e.g., at least 90%, at least 95% or 98%) or 100% sequence identity to at least 200 (e.g., at least 225, at least 250, at least 260, or at least 275) contiguous aas of a HLA-G*01:04 (HLA-G*01:04:01:01), HLA-G*01:06, HLA-G*01:07, HLA-G*01:08, HLA-G*01:09: HLA-G*01:10, HLA-G*01:11, HLA-G*01:12, HLA-G*01:14, HLA-G*01:15, HLA-G*01:16, HLA-G*01:17, HLA-G*01:18: HLA-G*01:19, HLA-G*01:20, or HLA-G*01:22 polypeptide sequence (e.g., as provided in FIG. 3H).
22. The unconjugated T-Cell-MP of any of aspects 1-4 or 20, wherein the MHC-H polypeptide sequence has at least 85% (e.g., at least 90%, at least 95% or 98%) or 100% sequence identity to at least 200 (e.g., at least 225, at least 250, at least 260, or at least 275) contiguous aas of the HLA-G allele consensus sequence:

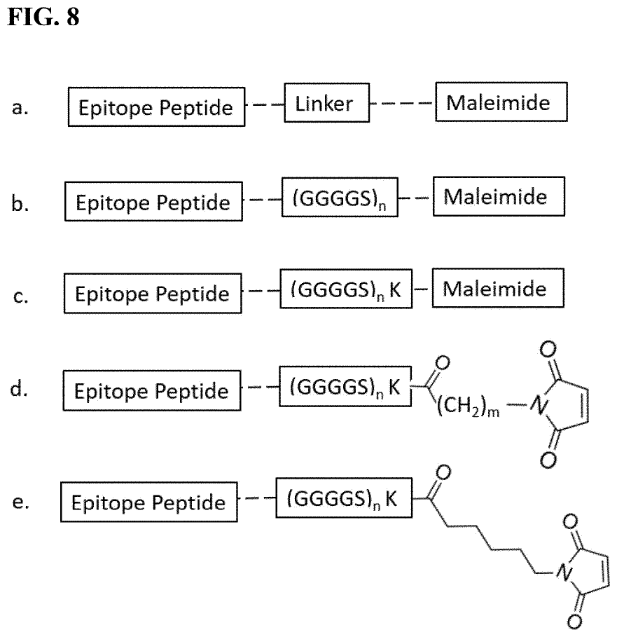

GSHSMRYFSA AVX1RPGRGEP RFIAMGX2VDD X3QFX4RFDSDS
ACPRMEPRAP WVEX5EGPEYW EEETRNTKAH AQTDRMNLQT
X6RG [symbol] YNQSEA SSHTLQWMIX7 CDLX8X9DGRLX10 RGYEQYAYDG
KDYLALNEDL RSWTAADT [symbol] A QISKRKCEAA NVAEQRRAX11L
EGTCVEWLX12R X13LENGKEX14LQ RADPX15KTHVT HHPVFDYEAT
LRCWALGFYP AEIILTWQX16D GEDQTQDVEL VETRP [symbol] GDGT
FQKWAAVVVP SGEEQRYX17CH VQHEGLPEPL MLRWX18QSSLP
TIPI,

wherein X1=S or F, X2=Y or H, X3=T, S, or M, X4=L or V; X5=Q or R, X6=P or L, X7=G or D, X8=G or V, X9=S or C, X10=L or I, X11=Y or H, X12=H or R, X13=Y or H, X14=M or T, X15=P or A, X16=R, W, or Q, X17=T or M, X18=K or E. (SEQ ID NO:60)
23. The unconjugated T-Cell-MP of any of aspects 1-22, wherein the MHC-H polypeptide sequence comprises at least one mutation (e.g., two, or three mutations) selected from the group consisting of: an alanine at position 84 (e.g., Y84A or R84A in the case of HLA-F), a cysteine at position 84 (e.g., Y84C or R84C in the case of HLA-F), a cysteine at position 139 (e.g., A139C or V139C in the case of HLA-F), and a cysteine at position 236 (e.g., A236C). See FIG. 3I for the location of those aa positions.
24. The unconjugated T-Cell-MP of any of aspects 1-23, wherein the MHC-H polypeptide sequence comprises a combination of mutations selected from the group consisting of: Y84A and A139C; Y84A and A236C; Y84C and A139C; Y84C and A236C; and Y84C, A139C and A236C.
25. The unconjugated T-Cell-MP of any of aspects 1-23, wherein the MHC-H polypeptide sequence comprises: a cysteine at position 84 (e.g., Y84C or R84C in the case of HLA-F), a cysteine at position 139 (e.g., A139C or V139C in the case of HLA-F), and optionally a cysteine at position 236 (e.g., A236C). See FIG. 3I for the location of those aa positions.
26. The unconjugated T-Cell-MP of any preceding aspect, wherein the β2M sequence has at least 90% (e.g., at least 95% or 98%) or 100% sequence identity to at least 50 (e.g., 60, 70, 80, 90, 96, 97, or 98 or all) contiguous aas of a mature human β2M polypeptide (e.g., aas 21-119 of NCBI accession number NP_004039.1 provided in FIG. 4).
27. The unconjugated T-Cell-MP of any preceding aspect, wherein the β2M sequence has up to 6 (e.g., 1, 2, 3, 4, or 5) aa substitutions within an aa segment of at least 70 (e.g., at least 80, 90, 96, 97, or 98 or all) contiguous aas of a mature human β2M polypeptide (e.g., aas 21-119 of NCBI accession number NP_004039.1 provided in FIG. 4).
28. The unconjugated T-Cell-MP of any of aspects 1-27, wherein the unconjugated T-Cell-MP comprises at least one linker sequence comprising, consisting essentially of, or consisting of: i) Gly and/or Ser; ii) Ala and Ser; iii) Gly, Ala, and Ser; iv) Gly, Ser, and Cys (e.g., a single Cys residue); v) Ala, Ser, and Cys (e.g., a single Cys residue); or vi) Gly, Ala, Ser, and Cys (e.g., a single Cys residue).
29. The unconjugated T-Cell-MP any of aspects 1-27, wherein the unconjugated T-Cell-MP comprises at least one linker (e.g., any of linkers L1-L6) that comprises one or more sequences selected from: polyG (e.g., polyglycine comprising 1-10 Gly residues), GA, AG, AS, SA, GS, GSGGS (SEQ ID NO:130), GGGS (SEQ ID NO:131), GGSG (SEQ ID NO:132), GGSGG (SEQ ID NO:133), GSGSG (SEQ ID NO:134), GSGGG (SEQ ID NO:135), GGGSG (SEQ ID NO:136), GSSSG (SEQ ID NO:137), GGGGS (SEQ ID NO:139), or AAAGG (SEQ ID NO:532), any of which may be repeated 2, 3, 4, 5, 6, 7, 8, 9, or 10 times.
30. The unconjugated T-Cell-MP of any preceding aspect, wherein the unconjugated T-Cell-MP comprises at least one linker comprising a G₄S or an AAAGG sequence that may be repeated from 1-10 times (e.g., repeated 1, 2, 3, 4, 5, 6, 7, 8, 9, or 10 times).
31. The unconjugated T-Cell-MP of any preceding aspect, wherein the scaffold polypeptide sequences are independently selected from non-interspecific sequences or interspecific sequences.
32. The unconjugated T-Cell-MP of aspect 31, wherein the interspecific and non-interspecific sequences are selected from the group consisting of: immunoglobulin heavy chain constant regions (Ig Fc e.g., CH2-CH3); collectin polypeptides, coiled-coil domains, leucine-zipper domains; Fos polypeptides; Jun polypeptides; Ig CH1; Ig C_{L} Ig C_{L} knob-in-hole without disulfide (KiH); knob-in hole with a stabilizing disulfide bond (KiHs-s); HA-TF; ZW-1; 7.8.60; DD-KK; EW-RVT; EW-RVTs-s; and A107 sequences.
33. The unconjugated T-Cell-MP of any preceding aspect, complexed to form a duplex T-Cell-MP or higher order T-Cell-MP comprising at least a first unconjugated T-Cell-MP and a second unconjugated T-Cell-MP of any of aspects 1-32, wherein:
   (i) the first unconjugated T-Cell-MP comprises a first β2M polypeptide sequence; a first class I MHC-H polypeptide sequence; and a first scaffold polypeptide; and
   (ii) the second unconjugated T-Cell-MP comprises a second β2M polypeptide sequence; a second class I MHC-H polypeptide sequence; and a second scaffold polypeptide; and wherein the first and second unconjugated T-Cell-MPs associate by binding interactions between the first and second scaffold polypeptides that optionally include one or more interchain covalent bonds (e.g., one or two disulfide bonds). See e.g., the duplexes in FIGS. 8 and 9.
34. The unconjugated T-Cell-MP or duplex T-Cell-MP of any preceding aspect, wherein the scaffold comprises a non-immunoglobulin polypeptide sequence.
35. The unconjugated T-Cell-MP or duplex T-Cell-MP of aspect 34, wherein the non-immunoglobulin polypeptide sequence is a non-interspecific polypeptide sequence (e.g., a non-interspecific coiled-coil or leucine zipper sequence).
36. The unconjugated T-Cell-MP or duplex T-Cell-MP of aspect 34, wherein the non-immunoglobulin polypeptide sequence is an interspecific polypeptide sequence (e.g., an interspecific coiled-coil or leucine zipper sequence; Fos polypeptides that pair with Jun protein sequences; or Jun polypeptides that pair with Fos protein sequences).
37. The unconjugated T-Cell-MP or duplex T-Cell-MP of any of aspects 1-33, wherein the scaffold comprises an immunoglobulin polypeptide sequence.
38. The unconjugated T-Cell-MP or duplex T-Cell-MP of aspect 37, where the immunoglobulin polypeptide sequence comprises one or more substitutions that reduce the binding with Ig Fc receptors and/or complement C1q protein relative to a T-Cell-MP where the immunoglobulin polypeptide sequence is unsubstituted.
39. The unconjugated T-Cell-MP or duplex T-Cell-MP of aspect 37 or 38, wherein the scaffold comprises a non-interspecific immunoglobulin polypeptide sequence.
40. The unconjugated T-Cell-MP or duplex T-Cell-MP of aspect 39, wherein the non-interspecific immunoglobulin polypeptide sequence comprises a human IgA Fc, IgD Fc, or IgE Fc (e.g., comprising an aa sequence having at least about 70% (e.g., at least about 75%, 80%, 85%, 90%, 95%, 98%, or 99%) or 100% aa sequence identity to at least 125 contiguous aas (e.g., at least 150, at least 175, at least 200, or at least 210 contiguous aas) or all aas of an aa sequence of an Ig Fc region depicted in FIGS. 2A-2C.
41. The unconjugated T-Cell-MP or duplex T-Cell-MP of aspect 39, wherein the non-interspecific immunoglobulin polypeptide sequence comprises a human IgG1 Fc, IgG2 Fc IgG3 Fc or IgG4 Fc (e.g., comprising an aa sequence having at least about 70% (e.g., at least about 75%, 80%, 85%, 90%, 95%, 98%, or 99%) or 100% aa sequence identity to at least 125 contiguous aas (e.g., at least 150, at least 175, at least 200, or at least 210 contiguous aas) or all aas of an aa sequence of an Ig Fc region depicted in FIGS. 2D-2G. For example, the non-interspecific immunoglobulin polypeptide sequence may comprise a human IgG1 Fc, IgG2 Fc IgG3 Fc or IgG4 Fc aa sequence having at least about 90% or at least about 95% aa sequence identity to at least 150 or 200 contiguous aas of an Ig Fc region depicted in FIGS. 2D-2G.
42. The unconjugated T-Cell-MP or duplex T-Cell-MP of any of aspects 39 or 41, wherein the non-interspecific immunoglobulin polypeptide sequence comprises a human IgG1 Fc (e.g., comprising an aa sequence having at least about 70% (e.g., at least about 75%, 80%, 85%, 90%, 95%, 98%, or 99%) or 100% aa sequence identity to at least 125 contiguous aas (e.g., at least 150, at least 175, at least 200, or at least 210 contiguous aas) or all aas of an aa sequence of the wild-type (wt.) Ig Fc sequence depicted in FIG. 2D. For example the non-interspecific immunoglobulin polypeptide sequence may comprise a human IgG1 Fc (e.g., comprising an aa sequence having at least about 90% or at least about 95% aa sequence identity to at least 150 or at least 200 contiguous aas of the wild-type (wt.) Ig Fc sequence depicted in FIG. 2D (SEQ ID NO:4).
43. The unconjugated T-Cell-MP or duplex T-Cell-MP of aspect 42, wherein the non-interspecific immunoglobulin polypeptide comprises at least one substitution at L234, L235, G236, G237, P238, S239, D270, N297, K322, P329, and/or P331 (respectively, aas L14, L15, G16, G17, P18, S19, D50, N77, K102, P109, and P111 of the wt. IgG1 aa sequence in FIG. 2D) or another substitution (e.g., a corresponding substitution) that reduces binding to the Fc receptor and/or the C1q protein relative to the same sequence without the substitutions.
44. The unconjugated T-Cell-MP or duplex T-Cell-MP of aspect 42, comprising: (i) a substitution of N297 (e.g., N297A); (ii) a substitution of any of aas 234 to 239; (iii) a substitution at L234; (iv) a substitution at L235; (v) a substitution at L234 and L235 (e.g., an L234A and L235A or “LALA” substitution); (vi) a substitution of P331; or (vii) substitutions of D270, K322, and/or P329; substitutions at L234 and/or L235, and a substitution at P331 (e.g., L234F, L235E, and P331S substitutions).
45. The unconjugated T-Cell-MP or duplex T-Cell-MP of aspect 39, wherein the scaffold sequence comprises an IgM heavy chain constant region.
46. The unconjugated T-Cell-MP or duplex T-Cell-MP of any of aspects 1-33, wherein the scaffold comprises an interspecific immunoglobulin polypeptide sequence.
47. The unconjugated T-Cell-MP or duplex T-Cell-MP of aspect 46, wherein the interspecific immunoglobulin sequence is selected from the group consisting of immunoglobulin heavy chain constant regions (Ig Fc CH2-CH3); Ig CH1; Ig C_{L} Ig C_{L} a knob-in-hole without disulfide (KiH), a knob-in hole with a stabilizing disulfide bond (KiHs-s), HA-TF, ZW-1, 7.8.60, DD-KK, EW-RVT, EW-RVTs-s, and A107 sequences.
48. The unconjugated T-Cell-MP or duplex T-Cell-MP of aspect 46, wherein the interspecific immunoglobulin sequence comprises a KIH or a KIHs-s polypeptide sequence.
49. The unconjugated T-Cell-MP or duplex T-Cell-MP of aspect 46, wherein the interspecific immunoglobulin comprises an EW-RVT or an EW-RVTs-s polypeptide sequence.
50. The unconjugated T-Cell-MP or duplex T-Cell-MP of aspect 46, wherein the interspecific immunoglobulin sequence comprises a HA-TF, ZW-1, 7.8.60, DD-KK, or A107 polypeptide sequence.
51. The unconjugated T-Cell-MP or duplex T-Cell-MP of any of aspects 46-50, further comprising one or more substitutions that reduce binding to the Fc receptor and/or the C1q protein (e.g., substitutions at IgG1 aa L234 and/or L235, or K322) relative to the same sequence without the substitutions.
52. The unconjugated T-Cell-MP or duplex T-Cell-MP of any of aspects 46-50, further comprising one or more substitutions that limit complement activation (e.g., reduce binding to the complement C1q protein such as by substitutions at IgG D270, N297, K322, P329, and/or P331) relative to the same sequence without the substitutions.
53. The unconjugated T-Cell-MP or duplex T-Cell-MP of any of aspects 46-52, wherein the interspecific immunoglobulin polypeptide sequence comprises a human IgG1 Fc comprising an aa sequence having at least about 70% (e.g., at least about 75%, 80%, 85%, 90%, or 95%) aa sequence identity to at least 125 contiguous aas (e.g., at least 150, at least 175, at least 200, or at least 210 contiguous aas) of the wt. Ig Gg1 Fc sequence in FIG. 2D.
54. The unconjugated T-Cell-MP or duplex T-Cell-MP of aspect 53, wherein the interspecific immunoglobulin polypeptide sequence comprises one or more Ig Fc regions, comprising at least one substitution at L234, L235, G236, G237, P238, 5239, D270, N297, K322, P329, and/or P331 (respectively, aas L14, L15, G16, G17, P18, S19, D50, N77, K102, P109, and P111 of the wt. IgG1 aa sequence in FIG. 2D) or another substitution (e.g., a corresponding substitution) that reduces binding to the Fc receptor and/or the C1q protein relative to the same sequence without the substitutions.
55. The unconjugated T-Cell-MP or duplex T-Cell-MP of aspect 53, comprising: (i) a substitution of N297 (e.g., N297A); (ii) a substitution of any of aas 234 to 239; (iii) a substitution at L234; (iv) a substitution at L235; (v) a substitution at L234 and L235 (e.g., an L234A and L235A or “LALA” substitution); (vi) a substitution of P331; or (vii) substitutions of D270, K322, and/or P329; substitutions at L234 and/or L235, and a substitution at P331 (e.g., L234F, L235E, and P331S substitutions).
56. The unconjugated T-Cell-MP or duplex T-Cell-MP of any of aspects 1-55, comprising at least one (e.g., at least two, or at least three) wt. MOD or variant MOD polypeptide sequences selected independently from the group consisting of: IL-1, IL-2, IL-4, IL-6, IL-7, IL-10, IL-12, IL-15, IL-17, IL-21, IL-23, CD7, CD30L, CD40, CD70, CD80, (B7-1), CD83, CD86 (B7-2), HVEM (CD270), ILT3 (immunoglobulin-like transcript 3), ILT4(immunoglobulin-like transcript 4), Fas ligand (FasL), ICAM (intercellular adhesion molecule), ICOS-L (inducible costimulatory ligand), JAG1 (CD339), lymphotoxin beta receptor, 3/TR6, OX40L (CD252), PD-L1, PD-L2, TGF-β1, TGF-β2, TGF-β3, 4-1BBL and anti-CD28 polypeptide sequences.
57. The unconjugated T-Cell-MP or duplex T-Cell-MP of any preceding aspect, comprising at least one (e.g., at least two, or at least three) wt. MOD or variant MOD polypeptide sequences selected independently from the group consisting of: 4-1BBL, anti-CD28, PD-L1, IL-2, CD80, CD86, OX40L (CD252), Fas ligand (FasL), ICOS-L, ICAM, CD30L, CD40, CD83, HVEM (CD270), JAG1 (CD339), CD70, CD80, CD86, TGF-β1, TGF-β2, and TGF-β3 polypeptide sequences.
58. The unconjugated T-Cell-MP or duplex T-Cell-MP of any preceding aspect, comprising at least one (e.g., at least two, or at least three) wt. MOD or variant MOD polypeptide sequences selected independently from the group consisting of 4-1BBL, PD-L1, IL-2, CD80, CD86, FasL wt. MOD or variant MOD polypeptide sequences and anti-CD28. For example, the unconjugated T-Cell-MP or duplex T-Cell-MP may comprise at least one wt. MOD and/or variant IL-2 MOD polypeptide sequence, and at least one wt. CD80, wt. CD86, variant CD80 or variant CD86 polypeptide sequence.
59. The unconjugated T-Cell-MP or duplex T-Cell-MP of any preceding aspect, comprising at least one wt. IL-2 or variant IL-2 MOD (e.g., comprising a H16A or T substitution and a F42A substitution) polypeptide sequence, or at least one pair of wt. IL-2 MOD or variant IL-2 MOD polypeptide sequences in tandem.
60. The unconjugated T-Cell-MP or duplex T-Cell-MP of aspect 59, further comprising at least one: (i) wt. or variant CD80 or CD86 MOD polypeptide sequence; (ii) wt. or variant PD-L1 MOD polypeptide sequence; and/or (iii) wt. or variant FasL MOD polypeptide sequence.
61. The unconjugated T-Cell-MP or duplex T-Cell-MP of any preceding aspect, further comprising an intrachain disulfide bond between a cysteine substituted into the carboxyl end portion of the α1 helix and a cysteine in the amino end portion of the α2-1 helix of the MHC-H polypeptide sequence.
62. The unconjugated T-Cell-MP or duplex T-Cell-MP of any preceding aspect, comprising an intrachain disulfide bond between a cysteine substituted into the carboxyl end portion of the α1 helix at position 84 and a cysteine in the amino end portion of the α2-1 helix at position 139 of the MHC-H polypeptide sequence;
   wherein the five residue clusters amino and carboxyl to position 84 (denoted aac 1 and aac 2, respectively) and, the five residue clusters amino and carboxyl to position 139 (denoted aac 3, and aac 4 respectively) may each be substituted with 1 to 5 independently selected naturally occurring aas, and the five residue clusters amino and carboxyl to position 236 (denoted aac 5 and aac 6, respectively) may each be substituted with 1 to 5 independently selected naturally occurring aas.
63. The unconjugated T-Cell-MP or duplex T-Cell-MP of aspect 62, wherein aac 1 to aac 6 may each be substituted with 1 to 5 independently selected naturally occurring aa other than proline.
64. The unconjugated T-Cell-MP or duplex T-Cell-MP of aspect 62, wherein the carboxyl end portion of the α1 helix comprises a first sequence CYNQSE and the amino end portion of the α2-1 helix of the MHC-H polypeptide sequence comprises a second sequence D(M/T)CAQ, and wherein the intrachain disulfide bond is formed between the cysteines in the first and second sequences. See aac 1 to aac 4 of FIG. 3I.
65. The duplex T-Cell-MP of any of aspects 33-64, wherein the first unconjugated T-Cell-MP and the second unconjugated T-Cell-MP are not linked by disulfide bonds.
66. The duplex T-Cell-MP of any of aspects 33-64, wherein the first unconjugated T-Cell-MP and the second unconjugated T-Cell-MP are covalently linked by at least one (e.g., two) disulfide bond(s).
67. The duplex T-Cell-MP of aspect 66, wherein the first unconjugated T-Cell-MP and the second unconjugated T-Cell-MP are covalently linked by at least one (e.g., two) disulfide bond(s) between the scaffold polypeptide sequences of the first T-Cell-MP and the second T-Cell-MP.
68. The duplex T-Cell-MP of any of aspects 33-66, wherein the sequences of at least one of (e.g., both) the first unconjugated T-Cell-MP and the second unconjugated T-Cell-MP do not comprise Ig CH1 domain polypeptide sequences.
69. The unconjugated duplex T-Cell-MP of any of aspects 33-35 and 37-45, wherein the first unconjugated T-Cell-MP and the second unconjugated T-Cell-MP are identical, and the unconjugated duplex T-Cell-MP is a homodimer. See, e.g., FIG. 6 structures A and B.
70. The unconjugated T-Cell-MP of aspect 69, comprising at least one (e.g., at least two, or at least three) wt. MOD or variant MOD polypeptide sequence selected independently from the group consisting of: IL-1, IL-2, IL-4, IL-6, IL-7, IL-10, IL-12, IL-15, IL-17, IL-21, IL-23, CD7, CD30L, CD40, CD70, CD80, (B7-1), CD83, CD86 (B7-2), HVEM (CD270), ILT3 (immunoglobulin-like transcript 3), ILT4(immunoglobulin-like transcript 4), Fas ligand (FasL), ICAM (intercellular adhesion molecule), ICOS-L (inducible costimulatory ligand), JAG1 (CD339), lymphotoxin beta receptor, 3/TR6, OX40L (CD252), PD-L1, PD-L2, TGF-β1, TGF-β2, TGF-β3, 4-1BBL polypeptide sequences and anti-CD28.
71. The unconjugated duplex T-Cell-MP of aspect 69, comprising at least one (e.g., at least two, or at least three) wt. MOD or variant MOD polypeptide sequence selected independently from the group consisting of: 4-1BBL, PD-L1, IL-2, CD80, CD86, FasL wt. MOD or variant MOD polypeptide sequences, and anti-CD28. For example, the unconjugated T-Cell-MP or duplex T-Cell-MP may comprise at least one IL-2 wt. MOD or variant MOD polypeptide sequence, and at least one CD80, CD86, variant CD80 or variant CD86 polypeptide sequence.
72. The unconjugated duplex T-Cell-MP of aspect 69, comprising at least one IL-2 wt. MOD or variant MOD (e.g., comprising a H16A or T substitution and a F42A substitution) polypeptide sequence, or at least one pair of IL-2 wt. MOD or variant MOD polypeptide sequences in tandem. 73. The unconjugated duplex T-Cell-MP of aspect 69, further comprising at least one: (i) CD80 and/or CD86 wt. MOD or variant MOD polypeptide sequence; (ii) at least one PD-L1 wt. MOD or variant MOD polypeptide sequence; and/or (iii) at least one FasL wt. MOD or variant MOD polypeptide sequence.
74. The unconjugated duplex T-Cell-MP of aspect 33-34, 36-38 and 46-68, wherein the scaffold polypeptides of the first unconjugated T-Cell-MP and the second unconjugated T-Cell-MP are a pair of interspecific polypeptide sequences and the unconjugated duplex T-Cell-MP is a heterodimer.
75. The unconjugated duplex T-Cell-MP of aspect 74, wherein at least one (e.g., at least two) of the first unconjugated T-Cell-MP and the second unconjugated T-Cell-MP comprises at least one wt. MOD or variant MOD polypeptide sequence selected independently from the group consisting of: IL-1, IL-2, IL-4, IL-6, IL-7, IL-10, IL-12, IL-15, IL-17, IL-21, IL-23, CD7, CD30L, CD40, CD70, CD80 (B7-1), CD83, CD86 (B7-2), HVEM (CD270), ILT3 (immunoglobulin-like transcript 3), ILT4 (immunoglobulin-like transcript 4), Fas ligand (FasL), ICAM (intercellular adhesion molecule), ICOS-L (inducible costimulatory ligand), JAG1 (CD339), lymphotoxin beta receptor, 3/TR6, OX40L (CD252), PD-L1, PD-L2, TGF-β1, TGF-β2, TGF-β3, anti-CD28, and 4-1BBL polypeptide sequences.
76. The unconjugated duplex T-Cell-MP of aspect 74, wherein at least one (e.g., at least two) of the first unconjugated T-Cell-MP and the second unconjugated T-Cell-MP comprises at least one wt. MOD or variant MOD polypeptide sequence selected independently from the group consisting of: 4-1BBL, anti-CD28, PD-L1, IL-2, CD80, CD86, and FasL wt. MOD or variant MOD polypeptide sequences. For example, the unconjugated duplex T-Cell-MP may comprise at least one IL-2 wt. MOD or variant MOD polypeptide sequence, and at least one anti-CD28, CD80, CD86, variant CD80 or variant CD86 polypeptide sequence.
77. The unconjugated duplex T-Cell-MP of aspect 74, wherein at least one (e.g., at least two) of the first unconjugated T-Cell-MP and the second unconjugated T-Cell-MP comprises at least one IL-2 wt. MOD or variant MOD polypeptide sequence, or at least one pair of IL-2 wt. MOD or variant MOD polypeptide sequences in tandem.
78. The unconjugated duplex T-Cell-MP of aspect 74, wherein at least one (e.g., at least two) of the first unconjugated T-Cell-MP and the second unconjugated T-Cell-MP comprises at least one: (i) CD80 and/or CD86 wt. MOD or variant MOD polypeptide sequence; (ii) at least one PD-L1 wt. MOD or variant MOD polypeptide sequence; and/or (iii) at least one FasL wt. MOD or variant MOD polypeptide sequence.
79. The unconjugated duplex T-Cell-MP of aspect 74, wherein at least one (e.g., at least two) of the first unconjugated T-Cell-MP and the second unconjugated T-Cell-MP comprises at least one CD80 and/or CD86 wt. MOD or variant MOD polypeptide sequence.
80. The unconjugated duplex T-Cell-MP of aspect 74, wherein at least one (e.g., at least two) of the first T-Cell-MP and the second T-Cell-MP comprises at least one PD-L1 wt. MOD or variant MOD polypeptide sequence.
81. The unconjugated duplex T-Cell-MP of any of aspects 74-80, wherein: (i) the first unconjugated T-Cell-MP and the second unconjugated T-Cell-MP do not comprise the same MODs; (ii) the first unconjugated T-Cell-MP and the second unconjugated T-Cell-MP do not comprise the same number of MODs; or (iii) the MODs are placed in different locations of the first unconjugated T-Cell-MP and the second unconjugated T-Cell-MP.
82. The T-Cell-MP of any of aspects 1-64, complexed to form a triplex T-Cell-MP of three heterodimers, a quadraplex T-Cell-MP of four heterodimers, a pentaplex T-Cell-MP of five heterodimers, or a hexaplex T-Cell-MP of six dimers.
83. The unconjugated T-Cell-MP or duplex T-Cell-MP of any of aspects 1-82, wherein each chemical conjugation site is jointly or independently selected from: a) amino acid chemical conjugation sites; b) non-natural amino acids and/or selenocysteines; c) peptide sequences that act as an enzymatic modification sequence (e.g., a sulfatase motif); d) carbohydrate or oligosaccharide moieties; and/or e) IgG nucleotide binding sites.
84. The unconjugated T-Cell-MP or duplex T-Cell-MP of any of aspects 1-83, wherein at least one (e.g., two or more) chemical conjugation site comprises an enzymatic modification sequence.
85. The unconjugated T-Cell-MP or duplex T-Cell-MP of any of aspects 1-84, wherein at least one (e.g., two or more) chemical conjugation site comprises a sulfatase motif.
86. The unconjugated T-Cell-MP or duplex T-Cell-MP of aspect 85, wherein the sulfatase motif comprises the sequence X1Z1X2Z2X3Z3 wherein:
Z1 is cysteine or serine; Z2 is either a proline or alanine residue; Z3 is a basic amino acid (arginine, lysine, or histidine, usually lysine), or an aliphatic amino acid (alanine, glycine, leucine, valine, isoleucine, or proline, usually A, G, L, V, or I);
X1 is present or absent and, when present, can be any amino acid, though usually an aliphatic amino acid, a sulfur-containing amino acid, or a polar, uncharged amino acid (i.e., other than an aromatic amino acid or a charged amino acid), usually L, M, V, S or T, more usually L, M, S or V, with the proviso that, when the sulfatase motif is at the N-terminus of the target polypeptide, X1 is present; and
X2 and X3 independently can be any amino acid, though usually an aliphatic amino acid, a polar, uncharged amino acid, or a sulfur containing amino acid (i.e., other than an aromatic amino acid or a charged amino acid), usually S, T, A, V, G or C, more usually S, T, A, V or G.
87. The unconjugated T-Cell-MP or duplex T-Cell-MP of aspect 86, wherein at least one Z1 residue has been converted into an fGly amino acid residue.
88. The unconjugated T-Cell-MP or duplex T-Cell-MP of any of aspects 1-84, wherein:
at least one (e.g., or more, two) of the chemical conjugation sites comprises a Sortase A enzyme site (e.g., comprising the amino acid sequence LP(X5)TG, LP(X5)TA, or LPETGG) positioned at the C-terminus of at least one (e.g., both) T-Cell-MP polypeptides; or
at least one of the chemical conjugation sites is a Sortase A enzyme site comprising an oligoglycine (e.g., (G)_{2, 3, 4, or 5}) or an oligo alanine (e.g., (A)_{2, 3, 4, or 5}) at the amino terminus of at least one of or both the first or second T-Cell-MP polypeptides.
89. The unconjugated T-Cell-MP or duplex T-Cell-MP of any of aspects 1-84, wherein at least one (e.g., two or more) chemical conjugation site comprises a transglutaminase site (e.g., selected from the group consisting of: LQG, LLQGG, LLQG, LSLSQG, and LLQLQG).
90. The unconjugated T-Cell-MP or duplex T-Cell-MP of any of aspects 1-84, wherein at least one (e.g., two or more) chemical conjugation site comprises a selenocysteine, or an amino acid sequence containing one or more independently selected non-natural amino acids.
91. The unconjugated T-Cell-MP or duplex T-Cell-MP of aspect 90, wherein at least one of the one or more non-natural amino acids (e.g., two or more) is selected from the group consisting of para-acetylphenylalanine, para-azido phenylalanine and propynyl-tyrosine.
92. The unconjugated T-Cell-MP or duplex T-Cell-MP of any of aspects 1-84, wherein at least one (e.g., two or more) chemical conjugation site comprises a carbohydrate, monosaccharide, disaccharide and/or oligosaccharide.
93. The unconjugated T-Cell-MP or duplex T-Cell-MP of any of aspects 1-84, wherein at least one (e.g., two or more) chemical conjugation site comprises one or more IgG nucleotide binding sites.
94. The unconjugated T-Cell-MP or duplex T-Cell-MP of any of aspects 1-84, wherein at least one (e.g., two or more) chemical conjugation site comprises an amino acid conjugation site (e.g., a cysteine provided in a T-Cell-MP by protein engineering of its sequence).
95. The unconjugated T-Cell-MP or duplex T-Cell-MP of aspect 94, wherein the at least one (e.g., two or more) chemical conjugation site comprises the epsilon amino group of a lysine provided in a T-Cell MP polypeptide sequence (e.g., provided in a T-Cell-MP by protein engineering of its polypeptide sequence).
96. The unconjugated T-Cell-MP or duplex T-Cell-MP of aspect 94, wherein the at least one (e.g., two or more) chemical conjugation site comprises a selenol group of selenocysteine and/or a sulfhydryl group of a cysteine provided in a T-Cell MP polypeptide sequence (e.g., provided in a T-Cell-MP by protein engineering of its polypeptide sequence).
97. The unconjugated T-Cell-MP or duplex T-Cell-MP of aspect 94, wherein the at least one chemical conjugation site comprises a sulfhydryl group of a cysteine provided in a T-Cell MP polypeptide sequence, or in the polypeptide sequence of each of the first T-Cell-MP and second T-Cell-MP of a duplex T-Cell-MP (e.g., provided in a T-Cell-MP by protein engineering of the polypeptide sequence(s)).
98. The unconjugated T-Cell-MP or duplex T-Cell-MP of any of aspects 1-97, wherein each chemical conjugation site (e.g., for the conjugation of an epitope) present in the unconjugated T-Cell-MP or duplexed unconjugated T-Cell-MP is selected to be the same (e.g., both are the sulfhydryl of a cysteine provided in the T-Cell-MP polypeptide sequences by protein engineering of the polypeptide sequences).
99. The unconjugated T-Cell-MP or duplex T-Cell-MP of any of aspects 1-98, wherein a chemical conjugation site (e.g., for the conjugation of an epitope) is located at the N- or C-terminus of a T-Cell-MP, or, if present, attached to or within a linker located at the N- or C-terminus of the T-Cell-MP.
100. The unconjugated T-Cell-MP or duplex T-Cell-MP of any of aspects 1-98, wherein a chemical conjugation site is located in a linker of the T-Cell-MP (e.g., an L1-L6 linker).
101. The unconjugated T-Cell-MP or duplex T-Cell-MP of any of aspects 1-98, wherein the one or more chemical conjugation site (e.g., for the conjugation of an epitope) is/are located in the MHC-H polypeptide sequence, the β2M polypeptide sequence, or a linker sequence joining the MHC-H and β2M polypeptide sequences (the L3 linker).
102. The unconjugated T-Cell-MP or duplex T-Cell-MP of any of aspects 1-98, wherein the one or more chemical conjugation site (e.g., for the conjugation of an epitope) is/are located in a linker sequence joining the MHC-H and β2M polypeptide sequences (the L3 linker).
103. The unconjugated T-Cell-MP or duplex T-Cell-MP of aspect 102, where the one or more chemical conjugation site is/are sulfhydryl of a cysteine present in the linker sequence joining the MHC-H and β2M polypeptide sequences.
104. The unconjugated T-Cell-MP or duplex T-Cell-MP of aspect 103, wherein the linker sequence joining the MHC-H and β2M polypeptide sequences further comprises a glycine, glycine and serine, alanine, alanine and serine, or alanine glycine and serine containing polypeptide sequence.
105. The unconjugated T-Cell-MP or duplex T-Cell-MP of aspect 103, wherein the linker sequence joining the MHC-H and β2M polypeptide sequences comprises the polypeptide sequence GGGS or GGGGS.
106. The unconjugated T-Cell-MP or duplex T-Cell-MP of aspect 103, wherein the linker sequence joining the MHC-H and β2M polypeptide sequences comprises a polypeptide sequence selected from the group consisting of: GCGGS(G4S) (SEQ ID NO:141) where the G4S unit may be repeated from 1 to 10 times (e.g., repeated 1, 2, 3, 4, 5, 6, 7, 8, 9, or 10 times),

(SEQ ID NO: 142)
GCGASGGGGSGGGGS,
(SEQ ID NO: 143)
GCGGSGGGGSGGGGSGGGGS
and
SEQ ID NO: 144)
GCGGSGGGGSGGGGS.

107. The unconjugated T-Cell-MP or duplex T-Cell-MP of any of aspects 101-106, wherein the linker sequence joining the MHC-H and β2M polypeptide sequences (the L3 linker) comprises from 15 to 50 amino acids.
108. The unconjugated T-Cell-MP or duplex T-Cell-MP of any of aspects 101-106, wherein the linker sequence joining the MHC-H and β2M polypeptide sequences (the L3 linker) comprises from 10 to 50 amino acids.
109. The unconjugated T-Cell-MP or duplex T-Cell-MP of any of aspects 1-98, wherein the one or more chemical conjugation site (e.g., for the conjugation of an epitope) is/are located in the T-Cell-MP MHC-H polypeptide sequence, which has at least 85% (e.g., at least 90%, 95%, 98% or 99%, or even 100%) aa sequence identity to at least 150, 175, 200, or 225 contiguous aas of a MHC-H sequence shown in FIGS. 3A-3I.
110. The unconjugated T-Cell-MP or duplex T-Cell-MP of aspect 109, wherein the one or more chemical conjugation sites comprise a cysteine or selenocysteine.
111. The unconjugated T-Cell-MP or duplex T-Cell-MP of aspect 110, wherein at least one cysteine or selenocysteine chemical conjugation site is located at position 2, 5, 7, 59, 84, 116, 139, 167, 168, 170, or 171 of a MHC-H polypeptide with the numbering as in FIGS. 3D-3I.
112. The unconjugated T-Cell-MP or duplex T-Cell-MP of any of aspects 1-98, wherein a chemical conjugation site (e.g., for the conjugation of an epitope) is located in the β2M polypeptide sequence, which has at least 85% (e.g., at least 90%, 95%, 98% or 99%, or even 100%) aa sequence identity to at least 50 (e.g., at least 60, 70, 80, 90, 96, 97, or 98 or all) contiguous aas of a mature β2M polypeptide sequence shown in FIG. 4 (e.g., the sequences shown in FIG. 4 starting at aa 21 and ending at their C-terminus).
113. The unconjugated T-Cell-MP or duplex T-Cell-MP of aspect 112, wherein the one or more chemical conjugation sites is/are located between aas 35-55 (e.g., 40 to 50) of the mature human β2M polypeptide sequence of FIG. 4 and has 0 to 15 aa substitutions.
114. The unconjugated T-Cell-MP or duplex T-Cell-MP of aspect 112, wherein at least one cysteine or selenocysteine chemical conjugation site is located at position 2, 44, 50, 77, 85, 88, 91, or 98 of the mature β2M polypeptides (aas 22, 64, 70, 97, 105, 108, 111, or 118 of the β2M sequences as shown in FIG. 4).
115. The unconjugated T-Cell-MP or duplex T-Cell-MP of any of aspects 1-98, wherein a chemical conjugation site (e.g., for the conjugation of an epitope) is located in the β2M polypeptide sequence, which has 1 to 15 (e.g., 1, 2, 3, 4, 5, 6, 7, 8, 9, 10, 11, 12, 13, 14, or 15) aa deletions, insertions and/or changes compared with a mature β2M polypeptide set forth in FIG. 4 (starting at aa 21 and ending at its C-terminus).
116. The unconjugated T-Cell-MP or duplex T-Cell-MP of any of aspects 112-115, wherein the chemical conjugation site is a cysteine.
117. The unconjugated T-Cell-MP or duplex T-Cell-MP of any of aspects 112-115, wherein the β2M polypeptide sequence is the mature human β2M sequence of FIG. 4.
118. The unconjugated duplex T-Cell-MP of any of aspects 33-117, wherein at least the first T-Cell-MP polypeptide sequence, and optionally the first and second T-Cell-MP polypeptide sequences comprise, from N-terminus to C-terminus:
   (i) one or more MOD polypeptide sequences optionally joined by L1 linkers;
   (ii) an L2 linker polypeptide sequence;
   (iii) a β2M polypeptide sequence;
   (iv) an L3 linker polypeptide sequence comprising from 10 to 50 (e.g., from 10 to 20, from 10 to 25, from 15 to 25, from 20 to 30, from 25 to 35, from 25 to 50, from 30 to 35, from 35 to 45, or from 40 to 50) amino acids;
   (v) a class I MHC-H polypeptide sequence comprising cysteines substituted at positions 84 and 139 (see FIGS. 3E-3H, e.g., Y84C and A139C substitutions) and forming a disulfide bond;
   (vi) an L4 linker polypeptide sequence;
   (vii) an interspecific or non-interspecific immunoglobulin Fc scaffold sequence;
   (viii) an optional L5 linker polypeptide sequence; and
   (ix) optionally one or more MOD polypeptide sequences (e.g., two or more MOD polypeptide sequences, such as in tandem, optionally joined by L6 linkers);
wherein at least one of the β2M polypeptide sequence, the L3 linker polypeptide sequence, or the MHC-H polypeptide sequence comprises a chemical conjugation site (e.g., added by protein engineering) for the direct or indirect (e.g., through a linker) covalent attachment of an epitope (e.g., a peptide, phosphopeptide, glycopeptide, lipopeptide or carbohydrate epitope); and wherein the first and second T-Cell-MPs are covalently linked through at least one disulfide bond between their Ig Fc scaffold sequences.
119. The unconjugated duplex T-Cell-MP of any of aspects 33-117, wherein at least the first T-Cell-MP polypeptide sequence, and optionally the first and second T-Cell-MP polypeptide sequences comprise:
   (i) optionally one or more MOD polypeptide sequences optionally joined by L1 linkers;
   (ii) an optional L2 linker polypeptide sequence;
   (iii) a β2M polypeptide sequence;
   (iv) an L3 linker polypeptide sequence comprising from 10 to 50 amino acids;
   (v) a class I MHC-H polypeptide sequence comprising cysteines substituted at positions 84 and 139 (see FIGS. 3E-3H, e.g., Y84C and A139C substitutions) and forming a disulfide bond;
   (vi) an L4 linker polypeptide sequence;
   (vii) an interspecific or non-interspecific immunoglobulin Fc scaffold sequence;
   (viii) an L5 linker polypeptide sequence; and
   (ix) one or more MOD polypeptide sequences joined by L6 linker polypeptides;
wherein at least one of the β2M polypeptide sequence, the L3 linker polypeptide sequence, or the MHC-H polypeptide sequence comprises a chemical conjugation site (e.g., added by protein engineering) for the direct or indirect (e.g., through a linker) covalent attachment of an epitope (e.g., a peptide, phosphopeptide, glycopeptide, lipopeptide or carbohydrate epitope); and wherein the first and second T-Cell-MPs are covalently linked through at least one disulfide bond between their Ig Fc scaffold sequences.
120. The unconjugated duplex T-Cell-MP of aspects 118 or 119, wherein the chemical conjugation site(s) of the first and second T-Cell-MP polypeptides is within the L3 linker.
121. The unconjugated duplex T-Cell-MP of aspect 120, wherein the chemical conjugation site(s) of the first and second T-Cell-MP polypeptides is the sulfhydryl of a cysteine present in the L3 linker comprises, consists essentially (predominantly) of, or otherwise consists of a glycine, serine and/or alanine residues.
122. The unconjugated duplex T-Cell-MP of aspects 118 or 119, wherein the chemical conjugation site(s) of the first and second T-Cell-MP polypeptides is within the β2M polypeptide sequence (e.g., an E44C substitution in a mature β2M polypeptide sequence provided in FIG. 4).
123. The unconjugated duplex T-Cell-MP of aspect 122, wherein the chemical conjugation site(s) of the first and second T-Cell-MP polypeptides is the sulfhydryl of a cysteine provided at the β2M polypeptide sequence.
124. The unconjugated duplex T-Cell-MP of aspect 123, wherein the chemical conjugation site(s) of the first and second T-Cell-MP polypeptides is the sulfhydryl of a cysteine provided at the β2M polypeptide at position 44 of a mature β2M polypeptide sequence provided in FIG. 4.
125. The unconjugated duplex T-Cell-MP of any of aspects 118-124, wherein the one or more MOD polypeptide sequences comprise at least one (e.g., two or more) wt. IL-2 or variant IL-2 sequence (e.g., comprising a H16A or T substitution and a F42A substitution).
126. The unconjugated duplex T-Cell-MP of any of aspects 118-125, wherein the one or more MOD polypeptide sequences comprise at least one wt. or variant CD80 or CD86 sequence.
127. The unconjugated duplex T-Cell-MP of any of aspects 118-126, wherein the one or more MOD polypeptide sequences comprise at least one wt. or variant PD-L1 sequence.
128. The unconjugated duplex T-Cell-MP of any of aspects 118-127, wherein the one or more MOD polypeptide sequences comprise at least one wt. or variant 4-1BBL or PD-L1 sequence.
129. The unconjugated duplex T-Cell-MP of any of aspects 118-128, wherein:
   (i) the immunoglobulin Fc scaffold is non-interspecific scaffold polypeptide and the duplex is a homodimer comprising identical first and second T-Cell-MP polypeptides; or
   (ii) the first and second scaffold polypeptides are an interspecific pair of immunoglobulin Fc scaffold polypeptides (e.g., a KIH or KIH-ss pair), and the duplex is a heterodimer.
130. The unconjugated duplex T-Cell-MP of aspect 129, wherein the first and second scaffold polypeptides are an interspecific pair of immunoglobulin Fc scaffold polypeptides, and the first T-Cell-MP polypeptide sequence comprises at least one MOD polypeptide sequence not present in the second T-Cell-MP polypeptide sequence.
131. The unconjugated T-Cell-MP or duplex T-Cell-MP of any preceding aspect further comprising an additional peptide and/or a payload covalently linked to a T-Cell-MP.
132. The unconjugated T-Cell-MP or duplex T-Cell-MP of aspect 131, wherein the additional peptide is an epitope tag or an affinity domain.
133. The unconjugated T-Cell-MP or duplex T-Cell-MP of aspect 131, wherein the additional peptide is a targeting sequence.
134. The unconjugated T-Cell-MP or duplex T-Cell-MP of aspect 133, wherein the targeting sequence is an antibody or an antigen binding fragment thereof, or a single chain T cell receptor.
135. The unconjugated T-Cell-MP or duplex T-Cell-MP of any of aspects 132-133, wherein the targeting sequence is directed to a protein or non-protein epitope of an infectious agent.
136. The unconjugated T-Cell-MP or duplex T-Cell-MP of aspect 135, where the infectious agent is a virus, bacteria, fungus, protozoa, or helminth.
137. The unconjugated T-Cell-MP or duplex T-Cell-MP of any of aspects 132-133, wherein the targeting sequence is directed to a self-antigen or allergen.
138. The unconjugated T-Cell-MP or duplex T-Cell-MP of any of aspects 132-133, wherein the targeting sequence is directed to a cancer-associated antigen (“CAA”).
139. The unconjugated T-Cell-MP or duplex T-Cell-MP of aspect 138, wherein the cancer associated antigen is selected from those recited in Section I.A.7.b.i.(a) “Cancer-Associated Antigens “CAAs”.
140. The unconjugated T-Cell-MP or duplex T-Cell-MP of aspect 138, wherein the targeting sequence is selected from the group consisting of an anti-CD51, anti-CD74, anti-CD22, anti-CD20, anti-CD20, anti-CD22, anti-CD38, anti-PD-1 receptor, anti-CTLA-4, anti-TROP-2, anti-mucin, anti-CEA, anti-CEACAM6, anti-colon-specific antigen-p, anti-alpha-fetoprotein, anti-IGF-1R, anti-CD19, anti-PSMA, anti-PSMA dimer, anti-carbonic anhydrase IX, anti-HLA-DR, anti-CD52, anti-EpCAM, anti-VEGF, anti-EGFR, anti-CD33, anti-HER2, anti-CD79b, anti-BCMA, and anti-mesothelin antibody or antigen binding fragment thereof.
141. The unconjugated T-Cell-MP or duplex T-Cell-MP of aspect 138, wherein the CAA is a peptide presented by an HLA as a peptide/HLA complex. See Section I.A.7.b.i.(b) “Peptide/HLA Complexes.”
142. The unconjugated T-Cell-MP or duplex T-Cell-MP of aspect 138, wherein the targeting sequence is selected from an anti-HER2, anti-CD19, anti-mesothelin, anti-TROP2, anti-BCMA, anti-MUC-1, anti-MUC16, or anti-Claudin antibody or antigen binding fragment thereof. See Section I.A.7.b.ii at (a) though (h).
143. The unconjugated T-Cell-MP or duplex T-Cell-MP of any preceding aspect further comprising a payload covalently attached to a T-Cell-MP.
144. The unconjugated T-Cell-MP or duplex T-Cell-MP of aspect 143, wherein the payload is a therapeutic agent, chemotherapeutic agent, diagnostic agent, or label.
145. The unconjugated T-Cell-MP or duplex T-Cell-MP of any preceding aspect, wherein at least one T-Cell-MP is conjugated to a non-peptide or peptide epitope at the chemical conjugation site in the β2M polypeptide sequence, the L3 polypeptide sequence, and/or the MHC-H polypeptide sequence to form a T-Cell-MP-epitope conjugate or a higher order T-Cell-MP-epitope conjugate complex, such as a duplex T-Cell-MP-epitope conjugate.
146. The T-Cell-MP-epitope conjugate or duplex T-Cell-MP-epitope conjugate of aspect 145, wherein the epitope is a cancer epitope, infectious agent epitope, self-epitope (autoantigen), or allergen epitope.
147. The T-Cell-MP-epitope conjugate or duplex T-Cell-MP-epitope conjugate of any of aspects 145-146, wherein the epitope is a peptide, glycopeptide, phosphopeptide, or lipopeptide that comprises from about 4 aas (aa) to about 25 aa (e.g., the epitope can have a length of from 4 aa to about 10 aa, from about 6 aa to about 12 aa, from about 10 aa to about 15 aa, from about 15 aa to about 20 aa, or from about 20 aa to about 25 aa).
148. The T-Cell-MP-epitope conjugate or duplex T-Cell-MP-epitope conjugate of aspect 147, wherein the peptide epitope is from about 6 aa to about 12 aa.
149. The T-Cell-MP-epitope conjugate or duplex T-Cell-MP-epitope conjugate of any of aspects 145-148, wherein the epitope is a cancer epitope.
150. The T-Cell-MP-epitope conjugate or duplex T-Cell-MP-epitope conjugate of aspect 149, wherein the cancer epitope is set forth in section I.A.8.d.i “Epitopes present in cancers—Cancer-Associated Antigens (“CAAs”).”
151. The T-Cell-MP-epitope conjugate or duplex T-Cell-MP-epitope conjugate of aspect 150, wherein the cancer epitope is an Alpha Feto Protein (AFP) epitope set forth in section I.A.8.d.i(a) “Alpha Feto Protein (AFP)”.
152. The T-Cell-MP-epitope conjugate or duplex T-Cell-MP-epitope conjugate of aspect 150, wherein the cancer epitope is an epitope of Wilms Tumor Antigen (WT-1) protein set forth in section I.A.8.d.i(b) “Wilms Tumor Antigen (WT-1)”.
153. The T-Cell-MP-epitope conjugate or duplex T-Cell-MP-epitope conjugate of aspect 150, wherein the cancer epitope is a Human Papilloma Virus I (HPV) epitope set forth in section I.A.8.d.i(c) “Human Papilloma Virus I (HPV)”.
154. The T-Cell-MP-epitope conjugate or duplex T-Cell-MP-epitope conjugate of aspect 150, wherein the cancer epitope is a Hepatitis B Virus (HBV) epitope set forth in section I.A.8.d.i(d) “Hepatitis B Virus (HBV)”.
155. The T-Cell-MP-epitope conjugate or duplex T-Cell-MP-epitope conjugate of any of aspects 145-148, wherein the epitope is a self-epitope.
156. The T-Cell-MP-epitope conjugate or duplex T-Cell-MP-epitope conjugate of any of aspects 145-148, wherein the epitope is an epitope of an allergen (e.g., an allergic protein).
157. The T-Cell-MP-epitope conjugate or duplex T-Cell-MP-epitope conjugate of aspect 156, wherein the allergen is selected from protein or non-protein components of: nuts (e.g., tree and/or peanuts), glutens, pollens, eggs (e.g. chicken, Gallus domesticus), shellfish, soy, fish, and insect venoms (e.g., bee and/or wasp venom antigens).
158. The T-Cell-MP-epitope conjugate or duplex T-Cell-MP-epitope conjugate of any of aspects 145-148, wherein the epitope is an epitope presented by an infectious agent.
159. The T-Cell-MP-epitope conjugate or duplex T-Cell-MP-epitope conjugate of aspect 158, wherein the infectious agent is a virus, bacterium, fungus, protozoan, or helminth 160. The T-Cell-MP-epitope conjugate or duplex T-Cell-MP-epitope conjugate of any of aspects 145-159, wherein the infectious agent is a virus and the epitope is an epitope presented by a viral infectious disease agent (e.g., a virus set forth in section I.A.8.d.ii “Infectious Agents”).
161. A method of treatment or prophylaxis of a disease (e.g., a cancer or infection) or condition (e.g., an allergy) comprising:
   (i) administering to a patient/subject (e.g., a patient in need thereof) an effective amount of one or more unconjugated T-Cell-MPs or unconjugated duplex T-Cell-MPs of any of aspects 1-144;
   (ii) administering to a patient/subject (e.g., a patient in need thereof) an effective amount of one or more T-Cell-MP-epitope conjugates or duplex T-Cell-MP-epitope conjugates of any of aspects 145-160;
   (iii) administering to a patient/subject (e.g., a patient in need thereof) an effective amount of one or more nucleic acids encoding an unconjugated T-Cell-MP or unconjugated duplex T-Cell-MPs according to any of aspects 1-144;
   (iv) contacting a cell or tissue in vitro, in vivo, or ex vivo with one or more unconjugated T-Cell-MP or unconjugated duplex T-Cell-MPs according to any of aspects 1-144 and administering the cell, tissue, or progeny thereof to a patient/subject (e.g., a patient in need thereof);
   (v) contacting a cell or tissue in vitro, in vivo, or ex vivo with one or more T-Cell-MP-epitope conjugates or duplex T-Cell-MP-epitope conjugates of any of aspects 145-160, and administering the cell, tissue, or progeny thereof to a patient/subject (e.g., a patient in need thereof);
   Or
   (vi) contacting a cell or tissue in vitro, in vivo, or ex vivo with one or more nucleic acids encoding a T-Cell-MP or duplex T-Cell-MP of any of aspects 1-133 and administering the cell, tissue, or progeny thereof to a patient/subject (e.g., a patient in need thereof).
162. A method of treatment or prophylaxis of a disease (e.g., a cancer or infection) or condition (e.g., an allergy) comprising
   (i) administering to a patient/subject (e.g., a patient in need thereof) an effective amount of one or more T-Cell-MP-epitope conjugates or duplex T-Cell-MP-epitope conjugates of any of aspects 145-160; or
   (ii) contacting a cell or tissue in vitro, in vivo, or ex vivo with one or more T-Cell-MP-epitope conjugates or duplex T-Cell-MP-epitope conjugates of any of aspects 145-160, and administering the cell, tissue, or progeny thereof to a patient/subject (e.g., a patient in need thereof).
163. The method of aspect 162, wherein the one or more T-Cell-MP-epitope conjugates or duplex T-Cell-MP-epitope conjugates comprises an unconjugated T-Cell-MP-epitope conjugate or duplex T-Cell-MP-epitope conjugate of any of aspects 118-130 conjugated to an epitope (e.g., a peptide, lipopeptide, phosphopeptide, carbohydrate or glycopeptide epitope) to form a T-Cell-MP-epitope conjugate or duplex T-Cell-MP-epitope conjugate.
164. The method of any of aspects 161-163, wherein the T-Cell-MP-epitope conjugate or duplex T-Cell-MP-epitope conjugate further comprises at least one targeting sequence (e.g., a targeting sequence specific for a cell or tissue).
165. The method of any of aspects 131-164, wherein the administration is to a mammalian patient or subject.
166. The method of aspect 165, wherein the patient or subject is human.
167. The method of aspect 165, wherein the patient or subject is non-human (e.g., rodent, lagomorph, bovine, canine, feline, rodent, murine, caprine, simian, ovine, equine, lappine, porcine, etc.).
168. The method of any of aspects 161-167, wherein the disease or condition is a cancer, and wherein, when a targeting sequence is present, it is a CTP (e.g., anti-HER2, anti-CD19, anti-mesothelin, anti-TROP2, anti-BCMA, anti-MUC-1, anti-MUC16).
169. The method of any of aspects 161-168, wherein the epitope is a cancer epitope.
170. The method of any of aspects 161-167, wherein the disease or condition is an infection.
171. The method of any of aspects 161-167, wherein the disease is a viral infection.
172. The method of any of aspects 161-167, wherein the disease is a bacterial, fungal or protozoan infection.
173. The method of any of aspects 161-172, further comprising administering one or more therapeutic agents that enhance CD 8+ T cell functions (e.g., effector function) and/or treat the disease or condition.
174. The method of aspect 173, wherein the therapeutic agent that enhances CD 8+ function and/or treats the disease or condition comprises an anti-TGF-β antibody such as Metelimumab (CAT192) directed against TGF-β1 and Fresolimub directed against TGF-β1 and TGF-β2, or a TGF-β trap (optionally subject to the proviso that the T-Cell-MP or duplexed T-Cell-MP does not comprise an aa sequence to which the antibodies or TGF-β trap bind such as a TGF-β1 or TGF-β2 wt. MOD or variant MOD aa sequence).
175. The method of any of aspects 173-174, wherein the therapeutic agent that enhances CD 8+ function and/or treats the disease or condition comprises one or more antibodies directed against: B lymphocyte antigens (e.g., ibritumomab tiuxetan, obinutuzumab, ofatumumab, rituximab to CD20, brentuximab vedotin directed against CD30, and alemtuzumab to CD52); EGFR (e.g., cetuximab, panitumumab, and necitumumab); VEGF (e.g., bevacizumab); VEGFR2 (e.g., ramucirumab); HER2 (e.g., pertuzumab, trastuzumab, and ado-trastuzumab); PD-1 (e.g., nivolumab and pembrolizumab targeting a check point inhibition); RANKL (e.g., denosumab); CTLA-4 (e.g., ipilimumab targeting check point inhibition); IL-6 (e.g., siltuximab); disialoganglioside (GD2) (e.g., dinutuximab); CD38 (e.g., daratumumab); SLAMF7 (Elotuzumab); both EpCAM and CD3 (e.g., catumaxomab); or both CD19 and CD3 (blinatumomab) (optionally subject to the proviso that the T-Cell-MP or duplexed T-Cell-MP does not comprise an aa sequence to which the antibodies bind).
176. The method of any of aspects 161-175, further comprising administering one or more additional therapeutic agents (e.g., chemotherapeutic, antibiotic, antifungal, antiviral, and/or anti-helminth agents). 177. The method of aspect 176, wherein the disease is a cancer and the method further comprises administering one or more chemotherapeutic agents.
178. The method of aspect 177, wherein the one or more chemotherapeutic agents are selected from the group consisting of: alkylating agents, cytoskeletal disruptors (taxane), epothilones, histone deacetylase inhibitors, topoisomerase I inhibitors, topoisomerase II inhibitors, kinase inhibitors, nucleotide analogs or precursor analogs, peptide antineoplastic antibiotics (e.g. bleomycin or actinomycin), platinum-based agents, retinoids, vinca alkaloids and their derivatives.
179. The method of aspect 176, wherein the one or more chemotherapeutic agents are selected from the group consisting of actinomycin all-trans retinoic acid, azacytidine, azathioprine, bleomycin, bortezomib, carboplatin, capecitabine, cisplatin, chlorambucil, cyclophosphamide, cytarabine, daunorubicin, docetaxel, doxifluridine, doxorubicin, epirubicin, epothilone, etoposide, fluorouracil, gemcitabine, hydroxyurea, idarubicin, imatinib, irinotecan, mechlorethamine, mercaptopurine, methotrexate, mitoxantrone, oxaliplatin, paclitaxel, pemetrexed, teniposide, tioguanine, topotecan, valrubicin, vemurafenib, vinblastine, vincristine, and vindesine.
180. The method of any of aspects 161-167, further comprising administering one or more therapeutic agents that suppress CD 8+ T cell functions (e.g., suppress effector function), suppress immune response, and/or treat the disease or condition.
181. The method of aspect 180, wherein the disease or condition is an autoimmune disease and the epitope is a self-antigen (self-epitope).
182. The method of aspect 180, wherein the disease or condition is an allergy and the epitope is an allergen.
183. The method of any of aspects 180-182, further comprising administering an NSAID (e.g., Cox-1 and/or Cox-2 inhibitors such as celecoxib, diclofenac, diflunisal, etodolac, ibuprofen, indomethacin, ketoprofen, and naproxen).
184. The method of any of aspects 180-183, further comprising administering a corticosteroid (e.g., cortisone, dexamethasone, hydrocortisone, ethamethasoneb, fludrocortisone, methylprednisolone, prednisone, prednisolone and triamcinolone).
185. The method of any of aspects 180-184, further comprising administering an agent that blocks one or more actions of tumor necrosis factor alpha (e.g., an anti-TNF alpha such as golimumab, infliximab, certolizumab, adalimumab or a TNF alpha decoy receptor such as etanercept) (optionally subject to the proviso that the T-Cell-MP or duplexed T-Cell-MP does not comprise tumor necrosis factor alpha wt. MOD or variant MOD and/or an aa sequence to which the agent that blocks one or more actions of TNF alpha binds).
186. The method of any of aspects 180-185, further comprising administering one or more agents that bind to the IL-1 receptor competitively with IL-1 (e.g., anakinra) (optionally subject to the proviso that the T-Cell-MP or duplexed T-Cell-MP does not comprise an IL-1 wt. MOD or variant MOD and/or an aa sequence to which the agent binds).
187. The method of any of aspects 180-186, further comprising administering one or more agents that bind to the IL-6 receptor and inhibits IL-6 from signaling through the receptor (e.g., tocilizumab), (optionally subject to the proviso that the T-Cell-MP or duplexed T-Cell-MP does not comprise an IL-6 wt. MOD or variant MOD and/or an aa sequence to which the agent binds).
188. The method of any of aspects 180-187, further comprising administering one or more agents that bind to CD80 and/or CD86 receptors and inhibit T cell proliferation and/or B cell immune response (e.g., abatacept) (optionally subject to the proviso that the T-Cell-MP or duplexed T-Cell-MP does not comprise a CD80 and/or CD86 wt. MOD or variant MOD and/or an aa sequence to which the agent binds).
189. The method of any of aspects 180-188, further comprising administering one or more agents that bind to CD20 resulting in B-Cell death (e.g., rituximab) (subject to the proviso that the T-Cell-MP or duplexed T-Cell-MP does not comprise a CD20 wt. MOD or variant MOD, and/or an aa sequence to which the agent binds).
190. The method of any of aspects 180-189, wherein the T-Cell-MP or duplex T-Cell-MP, or the nucleic acid encoding a T-Cell-MP or duplex T-Cell-MP is administered in a composition comprising the T-Cell-MP or duplex T-Cell-MP and at least one pharmaceutically acceptable excipient.
191. A nucleic acid sequence encoding an unconjugated T-Cell-MP of any of aspects 1 to 144 optionally comprising an additional polypeptide.
192. The nucleic acid sequence of aspect 191, operably linked to a promoter.
193. One or more nucleic acids comprising a nucleic acid sequence encoding an unconjugated duplex T-Cell-MP according to any of aspects 1-144, wherein at least one of the duplexed molecules optionally comprises an additional polypeptide.
194. The one or more nucleic acids of aspect 193, wherein the nucleic acid sequence encoding the unconjugated duplex T-Cell-MP is operably linked to a promoter.
195. A method of producing cells expressing a T-Cell-MP or duplex T-Cell-MP, the method comprising introducing one or more nucleic acids according to any of aspects 191-194 into the cells in vitro or ex vivo; selecting for cells that produce the unconjugated T-Cell-MP or unconjugated duplex T-Cell-MP; and optionally selecting for cells comprising all or part of the one or more nucleic acids either unintegrated or integrated into at least one cellular chromosome.
196. The method of aspect 195, wherein the cells are cells of a mammalian cell line selected from the HeLa cells, CHO cells, 293 cells (HEK-293 cells), Vero cells, NIH 3T3 cells, Huh-7 cells, BHK cells, PC12, COS cells, COS-7 cells, RAT1 cells, mouse L cells, human embryonic kidney (HEK) cells, and HLHepG2 cells.
197. One or more cells transiently or stably expressing a T-Cell-MP or duplex T-Cell-MP prepared by the method of aspect 195 or 196.
198. The cells of aspect 197, wherein the cells express from about 20 to about 200 (e.g., 20-40, 40-80, 80-100, 100-120, 120-140, 140-160. 160-180 or 180-200) mg/liter or more of the unconjugated T-Cell-MP.
199. The cell of aspect 198, wherein the cells express from about 20 to about 200 mg/liter or more of the unconjugated T-Cell-MP without a substantial reduction (less than a 5%, 10%, or 15% reduction) in cell viability relative to otherwise identical cells not expressing the T-Cell-MP or duplex T-Cell-MP.
200. A method of selectively delivering one or more (e.g., two or more) wt. MOD polypeptides and/or variant MOD polypeptides to one or more cells or tissues of a patient or subject, the method comprising:
   (i) administering to a patient/subject (e.g., a patient in need thereof) an effective amount of one or more T-Cell-MPs or duplex T-Cell-MPs of any of aspects 1-160;
   wherein at least one or more T-Cell-MPs of the one or more T-Cell-MPs or duplex T-Cell-MPs comprises a targeting sequence specific to the one or more cells or tissues; and
   wherein the T-Cell-MP or duplex T-Cell-MP comprises one or more (e.g., two or more) wt. MODs and/or variant MODs.
201. A method of selectively delivering one or more (e.g., two or more) wt. MOD polypeptides and/or variant MOD polypeptides to one or more T cells or tissues of a patient or subject, the method comprising:
   (i) administering to a patient/subject (e.g., a patient in need thereof) an effective amount of one or more T-Cell-MP-epitope conjugates or duplex T-Cell-MP-epitope conjugates of any of aspects 145-160;
   wherein at least one T cell present in the one or more T cells, or tissues is selective (e.g., specific) for the epitope conjugated to the T-Cell-MP; and
   wherein the T-Cell-MP or duplex T-Cell-MP comprises one or more wt. MODs or variant MODs.
202. The method of aspect 200 or 201, wherein the one or more wt. MOD polypeptides and/or variant MOD polypeptides are selected independently from the group consisting of: 4-1BBL, PD-L1, IL-2, CD80, CD86, OX40L (CD252), Fas ligand (FasL), ICOS-L, ICAM, CD30L, CD40, CD83, HVEM (CD270), JAG1 (CD339), CD70, TGF-β1, TGF-β2, and TGF-β3 wt. MOD or variant MOD polypeptide sequences.
203. The method of aspect 200 or 201, wherein the one or more wt. MOD polypeptides and/or variant MOD polypeptides are selected independently from the group consisting of: 4-1BBL, PD-L1 IL-2, CD80, CD86, and FasL wt. MOD and variant MOD polypeptide sequences of any thereof.
204. The method of any of aspects 200 to 202, wherein the T-Cell-MP or duplex T-Cell-MP comprises at least one IL-2 wt. MOD or variant MOD polypeptide sequence, and at least one CD80, CD86, variant CD80 or variant CD86 polypeptide sequence.
205. The method of any of aspects 200 to 202, wherein the T-Cell-MP or duplex T-Cell-MP comprises at least one IL-2 wt. MOD or variant MOD polypeptide sequence, or at least one pair of IL-2 wt. MOD or variant MOD polypeptide sequences in tandem.
206. The method of any of aspects 200 to 202, wherein the T-Cell-MP or duplex T-Cell-MP comprises at least one CD80 and/or CD86 wt. MOD or variant MOD polypeptide sequence.
207. The method of any of aspects 200 to 202, wherein the T-Cell-MP or duplex T-Cell-MP comprises at least one PD-L1 wt. MOD or variant MOD polypeptide sequence.
208. The method of any of aspects 200 to 202, wherein the T-Cell-MP or duplex T-Cell-MP comprises at least one FasL wt. MOD or variant MOD polypeptide sequence.

### X. Examples

### Example 1

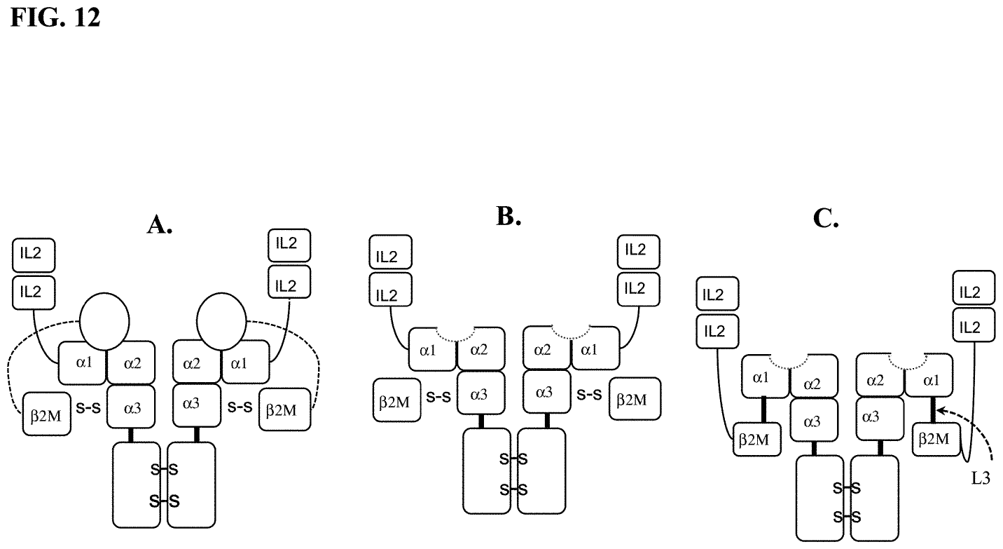

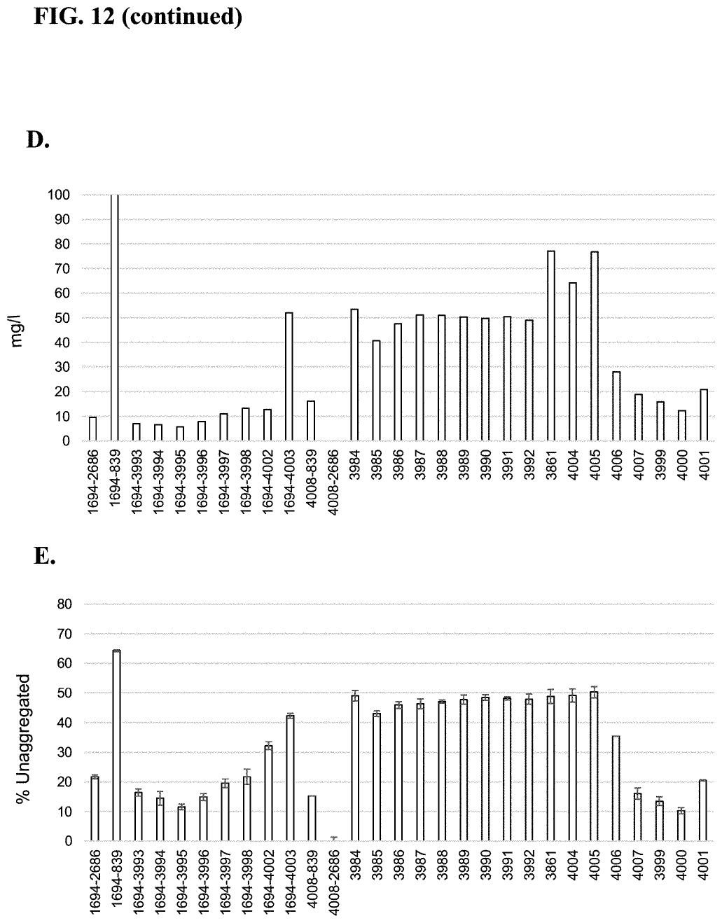

Nucleic acids were prepared encoding a series of constructs comprising a HLA-A*02:01(HLA-A02) class I heavy chain polypeptide sequence, a human β2M polypeptide sequence, and an IgG scaffold sequence, as core elements of split chain or single chain constructs shown as duplexes in FIG. 12 at A, B and C.

Each of the split chain constructs (structures A or B) has a first polypeptide sequence that comprises from the N-terminus to the C-terminus tandem human IL-2 polypeptide sequences (2×hIL2) with F42A,H16A substitutions, HLA-A*02:01 (A02) α1, α2, and α3 domains, and a human IgG1 scaffold with L234A and L235A substitutions. The 1694 first polypeptide appearing in most of the split chain constructs comprises an A236C,Y84C and A139C substitutions 2×hIL2 (F42A, H16A)-(G₄S)₄-HLA-A02 (A236C, Y84C, A139C)-AAAGG-IgG1 (L234A, L235A):

(SEQ ID NO: 493)
APTSSSTKKTQLQLEALLLDLQMILNGINNYKNPKLTRMLTAKFYMPKKAT
ELKHLQCLEEELKPLEEVLNLAQSKNFHLRPRDLISNINVIVLELKGSETT
FMCEYADETATIVEFLNRWITFCQSIISTLT [symbol]
APTSSSTKKTQLQLEALLLDLQMILNGINNYKNPKLTRMLTAKFYMPKKAT
ELKHLQCLEEELKPLEEVLNLAQSKNFHLRPRDLISNINVIVLELKGSETT
FMCEYADETATIVEFLNRWITFCQSIISTLT [symbol]
GSHSMRYFFTSVSRPGRGEPRFIAVGYVDDTQFVRFDSDAASQRMEPRAPW
IEQEGPEYWDGETRKVKAHSQTHRVDLGTLRG [symbol] YNQSEAGSHTVQRMYGCD
VGSDWRFLRGYHQYAYDGKDYIALKEDLRSWTAADM [symbol] AQTTKHKWEAAHVA
EQLRAYLEGTCVEWLRRYLENGKETLQRTDAPKTHMTHHAVSDHEATLRCW
ALSFYPAEITLTWQRDGEDQTQDTELVETRP [symbol] GDGTFQKWAAVVVPSGQEQ
RYTCHVQHEGLPKPLTLRWE [symbol] DKTHTCPPCPAPEAAGGPSVFLFPPK
PKDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQV
YTLPPSREEMTKNQVSLTCLVKGFYPSDIAVEWESNGQPENNYKTTPPVLD
SDGSFFLYSKLTVDKSRWQQGNVFSCSVMHEALHNHYTQKSLSLSPGK
(linker sequences are indicated in bold and
italics).

The 4008 polypeptide appearing in two split chain constructs parallels the 1694 construct, but comprises A236C, Y85C, and D137C substitutions in the HLA-A02 sequence—2×hIL2 (F42A, H16A)-(G4S)₄-HLA-A02 (A236C, Y85C, D137C)-AAAGG-IgG1 (L234A, L235A):

(SEQ ID NO: 494)
APTSSSTKKTQLQLEALLLDLQMILNGINNYKNPKLTRMLTAKFYMPKKAT
ELKHLQCLEEELKPLEEVLNLAQSKNFHLRPRDLISNINVIVLELKGSETT
FMCEYADETATIVEFLNRWITFCQSIISTLT [symbol]
APTSSSTKKTQLQLEALLLDLQMILNGINNYKNPKLTRMLTAKFYMPKKAT
ELKHLQCLEEELKPLEEVLNLAQSKNFHLRPRDLISNINVIVLELKGSETT
FMCEYADETATIVEFLNRWITFCQSIISTLT [symbol]
GSHSMRYFFTSVSRPGRGEPRFIAVGYVDDTQFVRFDSDAASQRMEPRAPW
IEQEGPEYWDGETRKVKAHSQTHRVDLGTLRGYCNQSEAGSHTVQRMYGCD
VGSDWRFLRGYHQYAYDGKDYIALKEDLRSWTAA [symbol] MAAQTTKHKWEAAHVA
EQLRAYLEGTCVEWLRRYLENGKETLQRTDAPKTHMTHHAVSDHEATLRCW
ALSFYPAEITLTWQRDGEDQTQDTELVETRPCGDGTFQKWAAVVVPSGQEQ
RYTCHVQHEGLPKPLTLRWE [symbol] DKTHTCPPCPAPEAAGGPSVFLFPPK
PKDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQV
YTLPPSREEMTKNQVSLTCLVKGFYPSDIAVEWESNGQPENNYKTTPPVLD
SDGSFFLYSKLTVDKSRWQQGNVFSCSVMHEALHNHYTQKSLSLSPGK.

Each of the split chain constructs (structures A and B) in FIG. 12 comprises a second polypeptide comprising a β2M polypeptide sequence having R12C and E44C substitutions: IQRTPKIQVYSCHPAENGKSNFLNCYVSGFHPSDIEVDLLKNGCRIEKVEHSDLSFSKDWSFYLLY YTEFTPTEKDEYACRVNHVTLSQPKIVKWDRDM (SEQ ID NO:495); to which ether the indicated linker, or CMV peptide epitope NLVPMVATV and linker, is added at their N-termini as indicated in the table provided below.

The unconjugated T-Cell-MP conjugates listed in FIG. 12 each comprise as a single polypeptide chain from N-terminus to C-terminus IL-2, β2M, HLA-A*02:01 (A02) α1, α2, and α3 domains, and human IgG1 polypeptide sequences. The aa sequence of the 3861 construct is provided below, and the remainder of the single chain T-Cell-MP constructs may be considered variations of the 3861 construct, which has tandem 2×IL-2 sequences with F42A and H16A substitutions-a (G₄S)₄ linker-β2M (E44C)-a (G₄S)₃ linker-HLA-A02 with Y84C, A139C— a AAAGG linker- and an IgG1 with L234A and L235A substitutions: APTSSSTKKTQLQLEALLLDLQMILNGINNYKNPKLTRMLTAKFYMPKKATEL KHLQCLEEELKPLEEVLNLAQSKNFHLRPRDLISNINVIVLELKGSETTFMCEYADETATIVEFLN RWITFCQSIISTLT [symbol] APTSSSTKKTQLQLEALLLDLQMILNGINNYK NPKLTRMLTAKFYMPKKATELKHLQCLEEELKPLEEVLNLAQSKNFHLRPRDLISNINVIVLELK GSETTFMCEYADETATIVEFLNRWITFCQSIISTLT [symbol] IQRTPKIQVY SRHPAENGKSNFLNCYVSGFHPSDIEVDLLKNGCRIEKVEHSDLSFSKDWSFYLLYYTEFTPTEK DEYACRVNHVTLSQPKIVKWDRDM [symbol] GSHSMRYFFTSVSRPGRGEPRFIA VGYVDDTQFVRFDSDAASQRMEPRAPWIEQEGPEYWDGETRKVKAHSQTHRVDLGTLRGCYN QSEAGSHTVQRMYGCDVGSDWRFLRGYHQYAYDGKDYIALKEDLRSWTAADMCAQTTKHKW EAAHVAEQLRAYLEGTCVEWLRRYLENGKETLQRTDAPKTHMTHHAVSDHEATLRCWALSFY PAEITLTWQRDGEDQTQDTELVETRPAGDGTFQKWAAVVVPSGQEQRYTCHVQHEGLPKPLTL RWE [symbol] DKTHTCPPCPAPEAAGGPSVFLFPPKPKDTLMISRTPEVTCVVVDVSHEDPEVKFN WYVDGVEVHNAKTKPREEQYNSTYRVVSVLTVLHQDWLNGKEYKCKVSNKALPAPIEKTISKA KGQPREPQVYTLPPSREEMTKNQVSLTCLVKGFYPSDIAVEWESNGQPENNYKTTPPVLDSDGSF FLYSKLTVDKSRWQQGNVFSCSVMHEALHNHYTQKSLSLSPG (SEQ ID NO:496).

Where tandem IL-2 sequences are present in the constructs of this example, they are separated by a (G₄S)₄ linker. Each of the sequences other than 3861 has variations in the linkers present between the IL-2 and β2M, and/or β2M and HLA-A02 sequences (the L3 linker) as indicated. Additionally, construct 3984 has only a single IL-2 sequence, and each of 3999-4002 have an additional aa substitution in the HLA-A02 polypeptide sequence as indicated in the table that follows.

| | | |
|---|---|---|
| | Form in | |
| Construct | FIG. 12 | Construct Content and Organization |

Split Chain Construct).

| | | | |
|---|---|---|---|
| 1694 | 2686 | B | β2M (R12C, E44C) |
| | 839 | A | CMV-GGGASGGGGSGGGGS-β2M (R12C) |
| | 3993 | B | GM-(G₄S)₃-β2M (R12C, E44C) |
| | 3994 | B | GMGGS-(G₄S)₂₋β2M (R12C, E44C) |
| | 3995 | B | GMS-(G₄S)2-β2M (R12C, E44C) |
| | 3996 | B | GMGGGS-(G₄S)-β2M (R12C, E44C) |
| | 3997 | B | GMGS-(G₄S)-β2M (R12C, E44C) |
| | 3998 | B | GM-(G₄S)-β2M (R12C, E44C) |
| | 4002 | A | CMV-(G₃AS)-(G4S)₂-β2M (R12C, E44C) |
| | 4003 | A | CMV-(G₃AS)-(G₄S)-FC1-(G₄S)-β2M (R12C, E44C) |
| 4008 | 839 | A | CMV-GGGASGGGGSGGGGS-β2M (R12C) |
| | 2686 | B | β2M (R12C, E44C) |

Unconjugated T-Cell-MP (Single Chain Construct)

| | | |
|---|---|---|
| 3984 | C | 1xhIL2(F42A, H16A)-(G₄S)₄-β2M (E44C)-(G₄S)₃-HLA-A02(Y84C, |
| | | A139C)-AAAGG-IgG1(L234A, L235A) |
| 3985 | C | hIL2 (F42A, H16A)-(G₄S)₄-hIL2 (F42A, H16A)-(G₄S)-GLGGS-(G₄S)₂- |
| | | β2M (E44C)-(G₄S)₃-HLA-A02(Y84C, A139C)-AAAGG-IgG1(L234A, |
| | | L235A) |
| 3986 | C | hIL2 (F42A, H16A)-(G₄S)₄-hIL2 (F42A, H16A)-(G₄S)₂-GLGGS-(G₄S)- |
| | | β2M (E44C)-(G₄S)₃-HLA-A02(Y84C,A139C)-AAAGG-IgG1(L234A, |
| | | L235A) |
| 3987 | C | hIL2 (F42A, H16A)-(G₄S)₄-hIL2 (F42A, H16A)-(G₄S)₃-GLGGS-β2M |
| | | (E44C)-(G₄S)₃-HLA-A02(Y84C, A139C)-AAAGG-IgG1(L234A, L235A) |
| 3988 | C | hIL2 (F42A, H16A)-(G₄S)₄-hIL2 (F42A, H16A)-(G₄S)-GMGGSGGGGS- |
| | | (G₄S)-β2M (E44C)-(G₄S)₃-HLA-A02(Y84C, A139C)-AAAGG- |
| | | IgG1(L234A, L235A) |
| 3989 | C | hIL2 (F42A, H16A)-(G₄S)₄-hIL2 (F42A, H16A)-(G₄S)-GGGMSGGGGS- |
| | | (G₄S)-β2M (E44C)-(G₄S)₃-HLA-A02(Y84C, A139C)-AAAGG- |
| | | IgG1(L234A, L235A) |
| 3990 | C | hIL2 (F42A, H16A)-(G₄S)₄-hIL2 (F42A, H16A)-(G4S)-GGGGSMGGGS- |
| | | (G4S)-β2M (E44C)-(G4S)3-HLA-A02(Y84C, A139C)-AAAGG- |
| | | IgG1(L234A, L235A) |
| 3991 | C | hIL2 (F42A, H16A)-(G₄S)₄-hIL2 (F42A, H16A)-(G₄S)-GGGGSGMGGS- |
| | | (G₄S)-β2M (E44C)-(G₄S)₃-HLA-A02(Y84C, A139C)-AAAGG- |
| | | IgG1(L234A, L235A) |
| 3992 | C | hIL2 (F42A, H16A)-(G₄S)₄-hIL2 (F42A, H16A)-(G₄S)-GGGGSGGGGM- |
| | | (G₄S)-β2M (E44C)-(G₄S)₃-HLA-A02(Y84C, A139C)-AAAGG- |
| | | IgG1(L234A, L235A) |
| 3861 | C | hIL2 (F42A, H16A)-(G₄S)₄-hIL2 (F42A, H16A)-(G₄S)₄-β2M (E44C)- |
| | | (G₄S)₃-HLA-A02(Y84C, A139C)-AAAGG-IgG1(L234A, L235A) |
| 4004 | C | hIL2 (F42A, H16A)-(G₄S)₄-hIL2 (F42A, H16A)-(G₄S)₅-β2M (E44C)- |
| | | (G₄S)₃-HLA-A02(Y84C, A139C)-AAAGG-IgG1(L234A, L235A) |
| 4005 | C | hIL2 (F42A, H16A)-(G₄S)₄-hIL2 (F42A, H16A)-(G₄S)₆-β2M (E44C)- |
| | | (G₄S)₃-HLA-A02(Y84C, A139C)-AAAGG-IgG1(L234A, L235A) |
| 4006 | C | hIL2 (F42A, H16A)-(G₄S)₄-hIL2 (F42A, H16A)-(G₄S)₄-β2M (E44C)- |
| | | (G₄S)₂-HLA-A02(Y84C, A139C)-AAAGG-IgG1(L234A, L235A) |
| 4007 | C | hIL2 (F42A, H16A)-(G₄S)₄-hIL2 (F42A, H16A)-(G₄S)₄-β2M (E44C)- |
| | | (G₄S)-HLA-A02(Y84C, A139C)-AAAGG-IgG1(L234A, L235A) |
| 3999 | C | hIL2 (F42A, H16A)-(G₄S)₄-hIL2 (F42A, H16A)-(G₄S)₄-β2M (E44C)- |
| | | (G₄S)₃-HLA-A02(Y84C, A139C, T143L)-AAAGG-IgG1(L234A, L235A) |
| 4000 | C | hIL2 (F42A, H16A)-(G₄S)₄-hIL2 (F42A, H16A)-(G₄S)₄-β2M (E44C)- |
| | | (G₄S)₃-HLA-A02(Y84C, A139C, T143M)-AAAGG-IgG1(L234A, |
| | | L235A) |
| 4001 | C | hIL2 (F42A, H16A)-(G₄S)₄-hIL2 (F42A, H16A)-(G₄S)₄-β2M (E44C)- |
| | | (G₄S)₃-HLA-A02(L81M, Y84C, A139C)-AAAGG-IgG1(L234A,L235A) |

The nucleic acids encoding the protein constructs were transfected into and expressed by CHO cells as soluble protein in the culture media. The level of protein expressed in the culture media after 7 days was determined by BLI assay using protein A to capture the expressed protein (FIG. 12 at D). The fraction of protein appearing in unaggregated duplex form is assessed by isolating the protein from the culture media using magnetic protein A beads. After washing, the bound protein is eluted from the beads by reducing the pH, and then subject to analytical size exclusion chromatography using UV detection on an AGILENT® chromatography system. The fraction of unaggregated protein reported in FIG. 12 at E is based on the area of the peak corresponding to the molecular weight of the duplex relative to the total area of the chromatographed protein.

The results indicate that unconjugated single chain T-Cell-MP constructs appear to be expressed more uniformly at higher levels than their unconjugated split chain construct counterparts.

### Example 2

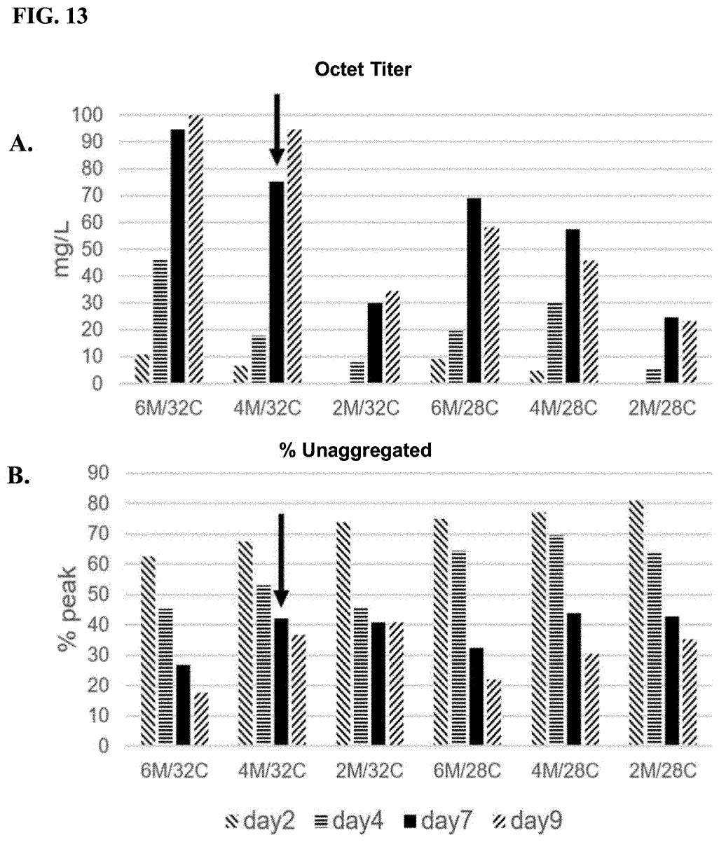

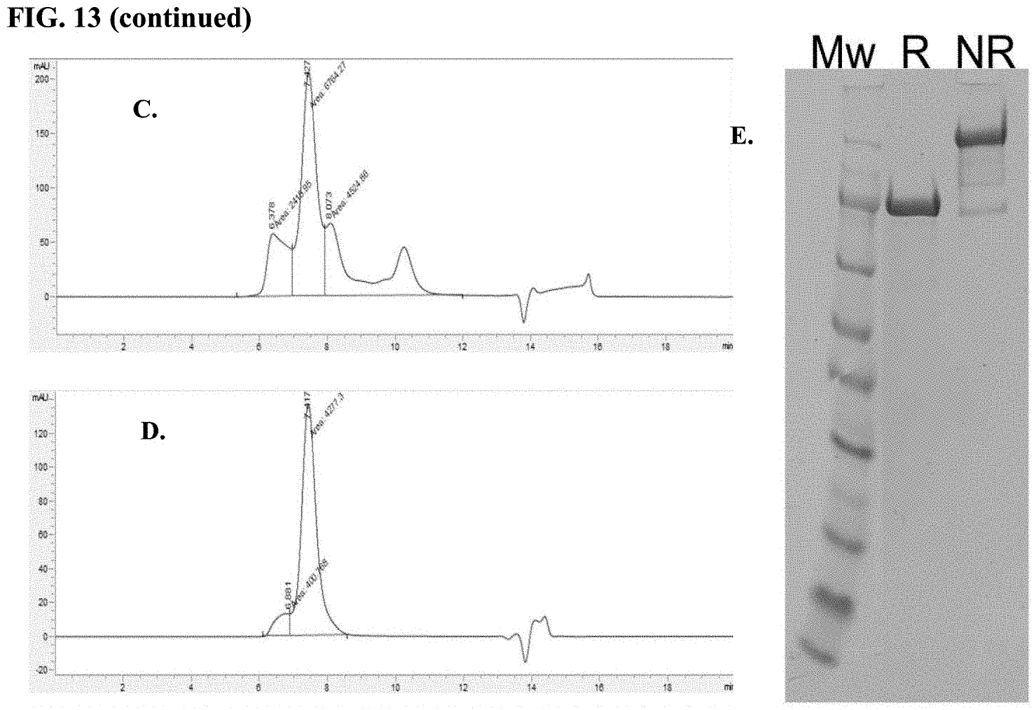

The effect of time in culture, cell culture density, and culture temperature on unconjugated T-Cell-MPs was examined by transiently expressing the construct 3861 (see Example 1) in CHO cells at 28 and 32° C. Transfection was accomplished with expiCHO® transfection kits (Gibco™/ThermoFisher Scientific, Skokie, Ill.) using a recombinant pTT5 vector into which the cassette encoding the polypeptide was cloned. The transfected cells were diluted to 2, 4 or 6 million cells per milliliter and T-Cell-MP 3861 expression levels and the fraction of unaggregated protein in duplex form determined at days 2, 4, 7, and/or 9 as indicated by removing a portion of the culture. Analyses were conducted as in Example 1 and are shown in FIG. 13 (at A and B) with the number of cells and culture temperature shown below each histogram set (e.g., six million cells at 32° C. denoted as 6M/32C). Also shown in FIG. 13 are size exclusion chromatograms (C and D) of the unconjugated 3861 T-Cell-MP harvested from a culture using protein A and after further purification by size exclusion chromatography (upper and lower chromatograms respectively). Coomassie blue stained SDS PAGE analysis (at E) confirms the purity and homogeneity of the purified material, samples of which were applied to the gel in reduced (R) and non-reduced form (NR).

### Example 3

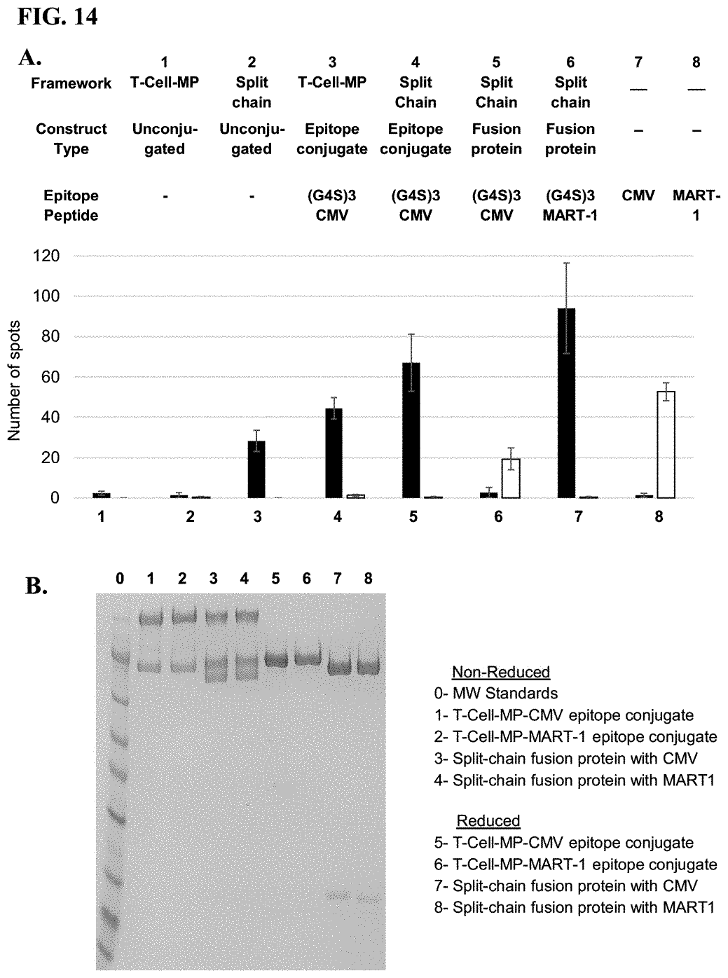

The specific interaction of T-Cell-MP epitope conjugates and control constructs with epitope specific T cells was assessed by incubating the molecules with T cells responsive to either the CMV peptide NLVPMVATV (black bars) or the Melan-A and Mucin Related Peptide (MART-1) ELAGIGILTV (white bars) in the histogram of Elispot data provided FIG. 14A. Control samples of the unconjugated 3861 T-Cell-MP duplex (see FIG. 12 at C for the general structure) group 1, and an unconjugated split chain construct comprising polypeptides 1694 and 2686 (duplexed as in FIG. 12 at B) group 2 were run in parallel with test samples. T-Cell-MP and split chain constructs conjugated to the E44C position of β2M through a (G₄S)₃ linker by a maleimide group are shown in groups 3 and 4. The effect of control construct split chain fusion proteins (FIG. 12 structure A) having a CMV or MART-1 polypeptide as part of the fusion protein are shown in groups 5 and 6 respectively. Control stimulation by CMV and MART-1 peptides is shown in groups 7 and 8 respectively. The histogram indicates the number of spots due to captured interferon gamma indicating activation of the T cells by the treatments.

The SDS-PAGE gel shown in FIG. 14 B provides an analysis of reducing and non-reducing samples of the epitope conjugates and fusion proteins, indicating their purity and homogeneity.

### Example 4

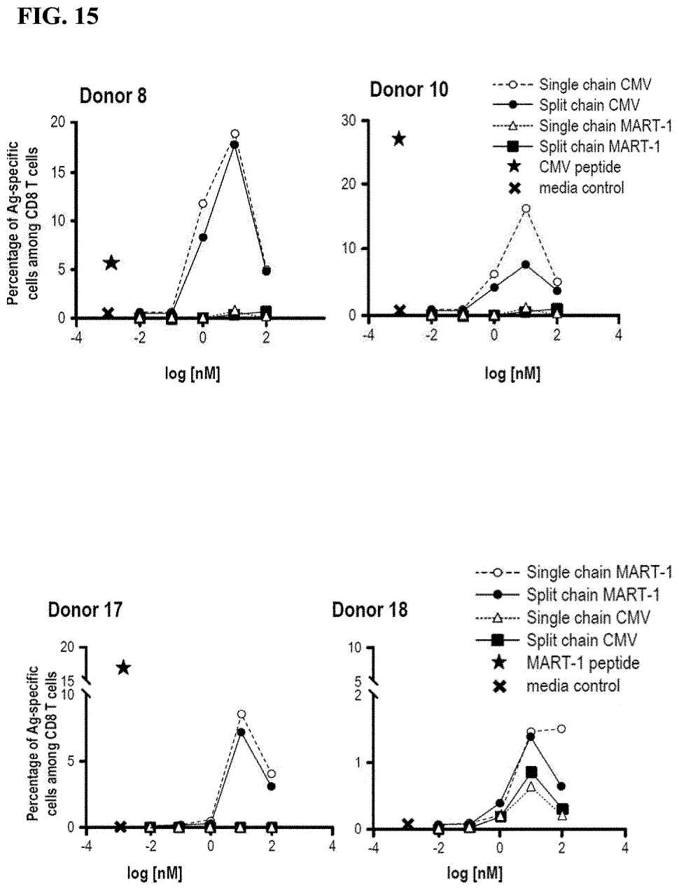

Ficoll-Paque® purified samples of leukocytes from CMV responsive donors (Donors 8, 10, 38, and 39) and MART-1 responsive donors (Donors 17 and 18) were prepared and used to demonstrate the ability of T-Cell-MP-epitope conjugates to expand T cells specific to CMV or MART-1 specific epitopes. MART-1 responsive Donor 18 also displays some responsiveness to the CMV peptide. Positive and negative control treatments included: treatment with split chain constructs conjugated to CMV and MART-1 peptides; treatment with the CMV or MART-1 peptides in culture media; and media only control treatment. For the experiments, leukocytes were suspended at 2.5×10⁶ cells per ml in ImmunoCult™ media (Stemcell Technologies, Vancouver, British Columbia) containing the indicated amounts of the control or T-Cell-MP-epitope conjugate or control treatments. After 10 days in culture the number of cells responsive to CMV or MART-1 were assessed by Flow cytometry using CMV or MART-1 tetramers purchased from MBL International Corp. The results indicate that both the T-Cell-MP and split chain constructs conjugated to the CMV peptide, and to a lesser degree CMV peptide, stimulate expansion of CMV specific T cells from CMV responsive donors in a concentration dependent manner T-Cell-MP and split chain constructs conjugated to the MART-1 peptide, and to a lesser degree the MART-1 peptide stimulate expansion of MART-1 specific T cells from MART-1 responsive donors in a concentration dependent manner. In each instance, CMV peptide conjugates selectively stimulated T cells from CMV responsive donors but not MART-1 responsive donors and vice versa. Free peptide in the absence of IL-2 failed to produce an effect the was equal to the effect observed with the T-Cell-MP epitope conjugates. Results are provided in FIG. 15.

The T-Cell-MP-epitope conjugate employed for the assays was a duplex of the 3186 polypeptide (see Example 1 and FIG. 12 structure C for the general form of the unconjugated duplex) conjugated at a cysteine (E44C) in the β2M polypeptide sequence to a either a CMV (NLVPMVATV) or MART-1 (ELAGIGILTV) (SEQ ID NO:533) peptide via a (G₄S)₃ linker bearing a maleimide group (e.g., for the CMV peptide NLVPMVATV-(G₄S)₃-lysine-epsilon amino-maleimide). The split chain epitope conjugate was a duplex of two split chain constructs each comprising a1694 and 2686 polypeptide (see Example 1 and FIG. 12 structure B for the general form of the unconjugated duplex), which was conjugated at a cysteine (E44C) in the β2M polypeptide sequence to a either a CMV (NLVPMVATV) or MART-1 (ELAGIGILTV) peptide via a (G₄S)₃ linker bearing a maleimide group. After reduction to remove any capping from the cysteine conjugation sites, the conjugation was conducted as described in for maleimide coupling reactions using at least two additions of the peptide bearing a maleimide group.

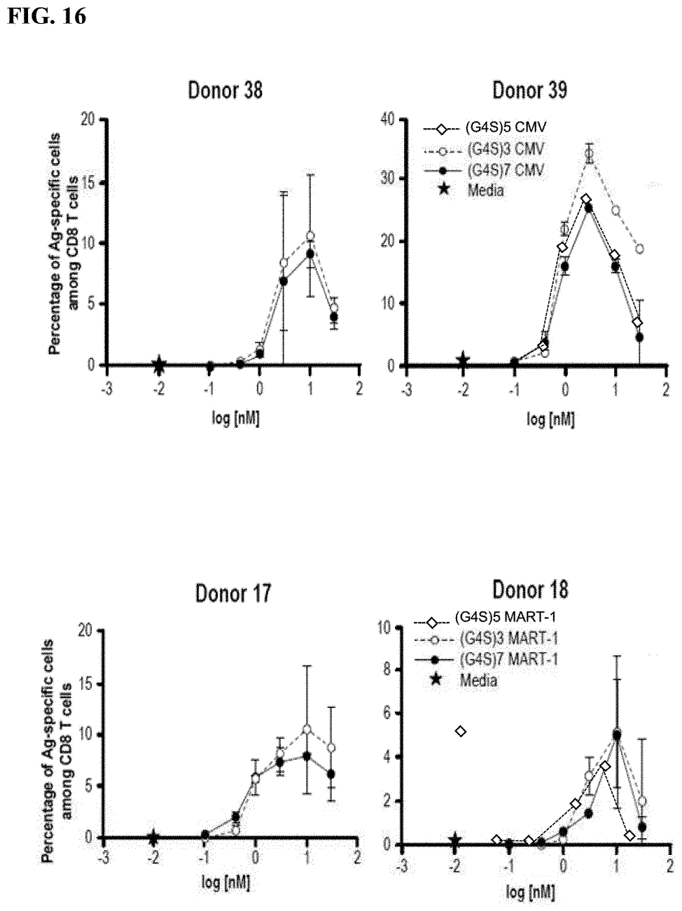

In an additional test, the effect of a construct bearing a (G₄S)₇ L3 linker (the linker between the β2M and HLA-A02 sequences), but otherwise identical to 3861, was compared with the 3861 polypeptide duplex (i.e., construct 4125 2×IL2(F42A, H16A)-(G₄S)₇-β2M (E44C)-(G₄S)₃-HLA-A02(Y84C, A139C)-AAAGG-IgG1(L234A, L235A)). Duplexes of both the 3861 and 4125 constructs were conjugated to a CMV or MART-1 peptide by a maleimide terminated (G₄S)₃ linker and tested side-by-side for the ability to expand T cells in an epitope specific manner. The assays were conducted as described above for the 3861 epitope conjugates, except only a media alone control was conducted. The results, shown in FIG. 16, indicate that extending the linker length did not substantively alter the expansion of T cells seen with the 3861 epitope conjugates.

### Example 5

In order to examine the effect of L3 linker length on the level of cell expression and the quality (fraction unaggregated) of T-Cell-MP proteins a series of nucleic acids encoding constructs 4125 through 4128 that are related to construct 3861 but with L3 linkers of increasing length were prepared and inserted into an expression vector (pTT5). A second set of constructs (4129-4133) bearing an additional R12C substitution in the β2M polypeptide (R12C, E44C) and an A236C substitution in the HLA-A02 peptide that can form an interchain disulfide bond was also prepared. The vectors were transfected into CHO cells with expiCHO® transfection kits and both the amount of protein expressed in the culture media and the fraction of unaggregated protein after purification using magnetic beads was assessed at days 4, 6, 8, and/or 11 as indicated. The specific constructs included those recited in the following table.

| Con- | Form in | L3 linker | |
|---|---|---|---|
| struct | FIG. 12 | (G₄S)n | Construct Content and Organization |
| 3861 | C | n = 3 | hIL2 (F42A, H16A)-(G₄S)₄-hIL2 (F42A, H16A)-(G₄S)₄-β2M |
| | | | (E44C)-(G₄S)₃-HLA-A02(Y84C, A139C)-AAAGG-IgG1(L234A, |
| | | | L235A) |
| 4128 | C | n = 4 | hIL2 (F42A, H16A)-(G₄S)₄-hIL2 (F42A, H16A)-(G₄S)₄-β2M |
| | | | (E44C)-(G₄S)₄-HLA-A02(Y84C,A139C)-AAAGG-IgG1(L234A, |
| | | | L235A) |
| 4127 | C | n = 5 | hIL2 (F42A, H16A)-(G₄S)₄-hIL2 (F42A, H16A)-(G₄S)₄-β2M |
| | | | (E44C)-(G₄S)₅-HLA-A02(Y84C, A139C)-AAAGG-IgG1(L234A, |
| | | | L235A) |
| 4126 | C | n = 6 | hIL2 (F42A, H16A)-(G₄S)₄-hIL2 (F42A, H16A)-(G₄S)₄-β2M |
| | | | (E44C)-(G₄S)₆-HLA-A02(Y84C, A139C)-AAAGG-IgG1(L234A, |
| | | | L235A) |
| 4125 | C | n = 7 | hIL2 (F42A, H16A)-(G₄S)₄-hIL2 (F42A, H16A)-(G₄S)₄-β2M |
| | | | (E44C)-(G₄S)₇-HLA-A02(Y84C, A139C)-AAAGG-IgG1(L234A, |
| | | | L235A) |
| 4129 | C | n = 7 | hIL2 (F42A, H16A)-(G₄S)₄-hIL2 (F42A, H16A)-(G₄S)₄-β2M (R12C, |
| | | | E44C)-(G₄S)₇-HLA-A02(Y84C, A139C, A236C)-AAAGG- |
| | | | IgG1(L234A, L235A) |
| 4130 | C | n = 6 | hIL2 (F42A, H16A)-(G₄S)₄-hIL2 (F42A, H16A)-(G₄S)₄-β2M (R12C, |
| | | | E44C)-(G₄S)₆-HLA-A02(Y84C, A139C, A236C)-AAAGG- |
| | | | IgG1(L234A, L235A) |
| 4131 | C | n = 5 | hIL2 (F42A, H16A)-(G₄S)₄-hIL2 (F42A, H16A)-(G₄S)₄-β2M (R12C, |
| | | | E44C)-(G₄S)₅-HLA-A02(Y84C, A139C, A236C)-AAAGG- |
| | | | IgG1(L234A, L235A) |
| 4132 | C | n = 4 | hIL2 (F42A, H16A)-(G₄S)₄-hIL2 (F42A, H16A)-(G₄S)₄-β2M (R12C, |
| | | | E44C)-(G₄S)₄-HLA-A02(Y84C, A139C, A236C)-AAAGG- |
| | | | IgG1(L234A, L235A) |
| 4133 | C | n = 3 | hIL2 (F42A, H16A)-(G₄S)₄-hIL2 (F42A, H16A)-(G₄S)₄-β2M (R12C, |
| | | | E44C)-(G₄S)₃-HLA-A02(Y84C, A139C, A236C)-AAAGG- |
| | | | IgG1(L234A, L235A) |

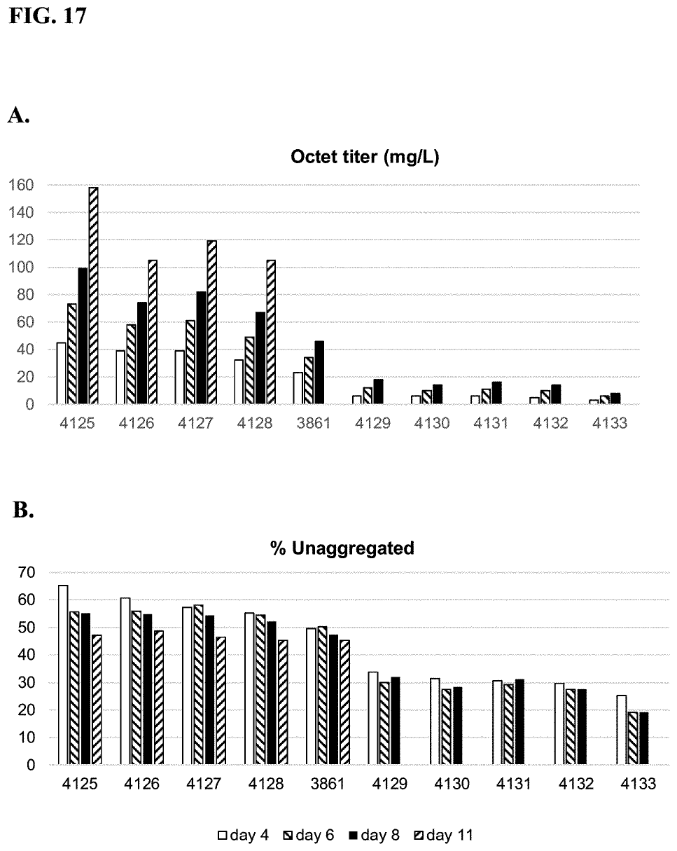

The amount of the expressed unconjugated T-Cell-MP constructs were determined by BLI assay using protein A for capture on a BioForte instrument using the methods described in Example 1. Results are provided in FIG. 17 histogram A.

The fraction of unconjugated T-Cell-MP that is unaggregated (present in duplex form) after purification on magnetic protein A beads was determined by size exclusion chromatography. The fraction was determined using the area of the chromatographic peak corresponding to the molecular weight of the duplex relative to the area under the chromatogram as described in Example 1. Results are shown in FIG. 17 histogram B.

Additional optimization indicates that higher yields are possible. Construct 4125 has been observed to reach 200 mg/ml and construct 4127 has been observed to reach 170 mg/ml in CHO culture cell media prior to isolation.

1. An unconjugated T cell modulatory polypeptide (T-Cell-MP), the polypeptide comprising:
(i) optionally one or more MOD polypeptide sequences, or two or more MOD polypeptide sequences in tandem, wherein when there are two or more MOD polypeptide sequences they are optionally joined to each other by independently selected L1 linkers;
(ii) an optional L2 linker polypeptide sequence joining the one or more MOD polypeptide sequences to a β2M polypeptide sequence;
(iii) the β2M polypeptide sequence;
(iv) an L3 linker polypeptide sequence from 10-50 aa in length;
(v) a class I MHC-H polypeptide sequence, wherein the MHC-H polypeptide comprises cysteine substitutions at positions 84 and 139 that form an intrachain disulfide bond;
(vi) an optional L4 linker polypeptide sequence;
(vii) a scaffold polypeptide sequence;
(viii) an optional L5 linker polypeptide sequence; and
(ix) optionally one or more MOD polypeptide sequences, or two or more MOD polypeptide sequences in tandem, wherein when there are two or more MOD polypeptide sequences they are optionally joined to each other by independently selected L6 linkers;
wherein the unconjugated T-Cell-MP comprises at least one MOD polypeptide sequence as part of element (i) or (ix)); and
wherein at least one of the β2M polypeptide sequence and/or the L3 linker polypeptide sequence comprises a chemical conjugation site for epitope conjugation.

2. The unconjugated T-Cell-MP of claim 1, the polypeptide comprising from N-terminus to C-terminus:
(i) optionally one or more MOD polypeptide sequences, or two or more MOD polypeptide sequences in tandem, wherein when there are two or more MOD polypeptide sequences they are optionally joined to each other by independently selected L1 linkers;
(ii) an optional L2 linker polypeptide sequence;
(iii) a β2M polypeptide sequence;
(iv) an L3 linker polypeptide sequence from 10-50 aa in length;
(v) a class I MHC-H polypeptide sequence, wherein the MHC-H polypeptide comprises cysteine substitutions at positions 84 and 139 that form an intrachain disulfide bond;
(vi) an optional L4 linker polypeptide sequence;
(vii) a scaffold polypeptide sequence;
(viii) an optional L5 linker polypeptide sequence; and
(ix) optionally one or more MOD polypeptide sequences, or two or more MOD polypeptide sequences, such as in tandem, wherein when there are two or more MOD polypeptide sequences they are optionally joined to each other by independently selected L6 linkers;
wherein the unconjugated T-Cell-MP comprises at least one MOD polypeptide sequence as part of element (i) or (ix); and
wherein at least one of the β2M polypeptide sequence and/or the L3 linker polypeptide sequence comprises a chemical conjugation site for epitope conjugation.

3. (canceled)

4. (canceled)

5. (canceled)

6. The unconjugated T-Cell-MP of claim 2, wherein the β2M polypeptide sequence has at least 90% or at least 95% sequence identity to at least 70 or at least 90 contiguous aas the mature human β2M polypeptide NP_004039.1, SEQ ID NO:61, provided in FIG. 4.

7. The unconjugated T-Cell-MP of claim 6, wherein the MHC-H polypeptide sequence comprises a human class I MHC-H chain polypeptide sequence selected from HLA-A, HLA-B, HLA-C, HLA-E, HLA-F, and HLA-G MHC-H polypeptide sequences having at least 85% sequence identity to at least 200 contiguous aas of a MHC-H polypeptide provided in any of FIGS. 3A-3H.

8. The unconjugated T-Cell-MP of claim 7, wherein the MHC-H polypeptide sequence comprises a disulfide bond between position 84 and position 139.

9. The unconjugated T-Cell-MP of claim 8, wherein the MHC-H polypeptide sequence comprises an amino acid sequence having at least 90% or at least 95% sequence identity to at least 200 contiguous aas of: HLA-A*0101 (SEQ ID NO:24), HLA-A*0201 (SEQ ID NO:27), HLA-A*0301 (SEQ ID NO:35), HLA-A*1101 (SEQ ID NO:32), HLA-A*2301 (SEQ ID NO:36). HLA-A*2402 (SEQ ID NO:33), HLA-A*2407 (SEQ ID NO:37), HLA-A*3303 (SEQ ID NO:34), HLA-A*3401 (SEQ ID NO:38), HLA-E (SEQ ID NO: 58), HLA-F (SEQ ID NO: 59), or HLA-G (SEQ ID NO:60).

10. The unconjugated T-Cell-MP of claim 9, comprising at least one, at least two, or at least three wt. MOD or variant MOD polypeptide sequences, wherein each MOD is selected independently from the group consisting of anti-CD28, 4-1BBL, PD-L1, IL-2, CD80, CD86, and FasL wt. MOD or variant MOD polypeptide sequences.

11. The unconjugated T-Cell-MP of claim 10, comprising at least one wt. or variant IL-2 MOD polypeptide sequence, or at least one pair of wt. or variant IL-2 MOD polypeptide sequences in tandem.

12. The unconjugated T-Cell-MP of claim 10, wherein the unconjugated T-Cell-MP comprises a single variant IL-2 MOD or a pair of variant IL-2 MOD polypeptide sequences in tandem, and wherein the variant IL-2 MOD polypeptide sequences comprise substitutions at positions F424 and H16.

13. The unconjugated T-Cell-MP of claim 10, wherein the scaffold polypeptide sequence is an interspecific or non-interspecific polypeptide sequence selected from the group consisting of: an immunoglobulin heavy chain constant region; a collectin polypeptide, a coiled-coil domain, a leucine-zipper domain; a Fos polypeptide; a Jun polypeptide; an Ig CH1 polypeptide; an Ig CL κ polypeptide; an Ig CL λ polypeptide; a knob-in-hole without disulfide (KiH) polypeptide; a knob-in hole with a stabilizing disulfide bond (KiHs-s) polypeptide; a HA-TF polypeptide; a ZW-1 polypeptide; a 7.8.60 polypeptide; a DD-KK polypeptide; an EW-RVT polypeptide; an EW-RVTs-s polypeptide; and an A107 polypeptide sequence.

14. The unconjugated T-Cell-MP of claim 13 complexed to form a duplex or higher order T-Cell-MP comprising:
at least a first unconjugated T-Cell-MP and a second unconjugated T-Cell-MP of claim 13, wherein
(i) the first unconjugated T-Cell-MP comprises a first β2M polypeptide sequence, a first class I MHC-H polypeptide sequence, and a first scaffold polypeptide sequence; and
(ii) the second unconjugated T-Cell-MP comprises a first β2M polypeptide sequence, a second class I MHC-H polypeptide sequence, and a second scaffold polypeptide sequence; and
wherein the first and second unconjugated T-Cell-MPs associate by binding interactions between the first and second scaffold polypeptide sequences that optionally include one or more interchain covalent bonds therebetween; and
wherein the duplex or higher order T-Cell-MP is homomeric or heteromeric.

15. The unconjugated T-Cell-MP or unconjugated duplex T-Cell-MP of claim 14, further comprising one or more substitutions that reduce binding to the Fc X receptor and/or the C1 q protein.

16. The unconjugated T-Cell-MP or unconjugated duplex T-Cell-MP of claim 14, wherein each chemical conjugation site is jointly or independently selected from: a) amino acid chemical conjugation sites; b) non-natural amino acids and/or selenocysteines; c) peptide sequences that act as an enzymatic modification sequence; d) carbohydrate or oligosaccharide moieties; and/or e) IgG nucleotide binding sites.

17. The unconjugated T-Cell-MP or unconjugated duplex T-Cell-MP of claim 16, wherein each chemical conjugation site for epitope conjugation is a cysteine introduced by protein engineering.

18. (canceled)

19. The unconjugated T-Cell-MP or unconjugated duplex T-Cell-MP of claim 17, wherein the cysteine introduced by protein engineering within the β2M polypeptide sequence is at position 44 of the β2M polypeptide.

20. The unconjugated T-Cell-MP or unconjugated duplex T-Cell-MP of claim 19, further comprising one or more targeting sequences.

21. The unconjugated duplex T-Cell-MP of claim 14 conjugated to a non-peptide or peptide epitope at the chemical conjugation site in the β2M polypeptide sequence to form a duplex T-Cell-MP-epitope conjugate; wherein the duplex T-Cell-MP-epitope conjugate optionally comprises a targeting sequence.

22. (canceled)

23. The duplex T-Cell-MP-epitope conjugate of claim 21, wherein the epitope is a cancer epitope (epitope presented by a cancer cell), infectious agent epitope (epitope of an infectious agent's antigen), self-epitope (epitope of an autoantigen), or allergen epitope (epitope of an allergen).

24. A method of treating disease comprising administering to a subject in need thereof an effective amount of a T-Cell-MP-epitope conjugate of claim 21.

25. The method of claim 24, wherein the epitope is a cancer epitope and the subject is suffering from a cancer.
